(19) United States
(12) Patent Application Publication
(10) Pub. No.: US 2026/0345568 A1
(43) Pub. Date: Nov. 5, 2026

(54) INHIBITING MONOACYLGLYCEROL LIPASE (MAGL)

(71) Applicant: PSY THERAPEUTICS, INC., Boston, MA (US)

(72) Inventors: Jacob Matthew Hooker, Winchester, MA (US); Alan John Cross, Thornton, PA (US)

(73) Assignee: PSY THERAPEUTICS, INC., Boston, MA (US)

(21) Appl. No.: 19/473,925

(22) PCT Filed: Apr. 11, 2024
(86) PCT No.: PCT/US2024/024039

§ 371 Date: Oct. 9, 2025

Related U.S. Application Data
(60) Provisional application No. 63/459,168, filed on Apr. 13, 2023.
(60) Provisional application No. 63/526,413, filed on Jul. 12, 2023.

(51) Int. Cl. A61K 31/4439; C07D 401/14; C07D 403/04

(52) U.S. Cl. CPC A61K 31/4439; C07D 401/14; C07D 403/04

(57) ABSTRACT

Provided herein are compounds for inhibiting monoacylglycerol lipase (MAGL), and related methods of making and using the compounds.

### CROSS-REFERENCE TO RELATED APPLICATIONS

This patent application claims the benefit under 35 U.S.C. § 119(e) of U.S. Provisional Patent Application No. 63/459,168 filed Apr. 13, 2023, and U.S. Provisional Patent Application No. 63/526,413 filed Jul. 12, 2023, each of which is incorporated herein by reference in its entirety.

### TECHNICAL FIELD

The present disclosure relates to compounds and methods for inhibiting monoacylglycerol lipase (MAGL), including compounds and methods for reversible inhibition of MAGL.

### BACKGROUND

MAGL is the principal enzyme responsible for the in vivo degradation of 2-arachidonoyl glycerol (2-AG), an endogenous ligand of the cannabinoid receptors (e.g., CB1 and CB2). MAGL inhibition increases accumulation of the CB1/2 receptor agonist 2-arachidonoyl glycerol (2-AG), and reduces arachidonic acid (AA) and prostaglandin levels in the brain and peripheral tissues. Irreversible MAGL inhibitor compounds, such as JZL-184, increase brain and peripheral 2-AG and reduce brain AA, however tolerance can develop with chronic irreversible MAGL inhibition. Covalent interactions with MAGL could lead to irreversible enzymatic inhibition, with potential for immune-mediated toxicity.

There remains a need for reversible MAGL inhibitors useful for the treatment of MAGL-mediated diseases or disorders, including the development of therapeutic compounds with improved control of dose and exposure. Such compounds can be developed through clinical trials as analgesic compounds for the treatment of pain management and/or treatment of various MAGL-mediated conditions.

Thus, there is a need in the art for compounds that can potently, selectively and reversibly inhibit MAGL and for means for treating conditions or disorders that are associated with or linked to endocannabinoid signaling activities. The present disclosure addresses these and other unfulfilled needs in the art.

### SUMMARY

In some embodiments, the disclosure provides a compound of Formula (I) or a pharmaceutically acceptable salt thereof:

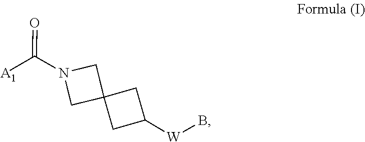

wherein:
   A₁ is

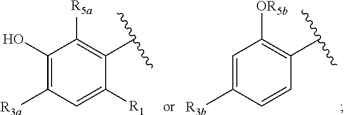

R₁ is hydrogen, halogen, or cyano;
R₃ₐ and R₅ₐ are each independently hydrogen, halogen, or optionally substituted lower alkyl;
R_{3b} is halogen;
R_{5b} is optionally substituted lower alkyl, optionally substituted lower cycloalkyl, or optionally substituted lower haloalkyl;
W is a pyrazolone, pyrrole or diazole, each independently optionally substituted with halogen, optionally substituted lower alkyl, optionally substituted lower cycloalkyl, optionally substituted lower alkoxy, or optionally substituted lower haloalkyl; and
B is an optionally substituted pyridone, or a 6-member heteroaryl comprising at least one nitrogen atom and optionally substituted with one or more halogen, optionally substituted lower alkyl, optionally substituted lower haloalkyl, or optionally substituted lower alkoxy.

In some embodiments, the disclosure provides a compound of Formula (I) or a pharmaceutically acceptable salt thereof, wherein W is selected from the group consisting of:

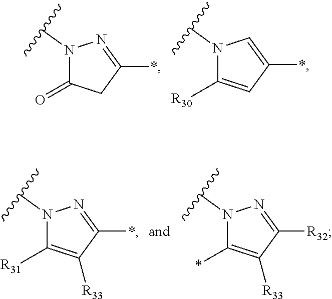

* is the bond to B;
B is an optionally substituted pyridone, or a pyridine optionally substituted with halogen, lower alkoxy, lower alkyl, or lower haloalkyl;
R₃₀ is hydrogen, halogen, lower alkyl, lower alkoxy, lower haloalkyl, or lower cycloalkyl; and
R₃₁, R₃₂ and R₃₃ are each independently hydrogen, halogen, lower alkyl, lower alkoxy, lower haloalkyl, or lower cycloalkyl, provided that at least one of R₃₁ and R₃₃ is not hydrogen, and provided that at least one of R₃₂ and R₃₃ is not hydrogen.

In some embodiments, the disclosure provides a compound of Formula (I) or a pharmaceutically acceptable salt thereof, wherein W is selected from the group consisting of:

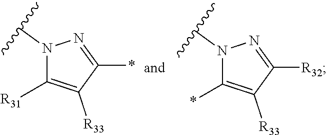

*is the bond to B; and
R₃₁, R₃₂ and R₃₃ are each independently hydrogen, halogen, lower alkyl, lower alkoxy, lower haloalkyl, or lower cycloalkyl, provided that at least one of R₃₁ and R₃₃ is not hydrogen, and provided that at least one of R₃₂ and R₃₃ is not hydrogen.

In some embodiments, the disclosure provides a compound of Formula (I) or a pharmaceutically acceptable salt thereof, wherein W is a diazole optionally substituted with one of methyl, cyclopropyl, or CF₃; and B is an optionally substituted pyridone, or a pyridine optionally substituted with fluoro, chloro, methoxy, methyl or CF₃. In some embodiments, the disclosure provides a compound of Formula (I) or a pharmaceutically acceptable salt thereof, wherein R₁ is hydrogen, fluoro, or cyano; R₃ₐ and R₅ₐ are each independently hydrogen, fluoro, or methyl; R_{3b} is fluoro; and R_{5b} is cyclopropyl, or lower alkyl optionally substituted with one or more fluoro.

In some embodiments, the disclosure provides a compound of Formula (I) or a pharmaceutically acceptable salt thereof, wherein W is

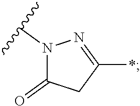

and * is the bond to B. In some embodiments, the disclosure provides a compound of Formula (I) or a pharmaceutically acceptable salt thereof, wherein W is

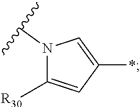

* is the bond to B; and R₃₀ is hydrogen, halogen, lower alkyl, lower alkoxy, lower haloalkyl, or lower cycloalkyl. In some embodiments, the disclosure provides a compound of Formula (I) or a pharmaceutically acceptable salt thereof, wherein R₃₀ is methoxy.

In some embodiments, the disclosure provides a compound of Formula (I) or a pharmaceutically acceptable salt thereof, wherein W is

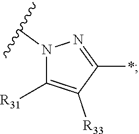

* is the bond to B; and R₃₁ and R₃₃ are each independently hydrogen, halogen, lower alkyl, lower alkoxy, lower haloalkyl, or lower cycloalkyl, provided that at least one of R₃₁ and R₃₃ is not hydrogen. In some embodiments, the disclosure provides a compound of Formula (I) or a pharmaceutically acceptable salt thereof, wherein R₃₁ and R₃₃ are each independently hydrogen, methyl, CF₃, or cyclopropyl, provided that at least one of R₃₁ and R₃₃ is not hydrogen.

In some embodiments, the disclosure provides a compound of Formula (I) or a pharmaceutically acceptable salt thereof, wherein W is

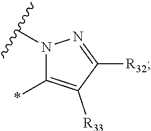

* is the bond to B; and R₃₂ and R₃₃ are each independently hydrogen, halogen, lower alkyl, lower alkoxy, lower haloalkyl, or lower cycloalkyl, provided that at least one of R₃₂ and R₃₃ is not hydrogen. In some embodiments, the disclosure provides a compound of Formula (I) or a pharmaceutically acceptable salt thereof, wherein R₃₂ and R₃₃ are each independently hydrogen, methyl, CF₃, or cyclopropyl, provided that at least one of R₃₂ and R₃₃ is not hydrogen.

In some embodiments, the disclosure provides a compound of Formula (I) or a pharmaceutically acceptable salt thereof, wherein B is optionally substituted 2-pyridone, or pyridine optionally substituted with fluoro, chloro, methyl, methoxy or CF₃. In some embodiments, the disclosure provides a compound of Formula (I) or a pharmaceutically acceptable salt thereof, wherein B is optionally substituted 2-pyridone. In some embodiments, the disclosure provides a compound of Formula (I) or a pharmaceutically acceptable salt thereof, wherein B is

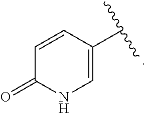 [SMILES: *c1ccc(=O)[nH]c1]

In some embodiments, the disclosure provides a compound of Formula (I) or a pharmaceutically acceptable salt thereof, wherein B is

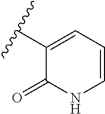 [SMILES: *c1ccc[nH]c1=O]

In some embodiments, the disclosure provides a compound of Formula (I) or a pharmaceutically acceptable salt thereof, wherein B is

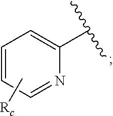

and R_{c} is hydrogen, fluoro, methoxy, or CF₃. In some embodiments, the disclosure provides a compound of Formula (I) or a pharmaceutically acceptable salt thereof, wherein B is

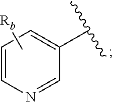

and R_{b} is hydrogen, fluoro, methoxy, or CF₃. In some embodiments, the disclosure provides a compound of Formula (I) or a pharmaceutically acceptable salt thereof, wherein B is

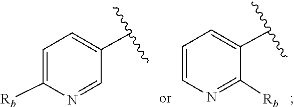

and R_{b} is hydrogen, fluoro or methoxy. In some embodiments, the disclosure provides a compound of Formula (I) or a pharmaceutically acceptable salt thereof, wherein B is

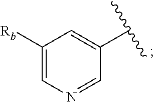

and R_{b} is hydrogen, fluoro or methoxy. In some embodiments, the disclosure provides a compound of Formula (I) or a pharmaceutically acceptable salt thereof, wherein B is

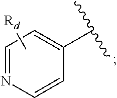

and R_{d} is hydrogen, fluoro, methoxy, or CF₃.

In some embodiments, the disclosure provides a compound of Formula (I) or a pharmaceutically acceptable salt thereof, wherein A₁ is

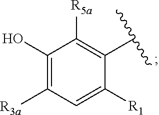

R₁ is hydrogen, halogen, or cyano; and R₃ₐ and R₅ₐ are each independently hydrogen, halogen, or optionally substituted lower alkyl. In some embodiments, the disclosure provides a compound of Formula (I) or a pharmaceutically acceptable salt thereof, wherein R₅ₐ is hydrogen. In some embodiments, the disclosure provides a compound of Formula (I) or a pharmaceutically acceptable salt thereof, wherein R₁ is fluoro. In some embodiments, the disclosure provides a compound of Formula (I) or a pharmaceutically acceptable salt thereof, wherein R₃ₐ is hydrogen, fluoro, or methyl. In some embodiments, the disclosure provides a compound of Formula (I) or a pharmaceutically acceptable salt thereof, wherein R₃ₐ is hydrogen. In some embodiments, the disclosure provides a compound of Formula (I) or a pharmaceutically acceptable salt thereof, wherein B is optionally substituted 2-pyridone, or pyridine optionally substituted with fluoro or methoxy.

In some embodiments, the disclosure provides a compound of Formula (I) or a pharmaceutically acceptable salt thereof, wherein A₁ is

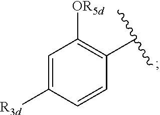

R_{3b} is halogen; R_{5b} is optionally substituted lower alkyl, optionally substituted lower cycloalkyl, or optionally substituted lower haloalkyl. In some embodiments, the disclosure provides a compound of Formula (I) or a pharmaceutically acceptable salt thereof, wherein R_{5b} is cyclopropyl, or lower alkyl optionally substituted with one or more fluoro. In some embodiments, the disclosure provides a compound of Formula (I) or a pharmaceutically acceptable salt thereof, wherein R_{3b} is fluoro. In some embodiments, the disclosure provides a compound of Formula (I) or a pharmaceutically acceptable salt thereof, wherein B is optionally substituted 2-pyridone, or pyridine optionally substituted with fluoro or methoxy.

In some embodiments, the disclosure provides a compound of Formula (I-A-1) or a pharmaceutically acceptable salt thereof.

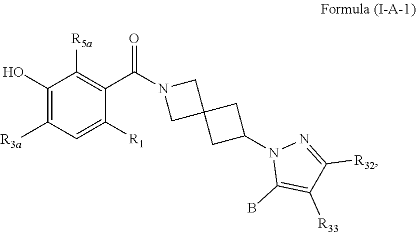

wherein:
R₁ is halogen or cyano;
R₃ₐ is hydrogen, optionally substituted lower alkyl, or halogen;
R₅ₐ is hydrogen, halogen, or optionally substituted lower alkyl;
B is

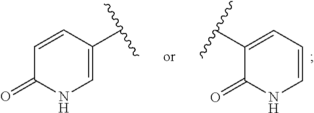 [SMILES: *c1ccc(=O)[nH]c1 ; *c1ccc[nH]c1=O]

and
R₃₂ and R₃₃ are each independently hydrogen, optionally substituted lower alkyl, optionally substituted lower haloalkyl, or optionally substituted lower cycloalkyl, provided that at least one of R₃₂ and R₃₃ is not hydrogen.

In some embodiments, the disclosure provides a compound of Formula (I-A-1) or a pharmaceutically acceptable salt thereof, wherein R₁ is F; R₃ₐ is hydrogen; R₅ₐ is hydrogen; and R₃₂ and R₃₃ are each independently hydrogen, methyl, CF₃, or cyclopropyl, provided that at least one of R₃₂ and R₃₃ is not hydrogen. In some embodiments, the disclosure provides a compound of Formula (I-A-1) or a pharmaceutically acceptable salt thereof, wherein R₃₂ is CF₃ and R₃₃ is hydrogen, or R₃₂ is hydrogen and R₃₃ is CF;

In some embodiments, the disclosure provides a compound of Formula (I-B-1) or a pharmaceutically acceptable salt thereof:

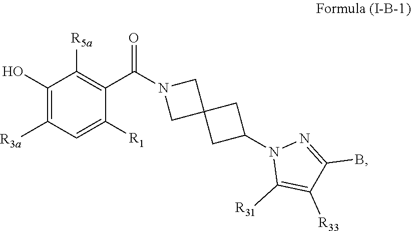

wherein:
R₁ is halogen or cyano;
R₃ₐ is hydrogen, optionally substituted lower alkyl, or halogen;
R₅ₐ is hydrogen, halogen or optionally substituted lower alkyl;
B is

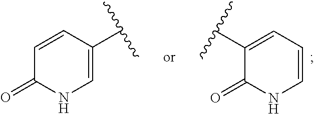 [SMILES: *c1ccc(=O)[nH]c1 ; *c1ccc[nH]c1=O]

R₃₁ and R₃₃ are each independently hydrogen, optionally substituted lower alkyl, optionally substituted lower haloalkyl, or optionally substituted lower cycloalkyl, provided that at least one of R₃₁ and R₃₃ is not hydrogen.

In some embodiments, the disclosure provides a compound of Formula (I-B-1) or a pharmaceutically acceptable salt thereof, wherein R₁ is F; R₃ₐ is hydrogen; R₅ₐ is hydrogen, and R₃₁ and R₃₃ are each independently hydrogen, methyl, CF₃, or cyclopropyl, provided that at least one of R₃₁ and R₃₃ is not hydrogen. In some embodiments, the disclosure provides a compound of Formula (I-B-1) or a pharmaceutically acceptable salt thereof, wherein R₃₁ is CF₃ and R₃₃ is hydrogen, or R₃₁ is hydrogen and R₃₃ is CF₃.

In some embodiments, the disclosure provides a compound of Formula (I-A-1) or a pharmaceutically acceptable salt thereof:

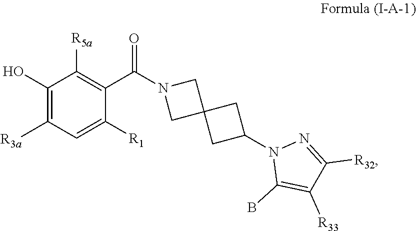

wherein:
R₁ is halogen or cyano;
R₃ₐ is hydrogen, optionally substituted lower alkyl, or halogen;
R₅ₐ is hydrogen, halogen or optionally substituted lower alkyl;
B is

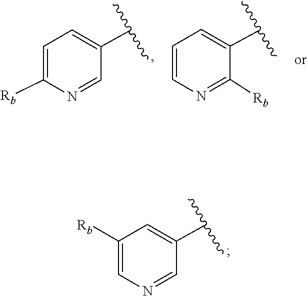

R_{b} is hydrogen, fluoro or methoxy; and
R₃₂ and R₃₃ are each independently hydrogen, optionally substituted lower alkyl, optionally substituted lower haloalkyl, or optionally substituted lower cycloalkyl, provided that at least one of R₃₂ and R₃₃ is not hydrogen.

In some embodiments, the disclosure provides a compound of Formula (I-A-1) or a pharmaceutically acceptable salt thereof, wherein R₁ is F; R₃ₐ is hydrogen; R₅ₐ is hydrogen; and R₃₂ and R₃₃ are each independently hydrogen, methyl, CF₃, or cyclopropyl, provided that at least one of R₃₂ and R₃₃ is not hydrogen; B is

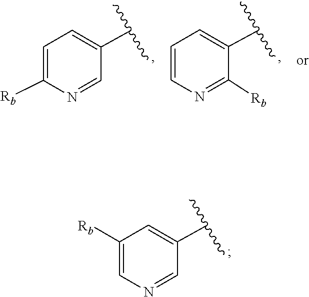

and R_{b} is hydrogen, fluoro or methoxy.

In some embodiments, the disclosure provides a compound of Formula (I-A-1) or a pharmaceutically acceptable salt thereof, wherein R₃₂ is CF₃ and R₃₃ is hydrogen, or R₃₂ is hydrogen and R₃₃ is CF₃.

In some embodiments, the disclosure provides a compound of Formula (I-B-1) or a pharmaceutically acceptable salt thereof:

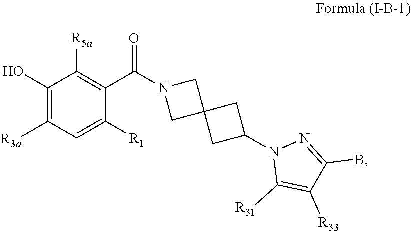

wherein:
R₁ is halogen or cyano;
R₃ is hydrogen, optionally substituted lower alkyl, or halogen;
R₅ₐ is hydrogen, halogen or optionally substituted lower alkyl;
B is

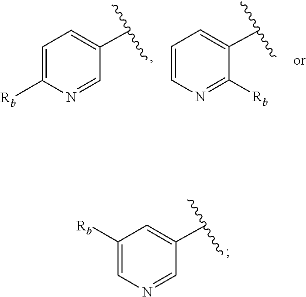

R_{b} is hydrogen, fluoro or methoxy; and
R₃₁ and R₃₃ are each independently hydrogen, optionally substituted lower alkyl, optionally substituted lower haloalkyl, or optionally substituted lower cycloalkyl, provided that at least one of R₃₁ and R₃₃ is not hydrogen.

In some embodiments, the disclosure provides a compound of Formula (I-B-1) or a pharmaceutically acceptable salt thereof, wherein Rₜ is F; R₃ₐ is hydrogen; R₅ₐ is hydrogen; R₃₁ and R₃₃ are each independently hydrogen, methyl, CF₃, or cyclopropyl, provided that at least one of R₃₁ and R₃₃ is not hydrogen, B is

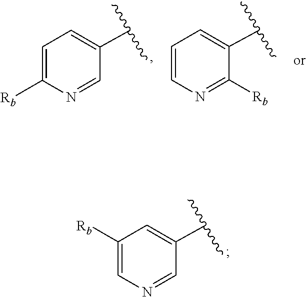

and R_{b} is hydrogen, fluoro or methoxy.

In some embodiments, the disclosure provides a compound of Formula (I-B-1) or a pharmaceutically acceptable salt thereof, wherein R₃₁ is CF₃ and R₃₃ is hydrogen, or R₃₁ is hydrogen and R₃₃ is CF₃.

In some embodiments, the disclosure provides a compound of Formula (II) or a pharmaceutically acceptable salt thereof:

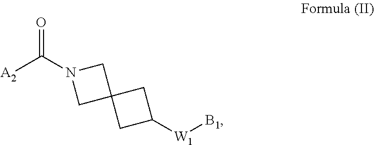

wherein:
A₂ is

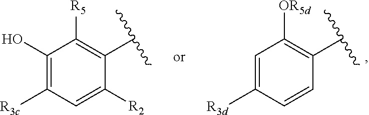

R₂ is hydrogen, halogen, or cyano;
R_{3c} and R_{5c} are each independently hydrogen, halogen, or optionally substituted lower alkyl;
R_{3d} is halogen;
R_{5d} is optionally substituted lower alkyl, optionally substituted lower cycloalkyl, or optionally substituted lower haloalkyl;
W₁ is

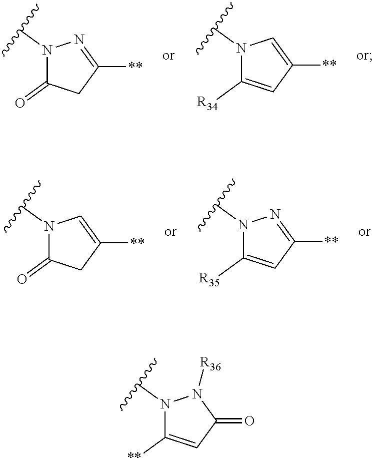

** is the bond to B₁;
R₃₄ is lower alkoxy, lower alkyl, lower haloalkyl or lower cycloalkyl;
R₃₅ is lower alkoxy, lower alkyl, lower haloalkyl or lower cycloalkyl;
R₃₆ is hydrogen or lower alkyl; and
B₁ is phenyl optionally substituted with halogen, optionally substituted lower alkoxy, optionally substituted lower alkyl, or optionally substituted lower haloalkyl.

In some embodiments, the disclosure provides a compound of Formula (II) or a pharmaceutically acceptable salt thereof, wherein W₁ is

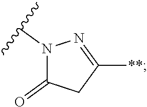

**is the bond to B₁; and B₁ is phenyl optionally substituted with halogen, optionally substituted lower alkoxy, optionally substituted lower alkyl, or optionally substituted lower haloalkyl.

In some embodiments, the disclosure provides a compound of Formula (H) or a pharmaceutically acceptable salt thereof, wherein W₁ is

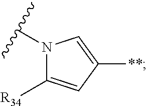

R₃₄ is lower alkoxy, lower alkyl, lower haloalkyl or lower cycloalkyl; ** is the bond to B₁; and B₁ is phenyl optionally substituted with halogen, optionally substituted lower alkoxy, optionally substituted lower alkyl, or optionally substituted lower haloalkyl. In some embodiments, the disclosure provides a compound of Formula (II) or a pharmaceutically acceptable salt thereof, wherein R_{3d} is F; and R_{5d} is methyl or cyclopropyl. In some embodiments, the disclosure provides a compound of Formula (II) or a pharmaceutically acceptable salt thereof, wherein R_{5d} is cyclopropyl. In some embodiments, the disclosure provides a compound of Formula (II) or a pharmaceutically acceptable salt thereof, wherein R₃₄ is methoxy.

In some embodiments, the compound is

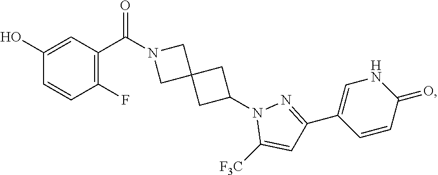 [SMILES: O=C(c1cc(O)ccc1F)N1CC2(CC(n3nc(-c4ccc(=O)[nH]c4)cc3C(F)(F)F)C2)C1]

or a pharmaceutically acceptable salt thereof. In some embodiments, the compound is

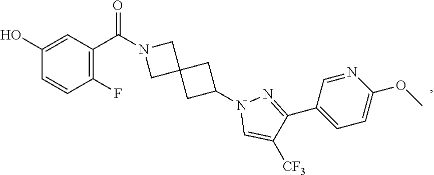 [SMILES: COc1ccc(-c2nn(C3CC4(C3)CN(C(=O)c3cc(O)ccc3F)C4)cc2C(F)(F)F)cn1]

or a pharmaceutically acceptable salt thereof. In some embodiments, the compound is

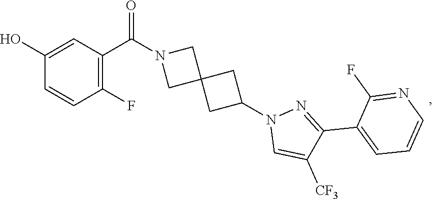 [SMILES: O=C(c1cc(O)ccc1F)N1CC2(CC(n3cc(C(F)(F)F)c(-c4cccnc4F)n3)C2)C1]

or a pharmaceutically acceptable salt thereof. In some embodiments, the compound is

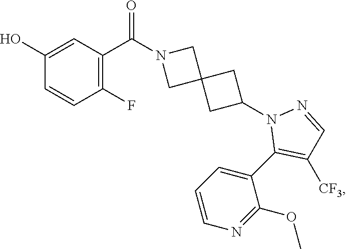 [SMILES: COc1ncccc1-c1c(C(F)(F)F)cnn1C1CC2(C1)CN(C(=O)c1cc(O)ccc1F)C2]

or a pharmaceutically acceptable salt thereof.

In some embodiments, the compound is

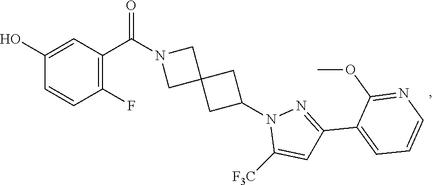 [SMILES: COc1ncccc1-c1cc(C(F)(F)F)n(C2CC3(C2)CN(C(=O)c2cc(O)ccc2F)C3)n1]

or a pharmaceutically acceptable salt thereof. In some embodiments, the compound is

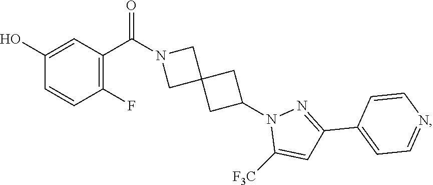 [SMILES: O=C(c1cc(O)ccc1F)N1CC2(CC(n3nc(-c4ccncc4)cc3C(F)(F)F)C2)C1]

or a pharmaceutically acceptable salt thereof. In some embodiments, the compound is

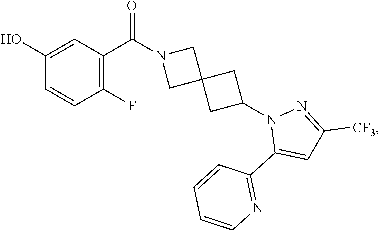 [SMILES: O=C(c1cc(O)ccc1F)N1CC2(CC(n3nc(C(F)(F)F)cc3-c3ccccn3)C2)C1]

or a pharmaceutically acceptable salt thereof. In some embodiments, the compound is

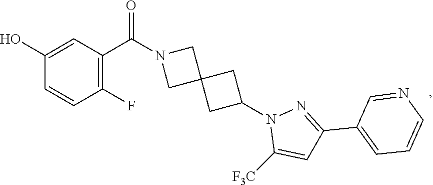 [SMILES: O=C(c1cc(O)ccc1F)N1CC2(CC(n3nc(-c4cccnc4)cc3C(F)(F)F)C2)C1]

or a pharmaceutically acceptable salt thereof. In some embodiments, the compound is

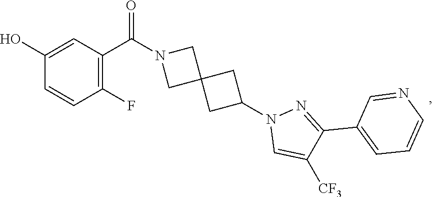 [SMILES: O=C(c1cc(O)ccc1F)N1CC2(CC(n3cc(C(F)(F)F)c(-c4cccnc4)n3)C2)C1]

or a pharmaceutically acceptable salt thereof. In some embodiments, the compound is

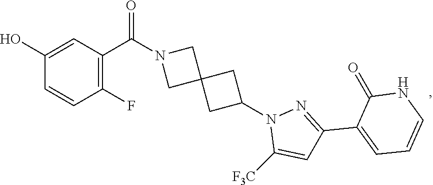 [SMILES: O=C(c1cc(O)ccc1F)N1CC2(CC(n3nc(-c4ccc[nH]c4=O)cc3C(F)(F)F)C2)C1]

or a pharmaceutically acceptable salt thereof. The compound of claim 1, wherein the compound is

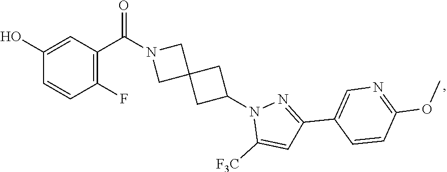 [SMILES: COc1ccc(-c2cc(C(F)(F)F)n(C3CC4(C3)CN(C(=O)c3cc(O)ccc3F)C4)n2)cn1]

or a pharmaceutically acceptable salt thereof.

In some embodiments, the compound is a compound of Formula (II-A-1) or a pharmaceutically acceptable salt thereof,

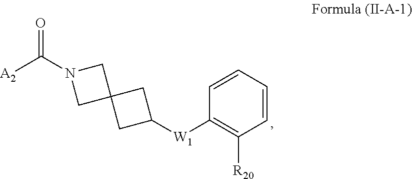

wherein:
   A₂ is

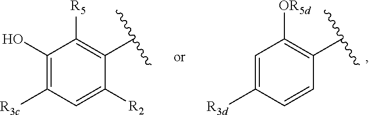

R₂ is hydrogen, halogen, or cyano;
R_{3c} and R_{5c} are each independently hydrogen, halogen, or lower alkyl;
R_{3d} is halogen;
R_{5d} is lower alkyl, lower cycloalkyl, or lower haloalkyl;
W₁ is

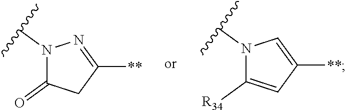

** is the bond to B₁;
R₃₄ is lower alkoxy; and
R₂₀ is hydrogen, halogen, lower alkyl or lower haloalkyl.

In some embodiments, the compound is a compound of Formula (II-A-1) or a pharmaceutically acceptable salt thereof, wherein R₂₀ is fluoro or CF₃. In some embodiments, the compound is a compound of Formula (II-A-1) or a pharmaceutically acceptable salt thereof, wherein R₂ and R_{3d} are both fluoro. In some embodiments, the compound is a compound of Formula (II-A-1) or a pharmaceutically acceptable salt thereof, wherein R_{3c} and R_{5c} are both hydrogen. In some embodiments, the compound is a compound of Formula (II-A-1) or a pharmaceutically acceptable salt thereof, wherein R_{5d} is cyclopropyl. In some embodiments, the compound is a compound of Formula (II-A-1) or a pharmaceutically acceptable salt thereof, wherein A₂ is

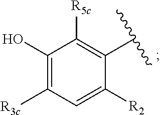

R₂ is hydrogen, halogen, or cyano; and R_{3c} and R_{5c} are each independently hydrogen, halogen, or lower alkyl. In some embodiments, the compound is a compound of Formula (II-A-1) or a pharmaceutically acceptable salt thereof, wherein A₂ is;

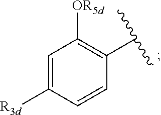

R_{3d} is halogen; and R_{5d} is lower alkyl, lower cycloalkyl or lower haloalkyl. In some embodiments, the compound is a compound of Formula (II-A-1) or a pharmaceutically acceptable salt thereof, wherein R₂₀ is fluoro. In some embodiments, the compound is a compound of Formula (II-A-1) or a pharmaceutically acceptable salt thereof, wherein R₂₀ is CF₃. In some embodiments, the compound is a compound of Formula (II-A-1) or a pharmaceutically acceptable salt thereof, wherein R₃₄ is methoxy. In some embodiments, the compound is a compound of Formula (II-A-1) or a pharmaceutically acceptable salt thereof, wherein R₃₁ is CF₃ and R₃₃ is hydrogen. In some embodiments, the compound is a compound of Formula (II-A-1) or a pharmaceutically acceptable salt thereof, wherein R₃₁ is hydrogen and R₃₃ is CF₃. In some embodiments, the compound is a compound of Formula (II-A-1) or a pharmaceutically acceptable salt thereof, wherein B is

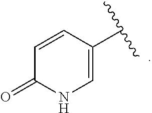 [SMILES: *c1ccc(=O)[nH]c1]

In some embodiments, the compound is a compound of Formula (II-A-1) or a pharmaceutically acceptable salt thereof, wherein B is

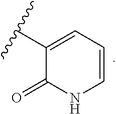 [SMILES: *c1ccc[nH]c1=O]

In some embodiments, the compound is a compound of Formula (0l-A-1) or a pharmaceutically acceptable salt thereof, wherein R₃₁ is CF₃ and R₃₃ is hydrogen. In some embodiments, the compound is a compound of Formula (II-A-1) or a pharmaceutically acceptable salt thereof, wherein R₃₁ is hydrogen and R₃₃ is CF₃. In some embodiments, the compound is a compound of Formula (II-A-1) or a pharmaceutically acceptable salt thereof, wherein B is

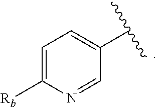

and R_{b} is hydrogen, fluoro or methoxy. In some embodiments, the compound is a compound of Formula (II-A-1) or a pharmaceutically acceptable salt thereof, wherein B is

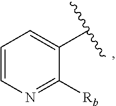

and R_{b} is hydrogen, fluoro or methoxy. In some embodiments, the compound is a compound of Formula (II-A-1) or a pharmaceutically acceptable salt thereof, wherein B is

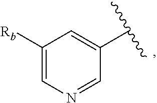

and R_{b} is hydrogen, fluoro or methoxy. In some embodiments, the compound is a compound of Formula (II-A-1) or a pharmaceutically acceptable salt thereof, wherein R_{b} is hydrogen. In some embodiments, the compound is a compound of Formula (II-A-1) or a pharmaceutically acceptable salt thereof, wherein R_{b} is fluoro. In some embodiments, the compound is a compound of Formula (II-A-1) or a pharmaceutically acceptable salt thereof, wherein R_{b} is methoxy. In some embodiments, the compound is a compound of Formula (II-A-1) or a pharmaceutically acceptable salt thereof, wherein R₃₁ is CF₃ and R₃₃ is hydrogen. In some embodiments, the compound is a compound of Formula (II-A-1) or a pharmaceutically acceptable salt thereof, wherein R₃₁ is hydrogen and R₃₃ is CF₃. In some embodiments, the compound is a compound of Formula (II-A-1) or a pharmaceutically acceptable salt thereof, wherein B is

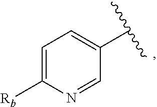

and R_{b} is hydrogen, fluoro or methoxy. In some embodiments, the compound is a compound of Formula (II-A-1) or a pharmaceutically acceptable salt thereof, wherein B is

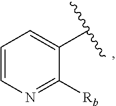

and R_{b} is hydrogen, fluoro or methoxy. In some embodiments, the compound is a compound of Formula (II-A-1) or a pharmaceutically acceptable salt thereof, wherein B is

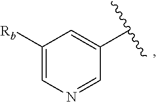

and R_{b} is hydrogen, fluoro or methoxy. In some embodiments, the compound is a compound of Formula (II-A-1) or a pharmaceutically acceptable salt thereof, wherein R_{b} is hydrogen. In some embodiments, the compound is a compound of Formula (II-A-1) or a pharmaceutically acceptable salt thereof, wherein R_{b} is fluoro. In some embodiments, the compound is a compound of Formula (II-A-1) or a pharmaceutically acceptable salt thereof, wherein R_{b} is methoxy.

In some embodiments, the compound is a compound of Formula (I) or a pharmaceutically acceptable salt thereof,

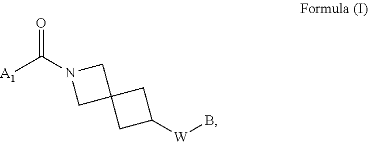

wherein:
   A₁ is

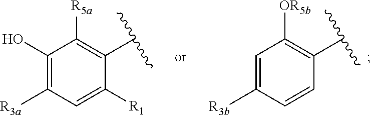

R₁ is hydrogen, halogen, or cyano;
R₃ₐ and R₅ₐ are each independently hydrogen, halogen, or optionally substituted lower alkyl;
R_{3b} is halogen;
R_{5b} is optionally substituted lower alkyl, optionally substituted lower cycloalkyl, or optionally substituted lower haloalkyl;
W is a pyrazolone, pyrrole or diazole, each independently optionally substituted with halogen, optionally substituted lower alkyl, optionally substituted lower cycloalkyl, optionally substituted lower alkoxy, or optionally substituted lower haloalkyl; and
B is 2-piperidinone or 2-dihydropyridinone optionally substituted pyridone, or a 6-member heteroaryl comprising at least one nitrogen atom and optionally substituted with one or more halogen, optionally substituted lower alkyl, optionally substituted lower haloalkyl, or optionally substituted lower alkoxy.

In some embodiments, the compound is a compound of Formula (I), or a pharmaceutically acceptable salt thereof, wherein R₁ is fluoro; R₃ₐ and R₅ₐ are each independently hydrogen; and R_{3b} is fluoro; and R_{5b} is cyclopropyl. In some embodiments, the compound is a compound of Formula (I) or a pharmaceutically acceptable salt thereof, wherein B is unsubstituted. In some embodiments, the compound is a compound of Formula (I) or a pharmaceutically acceptable salt thereof, wherein B is

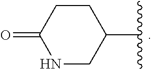 [SMILES: *C1CCC(=O)NC1]

In some embodiments, the compound is a compound of Formula (I) or a pharmaceutically acceptable salt thereof, wherein B is

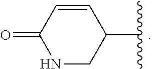 [SMILES: *C1C=CC(=O)NC1]

In some embodiments, the compound is a compound of Formula (I) or a pharmaceutically acceptable salt thereof, wherein B is

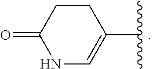 [SMILES: *C1=CNC(=O)CC1]

A compound of Formula (IV) or a pharmaceutically acceptable salt thereof,

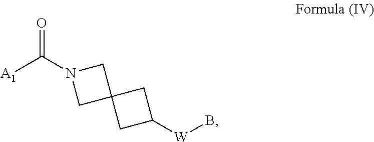

wherein:
   A₁ is

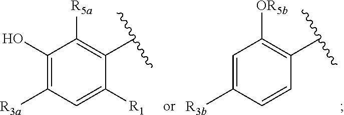

R₁ is hydrogen, halogen, or cyano;
R₃ₐ and R₅ₐ are each independently hydrogen, halogen, or optionally substituted lower alkyl;
R_{3b} is halogen;
R_{5b} is optionally substituted lower alkyl, optionally substituted lower cycloalkyl, or optionally substituted lower haloalkyl;
W is

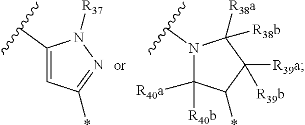

R₃₇, R₃₈ₐ, R_{38b}, R₃₉ₐ, R_{39b}, R₄₀ₐ, R_{40b}, is hydrogen, halogen, lower alkyl, lower alkoxy, lower haloalkyl, or lower cycloalkyl;
B is an optionally substituted 24yridine, phenyl or a 6-member heteroaryl comprising at least one nitrogen atom and optionally substituted with one or more halogen, optionally substituted lower alkyl, optionally substituted lower haloalkyl, or optionally substituted lower alkoxy.

In some embodiments, B is phenyl in compounds of Formula (IV), wherein B can be substituted with one or more halogen or haloalkyl; or B can be substituted with one of more fluoro. In some embodiments, B is

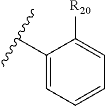

in compounds of Formula (IV), and R₂₀ can be halogen, haloalkyl, or alkyl, or R₂₀ can be F, methyl or CF₃.

In some embodiments, W is

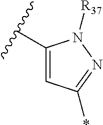

in compounds of Formula (IV), including compounds wherein R₃₇ is hydrogen, halogen, lower alkyl, lower alkoxy, lower haloalkyl, or lower cycloalkyl; compounds wherein R₃₇ is hydrogen, methyl, methoxy, CF₃, or cyclopropyl, compounds wherein R₃₇ is methoxy; compounds wherein R₃₇ is methyl; compounds wherein R₃₇ is hydrogen; compounds wherein R₃₇ is cyclopropyl; or compounds wherein R₃₇ is CF₃. In some embodiments, W is

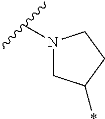 [SMILES: *C1CCN(*)C1]

in compounds of Formula (IV).

In some embodiments, W is

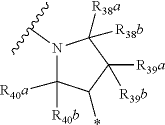

in compounds of Formula (IV), including compounds wherein R₃₈ₐ, R_{38b}, R₃₉ₐ, R_{39b}, R₄₀ₐ, R_{40b}, is hydrogen, halogen, methyl, methoxy, CF₃, or cyclopropyl; compounds wherein R₃₈ₐ, R_{39b}, and R₄₀ₐ, is hydrogen; compounds wherein one of R_{38b}, R_{39b}, and R_{40b}, is halogen, methyl, methoxy, CF₃, or cyclopropyl; and compounds wherein two of R_{38b}, R_{39b}, and R_{40b} are hydrogen.

In some embodiments A₁ is

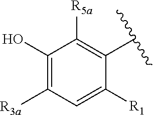

in compounds of Formula (IV), including compounds wherein R₁ is hydrogen, halogen, or cyano; and R₃ₐ and R₅ₐ are each independently hydrogen, halogen, or optionally substituted lower alkyl; compounds wherein R₅ₐ is hydrogen; compounds wherein R₁ is fluoro; compounds wherein R₃ₐ is hydrogen, fluoro, or methyl; compounds wherein R₃ₐ is hydrogen.

In some embodiments, A₁ is

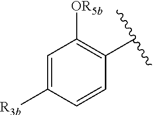

in compounds of Formula (IV), including compounds wherein R_{3b} is halogen, compounds wherein R_{5b} is optionally substituted lower alkyl, optionally substituted lower cycloalkyl, or optionally substituted lower haloalkyl; compounds wherein R_{5b} is cyclopropyl, or lower alkyl optionally substituted with one or more fluoro; compounds wherein R_{3b} is fluoro; or compounds wherein R_{5b} is cyclopropyl.

In some embodiments, the compound is a compound of Formula (V) or a pharmaceutically acceptable salt thereof,

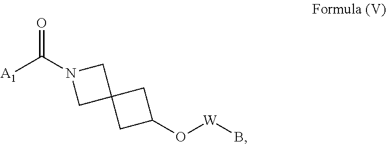

wherein:
   A₁ is

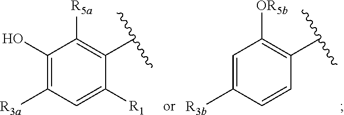

R₁ is hydrogen, halogen, or cyano;
R₃ₐ and R₅ₐ are each independently hydrogen, halogen, or optionally substituted lower alkyl;
R_{3b} is halogen;
R_{5b} is optionally substituted lower alkyl, optionally substituted lower cycloalkyl, or optionally substituted lower haloalkyl;
W is selected from the group consisting of:

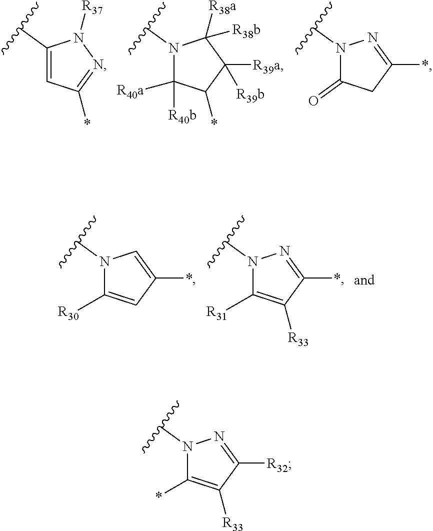

R₃₀, R₃₁, R₃₂, R₃₃, R₃₇, R₃₈ₐ, R_{38b}, R₃₉ₐ, R_{39b}, R₄₀ₐ, R_{40b}, are each independently hydrogen, halogen, lower alkyl, lower alkoxy, lower haloalkyl, or lower cycloalkyl;
B is an optionally substituted 28pyridine, phenyl or a 6-member heteroaryl comprising at least one nitrogen atom and optionally substituted with one or more halogen, optionally substituted lower alkyl, optionally substituted lower haloalkyl, or optionally substituted lower alkoxy.

In some embodiments, B is phenyl in compounds of Formula (V), wherein B can be substituted with one or more halogen or haloalkyl; or B can be substituted with one of more fluoro. In some embodiments, B is

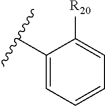

in compounds of Formula (V), and R₂₀ can be halogen, haloalkyl, or alkyl; or R₂₀ can be F methyl or CF₃.

In some embodiments, W is

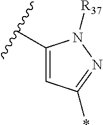

in compounds of Formula (V), including compounds wherein R₃₇ is hydrogen, halogen, lower alkyl, lower alkoxy, lower haloalkyl, or lower cycloalkyl; compounds wherein R₃₇ is hydrogen, methyl, methoxy, CF₃, or cyclopropyl; compounds wherein R₃₇ is methoxy; compounds wherein R₃₇ is methyl; compounds wherein R₃₇ is hydrogen; compounds wherein R₃₇ is cyclopropyl; or compounds wherein R₃₇ is CF₃.

In some embodiments A₁ is

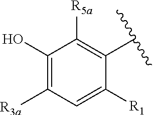

in compounds of Formula (V), including compounds wherein R₁ is hydrogen, halogen, or cyano; and R₃ₐ and R₅ₐ are each independently hydrogen, halogen, or optionally substituted lower alkyl; compounds wherein R₅ₐ is hydrogen; compounds wherein R₁ is fluoro; compounds wherein R₃ₐ is hydrogen, fluoro, or methyl; compounds wherein R₃ₐ is hydrogen.

In some embodiments, A₁ is

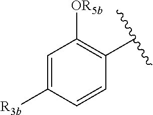

in compounds of Formula (V), including compounds wherein R_{3b} is halogen; compounds wherein R_{5b} is optionally substituted lower alkyl, optionally substituted lower cycloalkyl, or optionally substituted lower haloalkyl; compounds wherein R_{5b} is cyclopropyl, or lower alkyl optionally substituted with one or more fluoro; compounds wherein R_{3b} is fluoro; or compounds wherein R_{5b} is cyclopropyl.

In some embodiments, a pharmaceutical composition comprises a compound disclosed herein, or a pharmaceutically acceptable salt thereof as the active pharmaceutical ingredient (API).

In some embodiments, a method of inhibiting monoacylglycerol lipase (MAGL) in a subject comprising the step of administering to a subject in need thereof a therapeutically effective amount of a compound of any compound disclosed herein.

In some embodiments, a method of reversibly inhibiting monoacylglycerol lipase (MAGL) comprising the step of contacting a compound of any one the compounds disclosed herein with a cell expressing MAGL.

### BRIEF DESCRIPTION OF THE FIGURES

There are no Figures included in the disclosure.

### DETAILED DESCRIPTION

Applicants have discovered chemical compounds that are Reversible MAGL Inhibitor Compounds and Selective MAGL Inhibitor Compounds. In some embodiments, the compound is a Reversible MAGL Inhibitor Compound. In some embodiments, the compound is a Selective MAGL Inhibitor Compound. In some embodiments, the compound is both a Reversible MAGL Inhibitor Compound and a Selective MAGL Inhibitor Compound.

In some embodiments, the disclosure provides a compound of Formula (I) or a pharmaceutically acceptable salt thereof:

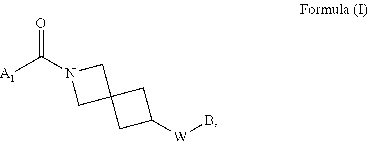

wherein:
A₁ is

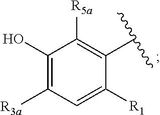

R₁ is hydrogen, halogen, or cyano;
R₃ₐ and R₅ₐ are each independently hydrogen, halogen, or optionally substituted lower alkyl;
W is a pyrazolone, pyrrole, or diazole, each independently optionally substituted with halogen, optionally substituted lower alkyl, optionally substituted lower cycloalkyl, optionally substituted lower alkoxy, or optionally substituted lower haloalkyl; and
B is an optionally substituted pyridone, dihydropyridone, piperidinone, or a 6-member heteroaryl comprising at least one nitrogen atom and optionally substituted with one or more halogen, optionally substituted lower alkyl, optionally substituted lower haloalkyl, or optionally substituted lower alkoxy.

In some embodiments, the disclosure provides a compound of Formula (I) or a pharmaceutically acceptable salt thereof, wherein R₁ is hydrogen, fluoro, or cyano; and R₃ₐ and R₅ₐ are each independently hydrogen, fluoro, or methyl.

In some embodiments, the disclosure provides a compound of Formula (I) or a pharmaceutically acceptable salt thereof:

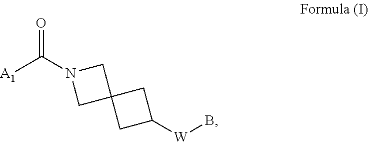

wherein:
A₁ is

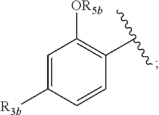

R_{3b} is halogen;
R_{5b} is optionally substituted lower alkyl, optionally substituted lower cycloalkyl, or optionally substituted lower haloalkyl;
W is a pyrazolone, pyrrole, or diazole each independently optionally substituted with halogen, optionally substituted lower alkyl, optionally substituted lower cycloalkyl, optionally substituted lower alkoxy, or optionally substituted lower haloalkyl; and
B is an optionally substituted pyridone, dihydropyridone, piperidinone or a 6-member heteroaryl comprising at least one nitrogen atom and optionally substituted with one or more halogen, optionally substituted lower alkyl, optionally substituted lower haloalkyl, or optionally substituted lower alkoxy.

In some embodiments, the disclosure provides a compound of Formula (I) or a pharmaceutically acceptable salt thereof, wherein R_{3b} is fluoro; and R_{5b} is cyclopropyl, or lower alkyl optionally substituted with one or more fluoro.

In some embodiments, the disclosure provides a compound of Formula (I) or a pharmaceutically acceptable salt thereof, wherein W is a diazole optionally substituted with one of methyl, cyclopropyl, or CF₃; and B is an optionally substituted pyridone, or a pyridine optionally substituted with fluoro, chloro, methoxy, methyl or CF₃.

In some embodiments, the disclosure provides a compound of Formula (I) or a pharmaceutically acceptable salt thereof, wherein R_{5b} is lower alkyl, lower cycloalkyl or lower haloalkyl; and R_{3b} is halogen. In some embodiments, the disclosure provides a compound of Formula (I) or a pharmaceutically acceptable salt thereof, wherein R_{5b} is lower cycloalkyl and R_{3b} is fluoro. In some embodiments, the disclosure provides a compound of Formula (I) or a pharmaceutically acceptable salt thereof, wherein W is selected from the group consisting of:

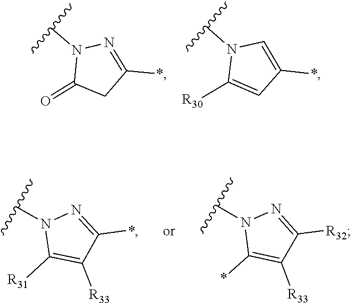

wherein * is the bond to B; and R₃₀, R₃₁, R₃₂ and R₃₃ are each independently hydrogen, halogen, lower alkyl, lower alkoxy, lower haloalkyl, or lower cycloalkyl, provided that at least one of R₃₁ and R₃₃ and at least one of R₃₂ and R₃₃ is not hydrogen.

In some embodiments, the disclosure provides a compound of Formula (I) or a pharmaceutically acceptable salt thereof, wherein R₃₀ is hydrogen, halogen, lower alkyl, lower alkoxy, lower haloalkyl, or lower cycloalkyl. In some embodiments, the disclosure provides a compound of Formula (I) or a pharmaceutically acceptable salt thereof, wherein R₃₀ is hydrogen, F, methyl, —CF₃, methoxy, or cyclopropyl. In some embodiments, the disclosure provides a compound of Formula (I) or a pharmaceutically acceptable salt thereof, wherein R₃₀ is —CF₃. In some embodiments, the disclosure provides a compound of Formula (I) or a pharmaceutically acceptable salt thereof, wherein R₃₀ is hydrogen. In some embodiments, the disclosure provides a compound of Formula (I) or a pharmaceutically acceptable salt thereof, wherein R₃₀ is methyl. In some embodiments, the disclosure provides a compound of Formula (I) or a pharmaceutically acceptable salt thereof, wherein R₃₀ is methoxy. In some embodiments, the disclosure provides a compound of Formula (I) or a pharmaceutically acceptable salt thereof, wherein R₃₀ is F.

In some embodiments, the disclosure provides a compound of Formula (I) or a pharmaceutically acceptable salt thereof, wherein R₃₁ is hydrogen, halogen, lower alkyl, lower alkoxy, lower haloalkyl, or lower cycloalkyl. In some embodiments, the disclosure provides a compound of Formula (I) or a pharmaceutically acceptable salt thereof, wherein R₃₁ is hydrogen, F, methyl, —CF₃, methoxy, or cyclopropyl. In some embodiments, the disclosure provides a compound of Formula (I) or a pharmaceutically acceptable salt thereof, wherein R₃₁ is —CF₃. In some embodiments, the disclosure provides a compound of Formula (I) or a pharmaceutically acceptable salt thereof, wherein R₃₁ is hydrogen. In some embodiments, the disclosure provides a compound of Formula (I) or a pharmaceutically acceptable salt thereof, wherein R₃₁ is methyl. In some embodiments, the disclosure provides a compound of Formula (I) or a pharmaceutically acceptable salt thereof, wherein R₃₁ is methoxy. In some embodiments, the disclosure provides a compound of Formula (I) or a pharmaceutically acceptable salt thereof, wherein R₃₁ is F.

In some embodiments, the disclosure provides a compound of Formula (I) or a pharmaceutically acceptable salt thereof, wherein R₃₂ is hydrogen, halogen, lower alkyl, lower alkoxy, lower haloalkyl, or lower cycloalkyl. In some embodiments, the disclosure provides a compound of Formula (I) or a pharmaceutically acceptable salt thereof, wherein R₃₂ is hydrogen, F, methyl, —CF₃, methoxy, or cyclopropyl. In some embodiments, the disclosure provides a compound of Formula (I) or a pharmaceutically acceptable salt thereof, wherein R₃₂ is —CF₃. In some embodiments, the disclosure provides a compound of Formula (I) or a pharmaceutically acceptable salt thereof, wherein R₃₂ is hydrogen. In some embodiments, the disclosure provides a compound of Formula (I) or a pharmaceutically acceptable salt thereof, wherein R₃₂ is methyl. In some embodiments, the disclosure provides a compound of Formula (I) or a pharmaceutically acceptable salt thereof, wherein R₃₂ is methoxy. In some embodiments, the disclosure provides a compound of Formula (I) or a pharmaceutically acceptable salt thereof, wherein R₃₂ is F.

In some embodiments, the disclosure provides a compound of Formula (I) or a pharmaceutically acceptable salt thereof, wherein R₃₂ is hydrogen, halogen, lower alkyl, lower alkoxy, lower haloalkyl, or lower cycloalkyl. In some embodiments, the disclosure provides a compound of Formula (I) or a pharmaceutically acceptable salt thereof, wherein R₃₃ is hydrogen, F, methyl, —CF₃, methoxy, or cyclopropyl. In some embodiments, the disclosure provides a compound of Formula (I) or a pharmaceutically acceptable salt thereof, wherein R₃₃ is —CF₃. In some embodiments, the disclosure provides a compound of Formula (I) or a pharmaceutically acceptable salt thereof, wherein R₃₃ is hydrogen. In some embodiments, the disclosure provides a compound of Formula (I) or a pharmaceutically acceptable salt thereof, wherein R₃₃ is methyl. In some embodiments, the disclosure provides a compound of Formula (I) or a pharmaceutically acceptable salt thereof, wherein R₃₃ is methoxy. In some embodiments, the disclosure provides a compound of Formula (I) or a pharmaceutically acceptable salt thereof, wherein R₃₃ is F.

In some embodiments, the disclosure provides a compound of Formula (I) or a pharmaceutically acceptable salt thereof, wherein R₃₁ is hydrogen and R₃₃ is lower haloalkyl. In some embodiments, the disclosure provides a compound of Formula (I) or a pharmaceutically acceptable salt thereof, wherein R₃₁ is hydrogen and R₃₃ is —CF₃. In some embodiments, the disclosure provides a compound of Formula (I) or a pharmaceutically acceptable salt thereof, wherein R₃₃ is hydrogen and R₃₁ is lower haloalkyl. In some embodiments, the disclosure provides a compound of Formula (I) or a pharmaceutically acceptable salt thereof, wherein R₃₃ is hydrogen and R₃₁ is —CF₃.

In some embodiments, the disclosure provides a compound of Formula (I) or a pharmaceutically acceptable salt thereof, wherein R₃₂ is hydrogen and R₃₃ is lower haloalkyl. In some embodiments, the disclosure provides a compound of Formula (I) or a pharmaceutically acceptable salt thereof, wherein R₃₂ is hydrogen and R₃₃ is —CF₃. In some embodiments, the disclosure provides a compound of Formula (I) or a pharmaceutically acceptable salt thereof, wherein R₃₃ is hydrogen and R₃₂ is lower haloalkyl. In some embodiments, the disclosure provides a compound of Formula (I) or a pharmaceutically acceptable salt thereof, wherein R₃₃ is hydrogen and R₂ is —CF₃.

In some embodiments, the disclosure provides a compound of Formula (I) or a pharmaceutically acceptable salt thereof, wherein W is.

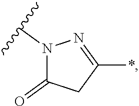

wherein * is the bond to B; and B is an optionally substituted pyridone, or a pyridine optionally substituted with halogen, lower alkoxy, lower alkyl, or lower haloalkyl.

In some embodiments, the disclosure provides a compound of Formula (I) or a pharmaceutically acceptable salt thereof, wherein W is.

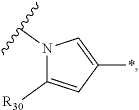

* is the bond to B; and B is an optionally substituted pyridone, or a pyridine optionally substituted with halogen, lower alkoxy, lower alkyl, or lower haloalkyl and R₃₀ is hydrogen, halogen, lower alkyl, lower haloalkyl, or lower cycloalkyl.

In some embodiments, the disclosure provides a compound of Formula (I) or a pharmaceutically acceptable salt thereof, wherein W is:

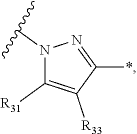

* is the bond to B; B is an optionally substituted pyridone, or a pyridine optionally substituted with halogen, lower alkoxy, lower alkyl, or lower haloalkyl; and R₃₁, and R₃₃ are each independently hydrogen, halogen, lower alkyl, lower haloalkyl, or lower cycloalkyl, provided that at least one of R₃₁ and R₃₃ is not hydrogen.

In some embodiments, the disclosure provides a compound of Formula (I) or a pharmaceutically acceptable salt thereof, wherein W is:

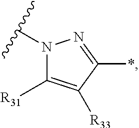

* is the bond to B; B is an optionally substituted pyridone, or a pyridine optionally substituted with halogen, lower alkoxy, lower alkyl, or lower haloalkyl; and R₃₁ and R₃₃ are each independently hydrogen, F, methyl, CF₃, or cyclopropyl, provided that at least one of R₃₁ and R₃₃ is not hydrogen.

In some embodiments, the disclosure provides a compound of Formula (I) or a pharmaceutically acceptable salt thereof, wherein W is.

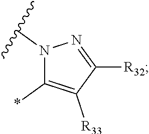

* is the bond to B; B is an optionally substituted pyridone, or a pyridine optionally substituted with halogen, lower alkoxy, lower alkyl, or lower haloalkyl; and R₃₂ and R₃₃ are each independently hydrogen, halogen, lower alkyl, lower haloalkyl, or lower cycloalkyl, and provided that at least one of R₃₂ and R₃₃ is not hydrogen. In some embodiments, the disclosure provides a compound of Formula (I) or a pharmaceutically acceptable salt thereof, wherein W is

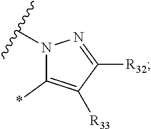

* is the bond to B; B is an optionally substituted pyridone, or a pyridine optionally substituted with halogen, lower alkoxy, lower alkyl, or lower haloalkyl; and R₃₂ and R₃₃ are each independently hydrogen, halogen, lower alkyl, lower haloalkyl, or lower cycloalkyl, provided that at least one of R₃₂ and R₃₃ is not hydrogen. In some embodiments, the disclosure provides a compound of Formula (I) or a pharmaceutically acceptable salt thereof, wherein R₃₂ and R₃₃ are each independently hydrogen, F, methyl, CF₃, or cyclopropyl, provided that at least one of R₃₂ and R₃₃ is not hydrogen.

In some embodiments, the disclosure provides a compound of Formula (I) or a pharmaceutically acceptable salt thereof, wherein B is optionally substituted 2-pyridone, or pyridine optionally substituted with fluoro, chloro, methyl, methoxy or CF₃. In some embodiments, the disclosure provides a compound of Formula (I) or a pharmaceutically acceptable salt thereof, wherein B is optionally substituted 2-pyridone. In some embodiments, the disclosure provides a compound of Formula (I) or a pharmaceutically acceptable salt thereof, wherein B is

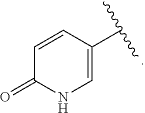 [SMILES: *c1ccc(=O)[nH]c1]

In some embodiments, the disclosure provides a compound of Formula (I) or a pharmaceutically acceptable salt thereof, wherein B is

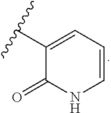 [SMILES: *c1ccc[nH]c1=O]

In some embodiments, the disclosure provides a compound of Formula (I) or a pharmaceutically acceptable salt thereof, wherein B is

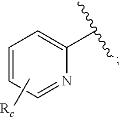

and R_{c} is hydrogen, fluoro, methoxy, or CF₃. In some embodiments, the disclosure provides a compound of Formula (I) or a pharmaceutically acceptable salt thereof, wherein B is

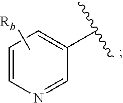

and R_{b} is hydrogen, fluoro, methoxy, or CF₃.

In some embodiments, the disclosure provides a compound of Formula (I) or a pharmaceutically acceptable salt thereof, wherein B is

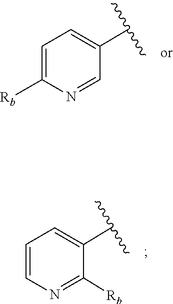

and R_{b} is hydrogen, fluoro or methoxy. In some embodiments, the disclosure provides a compound of Formula (I) or a pharmaceutically acceptable salt thereof, wherein B is

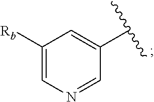

and R_{b} is hydrogen, fluoro or methoxy. In some embodiments, the disclosure provides a compound of Formula (I) or a pharmaceutically acceptable salt thereof, wherein B is

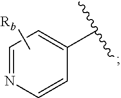

and R_{d} is hydrogen, fluoro, methoxy, or CF₃.

In some embodiments, the disclosure provides a compound of Formula (I) or a pharmaceutically acceptable salt thereof, wherein B is 2-piperidinone optionally substituted with fluoro, chloro, methyl, methoxy or CF₃. In some embodiments, the disclosure provides a compound of Formula (I) or a pharmaceutically acceptable salt thereof, wherein B is unsubstituted 2-piperidinone. In some embodiments, the disclosure provides a compound of Formula (I) or a pharmaceutically acceptable salt thereof, wherein B is

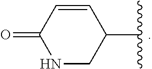 [SMILES: *C1C=CC(=O)NC1]

In some embodiments, the disclosure provides a compound of Formula (I) or a pharmaceutically acceptable salt thereof, wherein B is optionally substituted 2-dihydropyridinone optionally substituted with fluoro, chloro, methyl, methoxy or CF₃. In some embodiments, the disclosure provides a compound of Formula (I) or a pharmaceutically acceptable salt thereof, wherein B is optionally substituted 3,4-dihydropyridin-2(1H)-one optionally substituted with fluoro, chloro, methyl, methoxy or CF₃. In some embodiments, the disclosure provides a compound of Formula (I) or a pharmaceutically acceptable salt thereof, wherein B is optionally substituted 5,6-dihydropyridin-2(1H)-one optionally substituted with fluoro, chloro, methyl, methoxy or CF₃. In some embodiments, the disclosure provides a compound of Formula (I) or a pharmaceutically acceptable salt thereof, wherein B is unsubstituted 2-dihydropyridinone. In some embodiments, the disclosure provides a compound of Formula (I) or a pharmaceutically acceptable salt thereof, wherein B is unsubstituted 3,4-dihydropyridin-2(1H)-one. In some embodiments, the disclosure provides a compound of Formula (I) or a pharmaceutically acceptable salt thereof, wherein B is unsubstituted 5,6-dihydropyridin-2(1H)-one. In some embodiments, the disclosure provides a compound of Formula (I) or a pharmaceutically acceptable salt thereof, wherein B is

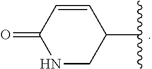 [SMILES: *C1C=CC(=O)NC1]

In some embodiments, the disclosure provides a compound of Formula (I) or a pharmaceutically acceptable salt thereof, wherein B is

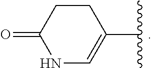 [SMILES: *C1=CNC(=O)CC1]

In some embodiments, the disclosure provides a compound of Formula (I) or a pharmaceutically acceptable salt thereof, wherein A₁ is

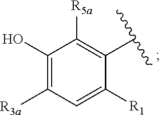

R₁ is hydrogen, halogen, or cyano; and R₃ₐ and R₅ₐ arm each independently hydrogen, halogen, or optionally substituted lower alkyl; and in some embodiments, the disclosure provides a compound of Formula (I) or a pharmaceutically acceptable salt thereof, wherein R₅ₐ is hydrogen; and, in some embodiments, the disclosure provides a compound of Formula (I) or a pharmaceutically acceptable salt thereof, wherein R₁ is fluoro; and, in some embodiments, the disclosure provides a compound of Formula (I) or a pharmaceutically acceptable salt thereof, wherein R₃ₐ is hydrogen, fluoro, or methyl; and, in some embodiments, the disclosure provides a compound of Formula (I) or a pharmaceutically acceptable salt thereof, wherein R₃ₐ is hydrogen; and in some embodiments, the disclosure provides a compound of Formula (I) or a pharmaceutically acceptable salt thereof, wherein B is optionally substituted 2-pyridone, or pyridine optionally substituted with fluoro or methoxy.

In some embodiments, the disclosure provides a compound of Formula (I) or a pharmaceutically acceptable salt thereof, wherein A₁ is

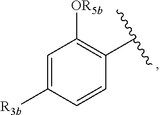

R_{3b} is halogen; and R_{5b} is optionally substituted lower alkyl, optionally substituted lower cycloalkyl, or optionally substituted lower haloalkyl; and, in some embodiments, the disclosure provides a compound of Formula (I) or a pharmaceutically acceptable salt thereof, wherein R_{5b} is cyclopropyl, or lower alkyl optionally substituted with one or more fluoro; and, in some embodiments, the disclosure provides a compound of Formula (I) or a pharmaceutically acceptable salt thereof, wherein R_{3b} is fluoro; and, in some embodiments, the disclosure provides a compound of Formula (I) or a pharmaceutically acceptable salt thereof, wherein B is optionally substituted 2-pyridone, or pyridine optionally substituted with fluoro or methoxy.

In some embodiments, the disclosure provides a compound of Formula (I-A-1) or a pharmaceutically acceptable salt thereof:

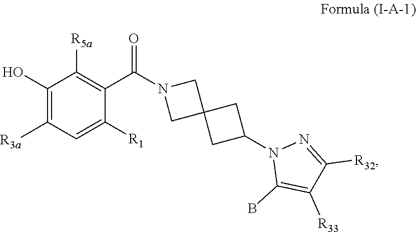

wherein:
R₁ is halogen or cyano;
R₃ₐ is hydrogen, optionally substituted lower alkyl, or halogen;
R₅ₐ is hydrogen, halogen, or optionally substituted lower alkyl;
B is pyridine, pyrimidine, or pyridinone, each independently optionally substituted with one or more R_{b};
R_{b} is hydrogen, halogen or alkoxy; and
R₃₂ and R₃₃ are each independently hydrogen, optionally substituted lower alkyl, optionally substituted lower haloalkyl, or optionally substituted lower cycloalkyl, provided that at least one of R₃₂ and R₃₃ is not hydrogen.

In some embodiments, the disclosure provides a compound of Formula (I-A-1) or a pharmaceutically acceptable salt thereof, wherein:
R₁ is halogen or cyano;
R₃ₐ is hydrogen, lower alkyl, or halogen;
R₅ₐ is hydrogen, halogen or lower alkyl;
B is pyridine or pyrimidine, each independently optionally substituted with one or more R_{b};
R_{b} is hydrogen, halogen or alkoxy; and
R₃₂ and R₃₃ are each independently hydrogen, lower alkyl, lower haloalkyl, or lower cycloalkyl, provided that at least one of R₃₂ and R₃₃ is not hydrogen.

In some embodiments, the disclosure provides a compound of Formula (I-A-1) or a pharmaceutically acceptable salt thereof, wherein:
R₁ is halogen or cyano;
R₃ₐ is hydrogen, lower alkyl, or halogen;
R₅ₐ is hydrogen, halogen or lower alkyl;
B is pyridine optionally substituted with one or more R_{b};
R_{b} is hydrogen, halogen or alkoxy; and
R₃₂ and R₃₃ are each independently hydrogen, lower alkyl, lower haloalkyl, or lower cycloalkyl, provided that at least one of R₃₂ and R₃₃ is not hydrogen.

In some embodiments, the disclosure provides a compound of Formula (I-A-1) or a pharmaceutically acceptable salt thereof, wherein R₁ is F, R₃ₐ is hydrogen; and R₅ₐ is hydrogen. In some embodiments, the disclosure provides a compound of Formula (I-A-1) or a pharmaceutically acceptable salt thereof, wherein R₃₂ and R₃₃ are each independently hydrogen, methyl optionally substituted with one or more F, or cyclopropyl, provided that at least one of R₃₂ and R₃₃ is not hydrogen, R₁ is F; R₃ₐ is hydrogen; and R₅ₐ is hydrogen.

In some embodiments, the disclosure provides a compound of Formula (I-A-1) or a pharmaceutically acceptable salt thereof, wherein R₃₂ and R₃₃ are each independently hydrogen, lower alkyl, lower haloalkyl, or lower cycloalkyl, provided that at least one of R₃₂ and R₃₃ is not hydrogen. In some embodiments, the disclosure provides a compound of Formula (I-A-1) or a pharmaceutically acceptable salt thereof, wherein R₃₂ is lower haloalkyl and R₃₃ is hydrogen. In some embodiments, the disclosure provides a compound of Formula (I-A-1) or a pharmaceutically acceptable salt thereof, wherein R₃₂ is —CF₃ and R₃₃ is hydrogen. In some embodiments, the disclosure provides a compound of Formula (I-A-1) or a pharmaceutically acceptable salt thereof, wherein R₃₃ is lower haloalkyl and R₃₂ is hydrogen. In some embodiments, the disclosure provides a compound of Formula (I-A-1) or a pharmaceutically acceptable salt thereof, wherein R₃₃ is —CF₃ and R₃₂ is hydrogen.

In some embodiments, the disclosure provides a compound of Formula (I-A-1) or a pharmaceutically acceptable salt thereof, wherein
B is

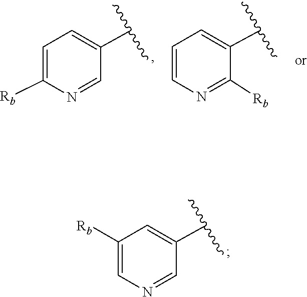

R_{b} is hydrogen, fluoro or methoxy; and
R₃₂ and R₃₃ are each independently hydrogen, lower alkyl, lower haloalkyl, or lower cycloalkyl, provided that at least one of R₃₂ and R₃₃ is not hydrogen.

In some embodiments, the disclosure provides a compound of Formula (I-A-1) or a pharmaceutically acceptable salt thereof, wherein R₁ is F; R₃ₐ is hydrogen; R₅ₐ is hydrogen; and R₃₂ and R₃₃ are each independently hydrogen, methyl, CF₃, or cyclopropyl, provided that at least one of R₃₂ and R₃₃ is not hydrogen; B is

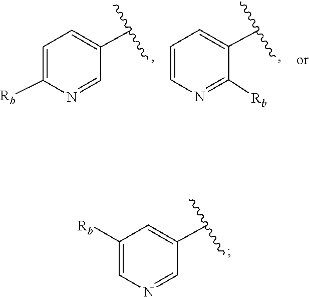

and R_{b} is hydrogen, fluoro or methoxy.

In some embodiments, the disclosure provides a compound of Formula (I-A-1) or a pharmaceutically acceptable salt thereof, wherein R₃₂ is CF₃ and R₃₃ is hydrogen, or R₃₂ is hydrogen and R₃₃ is CF.

In some embodiments, the disclosure provides a compound of Formula (I-A-1) or a pharmaceutically acceptable salt thereof, wherein R₁ is F; R₃ₐ is hydrogen; R₅ₐ is hydrogen; and R₃₂ and R₃₃ are each independently hydrogen, methyl, CF₃, or cyclopropyl, provided that at least one of R₃₂ and R₃₃ is not hydrogen; B is

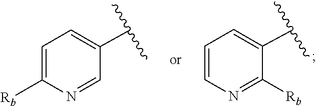

and R_{b} is hydrogen, fluoro or methoxy. In some embodiments, the disclosure provides a compound of Formula (I-A-1) or a pharmaceutically acceptable salt thereof, wherein R₃₂ is CF₃ and R is hydrogen, or R₃₂ is hydrogen and R₃₃ is CF₃.

In some embodiments, the disclosure provides a compound of Formula (I-A-1) or a pharmaceutically acceptable salt thereof, wherein R₁ is F; R₃ₐ is hydrogen; R₅ₐ is hydrogen; and R₃₂ and R₃₃ are each independently hydrogen, methyl, CF₃, or cyclopropyl, provided that at least one of R₃₂ and R₃₃ is not hydrogen; and, in some embodiments, the disclosure provides a compound of Formula (I-A-1) or a pharmaceutically acceptable salt thereof, wherein R₃₂ is CF₃ and R₃₃ is hydrogen, or R₃₂ is hydrogen and R₃₃ is CF₃.

In some embodiments, the disclosure provides a compound of Formula (I-A-1) or a pharmaceutically acceptable salt thereof, wherein B is

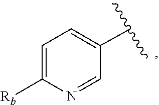

and R_{b} is hydrogen, fluoro or methoxy. In some embodiments, the disclosure provides a compound of Formula (I-A-1) or a pharmaceutically acceptable salt thereof, wherein B is

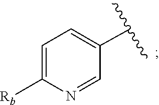

R_{b} is hydrogen, fluoro or methoxy, R₁ is F; R₃ₐ is hydrogen; and R₅ₐ is hydrogen.

In some embodiments, the disclosure provides a compound of Formula (I-A-1) or a pharmaceutically acceptable salt thereof, wherein B is

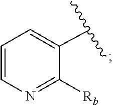

and R_{b} is hydrogen, fluoro or methoxy. In some embodiments, the disclosure provides a compound of Formula (I-A-1) or a pharmaceutically acceptable salt thereof, wherein B is

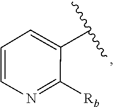

R_{b} is hydrogen, fluoro or methoxy; R₁ is F; R₃ₐ is hydrogen; and R₅ₐ is hydrogen.

In some embodiments, the disclosure provides a compound of Formula (I-A-1) or a pharmaceutically acceptable salt thereof, wherein B is

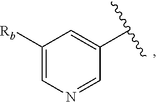

and R_{b} is hydrogen, fluoro or methoxy. In some embodiments, the disclosure provides a compound of Formula (I-A-1) or a pharmaceutically acceptable salt thereof, wherein B is

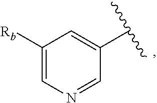

R_{b} is hydrogen, fluoro or methoxy, R₁ is F; R₃ₐ is hydrogen; and R₅ₐ is hydrogen.

In some embodiments, the disclosure provides a compound of Formula (I-A-1) or a pharmaceutically acceptable salt thereof, wherein R_{b} is hydrogen, F or lower alkoxy. In some embodiments, the disclosure provides a compound of Formula (I-A-1) or a pharmaceutically acceptable salt thereof, wherein R_{b} is hydrogen, F or methoxy. In some embodiments, the disclosure provides a compound of Formula (I-A-1) or a pharmaceutically acceptable salt thereof, wherein R_{b} is hydrogen. In some embodiments, the disclosure provides a compound of Formula (I-A-1) or a pharmaceutically acceptable salt thereof, wherein R_{b} is F. In some embodiments, the disclosure provides a compound of Formula (I-A-1) or a pharmaceutically acceptable salt thereof, wherein R_{b} is methoxy.

In some embodiments, the disclosure provides a compound of Formula (I-A-1) or a pharmaceutically acceptable salt thereof, wherein:
R₁ is F;
R₃ₐ is hydrogen;
R₅ₐ is hydrogen;
B is pyridine, optionally substituted with one or more R_{b};
R_{b} is hydrogen, F or methoxy; and
R₃₂ and R₃₃ are each independently hydrogen or —CF₃, provided that only one of R₃₂ and R₃₃ is hydrogen.

In some embodiments, the disclosure provides a compound of Formula (I-A-1) or a pharmaceutically acceptable salt thereof, wherein:
R₁ is F;
R₃ₐ is hydrogen;
R₅ₐ is hydrogen;
B is pyridine, optionally substituted with one R_{b};
R_{b} is hydrogen, F or methoxy; and
R₃₂ is —CF₃ and R₃₃ is hydrogen.

In some embodiments, the disclosure provides a compound of Formula (I-A-1) or a pharmaceutically acceptable salt thereof, wherein:
R₁ is F;
R₃ₐ is hydrogen;
R₅ₐ is hydrogen;
B is pyridine, optionally substituted with one R_{b};
R_{b} is hydrogen, F or methoxy; and
R₃₃ is —CF₃ and R₃₂ is hydrogen.

In some embodiments, the disclosure provides a compound of Formula (I-A-1) or a pharmaceutically acceptable salt thereof, wherein B is 2-pyridinone. In some embodiments, the disclosure provides a compound of Formula (I-A-1) or a pharmaceutically acceptable salt thereof, wherein B is

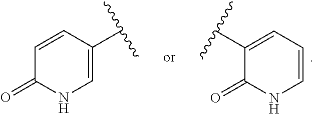 [SMILES: *c1ccc(=O)[nH]c1 ; *c1ccc[nH]c1=O]

In some embodiments, the disclosure provides a compound of Formula (I-A-1) or a pharmaceutically acceptable salt thereof, wherein:
R₁ is F;
R₃ₐ is hydrogen;
R₅ₐ is hydrogen;
B is

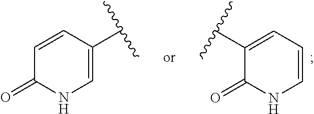 [SMILES: *c1ccc(=O)[nH]c1 ; *c1ccc[nH]c1=O]

and
R₃₂ and R₃₃ are each independently hydrogen or —CF₃, provided that only one of R₃₂ and R₃₃ is hydrogen.

In some embodiments, the disclosure provides a compound of Formula (I-A-1) or a pharmaceutically acceptable salt thereof, wherein:
R₁ is F;
R₃ₐ is hydrogen;
R₅ₐ is hydrogen;
B is

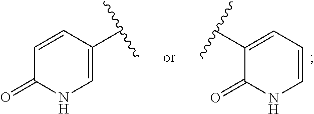 [SMILES: *c1ccc(=O)[nH]c1 ; *c1ccc[nH]c1=O]

and
R₃₂ is —CF₃ and R₃₃ is hydrogen.

In some embodiments, the disclosure provides a compound of Formula (I-A-1) or a pharmaceutically acceptable salt thereof, wherein:
R₁ is F;
R₃ₐ is hydrogen;
R₅ₐ is hydrogen;
B is

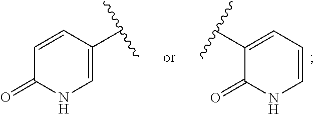 [SMILES: *c1ccc(=O)[nH]c1 ; *c1ccc[nH]c1=O]

and
R₃₃ is —CF₃ and R₃₂ is hydrogen.

In some embodiments, the compound is a compound of Formula (I-A-1) as provided in Table A below.

TABLE A
Compounds of Formula (I).

| Compound | R₁ | R₃ₐ | R₅ₐ | B | R₃₃ | R₃₂ |
|---|---|---|---|---|---|---|
| 1 | F | H | H | 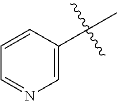 [SMILES: *c1cccnc1] | CF₃ | H |
| 3 | F | H | H | 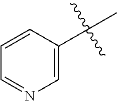 [SMILES: *c1cccnc1] | H | CF₃ |
| 5 | F | H | H | 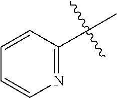 [SMILES: *c1ccccn1] | H | CF₃ |
| 7 | F | H | H | 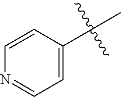 [SMILES: *c1ccncc1] | H | CF₃ |
| 9 | F | H | H | 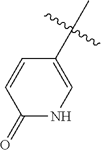 [SMILES: *c1ccc(=O)[nH]c1] | H | CF₃ |
| 11 | F | H | H | 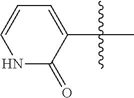 [SMILES: *c1ccc[nH]c1=O] | H | CF₃ |
| 13 | F | H | H | 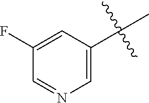 [SMILES: *c1cncc(F)c1] | H | CF₃ |
| 16 | F | H | H | 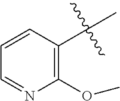 [SMILES: *c1cccnc1OC] | H | CF₃ |
| 18 | F | H | H | 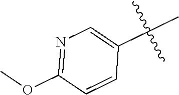 [SMILES: *c1ccc(OC)nc1] | H | CF₃ |
| 19 | F | H | H | 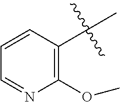 [SMILES: *c1cccnc1OC] | CF₃ | H |
| 21 | F | H | H | 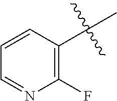 [SMILES: *c1cccnc1F] | CF₃ | H |
| 23 | F | H | H | 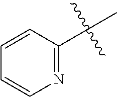 [SMILES: *c1ccccn1] | CF₃ | H |
| 25 | F | H | H | 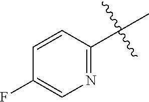 [SMILES: *c1ccc(F)cn1] | CF₃ | H |
| 27 | F | H | H | 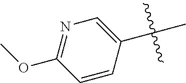 [SMILES: *c1ccc(OC)nc1] | CF₃ | H |
| 29 | F | H | H | 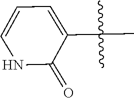 [SMILES: *c1ccc[nH]c1=O] | CF₃ | H |
| 31 | F | H | H | 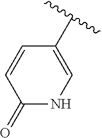 [SMILES: *c1ccc(=O)[nH]c1] | CF₃ | H |

In some embodiments, the disclosure provides a compound of Formula (I-B-1) or a pharmaceutically acceptable salt thereof.

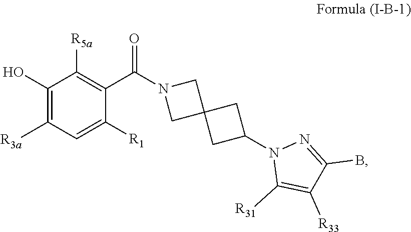

wherein:
   R₁ is halogen or cyano;
   R₃ₐ is hydrogen, optionally substituted lower alkyl, or halogen;
   R₅ₐ is hydrogen, halogen or optionally substituted lower alkyl;
   B is pyridine, pyrimidine, or pyridinone, each independently optionally substituted with one or more R_{b};
   R_{b} is hydrogen, halogen or alkoxy; and
   R₃₁ and R₃₃ are each independently hydrogen, optionally substituted lower alkyl, optionally substituted lower haloalkyl, or optionally substituted lower cycloalkyl, provided that at least one of R₃₁ and R₃₃ is not hydrogen.

In some embodiments, the disclosure provides a compound of Formula (I-B-1) or a pharmaceutically acceptable salt thereof, wherein:
R₁ is halogen or cyano;
R₃ₐ is hydrogen, lower alkyl, or halogen;
R₅ₐ is hydrogen, halogen or lower alkyl;
B is pyridine or pyrimidine, each independently optionally substituted with one or more R_{b};
R_{b} is hydrogen, halogen or alkoxy; and
R₃₁ and R₃₃ are each independently hydrogen, lower alkyl, lower haloalkyl, or lower cycloalkyl, provided that at least one of R₃₁ and R₃₃ is not hydrogen.

In some embodiments, the disclosure provides a compound of Formula (I-B-1) or a pharmaceutically acceptable salt thereof, wherein:
R₁ is halogen or cyano;
R₃ₐ is hydrogen, lower alkyl, or halogen;
R₅ₐ is hydrogen, halogen or lower alkyl;
B is pyridine optionally substituted with one or more R_{b};
R_{b} is hydrogen, halogen or alkoxy; and
R₃₁ and R₃₃ are each independently hydrogen, lower alkyl, lower haloalkyl, or lower cycloalkyl, provided that at least one of R₃₁ and R₃₃ is not hydrogen.

In some embodiments, the disclosure provides a compound of Formula (I-B-1) or a pharmaceutically acceptable salt thereof, wherein R₁ is F; R₃ₐ is hydrogen; and R₅ₐ is hydrogen. In some embodiments, the disclosure provides a compound of Formula (I-B-1) or a pharmaceutically acceptable salt thereof, wherein R₃₁ and R₃₃ are each independently hydrogen, methyl optionally substituted with one or more F, or cyclopropyl, provided that at least one of R₃₁ and R₃₃ is not hydrogen, R₁ is F; R₃ₐ is hydrogen; and R₅ₐ is hydrogen.

In some embodiments, the disclosure provides a compound of Formula (I-B-1) or a pharmaceutically acceptable salt thereof, wherein R₃₁ and R₃₃ are each independently hydrogen, lower alkyl, lower haloalkyl, or lower cycloalkyl, provided that at least one of R₃₁ and R₃₃ is not hydrogen. In some embodiments, the disclosure provides a compound of Formula (I-B-1) or a pharmaceutically acceptable salt thereof, wherein R₁ is F; R₃ₐ is hydrogen; R₅ₐ is hydrogen; and R₃₁ and R₃₃ are each independently hydrogen, methyl, CF₃, or cyclopropyl, provided that at least one of R₃₁ and R₃₃ is not hydrogen. In some embodiments, the disclosure provides a compound of Formula (I-B-1) or a pharmaceutically acceptable salt thereof, wherein R₃₁ is CF₃ and R₃₃ is hydrogen, or R₃₁ is hydrogen and R₃₃ is CF₃.

In some embodiments, the disclosure provides a compound of Formula (I-B-1) or a pharmaceutically acceptable salt thereof, wherein
B is

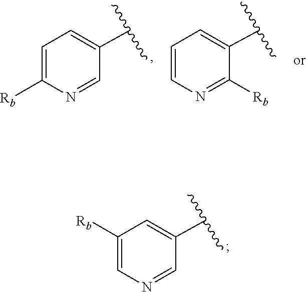

R_{b} is hydrogen, fluoro or methoxy; and
R₃₁ and R₃₃ are each independently hydrogen, lower alkyl, lower haloalkyl, or lower cycloalkyl, provided that at least one of R₃₁ and R₃₃ is not hydrogen.

In some embodiments, the disclosure provides a compound of Formula (I-B-1) or a pharmaceutically acceptable salt thereof, wherein R₁ is F; R₃ₐ is hydrogen; R₅ₐ is hydrogen; and R₃₁ and R₃₃ are each independently hydrogen, methyl, CF₃, or cyclopropyl, provided that at least one of R₃₁ and R₃₃ is not hydrogen; B is

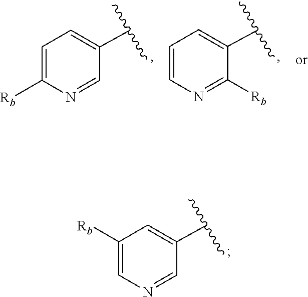

and R_{b} is hydrogen, fluoro or methoxy. In some embodiments, the disclosure provides a compound of Formula (I-B-1) or a pharmaceutically acceptable salt thereof, wherein R₃₁ is CF₃ and R₃₃ is hydrogen, or R₃₁ is hydrogen and R₃₃ is CF₃. In some embodiments, the disclosure provides a compound of Formula (I-B-1) or a pharmaceutically acceptable salt thereof, wherein R₁ is F; R₃ₐ is hydrogen; R₁ is hydrogen; and R₃₁ and R₃₃ are each independently hydrogen, methyl, CF₃, or cyclopropyl, provided that at least one of R₃₁ and R₃₃ is not hydrogen; B is

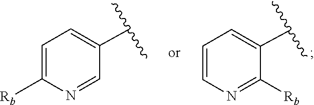

and R_{b} is hydrogen, fluoro or methoxy. In some embodiments, the disclosure provides a compound of Formula (I-B-1) or a pharmaceutically acceptable salt thereof, wherein R₃₁ is CF₃ and R₃₃ is hydrogen, or R₃₁ is hydrogen and R₃₃ is CF₃.

In some embodiments, the disclosure provides a compound of Formula (I-B-1) or a pharmaceutically acceptable salt thereof, wherein R₁ is F; R₃ₐ is hydrogen; R₅ₐ is hydrogen; and R₃₁ and R₃₃ are each independently hydrogen, methyl, CF₃, or cyclopropyl, provided that at least one of R₃₁ and R₃₃ is not hydrogen; and, in some embodiments, the disclosure provides a compound of Formula (I-B-1) or a pharmaceutically acceptable salt thereof, wherein R₃₁ is CF₃ and R₃₃ is hydrogen, or R₃₁ is hydrogen and R₃₃ is CF₃.

In some embodiments, the disclosure provides a compound of Formula (I-B-1) or a pharmaceutically acceptable salt thereof, wherein B is

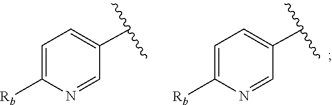

and R_{b} is hydrogen, fluoro or methoxy. In some embodiments, the disclosure provides a compound of Formula (I-B-1) or a pharmaceutically acceptable salt thereof, wherein B is

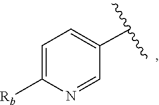

R_{b} is hydrogen, fluoro or methoxy, R₁ is F; R₃ₐ is hydrogen; and R₅ₐ is hydrogen.

In some embodiments, the disclosure provides a compound of Formula (I-B-1) or a pharmaceutically acceptable salt thereof, wherein B is

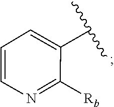

and R_{b} is hydrogen, fluoro or methoxy. In some embodiments, the disclosure provides a compound of Formula (I-B-1) or a pharmaceutically acceptable salt thereof, wherein B is

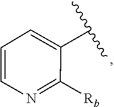

R_{b} is hydrogen, fluoro or methoxy R₁ is F; R₃ₐ is hydrogen; and R₅ₐ is hydrogen.

In some embodiments, the disclosure provides a compound of Formula (I-B-1) or a pharmaceutically acceptable salt thereof, wherein B is

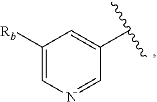

and R_{b} is hydrogen, fluoro or methoxy. In some embodiments, the disclosure provides a compound of Formula (I-B-1) or a pharmaceutically acceptable salt thereof, wherein B is

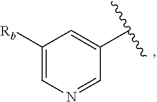

R_{b} is hydrogen, fluoro or methoxy, R₁ is F; R₃ₐ is hydrogen; and R₅ₐ is hydrogen.

In some embodiments, the disclosure provides a compound of Formula (I-B-1) or a pharmaceutically acceptable salt thereof, wherein R_{b} is hydrogen, F or lower alkoxy. In some embodiments, the disclosure provides a compound of Formula (I-B-1) or a pharmaceutically acceptable salt thereof, wherein R_{b} is hydrogen, F or methoxy. In some embodiments, the disclosure provides a compound of Formula (I-B-1) or a pharmaceutically acceptable salt thereof, wherein R_{b} is hydrogen. In some embodiments, the disclosure provides a compound of Formula (I-B-1) or a pharmaceutically acceptable salt thereof, wherein R_{b} is F. In some embodiments, the disclosure provides a compound of Formula (I-B-1) or a pharmaceutically acceptable salt thereof, wherein R_{b} is methoxy.

In some embodiments, the disclosure provides a compound of Formula (I-B-1) or a pharmaceutically acceptable salt thereof, wherein:
R₁ is F;
R₃ₐ is hydrogen;
R₅ₐ is hydrogen;
B is pyridine, optionally substituted with one or more R_{b};
R_{b} is hydrogen, F or methoxy; and
R₃₁ and R₃₃ are each independently hydrogen or —CF₃, provided that only one of R₃₁ and R₃₃ is hydrogen.

In some embodiments, the disclosure provides a compound of Formula (I-B-1) or a pharmaceutically acceptable salt thereof, wherein:
R₁ is F;
R₃ₐ is hydrogen;
R₅ₐ is hydrogen;
B is pyridine, optionally substituted with one R_{b};
R_{b} is hydrogen, F or methoxy; and
R₃₁ is —CF₃ and R₃₃ is hydrogen.

In some embodiments, the disclosure provides a compound of Formula (I-B-1) or a pharmaceutically acceptable salt thereof, wherein:
R₁ is F;
R₃ₐ is hydrogen;
R₅ₐ is hydrogen;
B is pyridine, optionally substituted with one R_{b};
R_{b} is hydrogen, F or methoxy; and
R₃₃ is —CF₃ and R₃₁ is hydrogen.

In some embodiments, the disclosure provides a compound of Formula (I-B-1) or a pharmaceutically acceptable salt thereof, wherein B is 2-pyridinone. In some embodiments, the disclosure provides a compound of Formula (I-B-1) or a pharmaceutically acceptable salt thereof, wherein B is

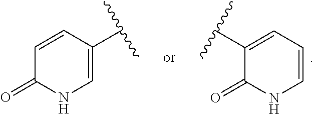 [SMILES: *c1ccc(=O)[nH]c1 ; *c1ccc[nH]c1=O]

In some embodiments, the disclosure provides a compound of Formula (I-B-1) or a pharmaceutically acceptable salt thereof, wherein:
R₁ is F;
R₃ₐ is hydrogen;
R₅ₐ is hydrogen;
B is

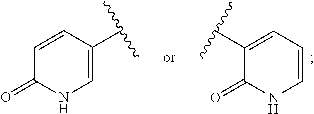 [SMILES: *c1ccc(=O)[nH]c1 ; *c1ccc[nH]c1=O]

and
R₃₁ and R₃₃ are each independently hydrogen or —CF₃, provided that only one of R₃₁ and R₃₃ is hydrogen.

In some embodiments, the disclosure provides a compound of Formula (I-B-1) or a pharmaceutically acceptable salt thereof, wherein:
R₁ is F;
R₃ₐ is hydrogen;
R₅ₐ is hydrogen;
B is

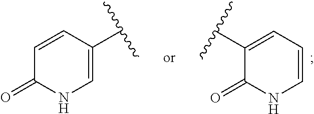 [SMILES: *c1ccc(=O)[nH]c1 ; *c1ccc[nH]c1=O]

and
R₃₁ is —CF₃ and R₃₃ is hydrogen.

In some embodiments, the disclosure provides a compound of Formula (I-B-1) or a pharmaceutically acceptable salt thereof, wherein.
R₁ is F;
R₃ₐ is hydrogen;
R_{5b} is hydrogen;
B is

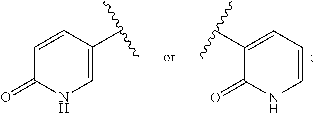 [SMILES: *c1ccc(=O)[nH]c1 ; *c1ccc[nH]c1=O]

and
R₃₃ is —CF₃ and R₃₁ is hydrogen.

In some embodiments, the compound is a compound of Formula (I-B-1) as provided in Table B below.

TABLE B
Compounds of Formula (I-B-1).

| Compound | R₁ | R₃ₐ | R₅ₐ | B | R₃₃ | R₃₁ |
|---|---|---|---|---|---|---|
| 2 | F | H | H | 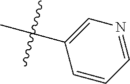 [SMILES: *c1cccnc1] | CF₃ | H |
| 4 | F | H | H | 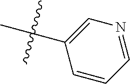 [SMILES: *c1cccnc1] | H | CF₃ |
| 6 | F | H | H | 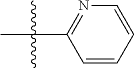 [SMILES: *c1ccccn1] | H | CF₃ |
| 8 | F | H | H | 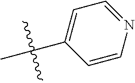 [SMILES: *c1ccncc1] | H | CF₃ |
| 10 | F | H | H | 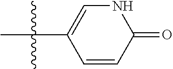 [SMILES: *c1ccc(=O)[nH]c1] | H | CF₃ |
| 12 | F | H | H | 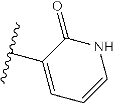 [SMILES: *c1ccc[nH]c1=O] | H | CF₃ |
| 14 | F | H | H | 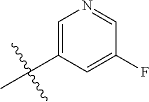 [SMILES: *c1cncc(F)c1] | H | CF₃ |
| 15 | F | H | H | 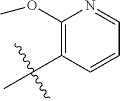 [SMILES: *c1cccnc1OC] | H | CF₃ |
| 17 | F | H | H | 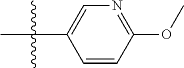 [SMILES: *c1ccc(OC)nc1] | H | CF₃ |
| 20 | F | H | H | 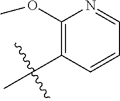 [SMILES: *c1cccnc1OC] | CF₃ | H |
| 22 | F | H | H | 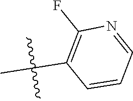 [SMILES: *c1cccnc1F] | CF₃ | H |
| 24 | F | H | H | 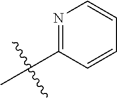 [SMILES: *c1ccccn1] | CF₃ | H |
| 26 | F | H | H | 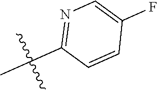 [SMILES: *c1ccc(F)cn1] | CF₃ | H |
| 28 | F | H | H | 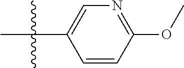 [SMILES: *c1ccc(OC)nc1] | CF₃ | H |
| 30 | F | H | H | 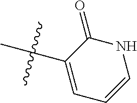 [SMILES: *c1ccc[nH]c1=O] | CF₃ | H |
| 32 | F | H | H | 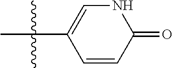 [SMILES: *c1ccc(=O)[nH]c1] | CF₃ | H |

In some embodiments, the disclosure provides a compound of Formula (II) or a pharmaceutically acceptable salt thereof:

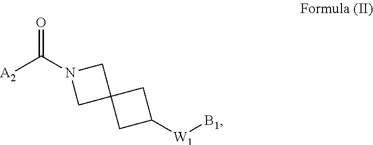

wherein:
A₂ is

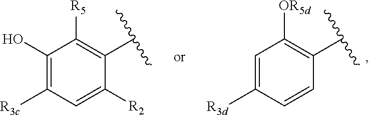

R₂ is hydrogen, halogen, or cyano;
R_{3c} and R_{5c} are each independently hydrogen, halogen, or optionally substituted lower alkyl;
R_{3d} is halogen;
R_{5d} is optionally substituted lower alkyl, optionally substituted lower cycloalkyl, or optionally substituted lower haloalkyl;
W₁ is

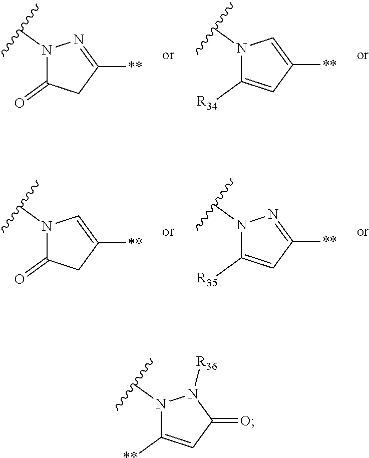

** is the bond to B₁:
R₃₄ is lower alkoxy, lower alkyl, lower haloalkyl or lower cycloalkyl;
R₃₅ is lower alkoxy, lower alkyl, lower haloalkyl or lower cycloalkyl;
R₃₆ is hydrogen or lower alkyl; and
   B₁ is phenyl optionally substituted with halogen, optionally substituted lower alkoxy, optionally substituted lower alkyl, or optionally substituted lower haloalkyl.

In some embodiments, the disclosure provides a compound of Formula (II) or a pharmaceutically acceptable salt thereof, wherein A₂ is

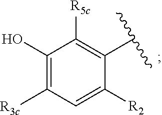

R_{3c} and R_{5c} are each independently hydrogen, halogen, or optionally substituted lower alkyl; and R₂ is hydrogen, halogen, or cyano. In some embodiments, the disclosure provides a compound of Formula (II) or a pharmaceutically acceptable salt thereof, wherein A₂ is

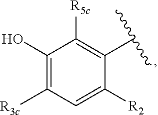

R_{3c} and R_{5c} are each independently hydrogen, halogen, or optionally substituted lower alkyl; and R₂ is F. In some embodiments, the disclosure provides a compound of Formula (II) or a pharmaceutically acceptable salt thereof, wherein A₂ is

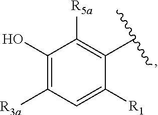

R_{3c} is hydrogen, R_{5c} is hydrogen, and R₂ is F.

In some embodiments, the disclosure provides a compound of Formula (II) or a pharmaceutically acceptable salt thereof, wherein A₂ is

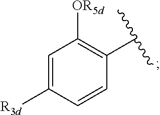

R_{5d} is optionally substituted lower alkyl, optionally substituted lower cycloalkyl, or optionally substituted lower haloalkyl; and R_{3d} is halogen. In some embodiments, the disclosure provides a compound of Formula (II) or a pharmaceutically acceptable salt thereof, wherein A₂ is

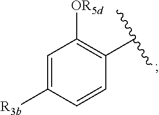

R_{5d} is optionally substituted lower alkyl, optionally substituted lower cycloalkyl, or optionally substituted lower haloalkyl; and R_{3d} is F. In some embodiments, the disclosure provides a compound of Formula (II) or a pharmaceutically acceptable salt thereof, wherein A₂ is

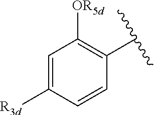

R_{3d} is halogen; and R_{5d} is cyclopropyl. In some embodiments, the disclosure provides a compound of Formula (II) or a pharmaceutically acceptable salt thereof, wherein A₂ is

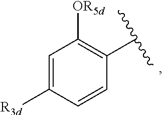

R_{3d} is F and R_{5d} is lower cycloalkyl. In some embodiments, the disclosure provides a compound of Formula (II) or a pharmaceutically acceptable salt thereof, wherein A₂ is

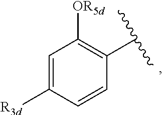

R_{3d} is F and R_{5d} is cyclopropyl.

In some embodiments, the disclosure provides a compound of Formula (II) or a pharmaceutically acceptable salt thereof, wherein R_{3d} is F; and R_{5d} is methyl or cyclopropyl. In some embodiments, the disclosure provides a compound of Formula (II) or a pharmaceutically acceptable salt thereof, wherein R_{5d} is cyclopropyl.

In some embodiments, the disclosure provides a compound of Formula (II) or a pharmaceutically acceptable salt thereof, wherein R₃₄ is methoxy. In some embodiments, the disclosure provides a compound of Formula (II) or a pharmaceutically acceptable salt thereof, wherein W₁ is

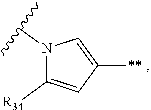

** is the bond to B₁ and R₃₄ is methoxy.

In some embodiments, the disclosure provides a compound of Formula (II) or a pharmaceutically acceptable salt thereof, wherein W₁ is

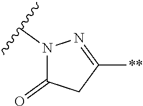

and ** is the bond to B₁.

In some embodiments, the disclosure provides a compound of Formula (II) or a pharmaceutically acceptable salt thereof, wherein W₁ is

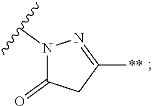

and ** is the bond to B₁.

In some embodiments, the disclosure provides a compound of Formula (II) or a pharmaceutically acceptable salt thereof, wherein W₁ is

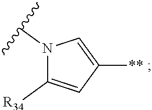

and R₃₄ is lower alkoxy, lower alkyl, lower haloalkyl or lower cycloalkyl; and ** is the bond to B₁. In some embodiments, the disclosure provides a compound of Formula (II) or a pharmaceutically acceptable salt thereof, wherein W₁ is

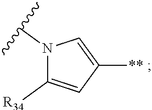

and R₃₄ is methoxy, lower alkyl, —CF₃ or cyclopropyl; and ** is the bond to B₁. In some embodiments, the disclosure provides a compound of Formula (II) or a pharmaceutically acceptable salt thereof, wherein W₁ is

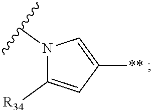

** is the bond to B₁; and R₃₄ is methoxy.

In some embodiments, the disclosure provides a compound of Formula (II) or a pharmaceutically acceptable salt thereof, wherein W₁ is

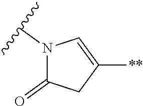

and ** is the bond to B₁.

In some embodiments, the disclosure provides a compound of Formula (II) or a pharmaceutically acceptable salt thereof, wherein W₁ is

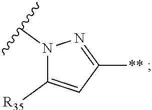

R₃₅ is lower alkoxy, lower alkyl, lower haloalkyl or lower cycloalkyl; and ** is the bond to B₁. In some embodiments, the disclosure provides a compound of Formula (II) or a pharmaceutically acceptable salt thereof, wherein W₁ is

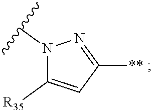

R₃₅ is methoxy; and ** is the bond to B₁.

In some embodiments, the disclosure provides a compound of Formula (II) or a pharmaceutically acceptable salt thereof, wherein W₁ is

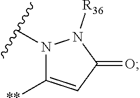

R₃₆ is hydrogen or lower alkyl; and ** is the bond to B₁. In some embodiments, the disclosure provides a compound of Formula (II) or a pharmaceutically acceptable salt thereof, wherein W₁ is

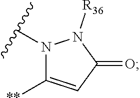

R₃₆ is hydrogen or methyl; and ** is the bond to B₁.

In some embodiments, the disclosure provides a compound of Formula (II) or a pharmaceutically acceptable salt thereof, wherein B₁ is

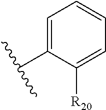

and R₂₀ is hydrogen, halogen, optionally substituted lower alkoxy, optionally substituted lower alkyl, or optionally substituted lower haloalkyl. In some embodiments, the disclosure provides a compound of Formula (II) or a pharmaceutically acceptable salt thereof, wherein B₁ is

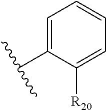

and R₂₀ is hydrogen, F, methoxy, methyl, or —CF₃. In some embodiments, the disclosure provides a compound of Formula (II) or a pharmaceutically acceptable salt thereof, wherein B₁ is

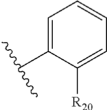

R₂₀ is F. In some embodiments, the disclosure provides a compound of Formula (II) or a pharmaceutically acceptable salt thereof, wherein B₁ is

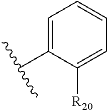

and R₂₀ is —CF₃.

In some embodiments, the disclosure provides a compound of Formula (II) or a pharmaceutically acceptable salt thereof, wherein:
A₂ is

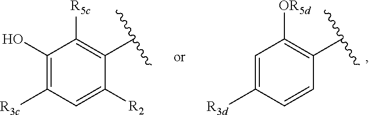

R₂ is F;
R_{3c} and R_{5c} are each independently hydrogen;
R_{3d} is F;
R_{5d} is optionally substituted lower alkyl, optionally substituted lower cycloalkyl, or optionally substituted lower haloalkyl;
   W₁ is

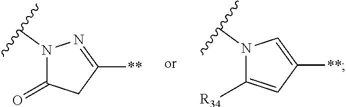

** is the bond to B₁;
   R₃₄ is lower alkoxy, lower alkyl, lower haloalkyl or lower cycloalkyl; and B₁ is phenyl optionally substituted with halogen, optionally substituted lower alkoxy, optionally substituted lower alkyl, or optionally substituted lower haloalkyl.

In some embodiments, the disclosure provides a compound of Formula (II) or a pharmaceutically acceptable salt thereof, wherein:
A₂ is

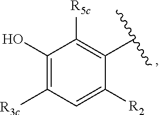

R₂ is F;
R_{3c} and R_{5c} are each independently hydrogen;
   W₁ is

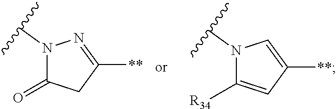

** is the bond to B₁;
R₃₄ is methoxy; and
B₁ is phenyl optionally substituted with halogen, optionally substituted lower alkoxy, optionally substituted lower alkyl, or optionally substituted lower haloalkyl.

In some embodiments, the disclosure provides a compound of Formula (II-A-1) or a pharmaceutically acceptable salt thereof:

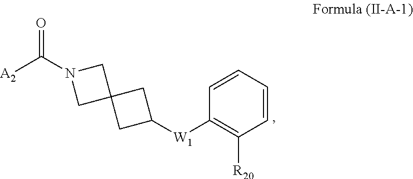

wherein:
A₂ is

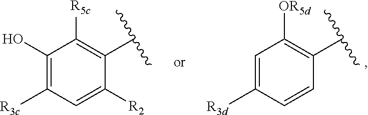

R₂ is hydrogen, halogen, or cyano;
R_{3c} and R_{5c} are each independently hydrogen, halogen, or lower alkyl;
R_{3d} is halogen,
R_{5d} is lower alkyl, lower cycloalkyl, or lower haloalkyl;
W₁ is

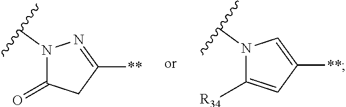

** is the bond to B₁;
R₃₄ is lower alkoxy; and
R₂₀ is hydrogen, halogen, lower alkyl or lower haloalkyl.

In some embodiments, the compound is a compound of Formula (II-A-1) as provided in Table C below.

TABLE C
Compounds of Formula (II-A-1).

| Compound | R_{5d} | R₂₀ | R₃₄ |
|---|---|---|---|
| 34 | 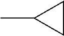 | F | OMe |
| 36 | 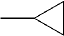 | F | |
| 38 | 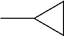 | CF₃ | OMe |
| 40 | 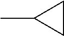 | CF₃ | |

In some embodiments, the disclosure provides a compound, wherein the compound is

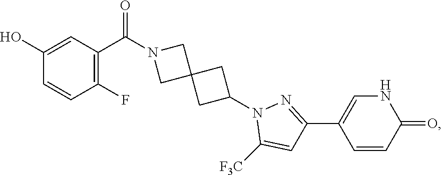 [SMILES: O=C(c1cc(O)ccc1F)N1CC2(CC(n3nc(-c4ccc(=O)[nH]c4)cc3C(F)(F)F)C2)C1]

or a pharmaceutically acceptable salt thereof.

In some embodiments, the disclosure provides a compound, wherein the compound is

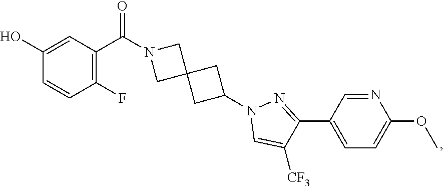 [SMILES: COc1ccc(-c2nn(C3CC4(C3)CN(C(=O)c3cc(O)ccc3F)C4)cc2C(F)(F)F)cn1]

or a pharmaceutically acceptable salt thereof.

In some embodiments, the disclosure provides a compound, wherein the compound is

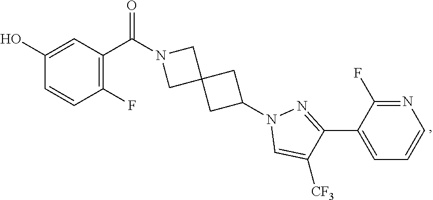 [SMILES: O=C(c1cc(O)ccc1F)N1CC2(CC(n3cc(C(F)(F)F)c(-c4cccnc4F)n3)C2)C1]

or a pharmaceutically acceptable salt thereof.

In some embodiments, the disclosure provides a compound, wherein the compound is

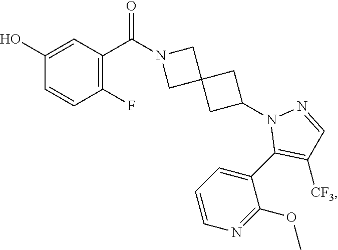 [SMILES: COc1ncccc1-c1c(C(F)(F)F)cnn1C1CC2(C1)CN(C(=O)c1cc(O)ccc1F)C2]

or a pharmaceutically acceptable salt thereof.

In some embodiments, the disclosure provides a compound, wherein the compound is

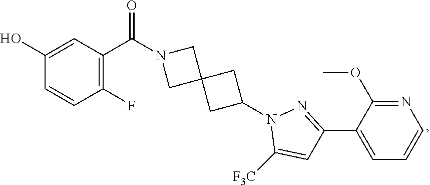 [SMILES: COc1ncccc1-c1cc(C(F)(F)F)n(C2CC3(C2)CN(C(=O)c2cc(O)ccc2F)C3)n1]

or a pharmaceutically acceptable salt thereof.

In some embodiments, the disclosure provides a compound, wherein the compound is

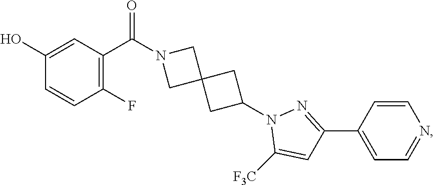 [SMILES: O=C(c1cc(O)ccc1F)N1CC2(CC(n3nc(-c4ccncc4)cc3C(F)(F)F)C2)C1]

or a pharmaceutically acceptable salt thereof.

In some embodiments, the disclosure provides a compound, wherein the compound is

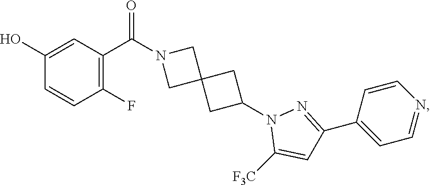 [SMILES: O=C(c1cc(O)ccc1F)N1CC2(CC(n3nc(-c4ccncc4)cc3C(F)(F)F)C2)C1]

or a pharmaceutically acceptable salt thereof.

In some embodiments, the disclosure provides a compound, wherein the compound is

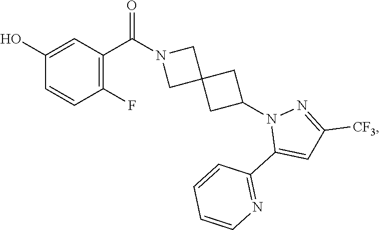 [SMILES: O=C(c1cc(O)ccc1F)N1CC2(CC(n3nc(C(F)(F)F)cc3-c3ccccn3)C2)C1]

or a pharmaceutically acceptable salt thereof.

In some embodiments, the disclosure provides a compound, wherein the compound is

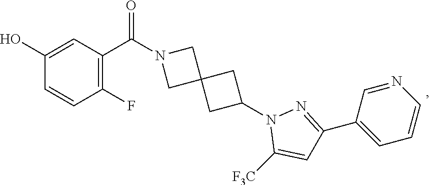 [SMILES: O=C(c1cc(O)ccc1F)N1CC2(CC(n3nc(-c4cccnc4)cc3C(F)(F)F)C2)C1]

or a pharmaceutically acceptable salt thereof.

In some embodiments, the disclosure provides a compound, wherein the compound is

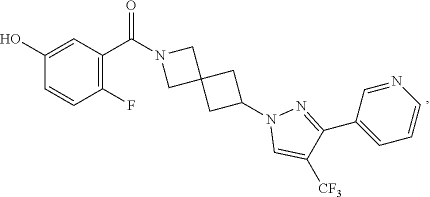 [SMILES: O=C(c1cc(O)ccc1F)N1CC2(CC(n3cc(C(F)(F)F)c(-c4cccnc4)n3)C2)C1]

or a pharmaceutically acceptable salt thereof.

In some embodiments, the disclosure provides a compound, wherein the compound is a compound selected from the group consisting of
a. (2-fluoro-5-hydroxyphenyl){6-[5-(2-pyridyl)-3-(trifluoromethyl)-1-pyrazolyl]-2-aza-2-spiro[3.3]heptyl}methanone (Compound 5);
b. 5-(1-{2-(2-fluoro-5-hydroxybenzoyl)-2-aza-6-spiro[3.3]heptyl}-5-(trifluoromethyl)-3-pyrazolyl)-2(1H)-pyridinone (Compound 10);
c. 3-(1-{2-(2-fluoro-5-hydroxybenzoyl)-2-aza-6-spiro[3.3]heptyl}-5-(trifluoromethyl)-3-pyrazolyl)-2(1H)-pyridinone (Compound 12);
d. (2-fluoro-5-hydroxyphenyl){6-[3-(2-methoxy-3-pyridyl)-5-(trifluoromethyl)-1-pyrazolyl]-2-aza-2-spiro[3.3]heptyl}methanone (Compound 15); or
e. (2-fluoro-5-hydroxyphenyl){6-[3-(6-methoxy-3-pyridyl)-5-(trifluoromethyl)-1-pyrazolyl]-2-aza-2-spiro[3.3]heptyl}methanone (Compound 17); or a pharmaceutically acceptable salt thereof.

In some embodiments, the disclosure provides the compound (2-fluoro-5-hydroxyphenyl){6-[5-(2-pyridyl)-3-(trifluoromethyl)-1-pyrazolyl]-2-aza-2-spiro[3.3]heptyl}methanone (Compound 5), or a pharmaceutically acceptable salt thereof.

In some embodiments, the disclosure provides the compound 5-(1-{2-(2-fluoro-5-hydroxybenzoyl)-2-aza-6-spiro[3.3]heptyl}-5-(trifluoromethyl)-3-pyrazolyl)-2(1H)-pyridinone (Compound 10), or a pharmaceutically acceptable salt thereof.

In some embodiments, the disclosure provides the compound 3-(1-{2-(2-fluoro-5-hydroxybenzoyl)-2-aza-6-spiro[3.3]heptyl}-5-(trifluoromethyl)-3-pyrazolyl)-2(1H)-pyridinone (Compound 12), or a pharmaceutically acceptable salt thereof.

In some embodiments, the disclosure provides the compound (2-fluoro-5-hydroxyphenyl){6-[3-(2-methoxy-3-pyridyl)-5-(trifluoromethyl)-1-pyrazolyl]-2-aza-2-spiro[3.3]heptyl}methanone (Compound 15), or a pharmaceutically acceptable salt thereof.

In some embodiments, the disclosure provides the compound (2-fluoro-5-hydroxyphenyl){6-[3-(6-methoxy-3-pyridyl)-5-(trifluoromethyl)-1-pyrazolyl]-2-aza-2-spiro[3.3]heptyl}methanone (Compound 17), or a pharmaceutically acceptable salt thereof.

In some embodiments, the disclosure provides a compound, wherein the compound is a compound selected from the group consisting of.
a. (2-fluoro-5-hydroxyphenyl){6-[3-(3-pyridyl)-4-(trifluoromethyl)-1-pyrazolyl]-2-aza-2-spiro[3.3]heptyl}methanone (Compound 2);
b. (2-fluoro-5-hydroxyphenyl){6-[3-(3-pyridyl)-5-(trifluoromethyl)-1-pyrazolyl]-2-aza-2-spiro[3.3]heptyl}methanone (Compound 4);
c. (2-fluoro-5-hydroxyphenyl){6-[5-(2-pyridyl)-3-(trifluoromethyl)-1-pyrazolyl]-2-aza-2-spiro[3.3]heptyl}methanone (Compound 5);
d. (2-fluoro-5-hydroxyphenyl){6-[3-(4-pyridyl)-5-(trifluoromethyl)-1-pyrazolyl]-2-aza-2-spiro[3.3]heptyl}methanone (Compound 8):
e. 5-(1-{2-(2-fluoro-5-hydroxybenzoyl)-2-aza-6-spiro[3.3]heptyl}-5-(trifluoromethyl)-3-pyrazolyl)-2(1H)-pyridinone (Compound 10);
f. (2-fluoro-5-hydroxyphenyl){6-[3-(2-methoxy-3-pyridyl)-5-(trifluoromethyl)-1-pyrazolyl]-2-aza-2-spiro[3.3]heptyl}methanone (Compound 15);
g. (2-fluoro-5-hydroxyphenyl){6-[3-(6-methoxy-3-pyridyl)-5-(trifluoromethyl)-1-pyrazolyl]-2-aza-2-spiro[3.3]heptyl}methanone (Compound 17);
h. (2-fluoro-5-hydroxyphenyl){6-[5-(2-methoxy-3-pyridyl)-4-(trifluoromethyl)-1-pyrazolyl]-2-aza-2-spiro[3.3]heptyl}methanone (Compound 19);
i. (2-fluoro-5-hydroxyphenyl){6-[3-(2-fluoro-3-pyridyl)-4-(trifluoromethyl)-1-pyrazolyl]-2-aza-2-spiro[3.3]heptyl}methanone (Compound 22); or
j. (2-fluoro-5-hydroxyphenyl){6-[3-(6-methoxy-3-pyridyl)-4-(trifluoromethyl)-1-pyrazolyl]-2-aza-2-spiro[3.3]heptyl}methanone (Compound 28); or a pharmaceutically acceptable salt thereof.

In some embodiments, the disclosure provides a compound, wherein the compound is selected from the group consisting of;
a. (2-cyclopropoxy-4-fluorophenyl){6-[4-(o-fluorophenyl)-2-methoxy-1-pyrrolyl]-2-aza-2-spiro[3.3]heptyl}methanone (Compound 34);
b. (2-cyclopropoxy-4-fluorophenyl){6-[3-(o-fluorophenyl)-5-methoxy-1-pyrazolyl]-2-aza-2-spiro[3.3]heptyl}methanone (Compound 35);
c. 1-[2-(2-cyclopropoxy-4-fluorobenzoyl)-2-azaspiro[3.3]heptan-6-yl]-3-(2-fluorophenyl)-4,5-dihydro-1H-pyrazol-5-one (Compound 36);
d. 1-{2-(2-cyclopropoxy-4-fluorobenzoyl)-2-aza-6-spiro[3.3]heptyl}-3-[o-(trifluoromethyl)phenyl]-2-pyrrolin-5-one (Compound 37),
e. (2-cyclopropoxy-4-fluorophenyl)(6-{2-methoxy-4-[o-(trifluoromethyl)phenyl]-1-pyrrolyl}-2-aza-2-spiro[3.3]heptyl)methanone (Compound 38);
f. (2-cyclopropoxy-4-fluorophenyl)(6-{5-methoxy-3-[o-(trifluoromethyl)phenyl]-1-pyrazolyl}-2-aza-2-spiro[3.3]heptyl)methanone (Compound 39);
g. 1-[2-(2-cyclopropoxy-4-fluorobenzoyl)-2-azaspiro[3.3]heptan-6-yl]-3-[2-(trifluoromethyl)phenyl]-4,5-dihydro-1H-pyrazol-5-one (Compound 40);
h. 1-(2-(2-cyclopropoxy-4-fluorobenzoyl)-2-aza-6-spiro[3.3]heptyl)-5-(o-fluorophenyl)-1,2-dihydro-3H-pyrazol-3-one (Compound 42); and
i. 1-{2-(2-cyclopropoxy-4-fluorobenzoyl)-2-aza-6-spiro[3.3]heptyl}-5-(o-fluorophenyl)-2-methyl-1,2-dihydro-3H-pyrazol-3-one (Compound 43); or a pharmaceutically acceptable salt thereof.

In some embodiments, the disclosure provides a compound, wherein the compound is selected from the group consisting of:

| Compound | Structure | IUPAC Name |
|---|---|---|
| 23 | 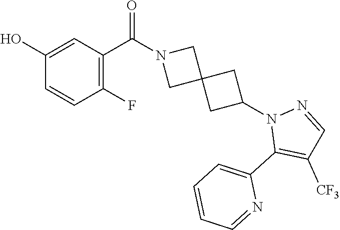 [SMILES: O=C(c1cc(O)ccc1F)N1CC2(CC(n3ncc(C(F)(F)F)c3-c3ccccn3)C2)C1] | (2-fluoro-5- hydroxyphenyl){6-[5-(2- pyridyl)-4-(trifluoromethyl)- 1-pyrazolyl]-2-aza-2- spiro[3.3]heptyl}methanone |
| 24 | 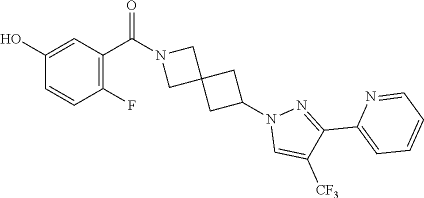 [SMILES: O=C(c1cc(O)ccc1F)N1CC2(CC(n3cc(C(F)(F)F)c(-c4ccccn4)n3)C2)C1] | (2-fluoro-5- hydroxyphenyl){6-[3-(2- pyridyl)-4-(trifluoromethyl)- 1-pyrazolyl]-2-aza-2- spiro[3.3]heptyl}methanone |
| 25 | 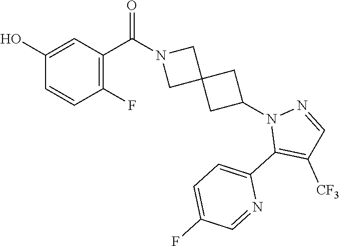 [SMILES: O=C(c1cc(O)ccc1F)N1CC2(CC(n3ncc(C(F)(F)F)c3-c3ccc(F)cn3)C2)C1] | (2-fluoro-5- hydroxyphenyl){6-[5-(5- fluoro-2-pyridyl)-4- pyrazolyl]-2-aza-2- spiro[3.3]heptyl}methanone |
| 26 | 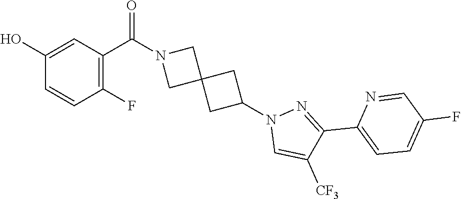 [SMILES: O=C(c1cc(O)ccc1F)N1CC2(CC(n3cc(C(F)(F)F)c(-c4ccc(F)cn4)n3)C2)C1] | (2-fluoro-5- hydroxyphenyl){6-[3-(5- fluoro-2-pyridyl)-4- (trifluoromethyl)-1- pyrazolyl]-2-aza-2- spiro[3.3]heptyl}methanone |

In some embodiments, the disclosure provides a compound, wherein the compound is selected from the group consisting of:

| Compound | Structure | IUPAC Name |
|---|---|---|
| 34 | 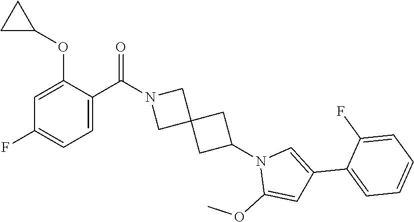 [SMILES: COc1cc(-c2ccccc2F)cn1C1CC2(C1)CN(C(=O)c1ccc(F)cc1OC1CC1)C2] | (2-cyclopropoxy-4- fluorophenyl){6-[4-(o- fluorophenyl)-2-methoxy-1- pyrrolyl]-2-aza-2- spiro[3.3]heptyl}methanone |
| 35 | 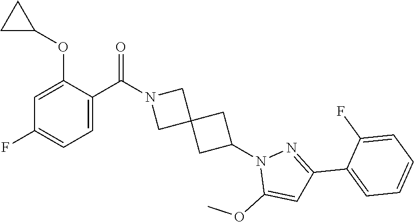 [SMILES: COc1cc(-c2ccccc2F)nn1C1CC2(C1)CN(C(=O)c1ccc(F)cc1OC1CC1)C2] | (2-cyclopropoxy-4- fluorophenyl){6-[3-(o- fluorophenyl)-5-methoxy-1- pyrazolyl]-2-aza-2- spiro[3.3]heptyl}methanone |
| 36 | 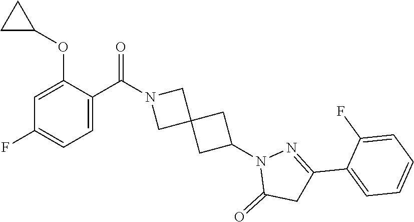 [SMILES: O=C(c1ccc(F)cc1OC1CC1)N1CC2(CC(N3N=C(c4ccccc4F)CC3=O)C2)C1] | 1-[2-(2-cyclopropoxy-4- fluorobenzoyl)-2- azaspiro[3.3]heptan-6-yl]-3- (2-fluorophenyl)-4,5- dihydro-1H-pyrazol-5-one |
| 37 | 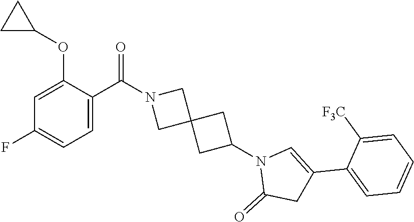 [SMILES: O=C(c1ccc(F)cc1OC1CC1)N1CC2(CC(N3C=C(c4ccccc4C(F)(F)F)CC3=O)C2)C1] | 1-{2-(2-cyclopropoxy-4- fluorobenzoyl)-2-aza-6- spiro[3.3]heptyl}-3-[o- (trifluoromethyl)phenyl]-2- pyrrolin-5-one |
| 38 | 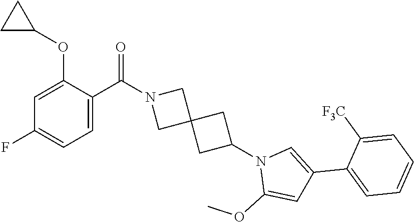 [SMILES: COc1cc(-c2ccccc2C(F)(F)F)cn1C1CC2(C1)CN(C(=O)c1ccc(F)cc1OC1CC1)C2] | (2-cyclopropoxy-4- fluorophenyl)(6-{2-methoxy- 4-[o- (trifluoromethyl)phenyl]-1- pyrrolyl}-2-aza-2- spiro[3.3]heptyl)methanone |
| 39 | 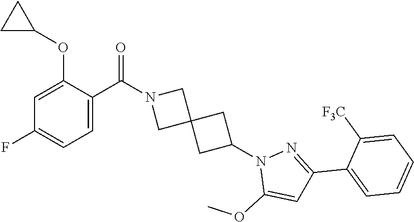 [SMILES: COc1cc(-c2ccccc2C(F)(F)F)nn1C1CC2(C1)CN(C(=O)c1ccc(F)cc1OC1CC1)C2] | (2-cyclopropoxy-4- fluorophenyl)(6-{5-methoxy- 3-[o-(trifluoromethyl) phenyl]-1-pyrazolyl}-2-aza- 2-spiro[3.3]heptyl)methanone |
| 40 | 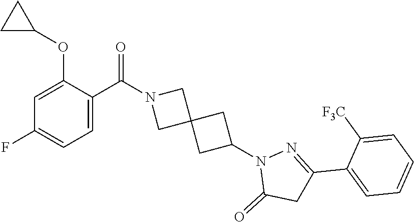 [SMILES: O=C(c1ccc(F)cc1OC1CC1)N1CC2(CC(N3N=C(c4ccccc4C(F)(F)F)CC3=O)C2)C1] | 1-[2-(2-cyclopropoxy-4- fluorobenzoyl)-2- azaspiro[3.3]heptan-6-yl]-3- [2-(trifluoromethyl)phenyl]- 4,5-dihydro-1H-pyrazol-5-one |

A compound of Formula (IV) or a pharmaceutically acceptable salt thereof,

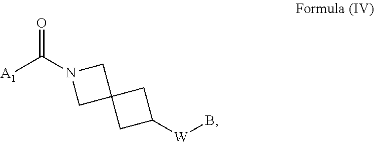

wherein:
   A₁ is

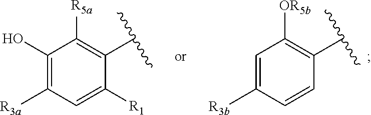

R₁ is hydrogen, halogen, or cyano;
R₃ₐ and R₅ₐ are each independently hydrogen, halogen, or optionally substituted lower alkyl;
R_{3b} is halogen;
R_{5b} is optionally substituted lower alkyl, optionally substituted lower cycloalkyl, or optionally substituted lower haloalkyl;
W is

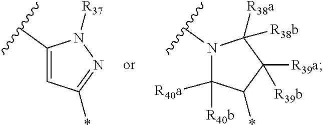

R₃₇, R₃₈ₐ, R_{38b}, R₃₉ₐ, R_{39b}, R₄₀ₐ, R_{40b}, is hydrogen, halogen, lower alkyl, lower alkoxy, lower haloalkyl, or lower cycloalkyl;
B is an optionally substituted 79yridine, phenyl or a 6-member heteroaryl comprising at least one nitrogen atom and optionally substituted with one or more halogen, optionally substituted lower alkyl, optionally substituted lower haloalkyl, or optionally substituted lower alkoxy.

In some embodiments, B is phenyl in compounds of Formula (IV), wherein B can be substituted with one or more halogen or haloalkyl; or B can be substituted with one of more fluoro. In some embodiments, B is

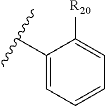

in compounds of Formula (IV), and R₂₀ can be halogen, haloalkyl or alkyl; or R₂₀ can be F, methyl or CF₃.

In some embodiments, W is

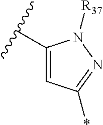

in compounds of Formula (IV), including compounds wherein R₃₇ is hydrogen, halogen, lower alkyl, lower alkoxy, lower haloalkyl, or lower cycloalkyl; compounds wherein R₃₇ is hydrogen, methyl, methoxy, CF₃, or cyclopropyl; compounds wherein R₃₇ is methoxy; compounds wherein R₃₇ is methyl; compounds wherein R₃₇ is hydrogen; compounds wherein R₃₇ is cyclopropyl; or compounds wherein R₃₇ is CF₃. In some embodiments, W is

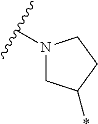 [SMILES: *C1CCN(*)C1]

in compounds of Formula (IV).

In some embodiments. W is

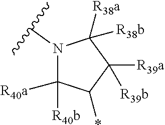

in compounds of Formula (IV), including compounds wherein R₃₈ₐ, R_{38b}, R₃₉ₐ, R_{39b}, R₄₀ₐ, R_{40b}, is hydrogen, halogen, methyl, methoxy, CF₃, or cyclopropyl; compounds wherein R₃₈ₐ, R₃₉ₐ, and R₄₀ₐ, is hydrogen; compounds wherein one of R_{38b}, R_{39b}, and R_{40b}, is halogen, methyl, methoxy, CF₃, or cyclopropyl; and compounds wherein two of R_{38b}, R_{39b}, and R_{40b} are hydrogen.

In some embodiments A₁ is

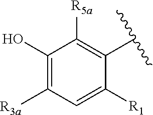

in compounds of Formula (IV), including compounds wherein R₁ is hydrogen, halogen, or cyano; and R₃ₐ and R₅ₐ are each independently hydrogen, halogen, or optionally substituted lower alkyl; compounds wherein R₅ₐ is hydrogen; compounds wherein R₁ is fluoro; compounds wherein R₃ₐ is hydrogen, fluoro, or methyl; compounds wherein R₃ₐ is hydrogen.

In some embodiments, A₁ is

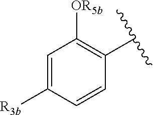

in compounds of Formula (IV), including compounds wherein R_{3b} is halogen; compounds wherein R_{5b} is optionally substituted lower alkyl, optionally substituted lower cycloalkyl, or optionally substituted lower haloalkyl; compounds wherein R_{5b} is cyclopropyl, or lower alkyl optionally substituted with one or more fluoro; compounds wherein R_{3b} is fluoro; or compounds wherein R_{5b} is cyclopropyl.

In some embodiments A₁ is

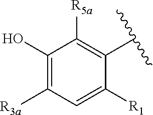

in compounds of Formula (IV), including compounds wherein R₁ is fluoro; and R₃ₐ and R₅ₐ are each independently hydrogen.

In some embodiments, Aₐ is

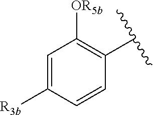

in compounds of Formula (IV), including compounds wherein R_{3b} is fluoro; and R_{5b} is cyclopropyl.

In some embodiments, the compound is a compound of Formula (IV) wherein A₁ is

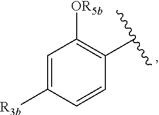

### W is

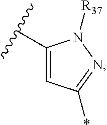

### and B is

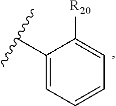

as provided in Table D-1 below.

TABLE D-1
Compounds of Formula (IV).

| Compound | R_{5b} | R_{3b} | R₃₇ | R₂₀ |
|---|---|---|---|---|
| 57 | 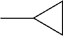 | F | H | F |
| 58 | 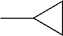 | F | Me | F |
| 59 | 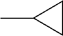 | CF₃ | Me | F |
| 60 | 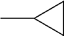 | CF₃ | Me | CF₃ |

In some embodiments, the compound is a compound of Formula (IV) wherein A₁ is

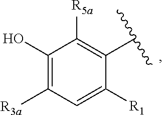

### W is

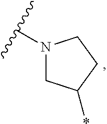 [SMILES: *C1CCN(*)C1]

### and B is

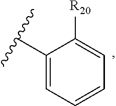

as provided in Table D-2 below.

TABLE D-2
Compounds of Formula (IV).

| Compound | R₅ₐ | R₃ₐ | R₂₀ |
|---|---|---|---|
| 61 | 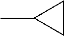 | F | H |
| 62 | 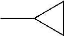 | F | Me |
| 63 | 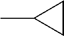 | F | F |
| 64 | 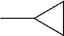 | F | CF₃ |

In some embodiments, the compound is a compound of Formula (IV) wherein A₁ is

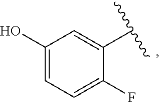 [SMILES: *c1cc(O)ccc1F]

### W is

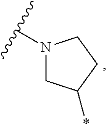 [SMILES: *C1CCN(*)C1]

### and B is

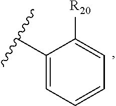

as provided in Table D-3 below.

TABLE D-3

| Compounds of Formula (IV). | |
|---|---|
| Compound | R₂₀ |
| 51 | CF₃ |
| 65 | Me |
| 66 | F |
| 67 | H |

In some embodiments, the compound is a compound of Formula (V) or a pharmaceutically acceptable salt thereof,

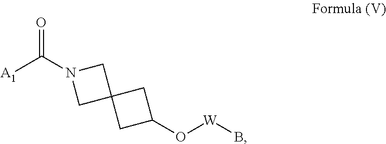

wherein:
   A₁ is

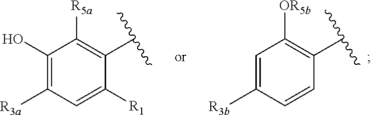

R₁ is hydrogen, halogen, or cyano;
R₃ₐ and R₅ₐ are each independently hydrogen, halogen, or optionally substituted lower alkyl;
R_{3b} is halogen;
R_{5b} is optionally substituted lower alkyl, optionally substituted lower cycloalkyl, or optionally substituted lower haloalkyl;
W is selected from the group consisting of:

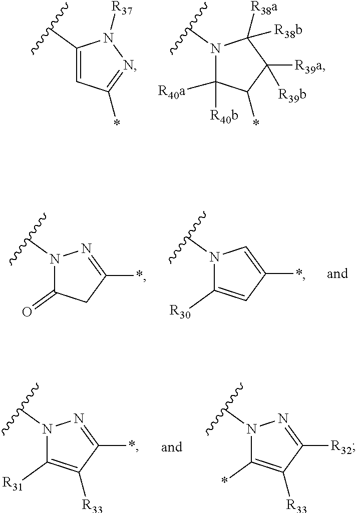

R₃₀, R₃₁, R₃₂, R₃₃, R₃₇, R₃₈ₐ, R_{38b}, R₃₉ₐ, R_{39b}, R₄₀ₐ, R_{40b}, are each independently hydrogen, halogen, lower alkyl, lower alkoxy, lower haloalkyl, or lower cycloalkyl;
B is an optionally substituted 86pyridine, phenyl or a 6-member heteroaryl comprising at least one nitrogen atom and optionally substituted with one or more halogen, optionally substituted lower alkyl, optionally substituted lower haloalkyl, or optionally substituted lower alkoxy.

In some embodiments, B is phenyl in compounds of Formula (V), wherein B can be substituted with one or more halogen or haloalkyl; or B can be substituted with one of more fluoro. In some embodiments, B is

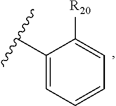

in compounds of Formula (V), and R₂₀ can be halogen, haloalkyl, or alkyl; or R₂₀ can be F, methyl or CF₃.

In some embodiments, W is

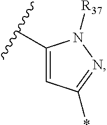

in compounds of Formula (V), including compounds wherein R₃₇ is hydrogen, halogen, lower alkyl, lower alkoxy, lower haloalkyl, or lower cycloalkyl; compounds wherein R₃₇ is hydrogen, methyl, methoxy, CF, or cyclopropyl; compounds wherein R₃₇ is methoxy; compounds wherein R₃₇ is methyl; compounds wherein R₃₇ is hydrogen; compounds wherein R₃₇ is cyclopropyl; or compounds wherein R₃₇ is CF₃.

In some embodiments A₁ is

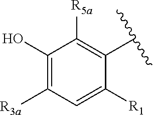

in compounds of Formula (V), including compounds wherein R₁ is hydrogen, halogen, or cyano; and R₃ₐ and R₅ₐ are each independently hydrogen, halogen, or optionally substituted lower alkyl; compounds wherein R₅ₐ is hydrogen; compounds wherein R₁ is fluoro; compounds wherein R₃ₐ is hydrogen, fluoro, or methyl; compounds wherein R₃ₐ is hydrogen.

In some embodiments, A₁ is

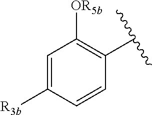

in compounds of Formula (V), including compounds wherein R_{3b} is halogen; compounds wherein R_{5b} is optionally substituted lower alkyl, optionally substituted lower cycloalkyl, or optionally substituted lower haloalkyl; compounds wherein R_{5b} is cyclopropyl, or lower alkyl optionally substituted with one or more fluoro; compounds wherein R_{3b} is fluoro; or compounds wherein R_{5b} is cyclopropyl.

In some embodiments A₁ is

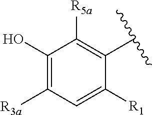

in compounds of Formula (V), including compounds wherein R₁ is fluoro; and R₃ₐ and R₅ₐ are each independently hydrogen.

In some embodiments, A₁ is

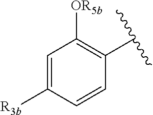

in compounds of Formula (V), including compounds wherein R_{3b} is fluoro; and R_{5b} is cyclopropyl.

In some embodiments, a compound of Formula (V) is a compound wherein B is

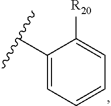

R₂₀ can be halogen, haloalkyl, or alkyl; or R₂₀ can be F, methyl or CF₃; W is

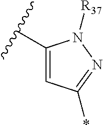

including compounds wherein R₃₇ is hydrogen, methyl, methoxy, CF₃, or cyclopropyl, and A₁ is

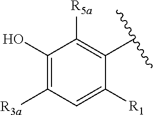

in compounds of Formula (V), including compounds wherein R₁ is hydrogen, halogen, or cyano or A₁ is

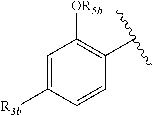

in compounds of Formula (V), including compounds wherein R_{3b} is halogen, compounds wherein R_{5b} is optionally substituted lower alkyl, optionally substituted lower cycloalkyl, or optionally substituted lower haloalkyl.

In some embodiments, a compound of Formula (V) is a compound wherein B is

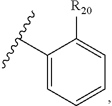

R₂₀ is F, methyl or CF₃; W is

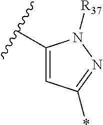

including compounds wherein R₃₇ is hydrogen, methyl, methoxy, CF₃, or cyclopropyl; and A₁ is

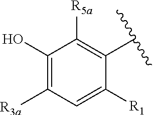

in compounds of Formula (V), including compounds wherein R₁ is fluoro and R_{3A} and R₅ₐ are both hydrogen, or A₁ is

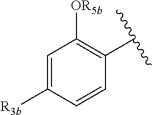

in compounds of Formula (V), including compounds wherein R_{3b} is fluoro; compounds wherein R_{5b} is cyclopropyl.

In some embodiments, the compound is a compound of Formula (V) wherein A₁ is

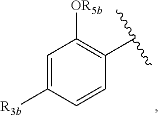

### W is

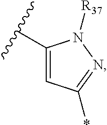

### and B is

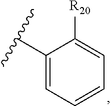

as provided in Table D-4 below.

TABLE D-4
Compounds of Formula (V).

| Compound | R_{5b} | R_{3b} | R₃₇ | R₂₀ |
|---|---|---|---|---|
| 45 | 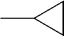 | F | H | F |
| 47 | 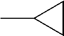 | F | Me | F |
| 48 | 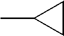 | F | H | CF₃ |
| 50 | 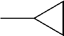 | F | Me | CF₃ |

### General Procedure for Preparing Compounds of Formula (I)

In general, compounds of formula (I) where W is

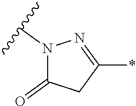 [SMILES: *C1=NN(*)C(=O)C1]

and B is an optionally substituted pyridine, can be made by following Example 9, a modified version of Example 2. In line with Steps-A and -B described in Example 2, 6-methoxynicotinic acid, Int-A, is treated with methoxyamine hydrochloride to generated Weinreb amide intermediate Int-B (Step-A), which subsequently undergoes Grignard addition with methylmagnesium bromide to yield ketone methyl ketone intermediate, Int-1 (Step-B). Unlike Step-1 of Example 2 where Claisen condensation of Int-1 is performed with ethyl trifluoroacetate to generate a 1,3-diketone intermediate, Int-1 conversely undergoes Claisen condensation with diethyl carbonate to afford the β-keto ester intermediate, Int-2. Cycloaddition of Int-2 with hydrazine hydrate generates the 346-methoxy-pyridin-3-yl) intermediate, Int-3 (Step-2) Simultaneous synthesis of 2-(5-(benzyloxy)-2-fluorobenzoyl)-2-azaspiro[3.3]heptan-6-yl methanesulfonate, Int-C, is executed by first reacting tert-butyl 6-hydroxy-2-azaspiro[3.3]heptane-2-carboxylate with methanesulfonyl chloride, then performing V-Boc-deprotection, followed by amide coupling with 5-(benzyloxy)-2-fluorobenzoic acid. Nucleophilic substitution of Int-C with Int-3 yields Int-4, which lastly undergoes N-benzyl deprotection via Pd/C hydrogenolysis to generate the final 1,3-disubstituted 2-pyrazin-5-one analog.

In general, compounds of formula (I) where W is

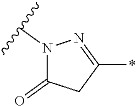 [SMILES: *C1=NN(*)C(=O)C1]

and B is a 2-pyridinone, can be made by following Example 10, a modified synthetic scheme based on Examples 5 and 9. Identical to Steps-A, —B, and —C of Example 5, 6-hydroxynicotinic acid, Int-A, reacts with methoxyamine hydrochloride to yield Weinreb amide intermediate Int-B, which subsequently undergoes O-benzylation, followed by Grignard addition with methylmagnesium bromide to generate methyl ketone intermediate Int-1. Congruent with Step-1 in Example 9, Int-1, undergoes Claisen condensation with diethyl carbonate to yield β-keto ester intermediate, Int-2, which subsequently undergoes cyclization with hydrazine hydrate to yield the 3-(6-benzyloxy-pyridin-3-yl) intermediate, Int-3. Nucleophilic addition of Int-3 to previously described mesylate intermediate, Int-C, affords the adduct Int-4. Liberation of the OH group in the A1 phenyl ring, and the 2-pyridinone B group is accomplished in a single step by Pd/C hydrogenolysis of Int-4, thereby affording the 1,3-disubstituted 2-pyrazin-5-one final analog.

In general, compounds of formula (I) where W is

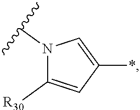

* is the bond to B; R₃₀ is hydrogen, halogen, lower alkyl, lower alkoxy, lower haloalkyl, or lower cycloalkyl; and B is an optionally substituted pyrimidine can be made by following Example 11. 6-methoxynicotinaldehyde, Int-A, undergoes aldol condensation with cyclopropyl methyl ketone to yield α,β-unsaturated ketone intermediate Int-1. Int-1 is then treated with aminoacetonitrile hydrochloride to yield cyanopyrroline intermediate Int-2. Microwave irradiation of Int-2 promotes cyano-elimination and spontaneous aromatization to generate pyrrole intermediate, Int-3. Nucleophilic substitution of previously described mesylate intermediate, Int-C, with Int-3 generates Int-4, which subsequently undergoes O-benzyl-deprotection to yield the 1,2,4-trisubstituted pyrrole analog.

In general, compounds of formula (I) where W is

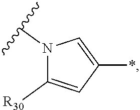

* is the bond to B; R₃₀ is hydrogen, halogen, lower alkyl, lower alkoxy, lower haloalkyl, or lower cycloalkyl; and B is a 2-pyridinone can be made by following the scheme described in Example 12. 5-bromo-2-chloropyridine, Int-A, undergoes an addition, elimination reaction with benzyl alcohol to yield 2-benzyloxy-5-bromopyridine, Int-1. Palladium-catalyzed cross-coupling of Int-1 with potassium vinyltrifluoroborate yields 2-benzyloxy-5-vinylpyridine, Int-2, which undergoes cyclization with tosylmethyl isocyanide to yield pyrrole intermediate Int-3. Treatment of Int-3 with previously described mesylate intermediate. Int-C, generates nucleophilic adduct Int-4, which subsequently undergoes O-benzyl-deprotection to yield the 1,3-disubstituted pyrrole analog.

### General Procedure for Preparing Compounds of Formula (I-A-1) and I-B-1

In general, analogs of formula of I-A-1 can be made by following the synthetic scheme described in Example 2, synthesis of compounds 17 and 18. 6-methoxynicotinic acid, Int-A, is treated with methoxyamine hydrocholoride to generate Weinreb amide intermediate, Int-B, which subsequently undergoes Grignard addition with methylmagnesium bromide to yield methyl ketone intermediate, Int-1. Claisen condensation of Int-1 with ethyl trifluoroacetate yields 1,3-diketone intermediate, Int-2, which subsequently undergoes intermolecular cyclization with hydrazine to yield pyrazole intermediate, Int-3. Nucleophilic addition of Int-3 to previously described mesylate intermediate, Int-C, yields two isomers: 3-(trifluoromethyl)-5-(6-methyoxylpyridin-3-yl)pyrazolyl adduct, Int-4, and 3-(6-methoxypyridin-3-yl)-5-(trifluoromethyl)pyrazolyl adduct, Int-4a. For compounds belonging to Formula-I-A-1, Int-4 is advanced for O-benzyl-deprotection to yield the final analog. For compounds belonging to Formula-I-B-1, Int-4a is carried out for O-benzyl-deprotection to yield the respective final analog.

The compounds described herein can exist as salts, such as with pharmaceutically acceptable acids. Accordingly, such salts of the compounds described herein are included. The term “pharmaceutically acceptable salt” is meant to include salts of the active compounds that are prepared with relatively nontoxic acids or bases, depending on the particular substituents found on the compounds described herein. When compounds of the present invention contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, oxalic, methanesulfonic, and the like. Also included are salts of amino acids such as arginate and the like, and salts of organic acids like glucuronic or galactunoric acids and the like (see, for example, Berge et al., “Pharmaceutical Salts”, Journal of Pharmaceutical Science. 1977, 66, 1-19). Certain specific compounds of the present invention contain both basic and acidic functionalities that allow the compounds to be converted into either base or acid addition salts.

The neutral forms of the compounds are preferably regenerated by contacting the salt with a base or acid and isolating the parent compound in the conventional manner. The parent form of the compound differs from the various salt forms in certain physical properties, such as solubility in polar solvents.

### Pharmaceutical Compositions

In certain embodiments, the present application is directed to a pharmaceutical composition comprising an active pharmaceutical ingredient. In certain embodiments, the pharmaceutical composition comprises a compound as disclosed herein as the active pharmaceutical ingredient (API) and a pharmaceutically acceptable carrier comprising one or more excipients. In some embodiments, the pharmaceutical composition optionally further comprises an additional therapeutic compound (i.e., agent) with the pharmaceutically acceptable carrier. The pharmaceutical composition can be a medicament.

Pharmaceutically acceptable carriers include those known in the art. The choice of a pharmaceutically acceptable carrier can depend, for example, on the desired route of administration of the composition. A pharmaceutical composition (preparation) can be administered to a subject by any of a number of routes of administration including, for example, parenteral administration (e.g. intravenously, subcutaneously, or intramuscularly), oral administration (for example, tablets, and capsules); absorption through the oral mucosa (e.g., sublingually) or transdermally (for example as a patch applied to the skin) or topically (for example, as a cream, ointment or spray applied to the skin).

In some embodiments, pharmaceutical compositions comprising compounds of Formula (I) or pharmaceutically acceptable salts thereof can be formulated for oral administration. For example, a compound provided herein can be combined with suitable compendial excipients to form an oral unit dosage form, such as a capsule or tablet, containing a target dose of a compound of Formula (I). The drug product can be prepared by first manufacturing the compound of Formula (I) as an active pharmaceutical ingredient (API), followed by roller compaction/milling with intragranular excipients and blending with extra granular excipients. A Drug Product can contain the selected compound of Formula (I) as the API and excipient components in a tablet in a desired dosage strength of a compound of Formula (I) The blended material can be compressed to form tablets and then film coated. The excipients can be selected from materials appropriate for inclusion in a pharmaceutical composition for an intended purpose and route of delivery including providing a desired manufacturing and stability properties and/or desired in vivo characteristics or other properties to the pharmaceutical composition. In some embodiments, the pharmaceutical composition can include a compound of Formula (I) as the API in combination with a filler (e.g., a form of microcrystalline cellulose), a dry binder or disintegrant (e.g., a cross-linked polymer), a glidant (e.g., colloidal silicon dioxide) and/or a lubricant (e.g., magnesium stearate). In some embodiments, the pharmaceutical composition can comprise a material such as an extended release or disintegrant involved in carrying or transporting the API pharmaceutical agent from one organ, or portion of the body, to another organ, or portion of the body of a subject, including materials to desirable control the absorption of the API in the intestine.

The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient that can be combined with a carrier material to produce a single dosage form will generally be that amount of the compound which produces a therapeutic effect. For use in the methods of this invention, active compounds can be given per se or as a pharmaceutical composition containing, for example, 0.1 to 99.5% (more preferably, 0.5 to 90%) of active ingredient in combination with a pharmaceutically acceptable carrier.

Methods of preparing these formulations or compositions include the step of bringing into association an active compound, such as a compound of the invention, with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

To prepare solid dosage forms for oral administration, the active ingredient is mixed with one or more pharmaceutically acceptable carriers, such as sodium citrate or dicalcium phosphate, and/or any of the following: (1) fillers or extenders, (2) binders, (3) humectants, (4) disintegrating agents, (5) solution retarding agents, (6) absorption accelerators, (7) wetting agents, (8) absorbents, (9) lubricants, (10) complexing agents, and (11) coloring agents. In the case of capsules (including sprinkle capsules and gelatin capsules), tablets and pills, the pharmaceutical compositions may also comprise buffering agents. Solid compositions of a similar type may also be employed as fillers in soft and hard-filled gelatin capsules using suitable excipients. The pharmaceutical compositions according to the present invention may contain conventional pharmaceutical carriers and/or auxiliary agents. In some embodiments, he pharmaceutical compositions according to the present invention may contain conventional carrier agents including a binder, a lubricant and/or a glidant selected from those products and materials generally used in pharmaceutical industry for preparation of pharmaceutical compositions for an intended route of administration.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

Liquid dosage forms useful for oral administration include pharmaceutically acceptable carriers and the active ingredient provided as a solid form for reconstitution prior to administration or as a liquid (e.g., solutions, suspensions, or emulsions). In addition to the active ingredient, a liquid dosage forms may contain inert diluents commonly used in the art. For example, formulations of pharmaceutically acceptable compositions for injection can include aqueous solutions such as water or physiologically buffered saline or other solvents or vehicles suitable for the intended route of administration. In some embodiments, the pharmaceutical composition is formulated for parenteral administration.

The therapeutically effective amount of a pharmaceutical composition can be determined by human clinical trials to determine the safe and effective dose for a patient with a relevant diagnosis. It is generally understood that the effective amount of the compound may vary according to the weight, sex, age, and medical history of the subject. Other factors which influence the effective amount may include, but are not limited to, the severity of the patient's condition, the disorder being treated, the stability of the compound, and, if desired, another type of therapeutic agent being administered with the compound of the invention. A larger total dose can be delivered by multiple administrations of the pharmaceutical composition at a dose and dose interval determined to be safe and effective for the patient.

The present disclosure includes the use of pharmaceutically acceptable salts of compounds of the invention in the compositions and methods of the present invention. Pharmaceutically-acceptable salts include, for example, acid-addition salts and base-addition salts. The acid that is added to a compound to form an acid-addition salt can be an organic acid or an inorganic acid A base that is added to a compound to form a base-addition salt can be an organic base or an inorganic base. In some embodiments, a pharmaceutically-acceptable salt is a metal salt, in some embodiments, a pharmaceutically-acceptable salt is an ammonium salt. For example, a pharmaceutically acceptable acid addition salt can exist as various solvates, such as with water, methanol, ethanol, dimethylformamide, and the like Mixtures of such solvates can also be prepared. The source of such solvate can be from the solvent of crystallization, inherent in the solvent of preparation or crystallization, or adventitious to such solvent.

### Methods of Use

The compounds described herein can modulate activity of monoacylglycerol lipase. For example, the compounds described herein can inhibit MAGL. Accordingly, in one aspect the disclosure provides a method for inhibiting MAGL, e.g., in a cell expressing MAGL. Generally, the method comprises administering to the cell a compound of Formula (I) described herein. It is noted that the inhibition can be reversible or irreversible. In some embodiments, the compounds are capable of reversibly inhibiting MAGL. As used herein, “reversible inhibition” means MAGL retains its activity once the compound is taken away or MAGL is no longer in contact with the compound. In other words, the activity of MAGL returns to the same level it was prior to use of the compound.

The compound can be administered to the cell, e.g. cell expressing MAGL in vitro or ex vivo. As used herein, administering the compound to the cell means contacting the cell with the compound so that the compound is taken up by the cell. Generally, the cell can be contacted with the compound in a cell culture e.g., in vitro or ex vivo, or the compound can be administrated to a subject, e.g., in vivo. The term “contacting” or “contact” as used herein in connection with contacting a cell includes subjecting the cells to an appropriate culture media, which comprises a compound of Formula (I). Where the cell is in vivo, “contacting” or “contact” includes administering the compound, e.g., in a pharmaceutical composition to a subject via an appropriate administration mute such that the compound contacts the cell in vivo.

As described herein, the compound of Formula (I) can be administered to a cell in vivo for modulating MAGL, e.g., inhibiting MAGL. Accordingly, in some embodiments, a therapeutically effective amount of a compound of Formula (I) can be administered to a subject for inhibiting monoacylglycerol lipase. For example, a therapeutically effective amount of a compound of Formula (I) can be administrated to a subject for treating a monoglycerol lipase mediated disease or disorder.

By a MAGL-mediated disease or disorder is meant a disease or disorder wherein activity of MAGL is a cause of the disease or disorder. Non-limiting example, of MAGL-mediated diseases or disorders include neurodegenerative diseases, neuropsychiatric disorders, pain and chronic inflammatory diseases, cancer and other conditions that can be treated with a reversible MAGL inhibitor of Formula (I).

In some embodiments, a compound of Formula (I) is useful for evaluation as a drug candidate for the treatment of neurodegenerative diseases such as Parkinson's disease, amyotrophic internal sclerosis, Alzheimer's disease or Huntington's disease. In some embodiments, a compound of Formula (I) is useful for evaluation as a drug candidate for the treatment of neuropsychiatric disorders such as head injury, epilepsy, cerebral ischemia and anxiety and/or depression. In some embodiments, a compound of Formula (I) is useful for evaluation as a drug candidate for the treatment of pain and inflammatory diseases or conditions. In some embodiments, a compound of Formula (I) is useful for evaluation as a drug candidate for the treatment of forms of cancer treatable by increasing 2-AG. In some embodiments, a compound of Formula (I) is useful for evaluation as a drug candidate for the treatment of obesity, fibrosis, emesis, asthma, fever and osteoporosis. In some embodiments, a compound of Formula (I) is useful for evaluation as a drug candidate for increasing levels of 2-AG in a subject in need thereof.

A subject can be one who has been previously diagnosed with or identified as suffering from or having a condition in need of treatment a MAGL-mediated disease or disorder or one or more complications related to such a condition, and optionally, have already undergone treatment for such a disease or disorder. Alternatively, a subject can also be one who has not been previously diagnosed as having a MAGL-mediated disease or disorder or one or more complications related to such a disease or disorder. A “subject in need” of treatment for a particular condition can be a subject having that condition, diagnosed as having that condition, or at risk of developing that condition.

In one embodiment, the subject is human. In another embodiment, the subject is an experimental animal or animal substitute as a disease model.

It is noted that the terms “administered” and “subjected” are used interchangeably in the context of treatment of a disease or disorder. In jurisdictions that forbid the patenting of methods that are practiced on the human body, the meaning of “administering” of a composition to a human subject shall be restricted to prescribing a controlled substance that a human subject will be administer to the subject by any technique (e.g., orally, inhalation, topical application, injection, insertion, etc.). The broadest reasonable interpretation that is consistent with laws or regulations defining patentable subject matter is intended. In jurisdictions that do not forbid the patenting of methods that are practiced on the human body, the “administering” of compositions includes both methods practiced on the human body and also the foregoing activities.

As used herein, the term “administer” refers to the placement of a composition into a subject by a method or route which results in at least partial localization of the composition at a desired site such that desired effect is produced. A compound or composition described herein can be administered by any appropriate route known in the art including, but not limited to, oral or parenteral routes, including intravenous, intramuscular, subcutaneous, transdermal, airway (aerosol), pulmonary, nasal, rectal, and topical (including buccal and sublingual) administration.

Exemplary modes of administration include, but are not limited to, injection, infusion, instillation, inhalation, or ingestion. “Injection” includes, without limitation, intravenous, intramuscular, intraarterial, intrathecal, intraventricular, intracapsular, intraorbital, intracardiac, intradermal, intraperitoneal, transtracheal, subcutaneous, subcuticular, intraarticular, sub capsular, subarachnoid, intraspinal, intracerebro spinal, and intrasternal injection and infusion. In some embodiments, administration will generally be local rather than systemic.

In preferred embodiments, the compositions are orally administered. Without limitations, oral administration can be in the form of solutions, suspensions, tablets, pills, capsules, sustained-release formulations, oral rinses, powders and the like.

The phrase “therapeutically-effective amount” as used herein means that amount of a compound, material, or composition comprising a compound described herein which is effective for producing some desired therapeutic effect in at least a sub-population of cells, e.g., modulate or inhibit activity of MAGL in a subject at a reasonable benefit/risk ratio applicable to any medical treatment. Thus, “therapeutically effective amount” means that amount which, when administered to a subject for treating a disease, is sufficient to affect such treatment for the disease.

Depending on the route of administration, effective doses can be calculated according to the body weight, body surface area, or organ size of the subject to be treated. Optimization of the appropriate dosages can readily be made by one skilled in the art in light of pharmacokinetic data observed in human clinical trials. Alternatively, or additionally, the dosage to be administered can be determined from studies using animal models for the particular type of condition to be treated, and/or from animal or human data obtained from agents which are known to exhibit similar pharmacological activities. The final dosage regimen will be determined by the attending surgeon or physician, considering various factors which modify the action of active agent, e.g., the agent's specific activity, the agent's specific half-life in vivo, the severity of the condition and the responsiveness of the patient, the age, condition, body weight, sex and diet of the patient, the severity of any present infection, time of administration, the use (or not) of other concomitant therapies, and other clinical factors.

The data obtained from the cell culture assays and animal studies can be used in formulating a range of dosage for use in humans. The dosage of such compounds lies preferably within a range of circulating concentrations that include the IC₅₀ with little or no toxicity. The dosage may vary within this range depending upon the dosage form employed and the route of use or administration utilized. The effective dose can be estimated initially from cell culture assays. A dose can be formulated in animal models to achieve a circulating plasma concentration range that includes the IC₅₀ (i.e., the concentration of the therapeutic which achieves a half-maximal inhibition of symptoms) as determined in cell culture. Levels in plasma can be measured, for example, by high performance liquid chromatography. The effects of any particular dosage can be monitored by a suitable bioassay.

It will be appreciated that methods of treatment of the present invention can be employed in combination with additional therapies. For example, a treatment according to the present disclosure can be co-administered with one or more desired therapeutics or medical procedures for treating a MAGL-mediated disease or disorder.

### Definitions

One skilled in the art will recognize many methods and materials similar or equivalent to those described herein, which could be used in the practice of the present invention. Other features and advantages of the invention will become apparent from the following detailed description, taken in conjunction with the accompanying drawings, which illustrate, by way of example, various features of embodiments of the invention. Indeed, the present invention is in no way limited to the methods and materials described.

Unless otherwise defined herein, scientific and technical terms used in this application shall have the meanings that are commonly understood by those of ordinary skill in the art.

For convenience, certain terms employed herein, in the specification, examples and appended claims are collected herein. Unless stated otherwise, or implicit from context, the following terms and phrases include the meanings provided below. Unless explicitly stated otherwise, or apparent from context, the terms and phrases below do not exclude the meaning that the term or phrase has acquired in the art to which it pertains. The definitions are provided to aid in describing particular embodiments, and are not intended to limit the claimed invention, because the scope of the invention is limited only by the claims. Further, unless otherwise required by context, singular terms shall include pluralities and plural terms shall include the singular.

Unless defined otherwise, all technical and scientific terms used herein have the same meaning as those commonly understood to one of ordinary skill in the art to which this invention pertains. Although any known methods, devices, and materials may be used in the practice or testing of the invention, the methods, devices, and materials in this regard are described herein.

As used herein the term “comprising” or “comprises” is used in reference to compositions, methods, systems, articles of manufacture, apparatus, and respective component(s) thereof, that are useful to an embodiment, yet open to the inclusion of unspecified elements, whether useful or not. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.). Although the open-ended term “comprising,” as a synonym of terms such as including, containing, or having, is used herein to describe and claim the invention, the present invention, or embodiments thereof, may alternatively be described using alternative terms such as “consisting of” or “consisting essentially of.”

Unless stated otherwise, the terms “a” and “an” and “the” and similar references used in the context of describing a particular embodiment of the application (especially in the context of claims) can be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (for example, “such as”) provided with respect to certain embodiments herein is intended merely to better illuminate the application and does not pose a limitation on the scope of the application otherwise claimed. The abbreviation, “e.g.” is derived from the Latin exempli gratia, and is used herein to indicate a non-limiting example. Thus, the abbreviation “e.g.” is synonymous with the term “for example.” No language in the specification should be construed as indicating any non-claimed element essential to the practice of the application.

In some embodiments, the numbers expressing quantities of reagents, properties such as concentration, reaction conditions, and so forth, used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

“Optional” or “optionally” means that the subsequently described circumstance may or may not occur, so that the description includes instances where the circumstance occurs and instances where it does not.

As used herein, the term “Selective MAGL Inhibitor Compound” refers to a compound that selectively inhibits MAGL with an IC₅₀ that is at least 10 times the IC₅₀ for its inhibition of fatty acid amide hydrolase (FAAH), and that has an IC₅₀ of 100 nM or less for MAGL inhibition (according to the MAGL Potency assay of Example A and the FAAH potency assay of Example 15).

As used herein, the term “Reversible MAGL Inhibitor Compound” the percent inhibition after dilution to the IC₅₀ concentration is 50+15% in the assay described for “determining MAGL reversible inhibition” section of Example 16 below.

As used herein, the term “Reversible Selective MAGL Inhibitor Compound” refers to a compound that is both a Selective MAGL Inhibitor Compound and a Reversible MAGL Inhibitor Compound, or a pharmaceutically acceptable salt thereof.

As used herein, the term “alkyl” refers to an aliphatic hydrocarbon group which can be straight or branched having 1 to about 10 carbon atoms in the chain, and which preferably have about 1 to about 6 carbons in the chain. “Lower alkyl” refers to an alkyl group having 1 to about 4 carbon atoms. “Higher alkyl” refers to an alkyl group having about 5 to about 10 carbon atoms. The alkyl group can be optionally substituted with one or more alkyl group substituents which can be the same or different, where “alkyl group substituent” includes halo, amino, aryl, hydroxy, alkoxy, aryloxy, alkyloxy, alkylthio, arylthio, aralkyloxy, aralkylthio, carboxy, alkoxycarbonyl, oxo and cycloalkyl. “Branched” refers to an alkyl group in which a lower alkyl group, such as methyl, ethyl or propyl, is attached to a linear alkyl chain. Exemplary alkyl groups include methyl, ethyl, i-propyl, n-butyl, t-butyl, n-pentyl, heptyl, octyl, decyl, dodecyl, tridecyl, tetradecyl, pentadecyl and hexadecyl. Useful alkyl groups include branched or straight chain alkyl groups of 6 to 50 carbon, and also include the lower alkyl groups of 1 to about 4 carbons and the higher alkyl groups of about 12 to about 16 carbons.

As used herein, the term “cycloalkyl” refers to a non-aromatic mono- or multicyclic ring system of about 3 to about 12 carbon atoms. Representative monocyclic cycloalkyl rings include cyclopropyl, cyclobutyl, and cyclohexyl. Useful multicyclic cycloalkyl rings include adamantyl. “Lower cycloalkyl” refers to an alkyl group having 3 to about 6 carbon atoms in the cycloalkyl ring, optionally substituted with halogen, alkyl, alkoxy or other substituents disclosed herein. “Higher alkyl” refers to an alkyl group having about 5 to about 10 carbon atoms.

“Aryl” refers to an aromatic carbocyclic radical containing about 3 to about 10 carbon atoms. The aryl group can be optionally substituted with one or more substituents, which can be the same or different, where “aryl group substituent” includes alkyl, alkenyl, alkynyl, hydroxy, alkoxy, carboxy, halo, nitro, trihalomethyl, cyano, alkoxycarbonyl, aryloxycarbonyl, aralkoxycarbonyl, acyloxy, acylamino, aroylamino, carbamoyl, alkylcarbamoyl, dialkylcarbamoyl, arylthio, alkylthio, and alkylene. Exemplary aryl groups include substituted or unsubstituted phenyl.

“Heterocyclyl” refers to a nonaromatic 3-8 membered monocyclic, or 8-12 membered bicyclic ring systems having 1-3 heteroatoms if monocyclic, or 1-6 heteroatoms if bicyclic, said heteroatoms selected from O, N, or S (e.g., carbon atoms and 1-3, or 1-6 heteroatoms of N, O, or S if monocyclic, or bicyclic, respectively). Cₓheterocyclyl and Cₓ-C_{y}heterocyclyl are typically used where X and Y indicate the number of carbon atoms in the ring system. In some embodiments, 1, 2 or 3 hydrogen atoms of each ring can be substituted by a substituent. Exemplary heterocyclyl groups include, but are not limited to piperazinyl, pyrrolidinyl, dioxanyl, morpholinyl, tetrahydrofuranyl, piperidyl, 4-morpholyl, 4-piperazinyl, pyrrolidinyl, perhydropyrrolizinyl, 1,4-diazaperhydroepinyl, 1,3-dioxanyl, 1,4-dioxanyl and the like.

“Heteroaryl” refers to an aromatic 3-8 membered monocyclic, or 8-12 membered fused bicyclic ring system having 1-3 heteroatoms if monocyclic, or 1-6 heteroatoms if bicyclic, said heteroatoms selected from O, N, or S (e.g., carbon atoms and 1-3, 1-6, or 1-9 heteroatoms of N, O, or S if monocyclic, bicyclic, or tricyclic, respectively.

Exemplary aryls and heteroaryls include, but are not limited to, phenyl, pyridinyl, pyrimidinyl, furanyl, thienyl, imidazolyl, thiazolyl, pyrazolyl, pyridazinyl, pyrazinyl, triazinyl, tetrazolyl, indolyl, benzyl, naphthyl, anthracenyl, azulenyl, fluorenyl, indanyl, indenyl, naphthyl, tetrahydronaphthyl, benzimidazolyl, benzofuranyl, benzothiofuranyl, benzothiophenyl, benzoxazolyl, benzoxazolinyl, benzthiazolyl, benztriazolyl, benztetrazolyl, benzisoxazolyl, benzisothiazolyl, benzimidazolinyl, carbazolyl, 4aH carbazolyl, carbolinyl, chromanyl, chromenyl, cinnolinyl, decahydroquinolinyl, 2H,6H-1,5,2-dithiazinyl, dihydrofuro[2,3 b]tetrahydrofuran, furanyl, furazanyl, imidazolidinyl, imidazolinyl, imidazolyl, 1H-indazolyl, indolenyl, indolinyl, indolizinyl, indolyl, 3H-indolyl, isatinoyl, isobenzofuranyl, isochromanyl, isoindazolyl, isoindolinyl, isoindolyl, isoquinolinyl, isothiazolyl, isoxazolyl, methylenedioxyphenyl, morpholinyl, naphthyridinyl, octahydroisoquindinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxindolyl, pyrimidinyl, phenanthridinyl, phenanthrolinyl, phenazinyl, phenothiazinyl, phenoxathinyl, phenoxazinyl, phthalazinyl, piperazinyl, piperidinyl, piperidonyl, 4-piperidonyl, piperonyl, pteridinyl, purinyl, pyranyl, pyrazinyl, pyrazolidinyl, pyrizolinyl, pyrazolyl, pyridazinyl, pyridooxazole, pyridoimidazole, pyridothiazole, pyridinyl, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydroquinolinyl, tetrazolyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl or xanthenyl, each of which can be optionally substituted.

As used herein, the term “halogen” or “halo” refers to an atom selected from fluorine, chlorine, bromine and iodine. The term “halogen radioisotope” or “halo isotope” refers to a radionuclide of an atom selected from fluorine, chlorine, bromine and iodine.

A “halogen-substituted moiety” or “halo-substituted moiety”, as an isolated group or part of a larger group, means an aliphatic, alicyclic, or aromatic moiety, as described herein, substituted by one or more “halo” atoms, as such terms are defined in this application.

The term “haloalkyl” as used herein refers to an alkyl structure with at least one substituent of fluorine, chorine, bromine or iodine, or with combinations thereof. Exemplary halo-substituted alkyl includes haloalkyl, dihaloalkyl, trihaloalkyl, perhaloalkyl and the like (e.g. halosubstituted (C₁-C₃)alkyl includes chloromethyl, dichloromethyl, difluoromethyl, trifluoromethyl (CF₃), perfluoroethyl, 2,2,2-trifluoroethyl, 2,2,2-tri fluoro-l,l-dichloroethyl, and the like).

As used herein, the term “amino” means —NH₂ or —NH₃⁺ where one or more hydrogens are optionally substituted with alkyl, and the alkyl is optionally further substituted with one or more halogen or other substituents disclosed herein. The term “alkylamino” means a nitrogen moiety having one straight or branched unsaturated aliphatic, cyclyl, or heterocyclyl radicals attached to the nitrogen, e.g., —NH(alkyl). The term “dialkylamino” means a nitrogen moiety having at two straight or branched unsaturated aliphatic, cyclyl, or heterocyclyl radicals attached to the nitrogen, e.g., —N(alkyl)(alkyl). The term “alkylamino” includes “alkenylamino,” “alkynylamino,” “cyclylamino,” and “heterocyclylamino.” The term “arylamino” means a nitrogen moiety having at least one aryl radical attached to the nitrogen. For example, —NHaryl, and —N(aryl)₂. The term “heteroarylamino” means a nitrogen moiety having at least one heteroaryl radical attached to the nitrogen. For example —NHheteroaryl, and —N(heteroaryl)₂. Optionally, two substituents together with the nitrogen can also form a ring. Unless indicated otherwise, the compounds described herein containing amino moieties can include protected derivatives thereof. Suitable protecting groups for amino moieties include acetyl, tertbutoxycarbonyl, benzyloxycarbonyl, and the like. Exemplary alkylamino includes, but is not limited to, NH(C₁-C₁₀alkyl), such as —NHCH₃, —NHCH₂CH₃, —NHCH₂CH₂CH₃, and —NHCH(CH₃)₂. Exemplary dialkylamino includes, but is not limited to, —N(C₁-C₁₀alkyl)₂, such as N(CH₃)₂, —N(CH₂CH₃)₂, —N(CH₂CH₂CH₃)₂, and —N(CH(CH₃)₂)₂.

The term “aminoalkyl” means an alkyl, alkenyl, and alkynyl as defined above, except where one or more substituted or unsubstituted nitrogen atoms (—N—) are positioned between carbon atoms of the alkyl, alkenyl, or alkynyl. For example, an (C₂-C₆) aminoalkyl refers to a chain comprising between 2 and 6 carbons and one or more nitrogen atoms positioned between the carbon atoms.

The terms “hydroxy” and “hydroxyl” mean the radical —OH.

The terms “alkoxyl” or “alkoxy” as used herein refers to an alkyl group, as defined above, having an oxygen radical attached thereto, and can be represented by one of —O-alkyl, —O-alkenyl, and —O-alkynyl. Aroxy can be represented by —O-aryl or O-heteroaryl, wherein aryl and heteroaryl are as defined herein. The alkoxy and aroxy groups can be substituted as described above for alkyl. Exemplary alkoxy groups include, but are not limited to O-methyl, O-ethyl, O-n-propyl, O-isopropyl, O-n-butyl, O-isobutyl, O-sec-butyl, O-tert-butyl, O-pentyl, O-hexyl, O-cyclopropyl, O-cyclobutyl, O-cyclopentyl, O-cyclohexyl and the like.

As used herein, the term “carbonyl” means the radical —C(O)—. It is noted that the carbonyl radical can be further substituted with a variety of substituents to form different carbonyl groups including acids, acid halides, amides, esters, ketones, and the like.

The term “carboxy” means the radical —C(O)O—. It is noted that compounds described herein containing carboxy moieties can include protected derivatives thereof, i.e., where the oxygen is substituted with a protecting group. Suitable protecting groups for carboxy moieties include benzyl, tert-butyl, and the like. As used herein, a carboxy group includes —COOH, i.e., carboxyl group.

The term “cyano” means the radical —CN.

The term “nitro” means the radical —NO₂.

The term, “heteroatom” refers to an atom that is not a carbon atom. Particular examples of heteroatoms include, but are not limited to nitrogen, oxygen, sulfur and halogens. A “heteroatom moiety” includes a moiety where the atom by which the moiety is attached is not a carbon. Examples of heteroatom moieties include —N═, —NR^{N}—, —N⁺(O⁻)═, —O—, —S— or —S(O)₂—, —OS(O)₂—, and —SS—, wherein R^{N} is H or a further substituent.

“Acyl” refers to an alkyl-CO— group, wherein alkyl is as previously described. Exemplary acyl groups comprise alkyl of 1 to about 30 carbon atoms. Exemplary acyl groups also include acetyl, propanoyl, 2-methylpropanoyl, butanoyl and palmitoyl.

“Alkoxycarbonyl” refers to an alkyl-O—CO— group. Exemplary alkoxycarbonyl groups include methoxycarbonyl, ethoxycarbonyl, butyloxycarbonyl, and t-butyloxycarbonyl.

“Carbamoyl” refers to an H2N—CO— group.

“Alkylcarbamoyl” refers to a R′RN—CO— group, wherein one of R and R′ is hydrogen and the other of R and R′ is alkyl as previously described.

“Dialkylcarbamoyl” refers to R′RN—CO— group, wherein each of R and R′ is independently alkyl as previously described.

The term “optionally substituted” means that the specified group or moiety is unsubstituted or is substituted with one or more substituents (typically 1, 2, 3, 4, 5 or 6 substituents) independently selected from the group of substituents listed below in the definition for “substituents” or otherwise specified. The term “substituents” refers to a group “substituted” on a substituted group at any atom of the substituted group. Suitable substituents include, without limitation, halogen, hydroxy, caboxy, oxo, nitro, haloalkyl, alkyl, alkenyl, alkynyl, alkaryl, aryl, heteroaryl, cyclyl, heterocyclyl, aralkyl, alkoxy, aryloxy, amino, acylamino, alkylcarbanoyl, arylcarbanoyl, aminoalkyl, alkoxycarbonyl, carboxy, hydroxyalkyl, alkanesulfonyl, arenesulfonyl, alkanesulfonamido, arenesulfonamido, aralkylsulfonamido, alkylcarbonyl, acyloxy, cyano or ureido. In some cases, two substituents, together with the carbons to which they are attached to can form a ring.

For example, any alkyl, alkenyl, cycloalkyl, heterocyclyl, heteroaryl or aryl is optionally substituted with 1, 2, 3, 4 or 5 groups selected from OH, CN, SH, SO2NH2, SO2(C1-C4)alkyl, SO2NH(C1-C4)alkyl, halogen, carbonyl, thiol, cyano, NH2, NH(C₁-C₄)alkyl, N[(C₁-C₄)alkyl]₂, C(O)NH₂, COOH, COOMe, acetyl, (C₁-C₈)alkyl, O(C₁-C₈)alkyl, O(C₁-C₈)haloalkyl, (C₂-C₈)alkenyl, (C₂-C₈)alkynyl, haloalkyl, thioalkyl, cyanomethylene, alkylaminyl, aryl, heteroaryl, substituted aryl, NH₂—C(O)-alkylene, NH(Me)-C(O)-alkylene, CH₂—C(O)— alkyl, C(O)— alkyl, alkylcarbonylaminyl, CH₂—[CH(OH)]ₘ—(CH₂)ₚ—OH, CH₂—[CH(OH)]ₘ—(CH₂)ₚ—NH₂ or CH₂-aryl-alkoxy; or wherein any alkyl, cycloalkyl or heterocyclyl is optionally substituted with oxo; “m” and “p” are independently 1, 2, 3, 4, 5 or 6.

In some embodiments, an optionally substituted group is substituted with 1 substituent. In some other embodiments, an optionally substituted group is substituted with 2 independently selected substituents, which can be same or different. In some other embodiments, an optionally substituted group is substituted with 3 independently selected substituents, which can be same, different or any combination of same and different. In still some other embodiments, an optionally substituted group is substituted with 4 independently selected substituents, which can be same, different or any combination of same and different. In yet some other embodiments, an optionally substituted group is substituted with 5 independently selected substituents, which can be same, different or any combination of same and different.

The terms “pyridone” and “pyridinone” have the same meaning and are used interchangeably herein.

Substituents may be protected as necessary and any of the protecting groups commonly used in the art may be employed. Non-limiting examples of protecting groups may be found, for example, in Greene et al., Protective Groups in Organic Synthesis, 3rd Ed. (New York. Wiley, 1999).

### ADDITIONAL EMBODIMENTS

Further, although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow. Further, to the extent not already indicated, it will be understood by those of ordinary skill in the art that any one of the various embodiments herein described and illustrated can be further modified to incorporate features shown in any of the other embodiments disclosed herein.
1. A compound of Formula (I) or a pharmaceutically acceptable salt thereof,

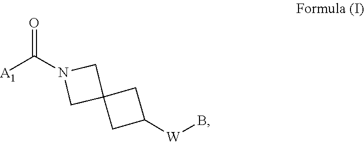

wherein.
   A₁ is

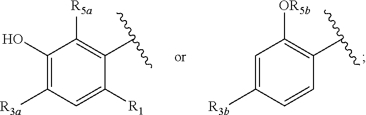

R₁ is hydrogen, halogen, or cyano;
R₃ₐ and R₅ₐ are each independently hydrogen, halogen, or optionally substituted lower alkyl;
R_{3b} is halogen;
R_{5b} is optionally substituted lower alkyl, optionally substituted lower cycloalkyl, or optionally substituted lower haloalkyl;
W is a pyrazolone, pyrrole or diazole, each independently optionally substituted with halogen, optionally substituted lower alkyl, optionally substituted lower cycloalkyl, optionally substituted lower alkoxy, or optionally substituted lower haloalkyl; and
B is an optionally substituted pyridone, or a 6-member heteroaryl comprising at least one nitrogen atom and optionally substituted with one or more halogen, optionally substituted lower alkyl, optionally substituted lower haloalkyl, or optionally substituted lower alkoxy.
2. The compound of embodiment 1, wherein W is selected from the group consisting of:

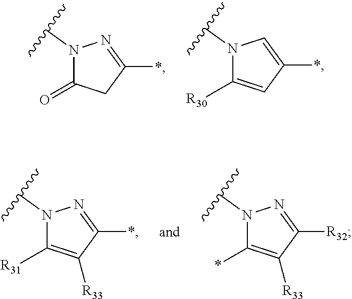

* is the bond to B;
B is an optionally substituted pyridone, or a pyridine optionally substituted with halogen, lower alkoxy, lower alkyl, or lower haloalkyl;
R₃₀ is hydrogen, halogen, lower alkyl, lower alkoxy, lower haloalkyl, or lower cycloalkyl; and
R₃₁, R₃₂ and R₃₃ are each independently hydrogen, halogen, lower alkyl, lower alkoxy, lower haloalkyl, or lower cycloalkyl, provided that at least one of R₃₁ and R₃₃ is not hydrogen, and provided that at least one of R₃₂ and R₃₃ is not hydrogen.
3. The compound of embodiment 1 or embodiment 2, wherein W is selected from the group consisting of:

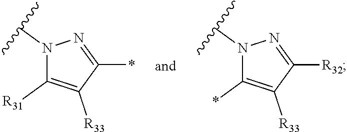

* is the bond to B, and
R₃₁, R₃₂ and R₃₃ are each independently hydrogen, halogen, lower alkyl, lower alkoxy, lower haloalkyl, or lower cycloalkyl, provided that at least one of R₃₁ and R₃₃ is not hydrogen, and provided that at least one of R₃₂ and R₃₃ is not hydrogen.
4. The compound of embodiment 1, wherein
   W is a diazole optionally substituted with one of methyl, cyclopropyl, or CF₃; and
   B is an optionally substituted pyridone, or a pyridine optionally substituted with fluoro, chloro, methoxy, methyl or CF₃.
5. The compound of embodiment 4, wherein
   R₁ is hydrogen, fluoro, or cyano;
   R₃ₐ and R₅ₐ are each independently hydrogen, fluoro, or methyl;
   R_{3b} is fluoro; and
   R_{5b} is cyclopropyl, or lower alkyl optionally substituted with one or more fluoro.
6. The compound of embodiment 1 or embodiment 2, wherein W is

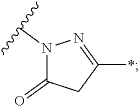

* is the bond to B.
7. The compound of embodiment 1 or embodiment 2, wherein W is

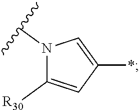

* is the bond to B; and
R₃₀ is hydrogen, halogen, lower alkyl, lower alkoxy, lower haloalkyl, or lower cycloalkyl.
8. The compound of embodiment 7, wherein R₃₀ is methoxy.
9. The compound of any one of embodiments 1-3, wherein W is

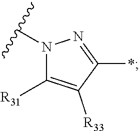

* is the bond to B; and
R₃₁ and R₃₃ are each independently hydrogen, halogen, lower alkyl, lower alkoxy, lower haloalkyl, or lower cycloalkyl, provided that at least one of R₃₁ and R₃₃ is not hydrogen.
10. The compound of embodiment 9, wherein R₃₁ and R₃₃ are each independently hydrogen, methyl. CF₃, or cyclopropyl, provided that at least one of R₃₁ and R₃₃ is not hydrogen.
11. The compound of anyone of embodiments 1-3, wherein W is

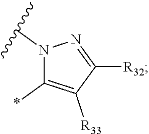

* is the bond to B; and
R₃₂ and R₃₃ are each independently hydrogen, halogen, lower alkyl, lower alkoxy, lower haloalkyl, or lower cycloalkyl, provided that at least one of R₃₂ and R₃₃ is not hydrogen.
12. The compound of embodiment 11, wherein R₃₂ and R₃₃ are each independently hydrogen, methyl. CF₃, or cyclopropyl, provided that at least one of R₃₂ and R₃₃ is not hydrogen.
13. The compound of any one of embodiments 1-12, wherein B is optionally substituted 2-pyridone, or pyridine optionally substituted with fluoro, chloro, methyl, methoxy or CF₃.
14. The compound of any one of embodiments 1-12, wherein B is optionally substituted 2-pyridone.
15. The compound of any one of embodiments 1-12, wherein B is

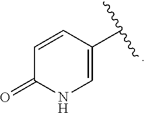 [SMILES: *c1ccc(=O)[nH]c1]

16. The compound of any one of embodiments 1-12, wherein B is

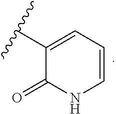 [SMILES: *c1ccc[nH]c1=O]

17. The compound of any one of embodiments 1-12, wherein B is

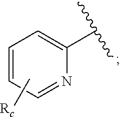

and
R_{c} is hydrogen, fluoro, methoxy, or CF₃.
18. The compound of any one of embodiments 1-12, wherein B is

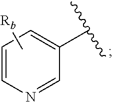

and
   R_{b} is hydrogen, fluoro, methoxy, or CF₃.
19. The compound of embodiment 18, wherein B is

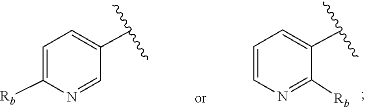

and R_{b} is hydrogen, fluoro or methoxy.
20. The compound of embodiment 18, wherein B is

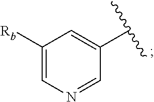

and R_{b} is hydrogen, fluoro or methoxy.
21. The compound of any one of embodiments 1-12, wherein B is

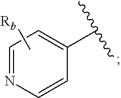

and R_{d} is hydrogen, fluoro, methoxy, or CF₃.
22. The compound ofany one of embodiments 1-21, wherein A₁ is

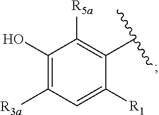

R₁ is hydrogen, halogen, or cyano, and
R₃ₐ and R₅ₐ are each independently hydrogen, halogen, or optionally substituted lower alkyl.
23 The compound of embodiment 22, wherein R₅ₐ is hydrogen.
24. The compound of embodiment 23, wherein R₁ is fluoro.
25. The compound of embodiment 24, wherein R₃ₐ is hydrogen, fluoro, or methyl.
26. The compound of embodiment 25, wherein R₃ₐ is hydrogen.
27. The compound of any one of embodiments 22-26, wherein B is optionally substituted 2-pyridone, or pyridine optionally substituted with fluoro or methoxy.
28. The compound of any one of embodiments 1-21, wherein A₁ is

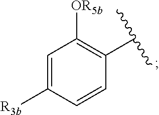

R_{3b} is halogen;
R_{5b} is optionally substituted lower alkyl, optionally substituted lower cycloalkyl, or optionally substituted lower haloalkyl.
29. The compound of embodiment 28, wherein R_{5b} is cyclopropyl, or lower alkyl optionally substituted with one or more fluoro.
30. The compound of embodiment 29, wherein R_{3b} is fluoro.
31. The compound of any one of embodiments 28-30, wherein B is optionally substituted 2-pyridone, or pyridine optionally substituted with fluoro or methoxy.
32. A compound of Formula (I-A-1) or a pharmaceutically acceptable salt thereof:

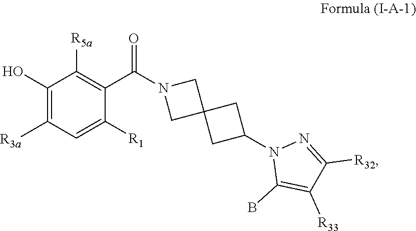

wherein:
R₁ is halogen or cyano;
R₃ₐ is hydrogen, optionally substituted lower alkyl, or halogen;
R₅ₐ is hydrogen, halogen, or optionally substituted lower alkyl;
B is

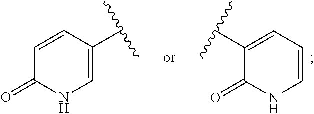 [SMILES: *c1ccc(=O)[nH]c1 ; *c1ccc[nH]c1=O]

and
   R₃₂ and R₃₃ are each independently hydrogen, optionally substituted lower alkyl, optionally substituted lower haloalkyl, or optionally substituted lower cycloalkyl, provided that at least one of R₃₂ and R₃₃ is not hydrogen.
33. The compound of embodiment 32, wherein
   R₁ is F;
   R₃ₐ is hydrogen;
   R₅ₐ is hydrogen; and
   R₃₂ and R₃₃ are each independently hydrogen, methyl, CF₃, or cyclopropyl, provided that at least one of R₃₂ and R₃₃ is not hydrogen.
34. The compound of embodiment 33, wherein R₃₂ is CF₃ and R₃₃ is hydrogen, or R₃ is hydrogen and R₃₃ is CF₃.
35. A compound of Formula (I-B-1) or a pharmaceutically acceptable salt thereof:

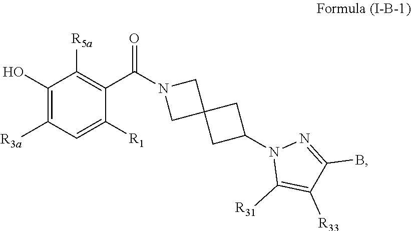

wherein:
R₁ is halogen or cyano;
R₃ₐ is hydrogen, optionally substituted lower alkyl, or halogen;
R₅ₐ is hydrogen, halogen or optionally substituted lower alkyl;
B is

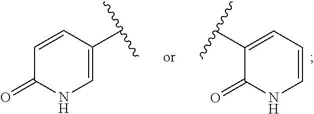 [SMILES: *c1ccc(=O)[nH]c1 ; *c1ccc[nH]c1=O]

R₃₁ and R₃₃ are each independently hydrogen, optionally substituted lower alkyl, optionally substituted lower haloalkyl, or optionally substituted lower cycloalkyl, provided that at least one of R₃₁ and R₃₃ is not hydrogen.
36. The compound of embodiment 35, wherein
   R₁ is F;
   R₃ₐ is hydrogen;
   R₅ₐ is hydrogen, and
   R₃₁ and R₃₃ are each independently hydrogen, methyl, CF₃, or cyclopropyl, provided that at least one of R₃₁ and R₃₃ is not hydrogen.
37. The compound of embodiment 36, wherein R₃₁ is CF₃ and R₃₃ is hydrogen, or R₃₁ is hydrogen and R₃₃ is CF₃.
38. A compound of Formula (I-A-1) or a pharmaceutically acceptable salt thereof:

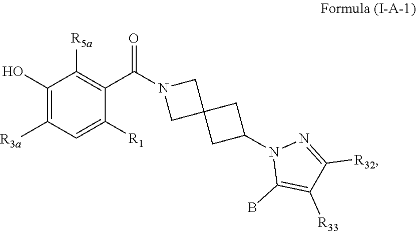

wherein:
R₁ is halogen or cyano;
R₃ₐ is hydrogen, optionally substituted lower alkyl, or halogen;
R₅ₐ is hydrogen, halogen or optionally substituted lower alkyl;
B is

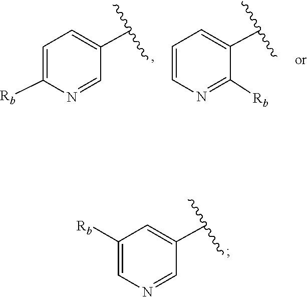

R_{b} is hydrogen, fluoro or methoxy; and
R₃₂ and R₃₃ are each independently hydrogen, optionally substituted lower alkyl, optionally substituted lower haloalkyl, or optionally substituted lower cycloalkyl, provided that at least one of R₃₂ and R₃₃ is not hydrogen.
39. The compound of embodiment 38, wherein
   R₁ is F;
   R₃ₐ is hydrogen;
   R₅ₐ is hydrogen; and
   R₃₂ and R₃₃ are each independently hydrogen, methyl, CF₃, or cyclopropyl, provided that at least one of R₃₂ and R₃₃ is not hydrogen;
   B is

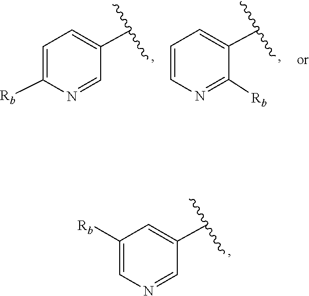

and R_{b} is hydrogen, fluoro or methoxy.
40. The compound of embodiment 39, wherein R₃₂ is CF₃ and R₃₃ is hydrogen, or R₃₂ is hydrogen and R₃₃ is CF₃.
41. A compound of Formula (I-B-1) or a pharmaceutically acceptable salt thereof:

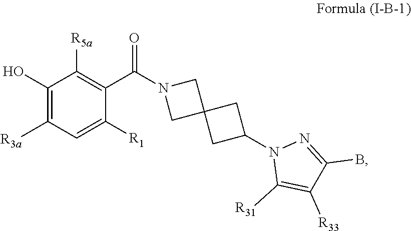

wherein:
R₁ is halogen or cyano;
R₃ is hydrogen, optionally substituted lower alkyl, or halogen;
R₅ₐ is hydrogen, halogen or optionally substituted lower alkyl;
B is

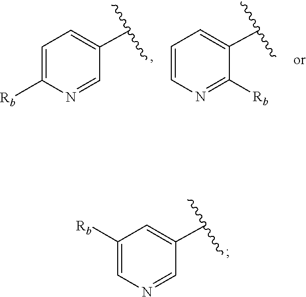

R_{b} is hydrogen, fluoro or methoxy; and
R₃₁ and R₃₃ are each independently hydrogen, optionally substituted lower alkyl, optionally substituted lower haloalkyl, or optionally substituted lower cycloalkyl, provided that at least one of R₃₁ and R₃₃ is not hydrogen.
42. The compound of embodiment 41, wherein
   R₁ is F;
   R₃ₐ is hydrogen;
   R₅ₐ is hydrogen;
   R₃₁ and R₃₃ are each independently hydrogen, methyl, CF₃, or cyclopropyl, provided that at least one of R₃₁ and R₃₃ is not hydrogen;
   B is

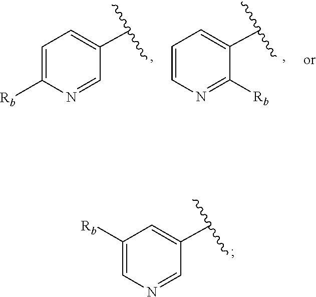

and
R_{b} is hydrogen, fluoro or methoxy.
43. The compound of embodiment 42, wherein R₃₁ is CF₃ and R₃₃ is hydrogen, or R₃₁ is hydrogen and R₃₃ is CF₃.
44. A compound of Formula (II) or a pharmaceutically acceptable salt thereof:

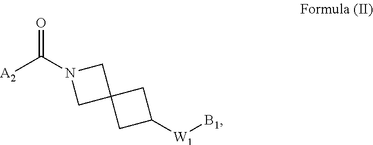

wherein:
A₂ is

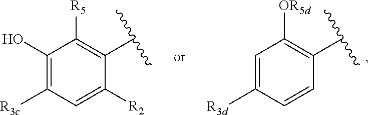

R₂ is hydrogen, halogen, or cyano;
R_{3c} and R_{5c} are each independently hydrogen, halogen, or optionally substituted lower alkyl;
R_{3d} is halogen;
R_{5d} is optionally substituted lower alkyl, optionally substituted lower cycloalkyl, or optionally substituted lower haloalkyl;
W₁ is

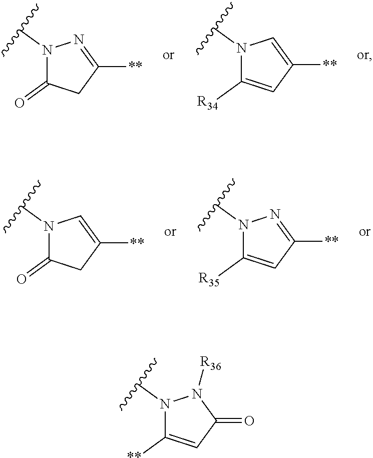

** is the bond to B₁;
R₃₄ is lower alkoxy, lower alkyl, lower haloalkyl or lower cycloalkyl;
R₃₅ is lower alkoxy, lower alkyl, lower haloalkyl or lower cycloalkyl;
R₃₆ is hydrogen or lower alkyl; and
B₁ is phenyl optionally substituted with halogen, optionally substituted lower alkoxy, optionally substituted lower alkyl, or optionally substituted lower haloalkyl.
45. The compound of embodiment 44, wherein W₁ is

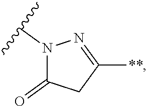

** is the bond to B₁; and
B₁ is phenyl optionally substituted with halogen, optionally substituted lower alkoxy, optionally substituted lower alkyl, or optionally substituted lower haloalkyl.
46. The compound of embodiment 44, wherein W₁ is

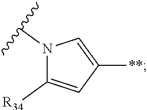

R₃₄ is lower alkoxy, lower alkyl, lower haloalkyl or lower cycloalkyl;
* is the bond to B₁; and
B₁ is phenyl optionally substituted with halogen, optionally substituted lower alkoxy, optionally substituted lower alkyl, or optionally substituted lower haloalkyl.
47. The compound of any one of embodiments 44-46, wherein R_{3d} is F, and R_{5d} is methyl or cyclopropyl.
48. The compound of embodiment 47, wherein R_{d} is cyclopropyl.
49. The compound of any one of embodiments 44 or 46, wherein R₃₄ is methoxy.
50. The compound of embodiment 1, wherein the compound is

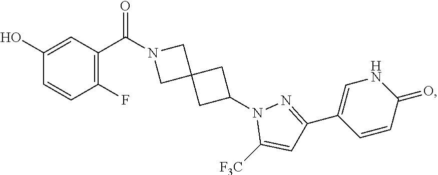 [SMILES: O=C(c1cc(O)ccc1F)N1CC2(CC(n3nc(-c4ccc(=O)[nH]c4)cc3C(F)(F)F)C2)C1]

or a pharmaceutically acceptable salt thereof
51. The compound of embodiment 1, wherein the compound is

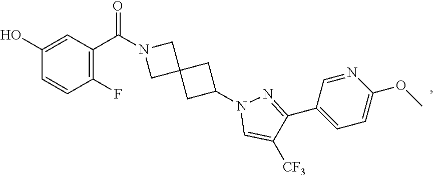 [SMILES: COc1ccc(-c2nn(C3CC4(C3)CN(C(=O)c3cc(O)ccc3F)C4)cc2C(F)(F)F)cn1]

or a pharmaceutically acceptable salt thereof.
52. The compound of embodiment 1, wherein the compound is

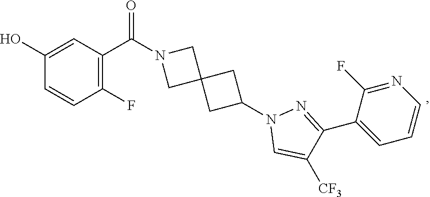 [SMILES: O=C(c1cc(O)ccc1F)N1CC2(CC(n3cc(C(F)(F)F)c(-c4cccnc4F)n3)C2)C1]

or a pharmaceutically acceptable salt thereof.
53. The compound of embodiment 1, wherein the compound is

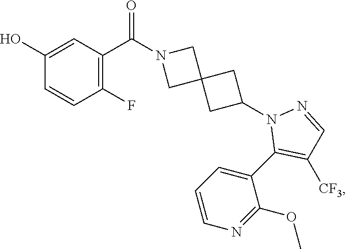 [SMILES: COc1ncccc1-c1c(C(F)(F)F)cnn1C1CC2(C1)CN(C(=O)c1cc(O)ccc1F)C2]

or a pharmaceutically acceptable salt thereof.
54. The compound of embodiment 1, wherein the compound is

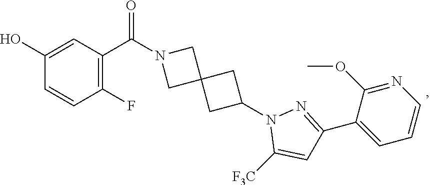 [SMILES: COc1ncccc1-c1cc(C(F)(F)F)n(C2CC3(C2)CN(C(=O)c2cc(O)ccc2F)C3)n1]

or a pharmaceutically acceptable salt thereof.
55. The compound of embodiment 1, wherein the compound is

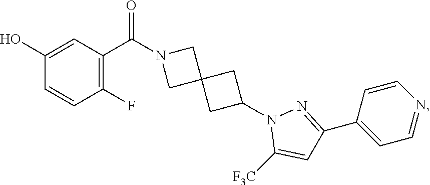 [SMILES: O=C(c1cc(O)ccc1F)N1CC2(CC(n3nc(-c4ccncc4)cc3C(F)(F)F)C2)C1]

or a pharmaceutically acceptable salt thereof.
56. The compound of embodiment 1, wherein the compound is

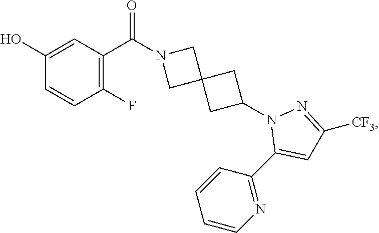 [SMILES: O=C(c1cc(O)ccc1F)N1CC2(CC(n3nc(C(F)(F)F)cc3-c3ccccn3)C2)C1]

or a pharmaceutically acceptable salt thereof.
57. The compound of embodiment 1, wherein the compound is

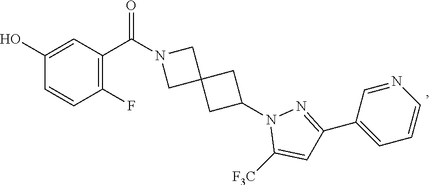 [SMILES: O=C(c1cc(O)ccc1F)N1CC2(CC(n3nc(-c4cccnc4)cc3C(F)(F)F)C2)C1]

or a pharmaceutically acceptable salt thereof.
58. The compound of embodiment 1, wherein the compound is

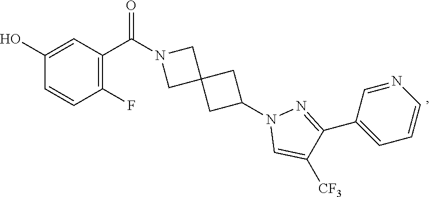 [SMILES: O=C(c1cc(O)ccc1F)N1CC2(CC(n3cc(C(F)(F)F)c(-c4cccnc4)n3)C2)C1]

or a pharmaceutically acceptable salt thereof.
59. The compound of embodiment 1, wherein the compound is

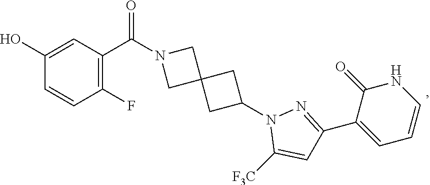 [SMILES: O=C(c1cc(O)ccc1F)N1CC2(CC(n3nc(-c4ccc[nH]c4=O)cc3C(F)(F)F)C2)C1]

or a pharmaceutically acceptable salt thereof.
60. The compound of embodiment 1, wherein the compound is

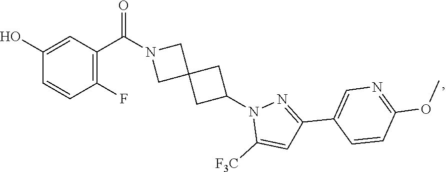 [SMILES: COc1ccc(-c2cc(C(F)(F)F)n(C3CC4(C3)CN(C(=O)c3cc(O)ccc3F)C4)n2)cn1]

or a pharmaceutically acceptable salt thereof.
61. A compound of Formula (II-A-1) or a pharmaceutically acceptable salt thereof,

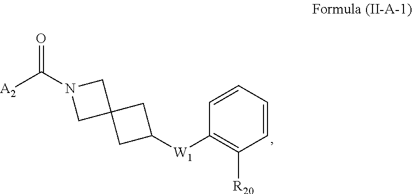

wherein:
   A₂ is

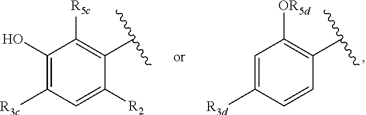

R₂ is hydrogen, halogen, or cyano;
R_{3c} and R_{5c} are each independently hydrogen, halogen, or lower alkyl;
R_{3d} is halogen;
R_{5d} is lower alkyl, lower cycloalkyl, or lower haloalkyl;
W₁ is

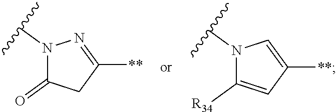

** is the bond to B₁;
R₃₄ is lower alkoxy; and
R₂₀ is hydrogen, halogen, lower alkyl or lower haloalkyl
62. The compound of embodiment 61, wherein R₂₀ is fluoro or CF₃.
63. The compound of embodiment 61 or embodiment 62, wherein R₂ and R_{3d} are both fluoro.
64. The compound of embodiment 63, wherein R_{3c} and R_{5c} are both hydrogen.
65. The compound of embodiment 62 or embodiment 63, wherein R_{5d} is cyclopropyl.
66. The compound of any one of embodiments 61-65, wherein A₂ is

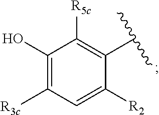

R₂ is hydrogen, halogen, or cyano; and
R_{3c} and R_{5c} are each independently hydrogen, halogen, or lower alkyl.
67. The compound of any one of embodiments 61-65, wherein A₂ is;

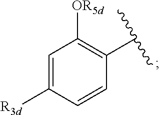

R_{3d} is halogen; and
R_{5d} is lower alkyl, lower cycloalkyl or lower haloalkyl.
68. The compound of any one of embodiments 61-67, wherein R₂₀ is fluoro.
69 The compound of any one of embodiments 61-67, wherein R₂₀ is CF₃.
70. The compound of any one of embodiments 61-69, wherein R₃₄ is methoxy.
71. The compound of any one of embodiments 35-37, wherein R₃₁ is CF₃ and R₃₃ is hydrogen.
72. The compound of any one of embodiments 35-37, wherein R₃₁ is hydrogen and R₃₃ is CF₃.
73. The compound of any one of embodiments 35-37 or 71-72, wherein B is

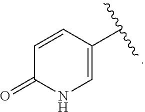 [SMILES: *c1ccc(=O)[nH]c1]

74. The compound of any one of embodiments 35-37 or 71-72, wherein B is

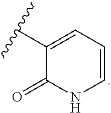 [SMILES: *c1ccc[nH]c1=O]

75. The compound of any one of embodiments 41-43, wherein R₃₁ is CF₃ and R₃₃ is hydrogen.
76. The compound of any one of embodiments 41-43, wherein R₁ is hydrogen and R₃₃ is CF₃.
77. The compound of any one of embodiments 41-43 or 75-76, wherein B is

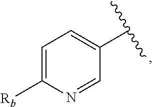

and R_{b} is hydrogen, fluoro or methoxy.
78. The compound of any one of embodiments 41-43 or 75-76, wherein B is

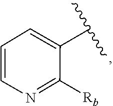

and R_{b} is hydrogen, fluoro or methoxy.
79. The compound of an one of embodiments 41-43 or 75-76, wherein B is

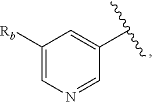

and Rₕ is hydrogen, fluoro or methoxy.
80. The compound of any one of embodiments 41-43, or 75-79, wherein R_{b} is hydrogen.
81. The compound of any one of embodiments 41-43, or 75-79, wherein R_{b} is fluoro.
82. The compound of any one of embodiments 41-43, or 75-79, wherein Rₕ is methoxy.
83. The compound of any one of embodiments 38-40, wherein R₃₁ is CF₃ and R₃₃ is hydrogen.
84. The compound of any one of embodiments 38-40, wherein R₃₁ is hydrogen and R₃₃ is CF₃.
85. The compound of any one of embodiments 38-40 or 83-84, wherein B is

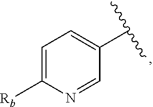

and R_{b} is hydrogen, fluoro or methoxy.
86. The compound of any one of embodiments 38-40 or 83-84, wherein B is

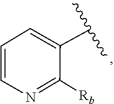

and R_{b} is hydrogen, fluoro or methoxy.
87 The compound of an one of embodiments 38-40 or 83-84, wherein B is

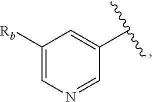

and R_{b} is hydrogen, fluoro or methoxy.
88. The compound of any one of embodiments 38-40, or 83-87, wherein R_{b} is hydrogen.
89. The compound of any one of embodiments 38-40, or 83-87, wherein R_{b} is fluoro.
90. The compound of any one of embodiments 38-40, or 83-87, wherein Rₕ is methoxy.
91. A compound of Formula (I) or a pharmaceutically acceptable salt thereof,

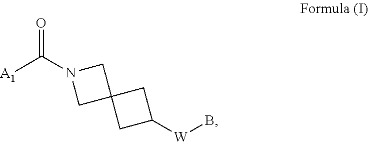

wherein:
   A₁ is

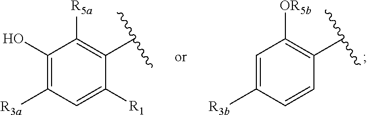

R₁ is hydrogen, halogen, or cyano;
R₃ₐ and R₅ₐ are each independently hydrogen, halogen, or optionally substituted lower alkyl;
R_{3b} is halogen;
R_{5b} is optionally substituted lower alkyl, optionally substituted lower cycloalkyl, or optionally substituted lower haloalkyl;
W is a pyrazolone, pyrrole or diazole, each independently optionally substituted with halogen, optionally substituted lower alkyl, optionally substituted lower cycloalkyl, optionally substituted lower alkoxy, or optionally substituted lower haloalkyl; and
B is 2-piperidinone or 2-dihydropyridinone optionally substituted pyridone, or a 6-member heteroaryl comprising at least one nitrogen atom and optionally substituted with one or more halogen, optionally substituted lower alkyl, optionally substituted lower haloalkyl, or optionally substituted lower alkoxy.
92. The compound of embodiment 91, wherein
   a. R₁ is fluoro,
   b. R₃ₐ and R₅ₐ are each independently hydrogen;
   c. R_{3b} is fluoro; and R_{5b} is cyclopropyl.
93. The compound of embodiment 92, wherein B is unsubstituted.
94. The compound of any one of embodiments 91-93, wherein B is

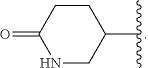 [SMILES: *C1CCC(=O)NC1]

95. The compound of any one of embodiments 91-93, wherein B is

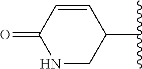 [SMILES: *C1C=CC(=O)NC1]

96. The compound of any one of embodiments 91-93, wherein B is

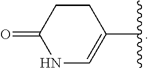 [SMILES: *C1=CNC(=O)CC1]

97. A compound of Formula (IV) or a pharmaceutically acceptable salt thereof,

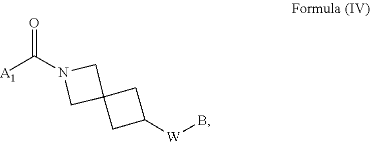

wherein:
   A₁ is

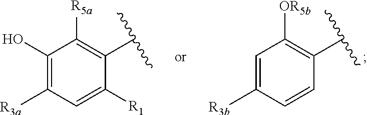

R₁ is hydrogen, halogen, or cyano;
R₃ₐ and R₅ₐ are each independently hydrogen, halogen, or optionally substituted lower alkyl;
R_{3b} is halogen;
R_{5b} is optionally substituted lower alkyl, optionally substituted lower cycloalkyl, or optionally substituted lower haloalkyl;
W is

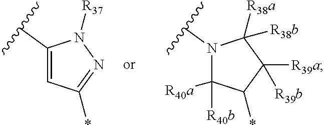

R₃₇, R₃₈ₐ, R_{38b}, R₃₉ₐ, R_{39b}, R₄₀ₐ, R_{40b}, is hydrogen, halogen, lower alkyl, lower alkoxy, lower haloalkyl, or lower cycloalkyl;
B is an optionally substituted 134yridine, phenyl or a 6-member heteroaryl comprising at least one nitrogen atom and optionally substituted with one or more halogen, optionally substituted lower alkyl, optionally substituted lower haloalkyl, or optionally substituted lower alkoxy.
98 The compound of embodiment 97, wherein B is phenyl.
99. The compound of embodiment 98, wherein B is substituted with one or more halogen or haloalkyl.
100. The compound of embodiment 99, wherein B is substituted with one of more fluoro.
101. The compound of embodiment 100, wherein B is

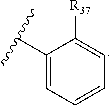

102. The compound of embodiment 101, wherein B is F, methyl or CF
103. The compound of any one of embodiments 97-102, wherein W is

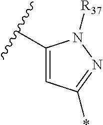

104. The compound of embodiment 103, wherein R₃₇ is hydrogen, halogen, lower alkyl, lower alkoxy, lower haloalkyl, or lower cycloalkyl.
105. The compound of embodiment 103, wherein R₃₇ is hydrogen, methyl, methoxy, CF₃, or cyclopropyl.
106. The compound of embodiment 103, wherein R₃₇ is methoxy.
107. The compound of embodiment 103, wherein R₃₇ is methyl.
108. The compound of embodiment 103, wherein R₃₇ is hydrogen.
109. The compound of embodiment 103, wherein R₃₇ is cyclopropyl.
110. The compound of embodiment 103, wherein R₃₇ is CF₃.
111. The compound of any one of embodiments 97-102, wherein W is

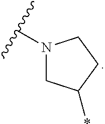 [SMILES: *C1CCN(*)C1]

112. The compound of any one of embodiments 97-102, wherein W is

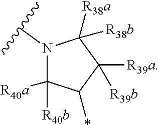

113. The compound of embodiment 111, wherein R₃₈ₐ, R_{38b}, R₃₉ₐ, R_{39b}, R₄₀ₐ, R_{40b}, is hydrogen, halogen, methyl, methoxy, CF₃, or cyclopropyl.
114. The compound of embodiment 111, wherein R₃₈ₐ, R₃₉ₐ, R₄₀ₐ, is hydrogen.
115. The compound of embodiment 114, wherein one of R_{38b}, R_{39b}, and R₄₀ₐ, is halogen, methyl, methoxy, CF₃, or cyclopropyl.
116. The compound of embodiment 115, wherein two of R_{38b}, R_{39b}, and R₄₀ₐ, are hydrogen.
117. The compound of any one of embodiments 97-116, wherein A₁ is

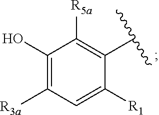

R₁ is hydrogen, halogen, or cyano; and
R₃ₐ and R₅ₐ are each independently hydrogen, halogen, or optionally substituted lower alkyl.
118. The compound of embodiment 117, wherein R₅ₐ is hydrogen.
119. The compound of any one of embodiments 117-118, wherein R₁ is fluoro.
120. The compound of any one of embodiments 117-119, wherein R₃ₐ is hydrogen, fluoro, or methyl.
121. The compound of embodiment 120, wherein R₃ₐ is hydrogen.
122. The compound of any one of embodiments 97-116, wherein A₁ is

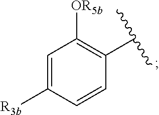

R_{3b} is halogen;
R_{5b} is optionally substituted lower alkyl, optionally substituted lower cycloalkyl, or optionally substituted lower haloalkyl.
123. The compound of embodiment 122, wherein R_{5b} is cyclopropyl, or lower alkyl optionally substituted with one or more fluoro.
124. The compound of any one of embodiments 122-123, wherein R_{3b} is fluoro.
125. The compound of embodiment 124, wherein R_{5b} is cyclopropyl.
126. A pharmaceutical composition comprising a compound of any one of embodiments 1-125, or a pharmaceutically acceptable salt thereof as the active pharmaceutical ingredient (API).
127. A method of inhibiting monoacylglycerol lipase (MAGL) in a subject comprising the step of administering to a subject in need thereof a therapeutically effective amount of a compound of any one of embodiments 1-125.
128. A method of reversibly inhibiting monoacylglycerol lipase (MAGL) comprising the step of contacting a compound of any one of embodiments 1-125 with a cell expressing MAGL.

### EXAMPLES

The following examples illustrate some embodiments and aspects of the invention. It will be apparent to those skilled in the relevant art that various modifications, additions, substitutions, and the like can be performed without altering the spirit or scope of the invention, and such modifications and variations are encompassed within the scope of the invention as defined in the claims which follow. The following examples do not in any way limit the invention.

### Example 1: Preparation of Synthetic Intermediates

### Intermediate-5: 2-(2-ethoxy-4-fluorobenzoyl)-2-azaspiro [3.3] heptane-6-carboxylic acid

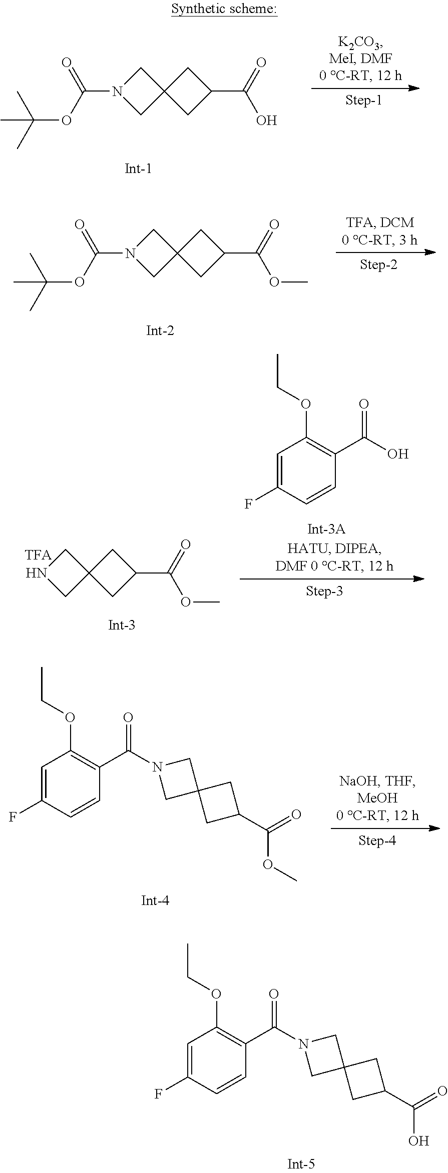 [SMILES: CC(C)(C)OC(=O)N1CC2(CC(C(=O)O)C2)C1 ; COC(=O)C1CC2(C1)CN(C(=O)OC(C)(C)C)C2 ; CCOc1cc(F)ccc1C(=O)O ; COC(=O)C1CC2(CNC2)C1 ; CCOc1cc(F)ccc1C(=O)N1CC2(CC(C(=O)OC)C2)C1 ; CCOc1cc(F)ccc1C(=O)N1CC2(CC(C(=O)O)C2)C1]

### Step-1: Synthesis of 2-(tert-butyl) 6-methyl 2-azaspiro [3.3] heptane-2,6-dicarboxylate (Int-2)

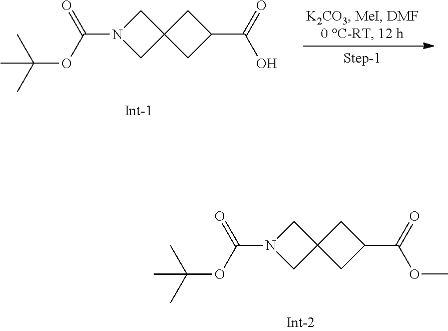 [SMILES: CC(C)(C)OC(=O)N1CC2(CC(C(=O)O)C2)C1 ; COC(=O)C1CC2(C1)CN(C(=O)OC(C)(C)C)C2]

To a stirred solution of 2-(ten-butoxycarbonyl)-2-azaspiro [3.3]heptane-6-carboxylic acid (3.0 gm, 12.44 mmol, 1.0 eq.) in N,N-Dimethyl formamide (30 mL), was added potassium carbonate (2.061 gm, 14.93 mmol, 1.2 eq.) at 0° C. The reaction was stirred at 0° C. for 10 min. Methyl Iodide (2.12 gm, 14.93 mmol, 1.2 eq.) was added dropwise and stirred the reaction mass at room temperature for 12 hrs. After completion of reaction as monitored by TLC, the reaction mixture was diluted with Ice cold water (30 mL) and extracted with Ethyl acetate (3×30 mL). The combined organic layer was washed with brine solution, dried over sodium sulfate and concentrated to obtain crude product, which was purified by combiflash using 30% Ethyl acetate in Hexane as eluent to afford 2-(tert-butyl) 6-methyl 2-azaspiro[3.3]heptane-2,6-dicarboxylate (Int-2) 3.10 gm, (Yield-97 79%). LCMS: 200.2/z [M−56]⁺

### Step-2: Synthesis of methyl 2-azaspiro [3.3] heptane-6-carboxylate (Int-3)

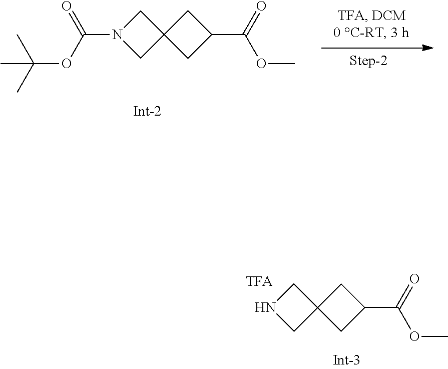 [SMILES: COC(=O)C1CC2(C1)CN(C(=O)OC(C)(C)C)C2 ; COC(=O)C1CC2(CNC2)C1]

To a stirred solution of 2-(tert-butyl) 6-methyl 2-azaspiro[3.3]heptane-2,6-dicarboxylate (Int-2) (3.0 gm, 8.0 mmol, 1.0 eq.) in Dichloromethane (30 mL) was added Trifluoroacetic acid (3.0 mL) at 0° C. and allowed to stirred the reaction at Room temperature for 3 hr; the progress of the reaction of the was monitored by TLC. After completion of reaction, the reaction mixture was evaporated under vacuum and basified with bicarbonate solution (15 mL) extracted with Ethyl acetate (3*30 mL) Ethyl acetate layer separated dried over sodium sulfate and concentrated to obtain crude product methyl 2-azaspiro[3.3]heptane-6-carboxylate (Int-3) 2.50 gm (Yield: quantitative); LCMS: 155.90/z [M+1]⁺

### Step-3: Synthesis of methyl 2-(2-ethoxy-4-fluorobenzoyl)-2-azaspiro [3.3] heptane-6-carboxylate (Int-4)

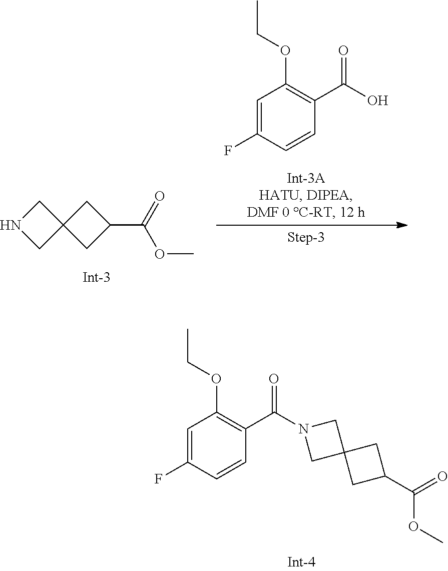 [SMILES: CCOc1cc(F)ccc1C(=O)O ; COC(=O)C1CC2(CNC2)C1 ; CCOc1cc(F)ccc1C(=O)N1CC2(CC(C(=O)OC)C2)C1]

To a stirred solution of 2-ethoxy-4-fluorobenzoic acid (2.0 gm, 10.86 mmol, 1 eq.) in N,N-Dimethyl formamide (20 mL) were added HATU (6.19 gm, 16.30 mmol, 1.5 eq.) DIPEA (4.20 gm, 32.6 mmol, 3.0 eq.) followed by methyl 2-azaspiro [3.3]heptane-6-carboxylate (Int-3) (1.33 gm, 10.86 mmol, 1.0 eq.) at 0° C. and allowed to stirred the reaction at room temperature for 12 hr; the progress of the reaction of was monitored by TLC. After completion of reaction, the reaction mixture was diluted with water (20 mL) and extracted with ethyl acetate (3×30 mL), washed with brine. The organic layer was dried over sodium sulphate and concentrated under vacuum to get crude material; which was purified by Combi-flash by using 70% Ethyl acetate in n Hexane as mobile phase to give desired product 2-(2-ethoxy-4-fluorobenzoyl)-2-azaspiro[3.3]heptane-6-carboxylic acid (Int-4) 2.4 gm (Yield: 73.52%), LCMS: 322.3 m/z [M+1]⁺.

### Step-4: Synthesis of 2-(2-ethoxy-4-fluorobenzoyl)-2-azaspiro [3.3] heptane-6-carboxylic acid (Int-5)

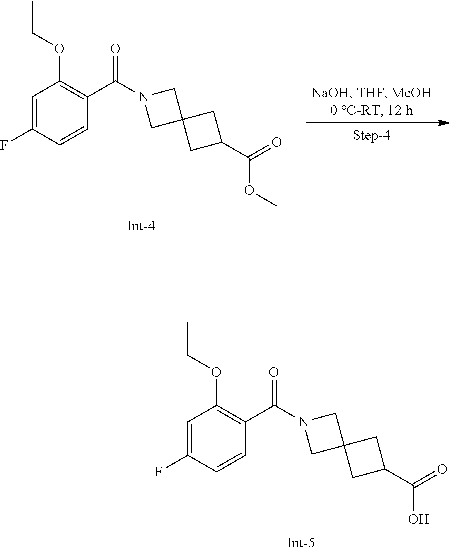 [SMILES: CCOc1cc(F)ccc1C(=O)N1CC2(CC(C(=O)OC)C2)C1 ; CCOc1cc(F)ccc1C(=O)N1CC2(CC(C(=O)O)C2)C1]

To a stirred solution of methyl 2-(2-ethoxy-4-fluorobenzoyl)-2-azaspiro [3.3]heptane-6-carboxylate (Int-4) (1.0 gm, 3.11 mmol, 1.0 eq.) in Tetrahydrofuran (10 mL), Methanol (5 mL), Water (10 mL) was added Sodium Hydroxide [NaOH](0.13 g, 6.23 mmol, 2.0 eq.) at 0° C. The reaction mixture was stirred at Room temperature for next 12 hr. The progress of the reaction was monitored by TLC; after completion of reaction, the reaction mixture was evaporated under vacuum. The crude product was acidified with 2N HCL (PH-4) The white solid was precipitated out which was filtered through Buchner funnel and dried under vacuum to give 2-(2-ethoxy-4-fluorobenzoyl)-2-azaspiro[3.3]heptane-6-carboxylic acid (Int-5), 0.75 g (Yield: 72.11%); LCMS: 308.3 m, [M+1]⁺

### Example 2: Synthesis of (2-fluoro-5-hydroxyphenyl) (6-(3-(6-methoxypyridin-3-yl)-5-trifluoro methyl)-1H-pyrazol-1-yl)-2-azaspiro [3.3]heptan-2-yl) methanone. [Compound 17] and (2-fluoro-5-hydroxyphenyl) (6-(5-(6-methoxypyridin-3-yl)-3-(trifluoro methyl)-1H-pyrazol-1-yl)-2-azaspiro [3.3] heptan-2-yl) methanone). [Compound 18]

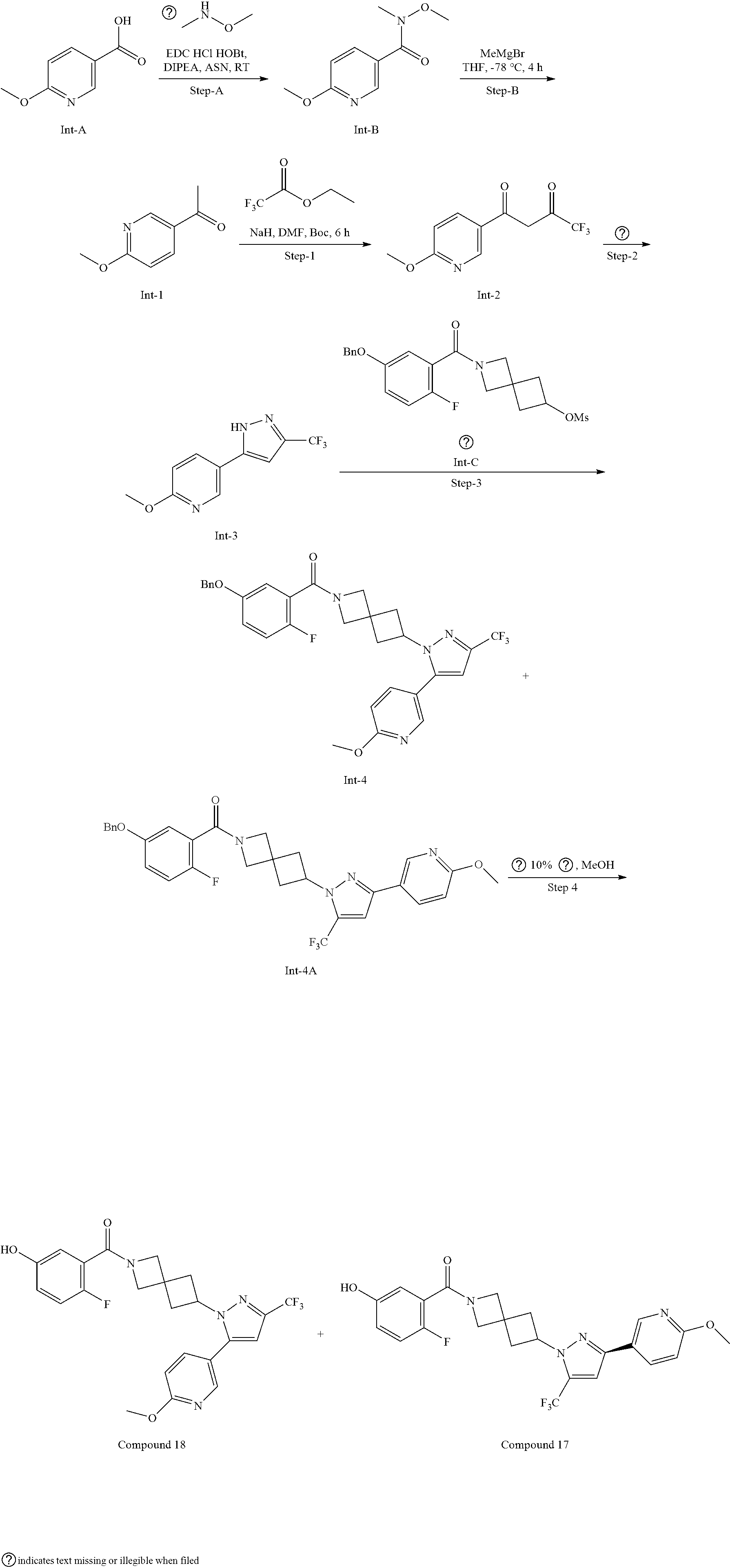

### Step-A: Synthesis of N,6-dimethoxy-N-methylnicotinamide (Int-B)

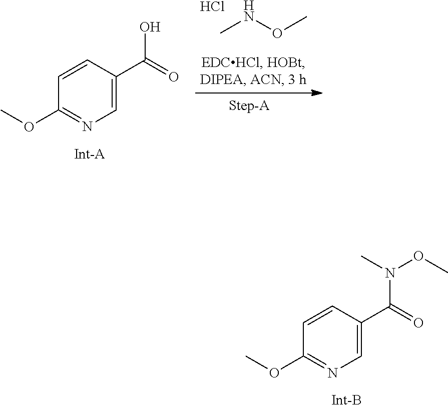

To a solution of the 6-methoxynicotinic acid (4.0 g, 26.26 mmol, 1.0 eq.) in acetonitrile (40 mL) was added N, N-Diisopropylethylamine (13.4 g, 78.78 mmol, 3.0 eq.) and stirred at 00 C. 1-Ethyl-3-(3-dimethylaminopropyl) carbodiimide (EDC-HCl) (7.5 g, 39.65 mmol, 1.5 eq.), l-Hydroxybenzotriazole (HOBt) (4.5 g, 33.96 mmol, 1.3 eq.), and the N,O-dimethyl hydroxylamine (3.3 g, 39.65 mmol, 1.5 eq.) were added to the reaction after 15 mins time gap. The reaction was stirred at room temperature for 3 h. After completion of the reaction (checked by TLC, LCMS), mixture was diluted with H2O (50 mL) and extracted with ethyl acetate (50 mL*3). The org layer was dried Na2SO4 and concentrated through vacuum to get crude compound of N,6-dimethoxy-N-methylnicotinamide (Int-B). (3.9 g, 76.10%). Which was used in next step without further purification. LCMS: m/z 197.4 [M+1]+. 1H NMR (400 MHz, DMSO-d6) δ 8.50 (d, J=1.9 Hz, 1H), 7.97 (dd, J=8.6, 2.2 Hz, 1H), 6.90 (d, J=8.6 Hz, 1H), 3.92 (s, 3H), 3.53 (s, 3H), 3.28 (s, 3H).

### Step-B: Synthesis of 146-methoxypyridin-3-yl) ethan-1-one (Int-1)

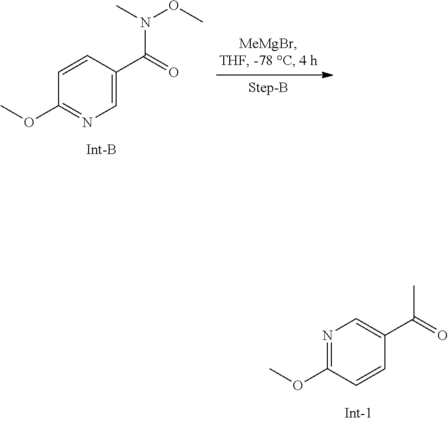

To a solution of the N,6-dimethoxy-N-methylnicotinamide (3.9 g, 19.89 mmol) in anhydrous tetrahydrofuran (78 mL) at −780 C under nitrogen was added dropwise MeMgBr (3M in ether, 16.57 mL, 49.724 mmol). The cold bath was removed and the reaction was stirred at room temperature for 4 h. The mixture was diluted with sat. NH4Cl and extracted with ethyl acetate (50 mL*3). The combined organics were washed with brine, dried Na2SO4, concentrated through vacuum. The resulting material was purified by flash chromatography, product eluting with 50-100% ethyl acetate in n-hexane to provide the product as a white solid 1-(6-methoxypyridin-3-yl) ethan-1-one (Int-1) (2.0 g, 66.56%). LCMS: m/z 152.1 [M+1]+. 1H NMR (400 MHz, DMSO-d6) δ 8.85 (s, 1H), 8.19 (d, J=8.7 Hz, 1H), 6.94 (d, J=8.6 Hz, 1H), 3.95 (s, 3H), 3.36 (s, 2.64-2.54 (m, 3H).

### Step-1 Synthesis of 4,4,4-Trifluoro-1-(6-methoxypyridin-3-yl) butane-1,3-dione (Int-2)

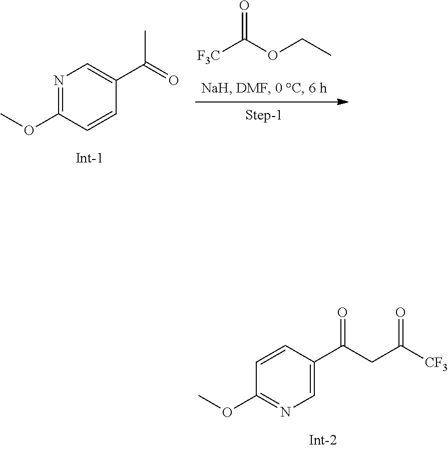

To a stirred solution of 1-(6-methoxypyridin-3-yl)ethan-1-one (6.5 g, 43.05 mmol, 1.0 eq) in N,N dimethyl formamide (50 mL) was added Sodium hydride (2.3 g, 64.57 mmol, 1.5 eq.) at 0° C. The reaction was stirred at 0° C. for 30 min. ethyl 2,2,2-trifluoroacetate (5.0 ml, 64.57 mmol, 1.5 eq.) was added dropwise and stirred the reaction mass at room temperature for 6 h. After completion of reaction as monitored by TLC, the reaction mixture was diluted with Ice cold water (5 mL) and extracted with Ethyl acetate (2*20 mL). The combined organic layer was washed with brine solution, dried over Na2SO4 and concentrated under vacuum to obtain crude product, which was purified by Combi flash using 10% Ethyl acetate in n-hexane as eluent to get 4,4,4-trifluoro-1-(6-methoxypyridin-3-yl) butane-1,3-dione (Int-2) (8.2 g, 77.15%). The compound was used in next step without further purification. LCMS: m/z 248.0 [M+1]+. 1H NMR (400 MHz, DMSO-d6) δ 8.59 (s, 1H), 8.06 (d, J=7.9 Hz, 1H), 6.90-6.78 (m, 1H), 6.28 (s, 1H), 5.82 (s, 1H), 3.90 (s, 3H).

### Step-2: Synthesis of 2-methoxy-5-(3-(trifluoromethyl)-1H-pyrazol-5-yl) pyridine (Int-3)

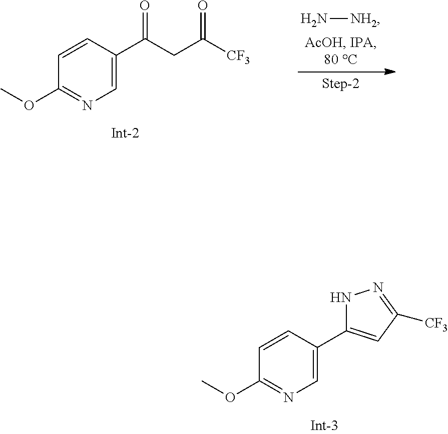

The 4,4,4-trifluoro-1-(6-methoxypyridin-3-yl)butane-1,3-dione (1.5 g, 6.07 mmol, 1.0 eq.) was added to the solution of the Hydrazine monohydrate (0.607 g, 12.14 mmol, 2.0 eq.) in isopropanol (15 mL). Acetic acid (1.0 ml) was added to the reaction mixture and heated at 80° C. for 12 h. Reaction was monitored by TLC. After completion of reaction, volatile matter was distilled off. Then water (40 mL) and saturated solution of sodium bicarbonate (20 mL) were added to the residue and the product was exhaustively extracted with ethyl acetate (50 mL*3). The organic layer was washed with water (40 mL) and dried over anhydrous Na₂SO₄ and concentrated to obtain crude product, which was purified by comb flash using 50% Ethyl acetate in Hexane as eluent to get 2-methoxy-5-(3-(trifluoromethyl)-1H-pyrazol-5-yl) pyridine (Int-3) (1.0 g, 67.76%). ¹H NMR (400 MHz, DMSO-d₆) δ 14.07 (s, 1H), 8.66 (s, 1H), 8.12 (d, J=7.8 Hz, 1H), 7.02 (s, 1H), 6.97 (s, 0.1=8.4 Hz, 1H), 3.90 (s, 1H).

### Step-3: Synthesis of (5-(benzyloxy)-2-fluorophenyl) (6-(5-(6-methoxypyridin-3-yl)-3-(trifluoromethyl)-1H-pyrazol-1-yl)-2-azaspiro [3.3] heptan-2-yl) methanone (Int-4) and (5-(benzyloxy)-2-fluorophenyl) (6-(3-(6-methoxypyridin-3-yl)-5-(trifluoromethyl)-1H-pyrazol-1-yl)-2-azaspiro [3.3] heptan-2-yl) methanone (Int-4A)

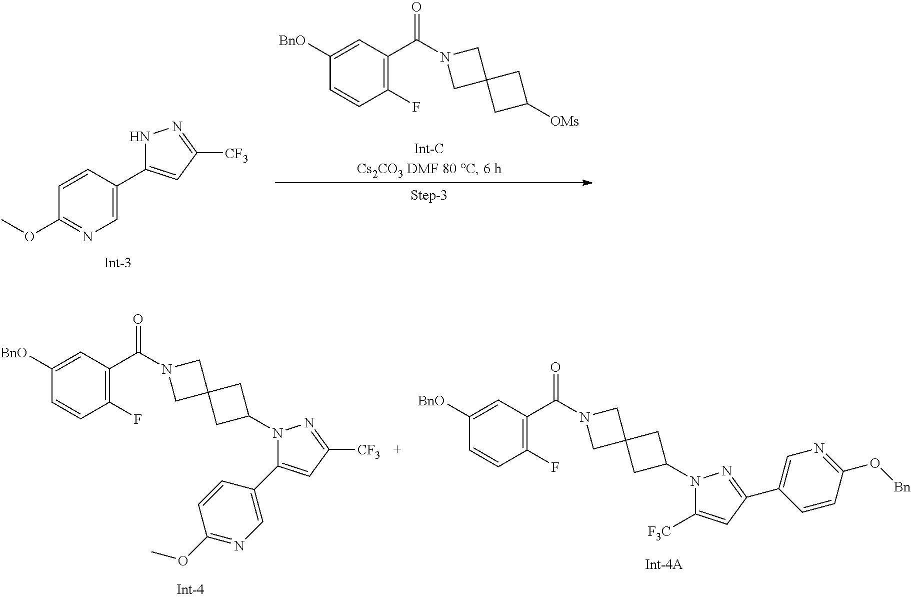

To a solution of 2-methoxy-5-(3-(trifluoromethyl)-1H-pyrazol-5-yl) pyridine (0.250 g, 1.028 mmol, 1.0 eq.) in N, N-Dimethylformamide (10 mL), 2-(5-(benzyloxy)-2-fluorobenzoyl)-2-azaspiro [3.3]heptan-6-yl methane sulfonate (0.430 g, 1.02 mmol, 1.0 eq.) and Cesium carbonate (1.0 g, 3.085 mmol, 3.0 eq.) were added. The reaction mixture was heated at 80° C. for 8 h. After cooling to room temperature t, the reaction mixture was poured into water (40 mL), and the solution was extracted with ethyl acetate (3×40 mL). The combined organic layers were washed with brine (40 mL), dried over Na₂SO₄, filtered and concentrated under reduced pressure to obtained residue. Which was purified by flash column chromatography on silica gel with 50% ethyl acetate in n-hexane to get pure compound, as a mixture of two isomer (5-(benzyloxy)-2-fluorophenyl) (6-(5-(6-methoxypyridin-3-yl)-3-(trifluoromethyl)-1H-pyrazol-1-yl)-2-azaspiro [3.3]heptan-2-yl) methanone (Int-4) and (5-(benzyloxy)-2-fluorophenyl) (6-(3-(6-methoxypyridin-3-yl)-5-(trifluoromethyl)-1H-pyrazol-1-yl)-2-azaspiro [3.3]heptan-2-yl) methanone (Int-4A) (5.0 g, 75.58%). LCMS: m/z 567.29 [M+1]+.

### Step-4: Synthesis of (2-fluoro-5-hydroxyphenyl) (6-(3-(6-methoxypyridin-3-yl)-5-(trifluoromethyl)-1H-pyrazol-1-yl)-2-azaspiro [3.3] heptan-2-yl) methanone (Compound 17) & (2-fluoro-5-hydroxyphenyl) (6-(5-(6-methoxypyridin-3-yl)-3-(trifluoromethyl)-1H-pyrazol-1-yl)-2-azaspiro [3.3] heptan-2-yl) methanone) (Compound-17 and Compound-18)

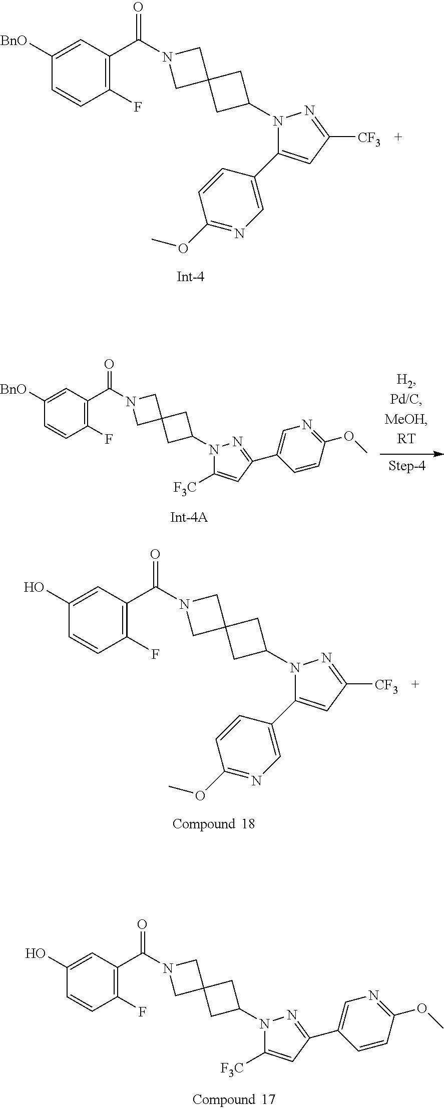

To a stirred solution of (5-(benzyloxy)-2-fluorophenyl) (6-(5-(6-methoxypyridin-3-yl)-3-(trifluoromethyl)-1H-pyrazol-1-yl)-2-azaspiro [3.3]heptan-2-yl) methanone (Int-4) and

(5-(benzyloxy)-2-fluorophenyl) (6-(3-(6-methoxypyridin-3-yl)-5-(trifluoromethyl)-1H1-pyrazol-1-yl)-2-azaspiro [3.3]heptan-2-yl) methanone (Int-4A) (0.200 g, 0.35 mmol, 1.0 eq.) in methanol (20 mL). 10% Pd/C with 50% moisture (0.100 g) was added to the reaction mixture and stirred under hydrogen gas balloon atmosphere for 12 h. After completion of the reaction (monitored by TLC), the reaction mixture was filtered through cellite pad and washed with Methanol (3*25 mL). The organic layer was evaporated under reduce pressure to get crude material. The crude material was purified by using Prep-HPLC 1% formic acid in water and acetonitrile to get two fractions.

Fraction-1: (2-fluoro-5-hydroxyphenyl) (6-(3-(6-methoxypyridin-3-yl)-5-(trifluoromethyl)-1H-pyrazol-1-yl)-2-azaspiro [3.3]heptan-2-yl) methanone (Compound 17) (0.014 g, 8.32%). LCMS: m/z 477.41 [M+H]+. 1H NMR (400 MHz, DMSO-d6) δ 9.73 (s, 1H), 8.27 (d, J=10.8 Hz, 1H), 7.86 (t, J=10.4 Hz 1H), 7.09-7.04 (m, 1H), 7.00 (s, 1H), 6.91 (s, 1H), 6.84 (t, J=4.4 Hz, 1H), 6.80 (s, 1H), 4.79-4.71 (m, 1H), 4.47-4.38 (m, 4H), 3.29 (s, 3H), 2.32 (d, J=17.6 Hz, 4H).

Fraction-2: (2-fluoro-5-hydroxyphenyl) (6-(5-(6-methoxypyridin-3-yl)-3-(trifluoromethyl)-1H-pyrazol-1-yl)-2-azaspiro [3.3]heptan-2-yl) methanone (Compound 18) (0.040 g, 23.85%). LCMS: m/z 477.41 [M+H]+. 1H NMR (400 MHz, DMSO-d6) δ 9.70 (s, 1H), 8.69 (d, J=16.6 Hz, 1H), 8.18-8.13 (dd, J=7.2 Hz, 1H), 7.45 (s, 1H), 7.11-7.09 (t, J=8.8 Hz, 1H), 6.92 (dd, J=17.9, 8.9 Hz, 1H), 6.81 (s, 1H), 6.80 (s, 1H), 4.91 (dt, J=16.1, 7.7 Hz, 1H), 4.15 (dd, J=19.7, 102 Hz, 4H), 3.91 (d, J=7.0 Hz, 3H), 2.86-2.77 (m, 4H).

### Example 3: Synthesis of (2-fluoro-5-hydroxyphenyl)(6-(3-(2-methoxypyridin-3-yl)-5-(trifluoro methyl)-1H1-pyrazol-1-yl)-2-azaspiro[3.3]heptan-2-yl)methanone [Compound 15] and (2-fluoro-5-hydroxyphenyl)(6-(S-(2-methoxypyridin-3-yl)-3-(trifluoro methyl)-1H-pyrazol-1-yl)-2-azaspiro(3.3 heptan-2-yl)methanone [Compound 16]

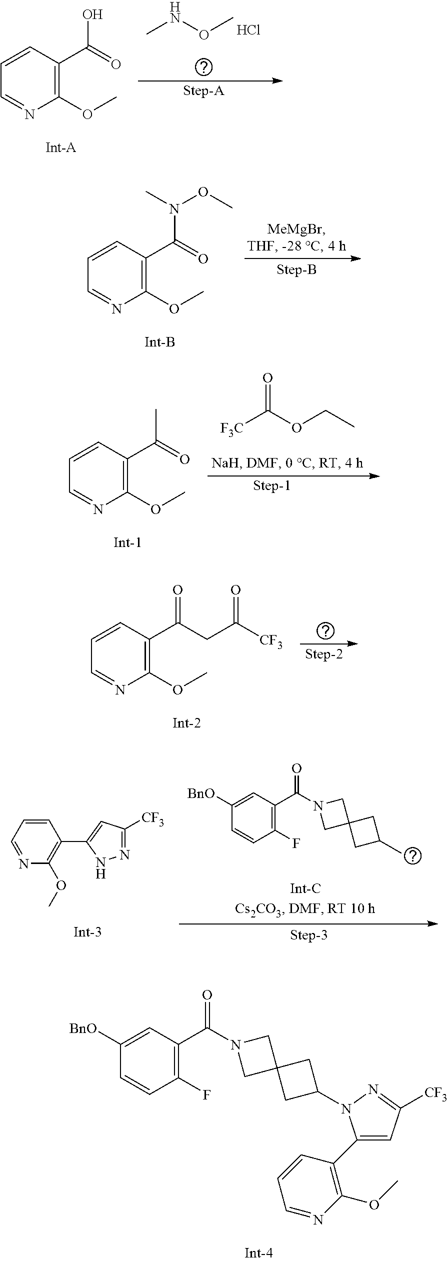

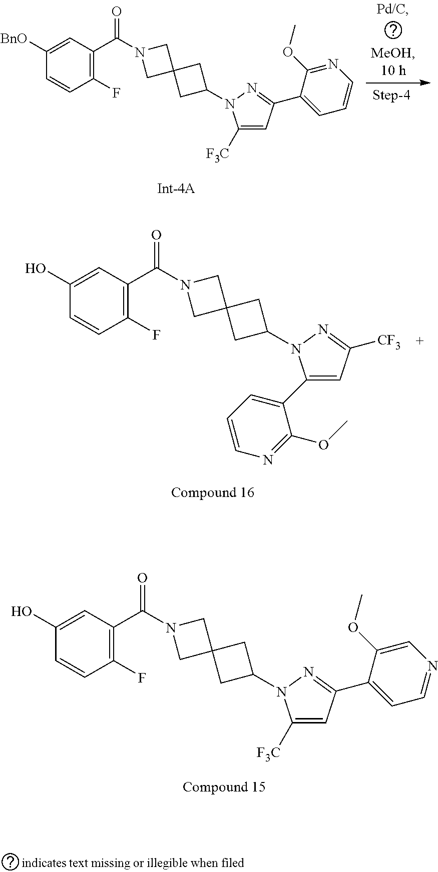

### Step-A: Synthesis of N,2-dimethoxy-N-methylnicotinamide (Int-B)

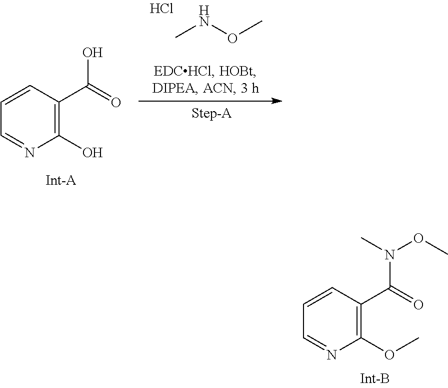

To a solution of the 2-methoxynicotinic acid (10.0 g, 65.31 mmol, 1.0 eq.) in acetonitrile (100 mL) was added N, N-Diisopropylethylamine (25.32 g, 195.95 mmol, 3.0 eq.) and stirred at 00 C. 1-Ethyl-3-(3-dimethylaminopropyl) carbodiimide (EDC-HCl) (18.78 g, 97.97 mmol, 1.5 eq.), 1-Hydroxybenzotriazole (HOBt) (11.47 g, 84.91 mmol, 1.3 eq.) and N,O-dimethyl hydroxylamine (8.23 g, 84.91 mmol, 1.3 eq.) were added to the reaction after 15 mins of each addition. The reaction was stirred at room temperature for 3 h. After completion of the reaction (checked by TLC, LCMS), mixture was diluted with H2O (150 mL) and extracted with ethyl acetate (250 mL*3). The organic layer was dried sodium sulphate and concentrated through vacuum to get crude compound. Which was purified by flash chromatography, product eluting with 15-20% ethyl acetate in n-hexane to provide the product as a N,2-dimethoxy-N-methylnicotinamide (Int-B). (10.0 g, 78.06%). LCMS: m/z 152.1 [M+H]+

### Step-B: Synthesis of 1-(2-methoxypyridin-3-yl) ethan-1-one (Int-1)

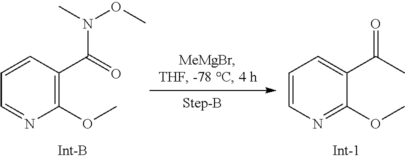

To a solution of the N,2-dimethoxy-N-methylnicotinamide (10.0 g, 50.76 mmol, 1.0 eq.) in anhydrous tetrahydrofuran (70 mL) at −780 C under nitrogen. MeMgBr (3M in ether, 42.3 mL, 126.90 mmol, 2.5 eq.) was added dropwise to the reaction and stirred for 1 h at −78° C. The cold bath was removed and the reaction was stirred at room temperature for 4 h. The mixture was diluted with sat. NH4Cl soln and extracted with ethyl acetate (150 mL*3). The combined organics were washed with brine, dried over sodium sulphate, concentrated through vacuum to obtained residue. Which was purified by flash chromatography, product eluting with 50-100% ethyl acetate in n-hexane to provide the product as a 1-(2-methoxypyridin-3-yl) ethan-1-one (Int-1) (7.2 g, 93.50%).

### Step-1: Synthesis of 4,4,4-trifluoro-1-(2-methoxypyridin-3-yl)butane-1,3-dione (Int-2)

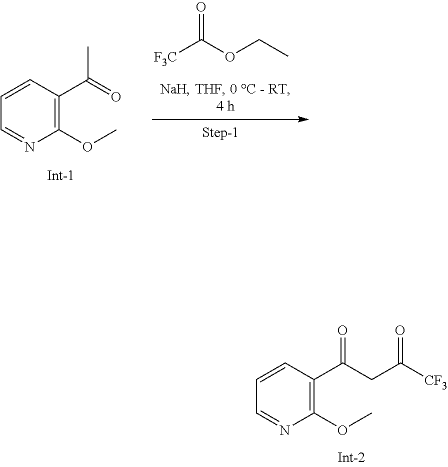

In a dry flask, a mixture of 1-(2-methoxypyridin-3-yl) ethan-1-one (7.2 g, 42.85 mmol, 1.0 eq.) in dry Tetrahydrofuran (72 mL) was stirred at 00 C. Sodium hydride (1.54 g, 64.28 mmol, 1.5 eq.) was added portion wise to the reaction mixture under N2 atmosphere. Reaction mixture was stirred for 10 min, then ethyl 2,2,2-trifluoroacetate (3.12 g, 64.28 mmol, 1.5 eq.) was dropwise added to the reaction mixture at 00 C. Reaction was stirred at room temperature for 4-5 h. After completion of reaction, the reaction mixture was poured into water (100 mL), and the solution was extracted with ethyl acetate (3×150 mL). The combined organic layers were washed with brine (50 mL), dried over sodium sulfate, filtered and concentrated under reduced pressure to obtained crude product. Which was used for next reaction. (13.70 g, yield: crude). LCMS: m/z 246.2 [M−H]+

### Step-2: Synthesis of 2-methoxy-3-(3-(trifluoromethyl)-1H-pyrazol-5-yl) pyridine (Int-3)

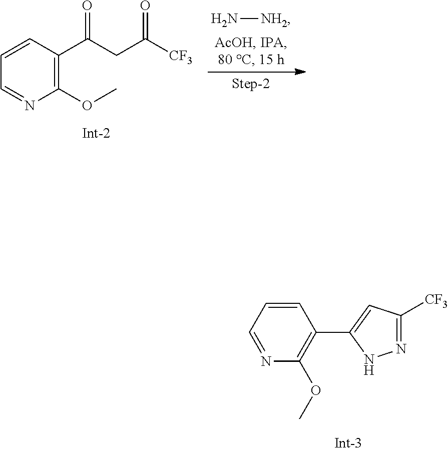

To a solution of 4,4,4-trifluoro-1-(2-methoxypyridin-3-yl)butane-1,3-dione (13.7 g, 55.46 mmol, 1.0 eq.) in ethanol (100 mL) was added hydrazine hydrate (4.15 g, 83.19 mmol, 1.5 eq.). The mixture was stirred at room temperature for 1 h and then heated at 800 C for 15 h. After completion of reaction (checked by TLC), reaction mass concentrated through vacuum to obtained crude product. Which was purified by flash chromatography. The product was eluted in using 30% ethyl acetate in n-hexane to give the 2-methoxy-3-(3-(trifluoromethyl)-1H-pyrazol-5-yl) pyridine. (7.0 g, 51.96%). LCMS: m/z 244.14 [M+H]+

### Step-3: Synthesis of (5-(benzyloxy)-2-fluorophenyl)(6-(5-(2-methoxypyridin-3-yl)-3-(trifluoromethyl)-11-pyrazol-1-yl)-2-azaspiro (3.31 heptan-2-yl) methanone (Int-4) and (5-(benzyloxy)-2-fluorophenyl)(6-(3-(2-methoxypyridin-3-yl)-5-(trifluoromethyl)-1H-pyrazol-1-yl)-2-azaspiro [3.3] heptan-2-yl) methanone (Int-4A)

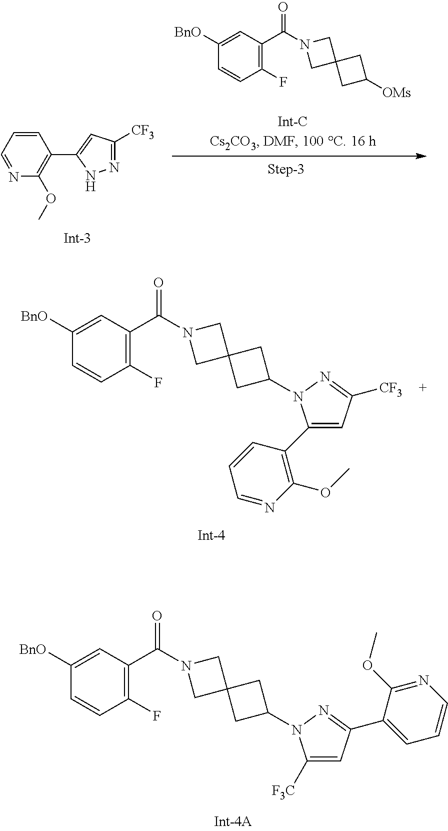

To a solution of 2-methoxy-3-(3-(trifluoromethyl)-1H-pyrazol-5-yl) pyridine (Int-3) (0.500 g, 2.05 mmol, 1.0 eq.) in N,N-Dimethylformamide (5.0 mL), 2-(5-(benzyloxy)-2-fluorobenzoyl)-2-azaspiro[3.3]heptan-6-yl methane sulfonate (Int-C) (0.948 g, 2.26 mmol, 1.1 eq.) and Cs2CO3 (1.00 g, 3.086 mmol, 1.5 eq.) were added, the reaction mixture was heated at 100° C. for 16 h. After completion of reaction (Checked by TLC), cooled to room temperature, the reaction mixture was poured into water (60 mL), and the solution was extracted with ethyl acetate (3×50 mL. The combined organic layers were washed with brine (50 mL), dried over sodium sulfate and concentrated under reduced pressure to get residue. Which was purified by flash column chromatography. The product eluted in 60% ethyl acetate in n-hexane to get mixture of two region isomers of (5-(benzyloxy)-2-fluorophenyl)(6-(5-(2-methoxy pyridin-3-yl)-3-(trifluoromethyl)-1H-pyrazol-1-yl)-2-azaspiro [3.3]heptan-2-yl) methanone (Int-4) and (5-(benzyloxy)-2-fluorophenyl)(6-(3-(2-methoxypyridin-3-yl)-5-(trifluoromethyl)-1H-pyrazol-1-yl)-2-azaspiro [3.3]heptan-2-yl) methanone (Int-4A). (0.700 g, 60.34%).

### Step-4: Synthesis of (2-fluoro-5-hydroxyphenyl)(6-(3-(2-methoxypyridin-3-yl)-5-(trifluoromethyl)-1H-pyrazol-1-yl)-2-azaspiro [3.3] heptan-2-yl) methanone (Compound-15) and (2-fluoro-5-hydroxyphenyl)(6-(5-(2-methoxy pyridin-3-yl)-3-(trifluoromethyl)-1H-pyrazol-1-yl)-2-azaspiro [3.3]heptan-2-yl)methanone (Compound-16)

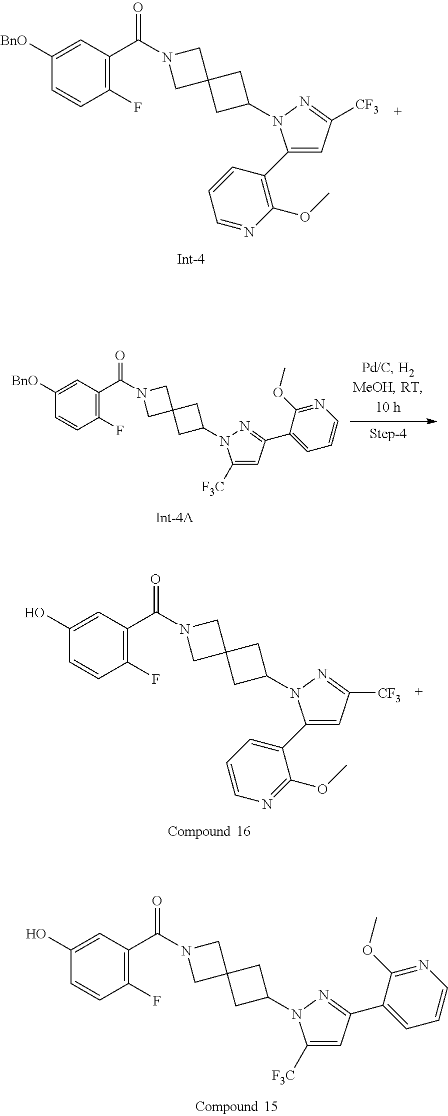

To a solution of (5-(benzyloxy)-2-fluorophenyl)(6-(5-(2-methoxypyridin-3-yl)-3-(trifluoromethyl)-1H-pyrazol-1-yl)-2-azaspiro [3.3]heptan-2-yl) methanone (Int-4) and (5-(benzyloxy)-2-fluorophenyl)(6-(3-(2-methoxypyridin-3-yl)-5-(trifluoromethyl)-1H-pyrazol-1-yl)-2-azaspiro [3.3]heptan-2-yl) methanone (Int-4A). (0.350 g, 0.617 mmol, 1.0 eq.) In methanol (3.5 mL), 10% Pd/C on 50% moisture (0.1 g) was added. Reaction was stirred under H2(g) atmosphere at room temperature for 10 h. After completion of reaction (checked by TLC), reaction mixture was filtered through celite bed. Filtrate-mL was distilled out to obtain crude product. Which was purified with prep-HPLC to obtain two fractions.

Fraction-1: (2-fluoro-5-hydroxyphenyl)(6-(3-(2-methoxypyridin-3-yl)-5-(trifluoromethyl)-1H-pyrazol-1-yl)-2-azaspiro[3.3]heptan-2-yl)methanone (Compound 15) (0.028 g, 9.52%). LCMS: m/z 477.40 [M+H]+. 1H NMR (400 MHz, DMSO-d6) 9.66 (d, J 2.2 Hz, 1H), 8.36 (m, 1H), 7.77 (t, J=7.2 Hz, 1H), 7.22-7.12 (m, 1H), 7.09 (dt, J=17.0, 7.9 Hz, 1H), 6.88-6.75 (m, 3H), 4.57 (dt, J=15.5, 7.8 Hz, 1H), 4.06 (d, J=7.3 Hz, 4H), 3.90 (d, J=13.9 Hz, 3H), 2.71-2.54 (m, 4H).

Fraction-2: (2-fluoro-5-hydroxyphenyl)(6-(5-(2-methoxypyridin-3-yl)-3-(trifluoromethyl)-1H-pyrazol-1-yl)-2-azaspiro[3.3]heptan-2-yl)methanone (Compound 16) (0.073 g, 24.82%). LCMS: m/z 477.10 [M+H]+. 1H NMR (400 MHz, DMSO-d6) δ 9.66 (d, J=2.2 Hz, 1H), 8.34 (dd, J=11.5, 4.2 Hz, 1H), 8.21 (m, 1H), 7.22-7.12 (m, 1H), 7.09-7.20 (dt, J=17.0, 7.9 Hz, 2H), 6.88-6.75 (m, 2H), 4.97 (dt, J=15.5, 7.8 Hz, 1H), 4.06-4.16 (d, J=7.3 Hz, 4H), 3.90 (d, J=13.9 Hz, 3H), 2.74 (d, J=12.2 Hz, 4H).

### Example 4: Synthesis of (2-fluoro-5-hydroxyphenyl)(6-(5-(5-fluoropyridin-3-yl)-3-(trifluoromethyl)-1H-pyrazol-1-yl)-2-azaspiro[3.3]heptan-2-yl)methanone, [Compound 13] and (2-fluoro-5-hydroxyphenyl)(6-(3-(5-fluoropyridin-3-yl)-5-(trifluoromethyl)-1H-pyrazol-1-yl)-2-azaspiro[3.3]heptan-2-yl)methanone [Compound 14]

### Synthetic Scheme:

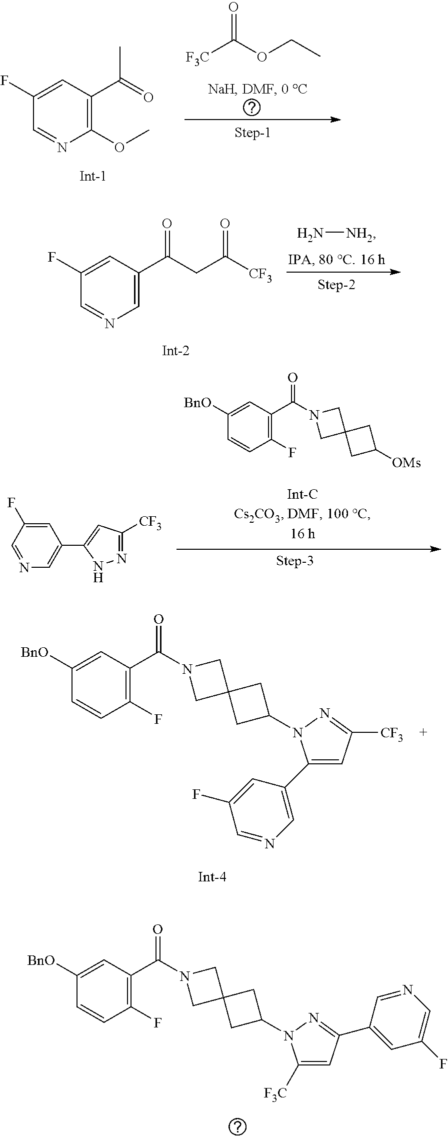

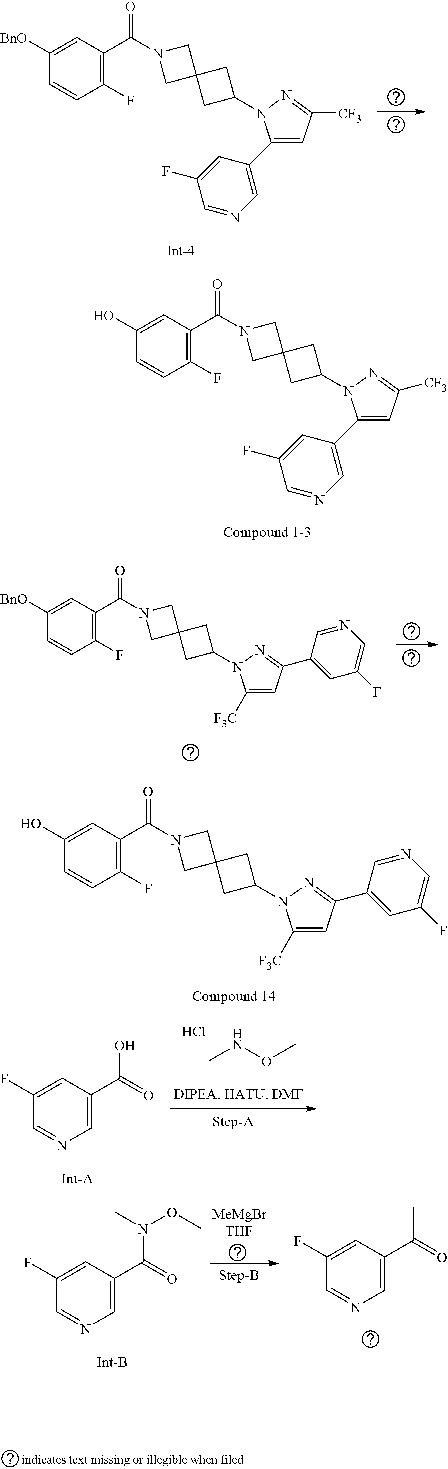

### Step-A: Synthesis of 5-fluoro-N-methoxy-N-methylnicotinamide (Int-B)

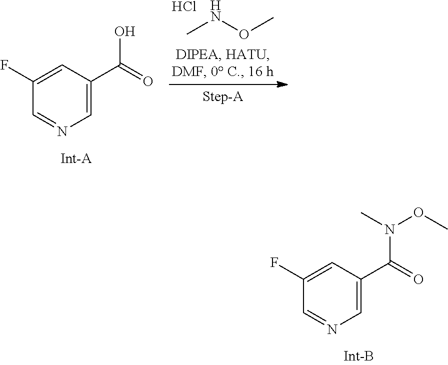

To a stirred solution of 5-fluoronicotinic acid (10.0, 70.87 mmol, 1.0 eq.), N,N-Di isopropyl ethylamine (27.4 g, 212.61 mmol, 3.0 eq.), N,O-dimethyl hydroxylamine (8.9 g, 92.13 mmol, 1.3 eq.) in N,N-dimethyl formamide (50 mL), was added HATU (35.61 g, 92.13 mmol, 1.3 eq.) at 0° C. Stir the reaction at room temperature for 16 h. After completion of reaction as monitored by TLC, the reaction mixture was poured in ice cold water (100 mL) and extracted with ethyl acetate (50 mL*3). The organic layer was dried over Sodium sulfate and concentrated under reduce pressure to get residue. The residue was purified by combiflash using 15-18% ethyl acetate in n-hexane as eluent to get 5-fluoro-N-methoxy-N-methylnicotinamide (Int-B) (11.7 g, 89.64%). LCMS: m/z 185.08 [M+1]+.

### Step-B: Synthesis of 1-(5-fluoropyridin-3-yl) ethan-1-one (Int-1)

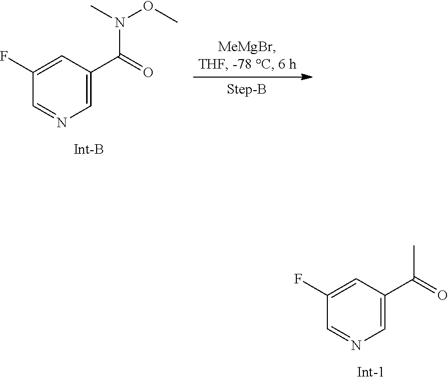

To a stirred solution of 5-fluoro-N-methoxy-N-methylnicotinamide (11.7, 63.58 mmol, 1.0 eq.) in tetrahydrofuran (30 mL), was slowly added Methyl Magnesium Bromide 3M soln in THF (31.8 mL, 95.38 mmol, 1.5 eq.) at −78° C. Stir the reaction at room temperature for 6 h. After completion of reaction as monitored by TLC, the reaction mixture was poured in ice cold water (100 mL) and extracted with ethyl acetate (50 mL*3). The organic layer was dried over Sodium sulfate and concentrated under reduce pressure to get residue. The residue was purified by combiflash using 5-8% Ethyl acetate in hexane as eluent to get 1-(5-fluoropyridin-3-yl) ethan-1-one (Int-1) (7.6 g, 85.99%). LCMS: m/z 140.10 [M+1]+.

### Step-1: Synthesis of 4,4,4-tri fluoro-1-(5-fluoropyridin-3-yl)butane-1,3-dione (Int-2)

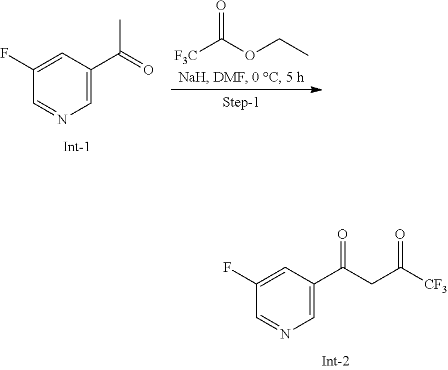

To a stirred solution of 1-(5-fluoropyridin-3-yl)ethan-1-one (7.6 g, 54.67 mmol, 1.0 eq.) in Tetrahydrofuran (50 mL) was added Sodium hydride (60% on paraffin) (3.28 g, 82.01 mmol, 1.5 eq.) at 0° C., followed by the dropwise addition of ethyl 2,2,2-trifluoroacetate (11.6 g, 82.01 mmol, 1.5 eq.). The reaction mass was stirred at room temperature for 4-5 h After completion of reaction as monitored by TLC. The reaction mixture was poured in water (100 mL), and extracted with ethyl acetate (50 mL*3). The organic layer was washed with brine (100 mL), dried over Sodium sulfate and concentrated under reduce pressure to get residue. The residue was purified by combiflash using 10% ethyl acetate in n-hexane as eluent to obtain 4,4,4-trifluoro-1-(5-fluoropyridin-3-yl)butane-1,3-dione (Int-2) (11.1 g, 86.42%). LCMS: m/z 233.90 [M−1]+.

### Step-2: Synthesis of 3-fluoro-5-(3-(trifluoromethyl)-1H-pyrazol-5-yl) pyridine (Int-3)

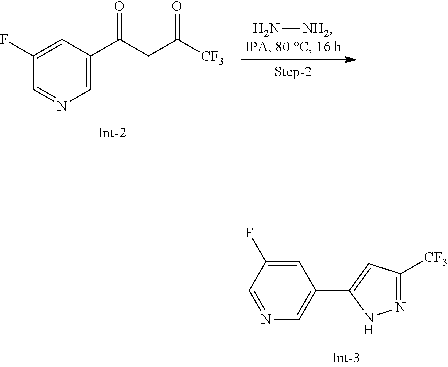

To a stirred solution of 4,4,4-trifluoro-1-(5-fluoropyridin-3-yl)butane-1,3-dione (11.1 g, 47.63 mmol, 1.0 eq.) in Isopropyl alcohol (110 mL) was added hydrazine hydrate (3.57 g, 71.45 mmol, 1.5 eq.) the reaction mass was heated at 80° C. for 16 h. After completion of reaction as monitored by TLC, the reaction mixture was concentrated to get residue. The residue was purified by combiflash using 15-18% ethyl acetate in n-hexane as eluent to get 3-fluoro-5-(3-(trifluoromethyl)-1H-pyrazol-5-yl)pyridine (Int-3) (8.6 g, 78.81%). LCMS: m/z 232.19 [M+1]+.

### Step-3: Synthesis of (5-(benzyloxy)-2-fluorophenyl)(6-(5-(5-fluoropyridin-3-yl)-3-(trifluoromethyl)-1H-pyrazol-1-yl)-2-azaspiro[3.3] heptan-2-yl)methanone (Int-4) and (5-(benzyloxy)-2-fluorophenyl)(6-(3-(5-fluoropyridin-3-yl)-5-(trifluoromethyl)-1H-pyrazol-1-yl)-2-azaspiro [3.3] heptan-2-yl) methanone (Int-4A)

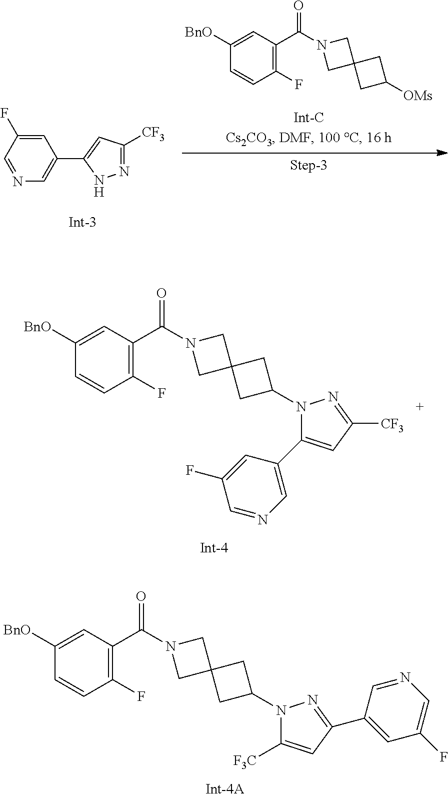 [SMILES: CS(=O)(=O)OC1CC2(C1)CN(C(=O)c1cc(OCc3ccccc3)ccc1F)C2 ; Fc1cncc(-c2cc(C(F)(F)F)n[nH]2)c1 ; O=C(c1cc(OCc2ccccc2)ccc1F)N1CC2(CC(n3nc(C(F)(F)F)cc3-c3cncc(F)c3)C2)C1 ; O=C(c1cc(OCc2ccccc2)ccc1F)N1CC2(CC(n3nc(-c4cncc(F)c4)cc3C(F)(F)F)C2)C1]

To a well stirred reaction mixture of 3-fluoro-5-(3-(trifluoromethyl)-1H-pyrazol-5-yl)pyridine (0.3 g, 1.40 mmol, 1.0 eq.), Cesium carbonate (0.90 g, 2.81 mmol, 2.0 eq.) in N,N-Dimethylformamide (5 mL) was added 2-(5-(benzyloxy)-2-fluorobenzoyl)-2-azaspiro[3.3]heptan-6-yl methane sulfonate (0.76 g, 1.83 mmol, 1.3 eq.). The reaction was heated at 100° C. for 16 h. After completion of reaction as monitored by TLC, the reaction mixture was poured in water (50 mL) and extracted with ethyl acetate (50*2 mL). The organic layer was washed with brine (30 mL), dried over Sodium sulfate and concentrated under reduce pressure to get residue. The residue was purified by combiflash using 30-40% ethyl acetate in n-hexane as eluent to get two fractions of (2-fluoro-5-hydroxyphenyl)(6-(5-(5-fluoropyridin-3-yl)-3-(trifluoromethyl)-1H-pyrazol-1-yl)-2-azaspiro[3.3]heptan-2-yl)methanone (Int-4) (0.5 g, 69.48%) LCMS: m/z 555.30 [M+1]+ and (2-fluoro-5-hydroxyphenyl)(6-(5-(5-fluoropyridin-3-yl)-3-(trifluoromethyl)-1H-pyrazol-1-yl)-2-azaspiro[3.3]heptan-2-yl)methanone (Int-4A) (0.27 g, 27.79%). LCMS: m/z 555.41 [M+1]+.

### Step-4: Synthesis of (2-fluoro-5-hydroxyphenyl)(6-(5-(5-fluoropyridin-3-yl)-3-(trifluoromethyl)-1H-pyrazol-1-yl)-2-azaspiro[3.3]heptan-2-yl)methanone (Compound-13)

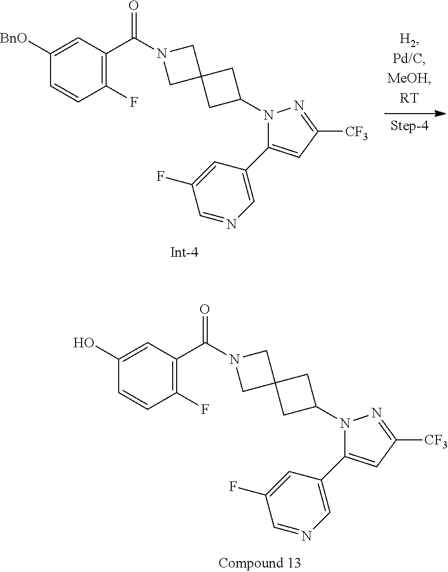 [SMILES: O=C(c1cc(OCc2ccccc2)ccc1F)N1CC2(CC(n3nc(C(F)(F)F)cc3-c3cncc(F)c3)C2)C1 ; O=C(c1cc(O)ccc1F)N1CC2(CC(n3nc(C(F)(F)F)cc3-c3cncc(F)c3)C2)C1]

To a well stirred reaction mixture of (2-fluoro-5-hydroxyphenyl)(6-(5-(5-fluoropyridin-3-yl)-3-(trifluoromethyl)-1H-pyrazol-1-yl)-2-azaspiro[3.3]heptan-2-yl)methanone (0.5 g, 1.07 mmol, 1.0 eq.) in Methanol (5 mL), was added 10% Pd/C on 50% moisture (0.8 g). Reaction was stirred under H_{2(g)} atmosphere at room temperature for 10 h. After completion of reaction as monitored by TLC, reaction mixture was filtered through celite bed. Filtrate was distilled out to obtain crude product. The crude product was purified by prep-HPLC to obtained (2-fluoro-5-hydroxyphenyl)(6-(5-(5-fluoropyridin-3-yl)-3-(trifluoromethyl)-1H-pyrazol-1-yl)-2-azaspiro[3.3]heptan-2-yl)methanone (Compound 13) (0.07 g, 16.72%). LCMS: m/z 465.30 [M+1]⁺. ¹H NMR (400 MHz, DMSO-d6) δ 9.65 (d, J=4.3 Hz, 1H), 8.99 (d, J=19.3 Hz, 1H), 8.59 (d, J=10.7 Hz, 1H), 8.16 (dd, J=20.6, 9.2 Hz, 1H), 7.63 (s, 1H), 7.08 (d, J=6.4 Hz, 1H), 6.80 (s, 1H), 6.75 (s, 1H), 4.93 (s, 1H), 4.13 (dd, J=18.0, 8.9 Hz, 4H), 2.84-2.78 (s, 4H).

### Step-4A: Synthesis of (2-fluoro-5-hydroxyphenyl)(6-(3-(5-fluoropyridin-3-yl)-5-(trifluoromethyl)-1H-pyrazol-1-yl)-2-azaspiro[3.3]heptan-2-yl)methanone (Compound-14)

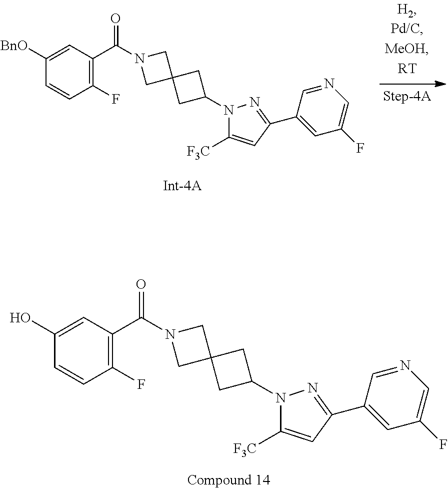 [SMILES: O=C(c1cc(OCc2ccccc2)ccc1F)N1CC2(CC(n3nc(-c4cncc(F)c4)cc3C(F)(F)F)C2)C1 ; O=C(c1cc(O)ccc1F)N1CC2(CC(n3nc(-c4cncc(F)c4)cc3C(F)(F)F)C2)C1]

To a well stirred reaction mixture of (2-fluoro-5-hydroxyphenyl)(6-(5-(5-fluoropyridin-3-yl)-3-(trifluoromethyl)-1H-pyrazol-1-yl)-2-azaspiro[3.3]heptan-2-yl)methanone (0.2 g, 0.43 mmol, 1.0 eq.) in Methanol (3 mL), was added 10% Pd/C on 50% moisture (0.02 g). Reaction was stirred under H_{2(g)} atmosphere at room temperature for 10 h. After completion of reaction as monitored by TLC, reaction mixture was filtered through celite bed. Filtrate was distilled out to obtain crude product. The crude product was purified by prep-HPLC to obtained (2-fluoro-5-hydroxyphenyl)(6-(3-(5-fluoropyridin-3-yl)-5-(trifluoromethyl)-1H-pyrazol-1-yl)-2-azaspiro[3.3]heptan-2-yl)methanone (Compound 14) (0.025 g, 14.93%). LCMS: m/z 465.40 [M+1]⁺. ¹H NMR (400 MHz, DMSO-d6) δ 9.66 (s, 1H), 8.74 (dd, J=11.4, 2.6 Hz, 1H), 8.57 (d, J=10.9 Hz, 1H), 8.00-7.89 (m, 1H), 7.07-7.05 (dd, J=18.4, 2.6 Hz, 1H), 6.87-6.73 (m, 2H), 4.93 (dq, J=25.0, 7.8 Hz, 1H), 4.05 (dd, J=16.7, 9.5 Hz, 4H), 2.70 (h, J=12.1 Hz, 4H).

### Example 5: Synthesis of 3-(1-(2-(2-fluoro-5-hydroxybenzoyl)-2-azaspiro [3.3] heptan-6-yl)-3-(trifluoro methyl)-H-pyrazol-5-yl) pyridin-2(1H)-one [Compound 11] and Compound 12: 3-(1-(2-(2-fluoro-5-hydroxybenzoyl)-2-azaspiro [ 3.3] heptan-6-yl)-5-(trifluoro methyl)-1H-pyrazol-3-yl) pyridin-2(1H)-one [Compound 12]

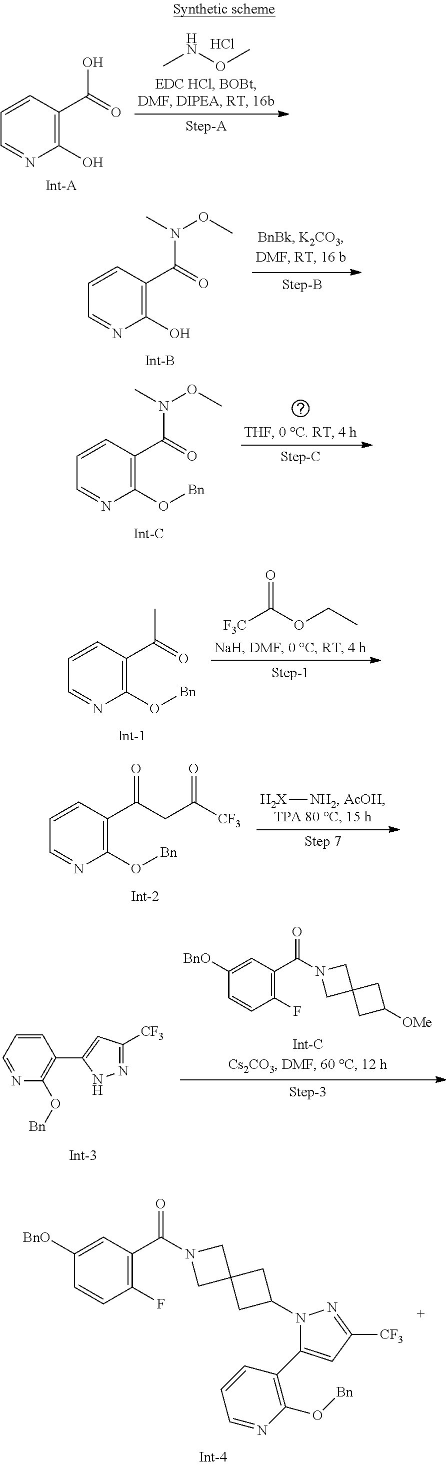

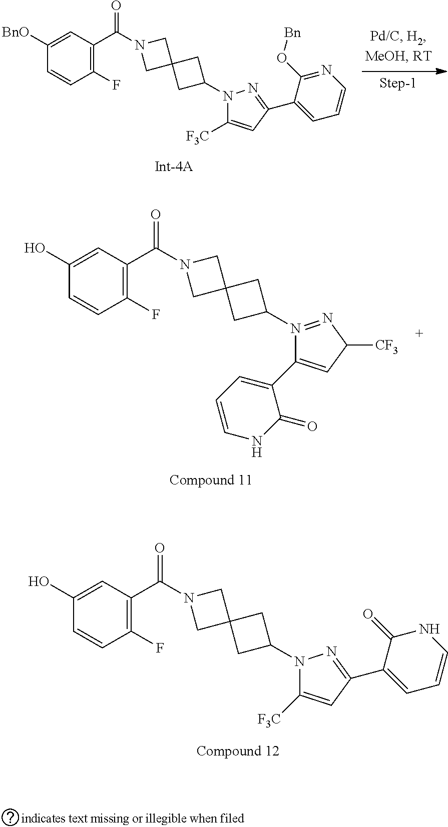

### Step-A. Synthesis of 2-hydroxy-N-methoxy-N-methylnicotinamide (Int-B)

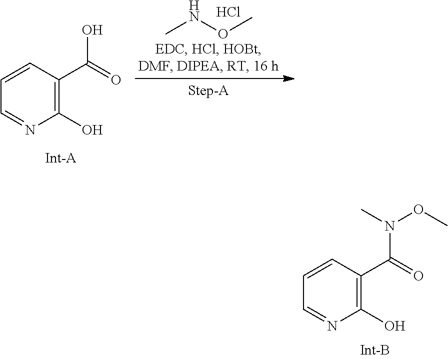

To a stirred solution of 4-fluoro-2-hydroxybenzoic acid (1.0 g, 7.1 mmol, 1.0 eq.) in N,N-dimethyl formamide (10 mL) at 0° C., N,N-diisopropylethyl amine (3.68 ml, 21.5 mmol, 3.0 eq.) and EDC·HCl (2.068 g, 10.7 mmol, 1.5 eq.) were added and allow to stirred for 15 mins. Followed by HOBT (1.263 g, 9.35 mmol, 1.3 eq.) and N,O-dimethyl hydroxylamine (0.912 g, 9.3 mmol, 1.3 eq.) were added to the reaction mass at 0° C. and resulting reaction mixture was stirred at room temperature for 16 h. After completion of reaction as monitored by TLC, the reaction mixture was diluted with water (50 mL) and extracted with ethyl acetate (3*20 mL). The organic layer was dried over sodium sulphate and concentrated under vacuum to get crude material which was purified by combiflash using 80% ethyl acetate in n-hexane as eluent to get 2-hydroxy-N-methoxy-N-methylnicotinamide (Int-B) (1.1 g, 84.61%).

### Step-B. Synthesis of 2-(benzyloxy)-N-methoxy-N-methyl nicotinamide (Int-C)

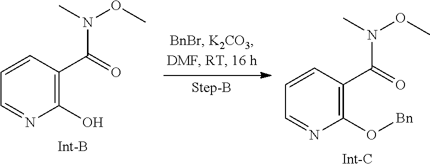 [SMILES: CON(C)C(=O)c1cccnc1O ; CON(C)C(=O)c1cccnc1OCc1ccccc1]

To a stirred solution of 2-hydroxy-N-methoxy-N-methylnicotinamide (1.0 g, 5.4 mmol, 1.0 eq.) in N,N-dimethyl formamide (10 mL), were added Potassium carbonate (2.27 g, 16.4 mmol, 3.0 eq.) at 0° C. Then benzyl bromide (1.40 g, 8.2 mmdl, 1.5 eq.) was dropwise added into reaction mixture at 0° C. and allowed to stir at room temperature for 3-4 h. After completion of reaction as monitored by TLC, the reaction mixture was diluted with water (50 mL) and extracted with ethyl acetate (3*20 mL). The organic layer was dried over sodium sulphate and concentrated under vacuum to get crude material which was purified by combiflash using 80% ethyl acetate in n-hexane as eluent to get (benzyloxy)-N-methoxy-N-methyl nicotinamide (Int-C) (1.0 g, 67.11%). LCMS: m/z 273.30 [M+1]⁺. ¹H NMR (400 MHz, DMSO-d₆) δ 7.90 (d, J=6.0 Hz, 1H), 7.50 (d, J=5.9 Hz, 1H), 7.38-7.28 (m, 5H), 6.31 (t, J=6.7 Hz, 1H), 5.14 (s, 2H), 4.03 (q, J=7.1 Hz, 1H), 3.33 (s, 3H), 3.16 (s, 3H).

### Step-C. Synthesis of 1-(2-(benzyloxy) pyridin-3-yl) ethan-1-one (Int-1)

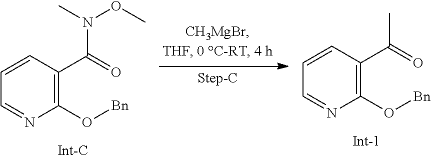 [SMILES: CON(C)C(=O)c1cccnc1OCc1ccccc1 ; CC(=O)c1cccnc1OCc1ccccc1]

To a stirred solution of 2-(benzyloxy)-N-methoxy-N-methyl nicotinamide in (1.0 g, 3.6 mmol, 1.0 eq.) in Tetrahydrofuran (10 mL) were dropwise added methyl magnesium bromide (3M in DEE) (2.45 mL, 7.3 mmol, 2.0 eq.) into reaction mixture at 0° C. Reaction mixture was stirred at room temperature for 3-4 h. After completion of reaction as monitored by TLC, the reaction mixture was diluted with water (50 mL) and extracted with ethyl acetate (3*20 mL). The organic layer was dried over sodium sulphate and concentrated under vacuum to get crude material which was purified by combiflash using 30% ethyl acetate in n-hexane as eluent to get 1-(2-(benzyloxy)pyridin-3-yl)ethan-1-one (Int-1) (0.690 g, 82.73%). LCMS: 228.21 m/z [M+1]⁺.

¹H NMR (400 MHz, DMSO-d₆) δ 8.21 (d, J=6.2 Hz, 1H), 8.01 (d, J=6.7 Hz, 1H), 7.33 (h, J 7.4 Hz, 5H), 6.43 (t, J=6.6 Hz, 1H), 5.19 (s, 2H), 2.53 (s, 3H).

### Step-1: Synthesis of 1-(2-(benzyloxy) pyridin-3-yl)-4,4,4-trifluorobutane-1,3-dione (Int-2)

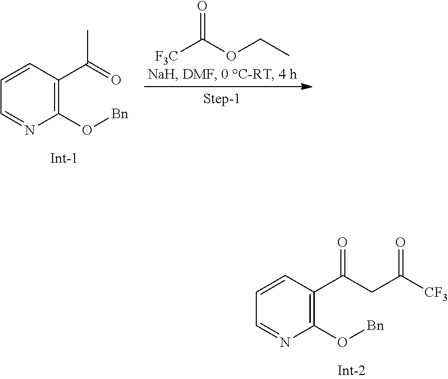

In a dry flask, a mixture of 1-(2-(benzyloxy) pyridin-3-yl) ethan-1-one (0.690 g, 3.0 mmol, 1.0 eq.) in dry Tetrahydrofuran (35 mL) was stirred at 0° C. Sodium hydride (0.182 g, 4.5 mmol, 1.5 eq.) was added portion wise into the well stirred reaction mixture under N₂ atmosphere. Reaction mixture was stirred for 10 min, then ethyl 2,2,2-trifluoroacetate (0.647 g, 4.5 mmol, 1.5 eq.) was dropwise added into reaction mixture at 0° C. Reaction mixture was stirred at room temperature for 4-5 h. After completion of reaction, the reaction mixture was poured into water (50 mL), and the solution was extracted with ethyl acetate (3×20 mL). The combined organic layers were washed with brine (20 mL), dried over sodium sulfate and concentrated under reduced pressure to obtained crude product. Which was used for next reaction. (0.98 gm, yield-crude). LCMS: 324.82 m/z [M+1]⁺.

### Step-2 Synthesis of 2-(benzyloxy)-3-(3-(trifluoromethyl)-1H-pyrazol-5-yl) pyridine (Int-3)

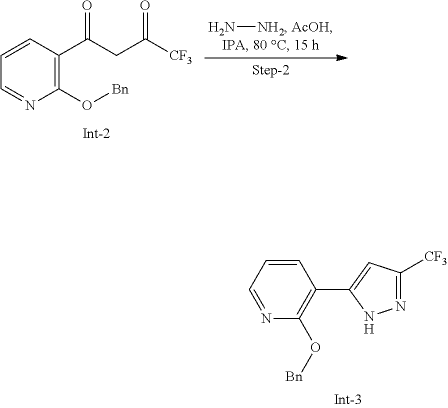

To a solution of 1-(2-(benzyloxy) pyridin-3-yl)-4,4,4-trifluorobutane-1,3-dione (0.981 g, 3.0 mmol, 1.0 eq.) in isopropanol (10 mL) was added hydrazine hydrate (0.227 g, 4.5 mmol, 1.5 eq.). Acetic acid was added as catalytic amount (0.01 g). The mixture was stirred at room temperature for 1 h and then at 80° C. for 15 h. After completion of reaction checked by TLC, solvent was removed using vacuum to obtained crude product, which was purified by chromatography using 30% ethyl acetate in n-hexane to give the 2-(benzyloxy)-3-(3-(trifluoromethyl)-1H-pyrazol-5-yl) pyridine (Int-3) (0.550 g, 56.81%). LCMS: 320.40 m/z [M+1]⁺.

### Step-3: Synthesis of (5-(benzyloxy)-2-fluorophenyl) (6-(5-(2-(benzyloxy) pyridin-3-yl)-3-(trifluoromethyl)-1H-pyrazol-1-yl)-2-azaspiro 13.31 heptan-2-yl) methanone (Int-4) and (5-(benzyloxy)-2-fluorophenyl)(6-(3-(2-(benzyloxy) pyridin-3-yl)-5-(trifluoromethyl)-1H-pyrazol-1-yl)-2-azaspiro [3.3] heptan-2-yl) methanone (Int-4A)

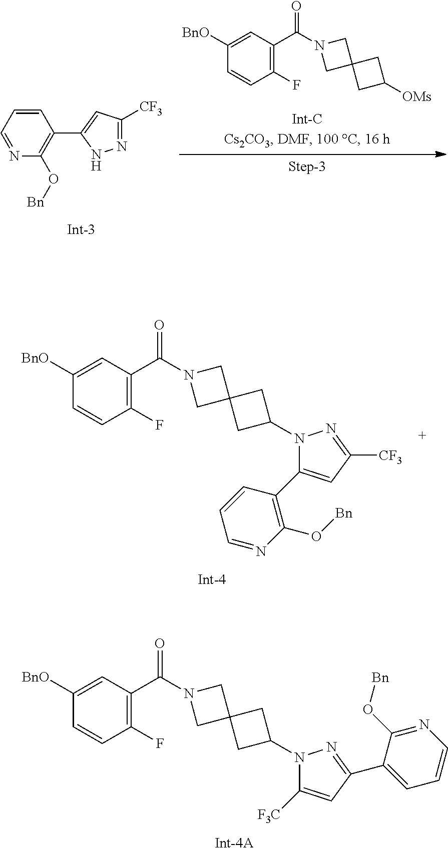 [SMILES: CS(=O)(=O)OC1CC2(C1)CN(C(=O)c1cc(OCc3ccccc3)ccc1F)C2 ; FC(F)(F)c1cc(-c2cccnc2OCc2ccccc2)[nH]n1 ; O=C(c1cc(OCc2ccccc2)ccc1F)N1CC2(CC(n3nc(C(F)(F)F)cc3-c3cccnc3OCc3ccccc3)C2)C1 ; O=C(c1cc(OCc2ccccc2)ccc1F)N1CC2(CC(n3nc(-c4cccnc4OCc4ccccc4)cc3C(F)(F)F)C2)C1]

To a solution of 2-(benzyloxy)-3-(3-(trifluoromethyl)-1H-pyrazol-5-yl) pyridine (Int-3) (0.550 g, 1.72 mmol, 1.0 eq.) in N,N-dimethyl formamide (6.0 mL), 2-(5-(benzyloxy)-2-fluorobenzoyl)-2-azaspiro[3.3]heptan-6-yl methane sulfonate (Int-C) (0.794 g, 1.8 mmol, 1.1 eq.) and Cs2CO3 (0.840 g, 2.5 mmol, 1.5 eq.) were added, the reaction mixture was heated at 100° C. for 16 h. After completion of reaction, checked by TLC, the reaction mixture was cooled to room temperature, poured into water (60 mL) and product extracted with ethyl acetate (3×50 mL). The combined organic layers were washed with brine (50 mL), dried over sodium sulfate and concentrated under reduced pressure. The residue was purified by flash column chromatography on silica gel with ethyl acetate in n-hexane to get mixture of two regio isomers (5-(benzyloxy)-2-fluorophenyl)(6-(5-(2-(benzyloxy)pyridin-3-yl)-3-(trifluoromethyl)-1H-pyrazol-1-yl)-2-azaspiro[3.3]heptan-2-yl)methanone (Int-4) (5-(benzyloxy)-2-fluorophenyl)(6-(3-(2-(benzyloxy)pyridin-3-yl)-5-(trifluoromethyl)-1H-pyrazol-1-yl)-2-azaspiro [3.3]heptan-2-yl) methanone (Int-4A) (1.0 g, 90.90%). LCMS: 643.6 m/z [M+1]⁺.

### Step-4: Synthesis of 3-(1-(2-(2-fluoro-5-hydroxybenzoyl)-2-azaspiro [3.3]heptan-6-yl)-3-(trifluoromethyl)-1H-pyrazol-5-yl) pyridin-2(1H)-one (Compound-1 l) and 3-(1-(2-(2-fluoro-5-hydroxybenzoyl)-2-azaspiro [3.3] heptan-6-yl)-5-(trifluoromethyl)-1H-pyrazol-3-yl) pyridin-2(1H-1)-one (Compound-12)

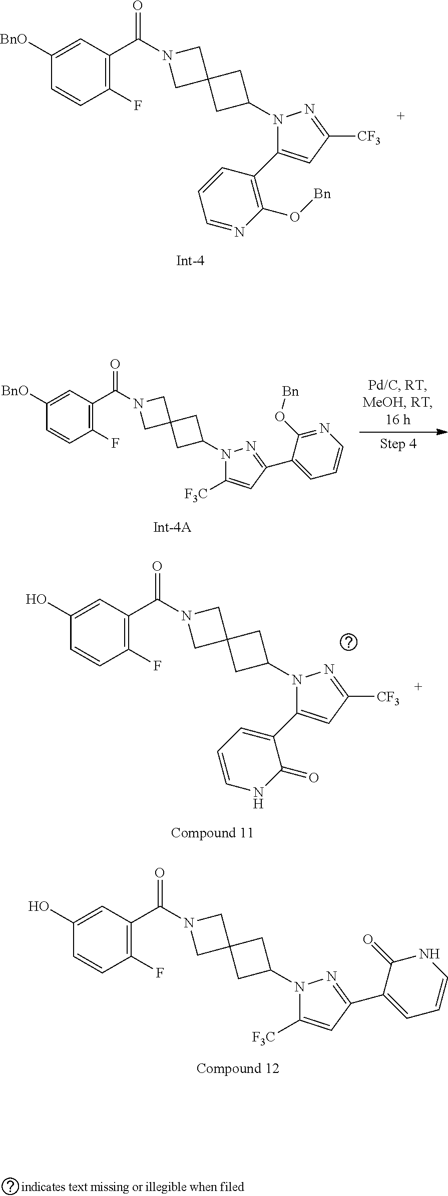 [SMILES: O=C(c1cc(OCc2ccccc2)ccc1F)N1CC2(CC(n3nc(C(F)(F)F)cc3-c3cccnc3OCc3ccccc3)C2)C1 ; O=C(c1cc(OCc2ccccc2)ccc1F)N1CC2(CC(n3nc(-c4cccnc4OCc4ccccc4)cc3C(F)(F)F)C2)C1 ; O=C(c1cc(O)ccc1F)N1CC2(CC(n3nc(C(F)(F)F)cc3-c3ccc[nH]c3=O)C2)C1 ; O=C(c1cc(O)ccc1F)N1CC2(CC(n3nc(-c4ccc[nH]c4=O)cc3C(F)(F)F)C2)C1]

To a solution of (5-(benzyloxy)-2-fluorophenyl)(6-(5-(2-(benzyloxy)pyridin-3-yl)-3-(trifluoromethyl)-1H-pyrazol-1-yl)-2-azaspiro[3.3]heptan-2-yl)methanone (Int-4)& (5-(benzyloxy)-2-fluorophenyl)(6-(3-(2-(benzyloxy) pyridin-3-yl)-5-(trifluoromethyl)-1H-pyrazol-1-yl)-2-azaspiro[3.3]heptan-2-yl) methanone (Int-4A) (1.0 g, 1.7 mmol, 1.0 eq.) in methanol (10 mL), 10% Pd/C with 50% moisture (0.50 g) was added. Reaction mixture stirred under H2 (g) pressure (5 psi) for 10 h at room temperature. After completion of reaction, reaction mixture was filtered through celite bed. Filtrate mL was distilled out to obtain crude product. Which was purified with prep-HPLC to obtain two fractions.

Fraction-1: 3-(1-(2-(2-fluoro-5-hydroxybenzoyl)-2-azaspiro [3.3]heptan-6-yl)-3-(trifluoromethyl)-1H-pyrazol-5-yl) pyridin-2(1H)-one (Compound 11) (0.031 g, 4.35%). LCMS: m/z 463.41 [M+H]⁺. ¹H NMR (400 MHz, DMSO-d₆) δ 11.92 (s, 1H), 9.64 (s, 1H), 8.20-8.11 (d, J=6.9 Hz, 1H), 7.48 (d, J=132 Hz, 2H), 7.09 (t, J=9.4 Hz, 1H), 6.80 (s, 2H), 6.40 (m, 1H), 4.94-4.85 (m, 1H), 4.13 (dd, J=23.9, 7.7 Hz, 4H), 2.84-2.74 (m, 4H).

Fraction-2: 3-(1-(2-(2-fluoro-5-hydroxybenzoyl)-2-azaspiro [3.3]heptan-6-yl)-5-(trifluoromethyl)-1H-pyrazol-3-yl) pyridin-2(1H)-one (Compound 12) (0.012 g, 1.68%). LCMS: m/z 463.30 [M+H]⁺. ¹H NMR (400 MHz, DMSO-d₆) δ 11.83 (s, 1H), 9.65 (s, 1H), 7.59 (dt, J=12.6, 6.4 Hz, 2H), 7.09 (t, J=8.1 Hz, 1H), 6.79 (d, J=2.4 Hz, 1H), 6.74 (s, 1H), 6.71 (s, 1H), 6.34 (dt, =13.3, 6.6 Hz, 1H), 4.75-4.61 (m, 1H), 4.06 (t, J=9.9 Hz, 4H), 2.67 (s, 4H).

### Example 6: Synthesis of 5-(1-(2-(2-fluoro-5-hydroxybenzoyl)-2-azaspiro [3.3]heptan-6-yl)-3-(trifluoromethyl)-1H-pyrazol-5-yl) pyridin-2(1H)-one [Compound 9] and 5-(1-(2-(2-fluoro-5-hydroxybenzoyl)-2-azaspiro [3.3] heptan-6-yl)-5-(trifluoromethyl)-1H-pyrazol-3-yl) pyridin-2(1H)-one [Compound 10]

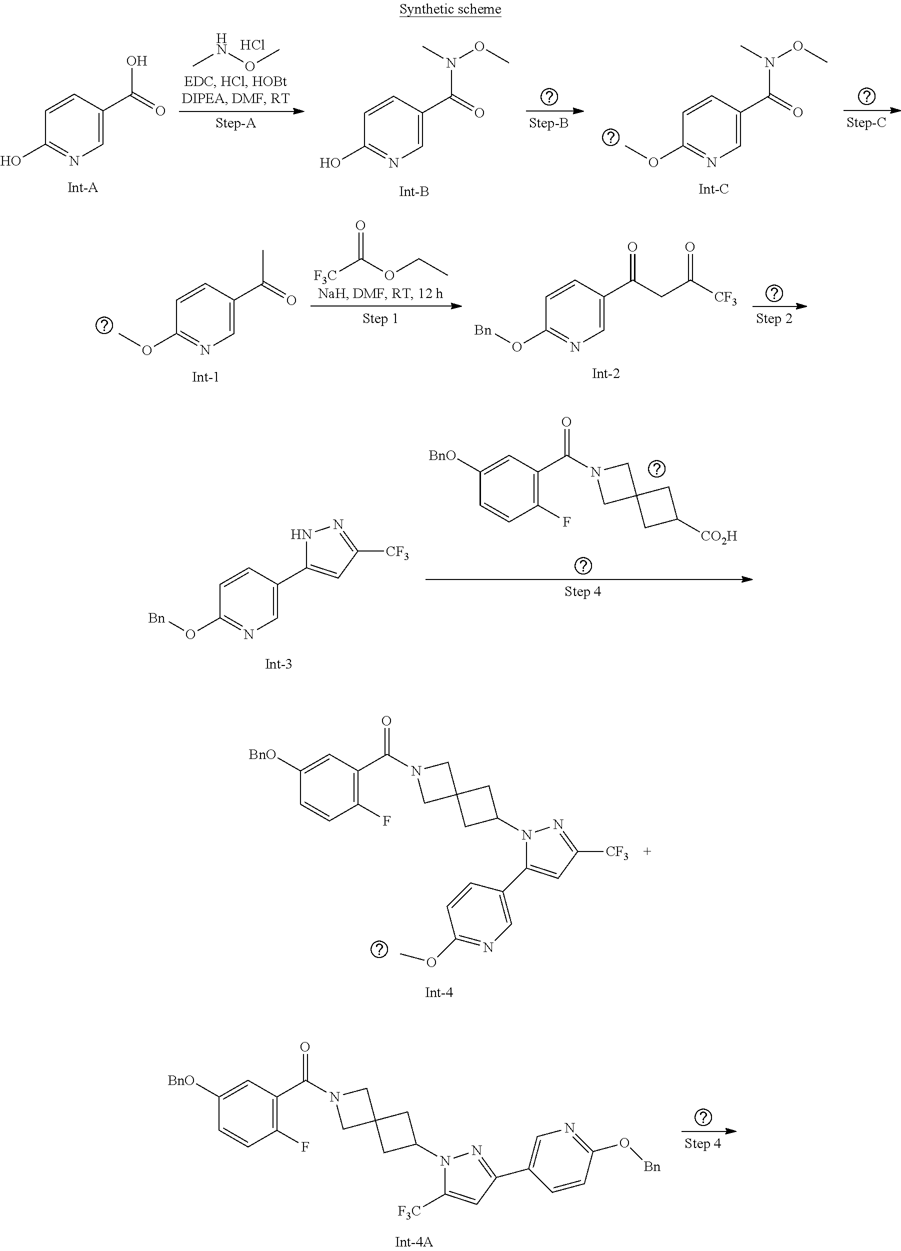

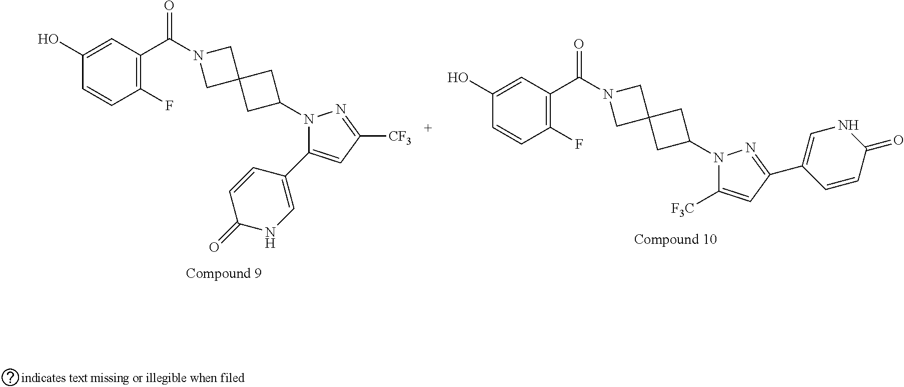 [SMILES: O=C(c1cc(O)ccc1F)N1CC2(CC(n3nc(C(F)(F)F)cc3-c3ccc(=O)[nH]c3)C2)C1 ; O=C(c1cc(O)ccc1F)N1CC2(CC(n3nc(-c4ccc(=O)[nH]c4)cc3C(F)(F)F)C2)C1]

### Step-A: Synthesis of 6-hydroxy-N-methoxy-N-methyl nicotinamide (Int-B)

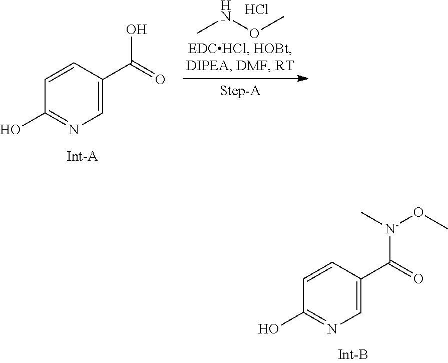

To a solution of the 6-hydroxynicotinic acid (5.0 g, 35.97 mmol, 1.0 eq.) in acetonitrile (40 mL) was added N, N-Diisopropylethylamine (13.96 g, 107.9 mmol, 3.0 eq.) and cooled to 00 C. I-Ethyl-3-(3-dimethylaminopropyl) carbodiimide (EDC-HCl) (10.34 g, 53.95 mmol, 1.5 eq.) was added and stirred for 15 min at 00 C. Followed by 1-Hydroxybenzotriazole (HOBt) (6.3 g, 46.76 mmol, 1.3 eq.), and N,O-dimethyl hydroxylamine (4.6 g, 46.76 mmol, 1.3 eq.) were added to the reaction mixture and stirred at room temperature for 2 h. After completion, the mixture was diluted with water (50 mL) and extracted with ethyl acetate (50 mL*3). The organic layer was dried over sodium sulphate and concentrated in vacuum to get crude compound of 6-hydroxy-N-methoxy-N-methylnicotinamide (Int-B) (6.5 g, yield: crude). LCMS: m/z 183.1 [M+1]+.

### Step-B: Synthesis of 6-(benzyloxy)-N-methoxy-N-methyl nicotinamide (Int-C)

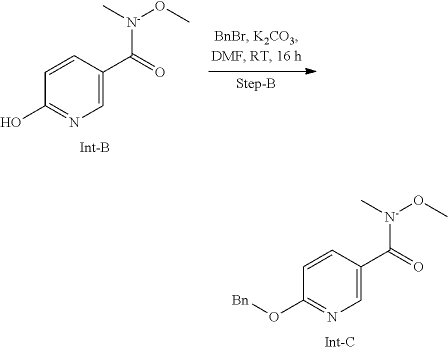

A solution of the 6-hydroxy-N-methoxy-N-methylnicotinamide (6.5 g, 35.71 mmol, 1.0 eq.) and potassium carbonate (7.39 g, 53.57 mmol, 1.5 eq.) in N, N dimethyl form amide (50 mL) was stirred at room temperature for 30 min. Benzyl bromide (18.32 g, 107.14 mmol) was added to the reaction mixture and stirred at room temperature for 16 h. The progress of reaction checked by TLC. After completion of reaction, reaction mixture was diluted with water and extracted with ethyl acetate (50 mL*3). The organic layer was dried over sodium sulphate and concentrated in vacuum to obtained crude product. Which was purified by flash chromatography using 50% Ethyl acetate in n-hexane as eluent to get 6-(benzyloxy)-N-methoxy-N-methyl nicotinamide (Int-C). (7.5 g, 77.20%). LCMS: m/z 273.3 [M+1]+.

### Step-C: Synthesis of 146-(benzyloxy)pyridin-3-yl)ethan-1-one (Int-1)

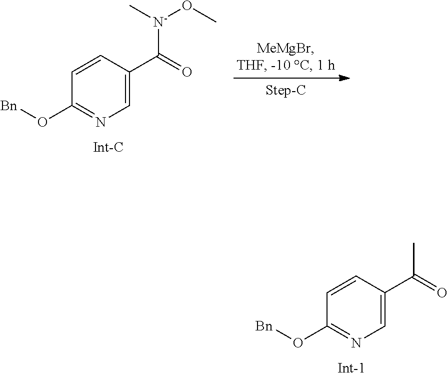

To a solution of the 6-(benzyloxy)-N-methoxy-N-methyl nicotinamide (7.5 g, 27.57 mmol, 1.0 eq.) in anhydrous tetrahydrofuran (75 mL) at −100 C under nitrogen was added dropwise MeMgBr (3M in ether (16.5 mL, 49.63 mmol, 1.8 eq.). The cold bath was removed and the reaction was stirred at room temperature for 1 h. The mixture was diluted with sat. NH4Cl soln and extracted with ethyl acetate (50 mL*3) The combined organics were washed with brine, dried over sodium sulphate, and concentrated. The resulting material was purified by flash chromatography using eluting with 90-100% ethyl acetate in n-hexane as eluent to get 1-(6-(benzyloxy)pyridin-3-yl)ethan-1-one (Int-1) as a white solid. (3.5 g, 55.92%). LCMS: m/z 228.2 [M+1]+.

### Step-1: Synthesis of 1-(6-(benzyloxy) pyridin-3-yl)-4,4,4-trifluorobutane-1,3-dione (Int-2)

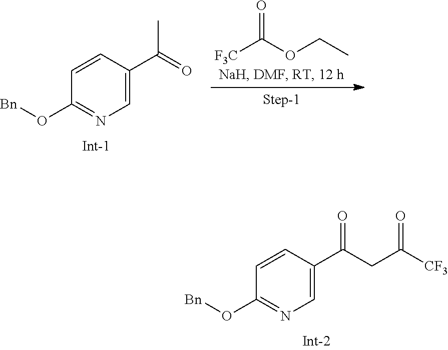

To a stirred solution of 1-(6-(benzyloxy) pyridin-3-yl) ethan-1-one (3.5 g, 15.42 mmol, 1.0 eq.) in N,N-dimethyl formamide (30 mL), was added Sodium hydride (0.55 gm, 23.13 mmol, 1.5 eq.) at 0° C. under N2 atmosphere. The reaction was stirred at 0° C. for 30 min. Then ethyl 2,2,2-trifluoroacetate (3.3 ml, 23.13 mmol, 1.5 eq.) was added dropwise to the reaction and stirred at room temperature for 12 h. After completion of reaction as monitored by TLC, the reaction mixture was diluted with Ice cold water (5 mL) and extracted with Ethyl acetate (2×20 mL) The combined organic layer was washed with brine solution, dried over sodium sulfate and concentrated to obtain crude product, which was purified by combiflash using 10% Ethyl acetate in n-hexane as eluent to get 1-(6-(benzyloxy) pyridin-3-yl)-4,4,4-trifluorobutane-1,3-dione (Int-2) (3.0 g, 70.30%). The compound was mixture of Keto-enol isomer, which was non separable by column chromatography. It used in next step without further purification. LCMS m/z 322.1 [M+1]+.

### Step-2: Synthesis of 2-(benzyloxy)-5-(3-(trifluoromethyl)-1H-pyrazol-5-yl) pyridine (Int-3)

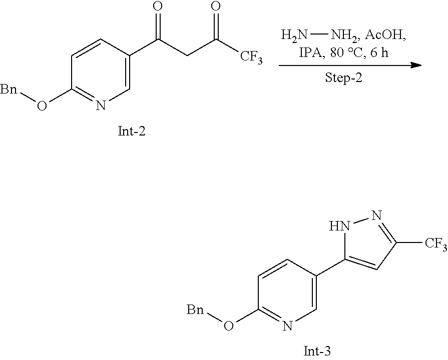

The 1-(6-(benzyloxy) pyridin-3-yl)-4,4,4-trifluorobutane-1,3-dione (3.5 g, 10.84 mmol, 1.0 eq.) was added to the solution of the Hydrazine monohydrate (1.0 g, 21.67 mmol, 2.0 eq.) in isopropanol (15 mL). Then, Acetic acid (1.0 mL) was added and the suspension was heated at 80° C. for 6 h. Progress of reaction checked by TLC. After completion conversion, detected by TLC. The volatile matter was distilled out and diluted with water (40 mL). The product was extracted with ethyl acetate (50 mL*3). The organic layer was washed with brine solⁿ (40 mL), dried over sodium sulphate and concentrated to obtain crude product, which was purified by combiflash using 50% Ethyl acetate in n-hexane as eluent to get 2-(benzyloxy)-5-(3-(trifluoromethyl)-1H-pyrazol-5-yl) pyridine (Int-3) (0.650 g, 18.80%). LCMS: m/z 320.1 [M+1]⁺. ¹H NMR (400 MHz, DMSO-d₆) δ 13.89 (s, 1H), 8.44 (d, J=2.4 Hz, 1H), 7.88 (dd, J=2.4, 7.2 Hz, 1H), 7.35-7.31 (m, 4H), 7.04 (s, 1H), 6.57 (t, J=9.6 Hz, 1H), 5.13 (s, 2H).

### Step-3: Synthesis of (5-(benzyloxy)-2-fluorophenyl) (6-(546-(benzyloxy) pyridin-3-yl)-3-(trifluoro methyl)-1H-pyrazol-1-yl)-2-azaspiro [3.3] heptan-2-yl) methanone (Int-4) and (5-(benzyloxy)-2-fluorophenyl) (6-(3-(6-(benzyloxy) pyridin-3-yl)-5-(trifluoromethyl)-1H-pyrazol-1-yl)-2-azaspiro 3.31 heptan-2-yl) methanone (Int-4A)

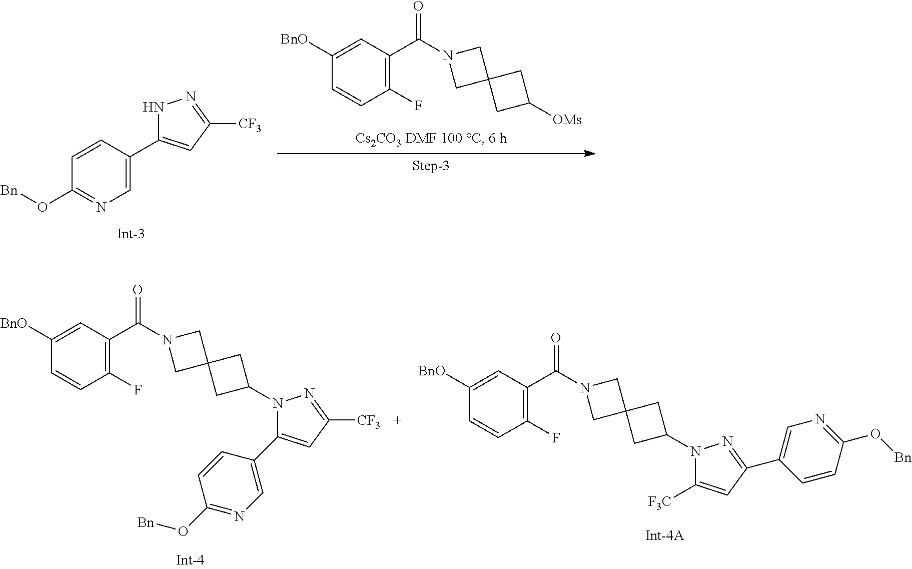 [SMILES: FC(F)(F)c1cc(-c2ccc(OCc3ccccc3)nc2)[nH]n1 ; CS(=O)(=O)OC1CC2(C1)CN(C(=O)c1cc(OCc3ccccc3)ccc1F)C2 ; O=C(c1cc(OCc2ccccc2)ccc1F)N1CC2(CC(n3nc(C(F)(F)F)cc3-c3ccc(OCc4ccccc4)nc3)C2)C1 ; O=C(c1cc(OCc2ccccc2)ccc1F)N1CC2(CC(n3nc(-c4ccc(OCc5ccccc5)nc4)cc3C(F)(F)F)C2)C1]

To a solution of 2-(benzyloxy)-5-(3-(trifluoromethyl)-1H-pyrazol-5-yl) pyridine (0.650 g, 2.03 mmol, 1.0 eq.) in N,N-Dimethylformamide (10 mL), 2-(5-(benzyloxy)-2-fluorobenzoyl)-2-azaspiro [3.3]heptan-6-yl methane sulfonate (0.939 g, 2.24 mml, 1.1 eq) and Cesium carbonate (0.993 g, 3.05 mmol, 1.5 eq.) were added. The reaction mixture was heated at 100° C. for 6 h. Progress of reaction checked by TLC. After completion of reaction, cooled to room temperature. The reaction mixture was poured into water (40 mL), product with ethyl acetate (3×40 mL). The combined organic layers were washed with brine (40 mL), dried over sodium sulphate, concentrated under vacuum to obtained residue. Which was purified by flash column chromatography on silica gel using 50% ethyl acetate in n-hexane as eluent to get mixture of two isomer (5-(benzyloxy)-2-fluorophenyl) (6-(5-(6-(benzyloxy) pyridin-3-yl)-3-(trifluoromethyl)-1H-pyrazol-1-yl)-2-azaspiro [3.3]heptan-2-yl) methanone (Int-4) and (5-(benzyloxy)-2-fluorophenyl) (6-(3-(6-(benzyloxy) pyridin-3-yl)-5-(trifluoromethyl)-1H-pyrazol-1-yl)-2-azaspiro [3.3]heptan-2-yl) methanone (Int-4A) (1.0 g, 76.43%). LCMS: m/z 643.60 [M+1]⁺.

### Step-4: Synthesis of 5-(1-(2-(2-fluoro-5-hydroxybenzoyl)-2-azaspiro [3.3] heptan-6-yl)-3-(trifluoromethyl)-1H-pyrazol-5-yl) pyridin-2(1H)-one (Compound-9) and 5-(1-(2-(2-fluoro-5-hydroxybenzoyl)-2-azaspiro [3.3]heptan-6-yl)-5-(trifluoromethyl)-1H-pyrazol-3-yl) pyridin-2(1H)-one (Compound-10)

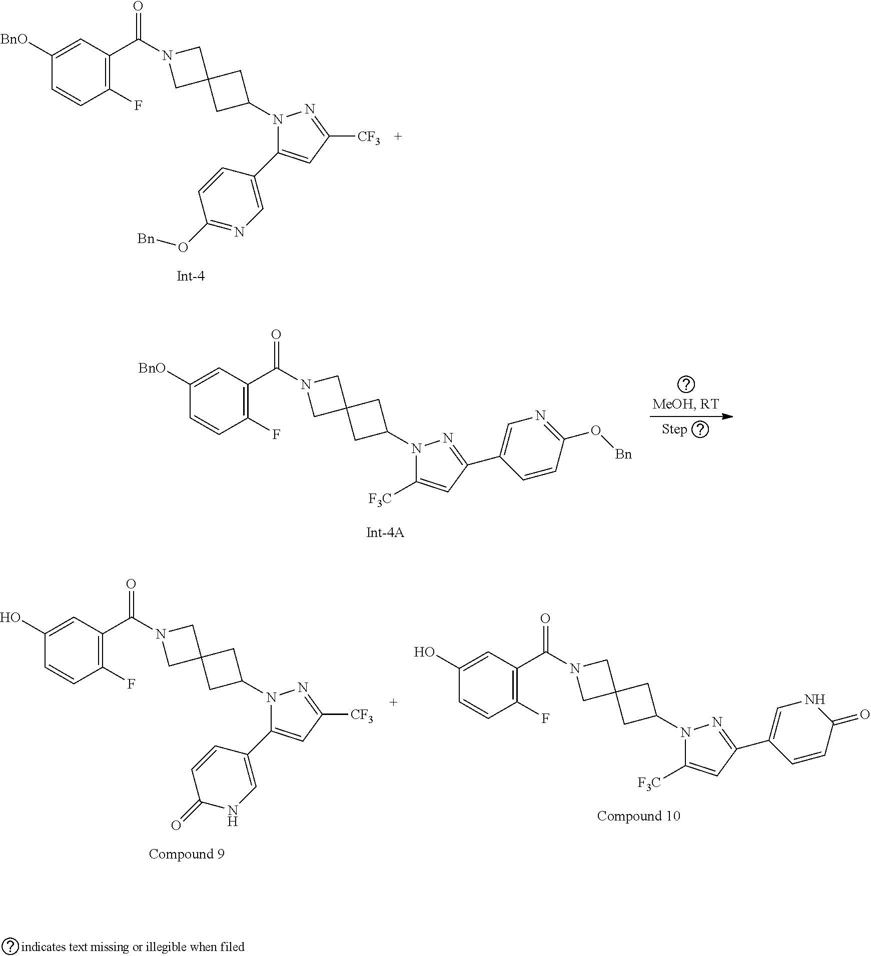 [SMILES: O=C(c1cc(OCc2ccccc2)ccc1F)N1CC2(CC(n3nc(C(F)(F)F)cc3-c3ccc(OCc4ccccc4)nc3)C2)C1 ; O=C(c1cc(OCc2ccccc2)ccc1F)N1CC2(CC(n3nc(-c4ccc(OCc5ccccc5)nc4)cc3C(F)(F)F)C2)C1 ; O=C(c1cc(O)ccc1F)N1CC2(CC(n3nc(C(F)(F)F)cc3-c3ccc(=O)[nH]c3)C2)C1 ; O=C(c1cc(O)ccc1F)N1CC2(CC(n3nc(-c4ccc(=O)[nH]c4)cc3C(F)(F)F)C2)C1]

To a stirred solution of (5-(benzyloxy)-2-fluorophenyl) (6-(5-(6-methoxypyridin-3-yl)-3-(trifluoromethyl)-1H-pyrazol-1-yl)-2-azaspiro [3.3]heptan-2-yl) methanone (Int-4) and (5-(benzyloxy)-2-fluorophenyl) (6-(3-(6-methoxypyridin-3-yl)-5-(trifluoromethyl)-1H-pyrazol-1-yl)-2-azaspiro [3.3]heptan-2-yl) methanone (Int-4A) (0.35 g, 0.545 mmol, 1.0 eq.) in methanol, was added 10% Pd/C with 50% moisture (0.10 g) at room temperature. Reaction was stirred under H_{2(g)} atmosphere for 12 h. After completion of the reaction (monitored by TLC), the reaction mixture was filtered through celite pad and washed with methanol (25 mL*3). The filtrate-mL was evaporated under vacuum to get residue. Which was purified by using Prep-HPLC with 1% formic acid in water and acetonitrile to get two fractions.

Fraction-1: 5-(1-(2-(2-fluoro-5-hydroxybenzoyl)-2-azaspiro [3.3]heptan-6-yl)-3-(trifluoromethyl)-11H-pyrazol-5-yl) pyridin-2(1H)-one (Compound 9) (0.104 g, 41.69%). LCMS: m/z 463.5 [M+]⁺. ¹H NMR (400 MH-z, DMSO-d6) δ 11.87 (s, 1H), 9.66 (s, 11H), 7.93 (dd, J=24.2, 10.6 Hz, 2H), 7.30 (s, 1H), 7.09 (t, J=9.2 Hz, 1H), 6.89-6.76 (m, 2H), 6.42 (dd, J=19.7, 9.8 Hz, 1H1), 4.89-4.79 (m, 1H), 4.11 (dd, J=23.3, 8.8 Hz, 4H1), 2.81-2.71 (m, 4H).

Fraction-2: 5-(1-(2-(2-fluoro-5-hydroxybenzoyl)-2-azaspiro [3.3]heptan-6-yl)-5-(trifluoromethyl)-1H-pyrazol-3-yl) pyridin-2(1H)-one (Compound 10) (0.053 g, 21.15%). LCMS: m/z 463.5 [M+H]⁺. ¹H NMR (400 MHz, DMSO-d₆) δ 10.52-9.60 (s, 2H), 7.61-7.44 (m, 2H), 7.09 (t, J=9.3 Hz, 1H), 6.90-6.76 (m, 3H), 6.45 (dd, J=15.1, 9.5 Hz, 1H), 4.84 (dt, J=22.7, 8.0 Hz, 1H), 4.08 (dd, J=20.4, 9.7 Hz, 4H), 2.69 (p, J=10.8 Hz, 4H).

### Example 7: Synthesis of (2-fluoro-5-hydroxyphenyl)(6-(5-(pyridin-4-yl)-3-(trifluoromethyl)-1H-pyrazol-1-yl)-2-azaspiro[3.3]heptan-2-yl)methanone and [Compound 7] and (2-fluoro-5-hydroxyphenyl)(6-(3-(pyridin-4-yl)-5-(trifluoromethyl)-1H-pyrazol-1-yl)-2-azaspiro[3.3]heptan-2-yl)methanone [Compound 8]

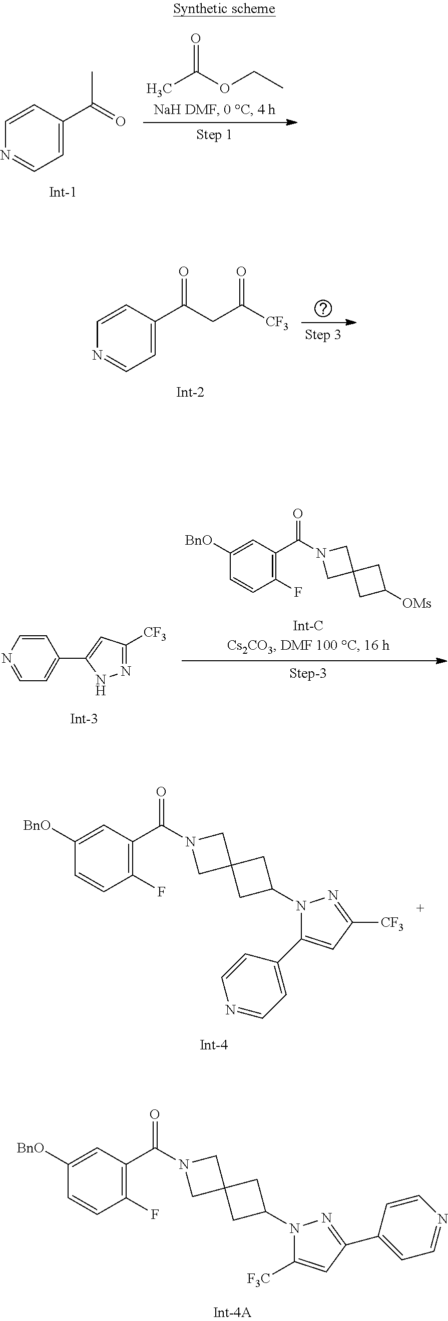

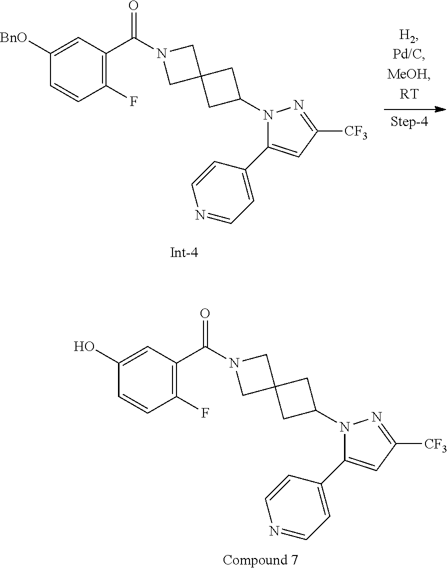 [SMILES: O=C(c1cc(OCc2ccccc2)ccc1F)N1CC2(CC(n3nc(C(F)(F)F)cc3-c3ccncc3)C2)C1 ; O=C(c1cc(O)ccc1F)N1CC2(CC(n3nc(C(F)(F)F)cc3-c3ccncc3)C2)C1]

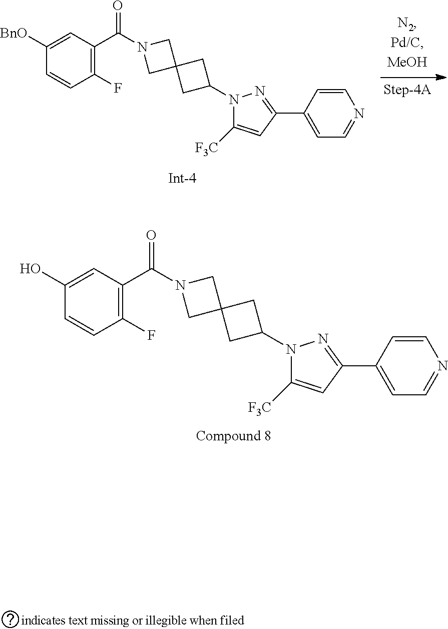 [SMILES: O=C(c1cc(OCc2ccccc2)ccc1F)N1CC2(CC(n3nc(-c4ccncc4)cc3C(F)(F)F)C2)C1 ; O=C(c1cc(O)ccc1F)N1CC2(CC(n3nc(-c4ccncc4)cc3C(F)(F)F)C2)C1]

### Step-1: Synthesis of 4,4,4-trifluoro-1-(pyridin-4-yl)butane-1,3-dione (Int-2)

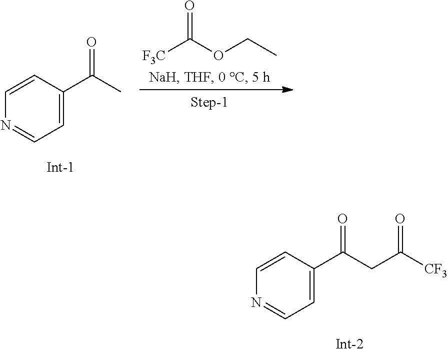

To a stirred solution of 1-(pyridin-4-yl)ethan-1-one (5.0 g, 41.27 mmol, 1.0 eq.) in Tetrahydrofuran (50 mL) was added Sodium hydride (2.47 g, 61.91 mmol, 1.5 eq.) at 0° C., followed by the dropwise addition of ethyl 2,2,2-trifluoroacetate (8.79 g, 61.91 mmol, 1.5 eq.). The reaction mass was stirred at room temperature for 4-5 h. After completion of reaction as monitored by TLC. The reaction mixture was poured in water (100 mL), and extracted with ethyl acetate (50 mL*3). The organic layer was washed with brine (100 mL), dried over Sodium sulfate and concentrated under reduce pressure to get residue. The residue was purified by combiflash using 10% Methanol in Dichloromethane as eluent to obtain 4,4,4-trifluoro-1-(pyridin-4-yl)butane-1,3-dione (Int-2) (7.6 g, 84.80%). LCMS: m/z 218.10 [M+1]+.

### Step-2: Synthesis of 4-(3-(trifluoromethyl)-1H-pyrazol-5-yl) pyridine (Int-3)

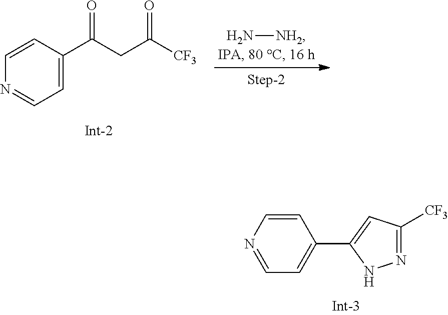

To a stirred solution of 4,4,4-trifluoro-1-(pyridin-4-yl)butane-1,3-dione (7.6 g, 35.02 mmol, 1.0 eq.) in Isopropyl alcohol (40 mL) was added hydrazine hydrate (2.62 g, 52.53 mmol, 1.5 eq.), the reaction mass was heated at 80° C. for 16 h. After completion of reaction as monitored by TLC, the reaction mixture was concentrated to get residue. The residue was purified by combiflash using 40-45% ethyl acetate in n-hexane as eluent to get 4-(3-(trifluoromethyl)-1H-pyrazol-5-yl) pyridine (Int-3) (6.8 g, 91.15%). LCMS: m/z 214.20 [M]+.

### Step-3: Synthesis of (5-(benzyloxy)-2-fluorophenyl) (6-(5-(pyridin-4-yl)-3-(trifluoromethyl)-1H-pyrazol-1-yl)-2-azaspiro [3.3] heptan-2-yl) methanone (Int-4) and (5-(benzyloxy)-2-fluorophenyl)(6-(3-(pyridin-4-yl)-5-(trifluoromethyl)-1H-pyrazol-1-yl)-2-azaspiro [3.3] heptan-2-yl) methanone (Int-4A)

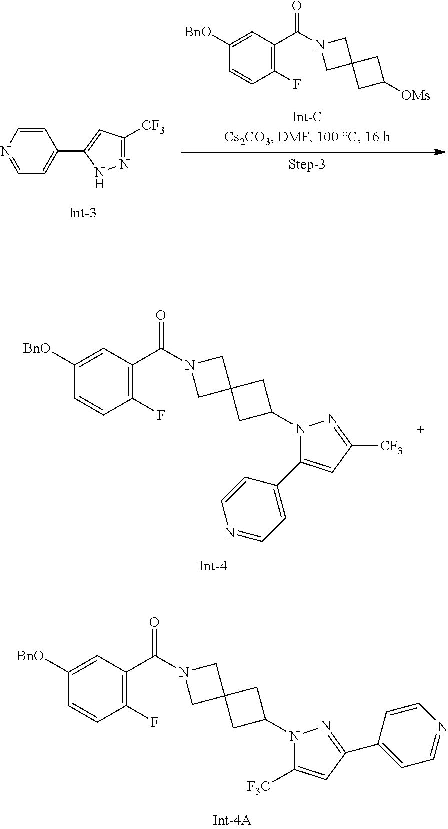 [SMILES: CS(=O)(=O)OC1CC2(C1)CN(C(=O)c1cc(OCc3ccccc3)ccc1F)C2 ; FC(F)(F)c1cc(-c2ccncc2)[nH]n1 ; O=C(c1cc(OCc2ccccc2)ccc1F)N1CC2(CC(n3nc(C(F)(F)F)cc3-c3ccncc3)C2)C1 ; O=C(c1cc(OCc2ccccc2)ccc1F)N1CC2(CC(n3nc(-c4ccncc4)cc3C(F)(F)F)C2)C1]

To a well stirred reaction mixture of 4-(3-(trifluoromethyl)-1H-pyrazol-5-yl) pyridine (0.350 g, 1.64 mmol, 1.0 eq.), Cesium carbonate (0.80 g, 2.46 mmol, 1.5 eq.) in N,N-Dimethylformamide (5 mL) was added 2-(5-(benzyloxy)-2-fluorobenzoyl)-2-azaspiro[3.3]heptan-6-yl methane sulfonate (0.893 g, 2.13 mmol, 1.3 eq.). The reaction was heated at 100° C. for 16 h. After completion of reaction as monitored by TLC, the reaction mixture was poured in water (50 mL) and extracted with ethyl acetate (50*2 mL). The organic layer was washed with brine (30 mL), dried over Sodium sulfate and concentrated under reduce pressure to get residue. The residue was purified by combiflash using 25-30% ethyl acetate in n-hexane as eluent to get two fractions (5-(benzyloxy)-2-fluorophenyl)(6-(5-(pyridin-4-yl)-3-(trifluoromethyl)-1H-pyrazol-1-yl)-2-azaspiro[3.3]heptan-2-yl)methanone (int-4) (0.61 g, 69.24%) and (5-(benzyloxy)-2-fluorophenyl)(6-(3-(pyridin-4-yl)-5-(trifluoromethyl)-1H-pyrazol-1-yl)-2-azaspiro[3.3]heptan-2-yl)methanone (int-4A) (0.24 g, 27.24%). LCMS: m/z 536.53 [M]+.

### Step-4. Synthesis of (2-fluoro-5-hydroxyphenyl)(6-(5-(pyridin-4-yl)-3-(trifluoromethyl)-1H-pyrazol-1-yl)-2-azaspiro[3.3]heptan-2-ylmethanone (Compound-7)

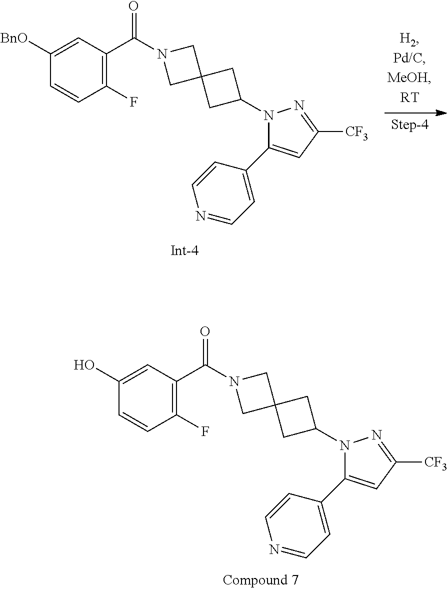 [SMILES: O=C(c1cc(OCc2ccccc2)ccc1F)N1CC2(CC(n3nc(C(F)(F)F)cc3-c3ccncc3)C2)C1 ; O=C(c1cc(O)ccc1F)N1CC2(CC(n3nc(C(F)(F)F)cc3-c3ccncc3)C2)C1]

To a well stirred reaction mixture of (5-(benzyloxy)-2-fluorophenyl)(6-(5-(pyridin-4-yl)-3-(trifluoromethyl)-1H-pyrazol-1-yl)-2-azaspiro[3.3]heptan-2-yl)methanone (0.61 g, 1.13 mmol, 1.0 eq.) in Methanol (5 mL), was added 10% Pd/C on 50% moisture (0.1 g). Reaction was stirred under H₂ g) atmosphere at room temperature for 10 h. After completion of reaction as monitored by TLC, reaction mixture was filtered through celite bed. Filtrate was distilled out to obtain crude product. The crude product was purified by prep-HPLC to obtained (2-fluoro-5-hydroxyphenyl)(6-(5-(pyridin-4-yl)-3-(trifluoromethyl)-1H-pyrazol-1-yl)-2-azaspiro[3.3]heptan-2-yl)methanone (Compound 7) (0.27 g, 53.20%). LCMS: m/z 445.91 [M−1]⁻. ¹H NMR (400 MHz, DMSO-d₆) δ 9.68 (s, 1H), 8.70-8.61 (m, 2H), 7.91-7.81 (m, 2H), 7.66 (s, 1H), 7.11 (t, J=9.2 Hz, 1H), 6.90-6.79 (m, 2H), 5.00-4.91 (m, 1H), 4.15 (dd, J=19.2, 6.3 Hz, 4H), 2.84 (d, J=7.6 Hz, 4H).

### Step-4A; Synthesis of (2-fluoro-5-hydroxyphenyl) (6-(3-(pyridin-4-yl)-5-(trifluoromethyl)-1H-pyrazol-1-yl)-2-azaspiro [3.3] heptan-2-yl) methanone (Compound-8)

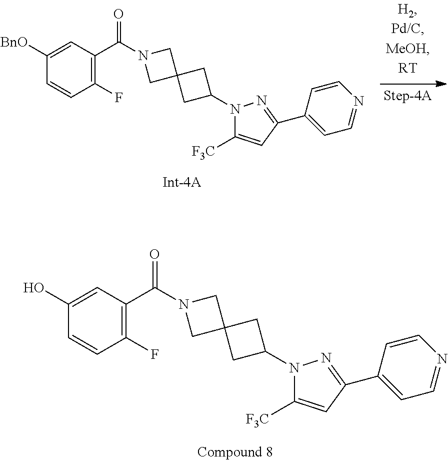 [SMILES: O=C(c1cc(OCc2ccccc2)ccc1F)N1CC2(CC(n3nc(-c4ccncc4)cc3C(F)(F)F)C2)C1 ; O=C(c1cc(O)ccc1F)N1CC2(CC(n3nc(-c4ccncc4)cc3C(F)(F)F)C2)C1]

To a well stirred reaction mixture of (5-(benzyloxy)-2-fluorophenyl) (6-(3-(pyridin-4-yl)-5-(trifluoromethyl)-1H-pyrazol-1-yl)-2-azaspiro [3.3]heptan-2-yl) methanone (0.24 g, 0.44 mmol, 1.0 eq.) in Methanol (3 mL), was added 10% Pd/C on 50% moisture (0.04 g). Reaction was stirred under H_{2(g)} atmosphere at room temperature for 10 h. After completion of reaction as monitored by TLC, reaction mixture was filtered through celite bed. Filtrate was distilled out to obtain crude product. The crude product was purified by prep-HPLC to obtained (2-fluoro-5-hydroxyphenyl)(6-(3-(pyridin-4-yl)-5-(trifluoromethyl)-1H-pyrazol-1-yl)-2-azaspiro[3.3]heptan-2-yl)methanone (Compound 8) (0.011 g, 5.51%). LCMS: m/z 447.30 [M+1]⁺. ¹H NMR (400 MHz, DMSO-d₆) δ 9.64 (s, 1H), 8.73 (d, J=14.4 Hz, 2H), 7.52 (d, J=11.5 Hz, 2H), 7.06 (d, J=16.3 Hz, 2H), 6.84 (s, 1H), 6.77 (s, 1H), 4.96 (s, 1H), 4.06 (dd, J=17.7, 9.9 Hz, 4H), 2.72 (s, 4H).

### Example 8: Synthesis of (2-fluoro-5-hydroxyphenyl)(6-(5-(pyridin-3-yl)-3-(trifluoromethyl)-1H-pyrazol-1-yl)-2-azaspiro[3.3]heptan-2-yl)methanone [Compound 3] and (2-fluoro-5-hydrorxyphenyl)(6-(3-(pyridin-3-yl)-5-(trifluoromethyl)-1H-pyrazol-1-yl)-2-azaspiro[3.3]heptan-2-yl)methanone [Compound 4]

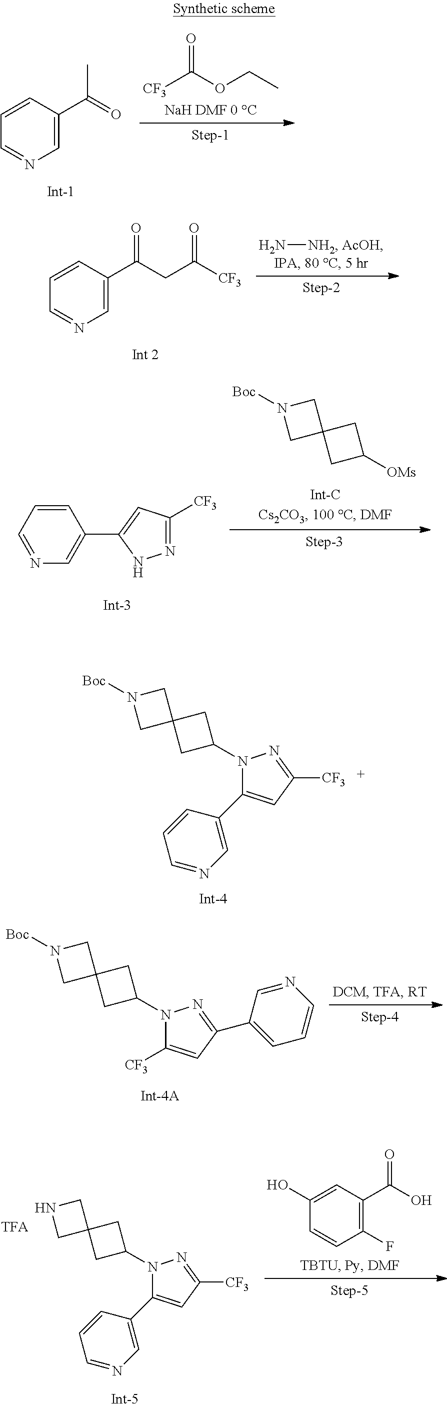

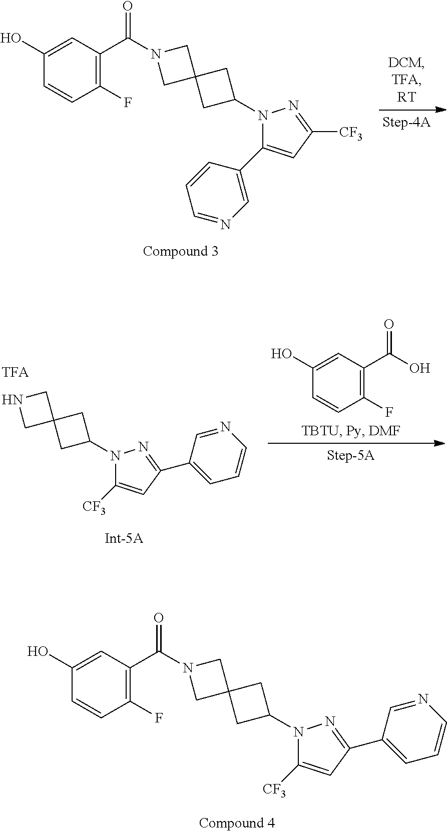 [SMILES: O=C(c1cc(O)ccc1F)N1CC2(CC(n3nc(C(F)(F)F)cc3-c3cccnc3)C2)C1 ; FC(F)(F)c1cc(-c2cccnc2)nn1C1CC2(CNC2)C1 ; O=C(O)c1cc(O)ccc1F ; O=C(c1cc(O)ccc1F)N1CC2(CC(n3nc(-c4cccnc4)cc3C(F)(F)F)C2)C1]

### Step-1: Synthesis of 4,4,4-trifluoro-1-(pyridin-3-yl)butane-1,3-dione (Int-2)

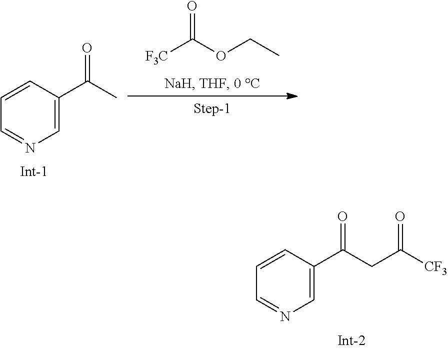

To a stirred solution of 1-(pyridin-3-yl)ethan-1-one (10.0 g, 82.54 mmol, 1.0 eq) in Tetrahydrofuran (50 mL) was added NaH 60% (4.95 g, 123.82 mmol, 1.5 eq.) at 0° C., followed by the dropwise addition of ethyl 2,2,2-trifluoroacetate (17.5 g, 123.82 mmol, 1.5 eq.). The reaction mass was stirred at room temperature for 4-5 hr. After completion of reaction as monitored by TLC. The reaction mixture was poured in water (100 mL), acidified with 10% dilute HCl and extracted with ethyl acetate (50 mL*3). The organic layer was washed with brine (100 mL), dried over sodium sulphate and concentrated under reduce pressure to get residue. The residue was purified by combiflash using 10-15% ethyl acetate in n-hexane as eluent to 4,4,4-trifluoro-1-(pyridin-3-yl) butane-1,3-dione (Int-2) (16.1 g, 89.82%). LCMS: m/z 236.4 [M+18]⁺. ¹H NMR (400 MHz, DMSO-d6) δ 8.94 (s, 1H), 8.63 (d, J=4.0 Hz, 1H), 8.13 (d, J=7.8 Hz, 1H), 7.45 (d, J=7.8 Hz, 1H), 5.97 (s, 1H).

### Step-2: Synthesis of 3-(3-(trifluoromethyl)-1H-pyrazol-5-yl)pyridine (Int-3)

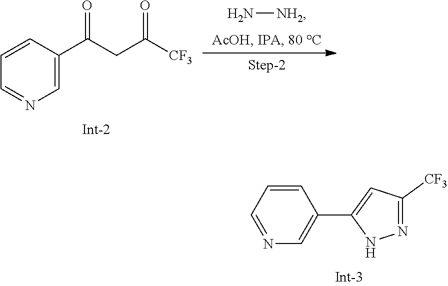

To a stirred solution of 4,4,4-trifluoro-1-(pyridin-3-yl)butane-1,3-dione (6.0 g, 27.90 mmol, 1.0 eq.) in IPA (30 mL) was added hydrazine hydrate (1.5 g, 41.86 mmol, 1.5 eq.) the reaction mass was heated at 80° C. for 16 hr. After completion of reaction as monitored by TLC, the reaction mixture was concentrated to get residue. The residue was purified by combiflash using 20-30% ethyl acetate in n-hexane as eluent to get 3-(3-(trifluoromethyl)-1H-pyrazol-5-yl) pyridine (Int-3) (5.1 g, 86.23%).

### Step-3: Synthesis of Tert-butyl 6-(5-(pyridin-3-yl)-3-(trifluoromethyl)-1H-pyrazol-1-yl)-2-azaspiro 13.31 heptane-2-carboxylate (Int-4) and tert-butyl 6-(3-(pyridin-3-yl)-5-(trifluoromethyl)-1H-pyrazol-1-yl)-2-azaspiro [3.3] heptane-2-carboxylate (Int-4A)

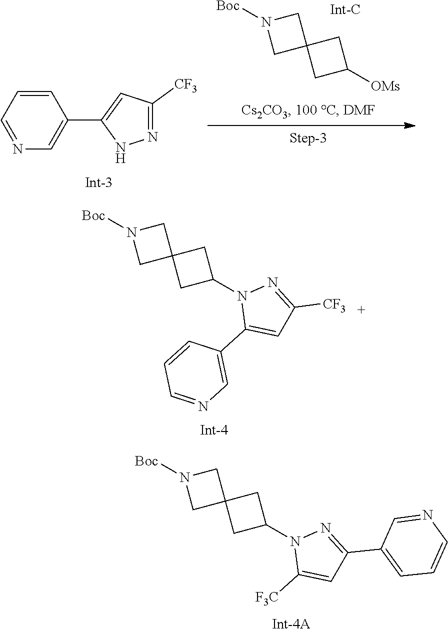 [SMILES: FC(F)(F)c1cc(-c2cccnc2)[nH]n1 ; CC(C)(C)OC(=O)N1CC2(CC(OS(C)(=O)=O)C2)C1 ; CC(C)(C)OC(=O)N1CC2(CC(n3nc(C(F)(F)F)cc3-c3cccnc3)C2)C1 ; CC(C)(C)OC(=O)N1CC2(CC(n3nc(-c4cccnc4)cc3C(F)(F)F)C2)C1]

To a well stirred reaction mixture of 3-(3-(trifluoromethyl)-1H-pyrazol-5-yl) pyridine (2.5 g, 11.62 mmol, 1.0 eq.), CS₂CO₃ (Cesium carbonate) (5.6 g, 17.44 mmol, 1.5 eq.) in N,N-dimethyl formamide (10 mL) was added tert-butyl 6-((methylsulfonyl)oxy)-2-azaspiro[3.3]heptane-2-carboxylate (3.38 g, 11.62 mmol, 1.0 eq.). The reaction was heated at 100° C. for 12 h. After completion of reaction as monitored by TLC, the reaction mixture was poured in water (100 mL) and extracted with ethyl acetate (50 mL*3). The organic layer was washed with brine (100 mL), dried over Sodium sulphate and concentrated under reduce pressure to get residue. The residue was purified by combiflash using 30-50% ethyl acetate in n-hexane as eluent to get two fraction of tert-butyl 6-(5-(pyridin-3-yl)-3-(trifluoromethyl)-1H-pyrazol-1-yl)-2-azaspiro[3.3]heptane-2-carboxylate (Int-4) (1.5 g, 31.31%); LCMS: m/z 409.24 [M+1]⁺ and tert-butyl 6-(3-(pyridin-3-yl)-5-(trifluoromethyl)-1H-pyrazol-1-yl)-2-azaspiro[3.3]heptane-2-carboxylate (Int-4A) (0.65 g, 13.57%); LCMS: m/z 409.3 [M+1]⁺.

### Step-4: Synthesis of 6-(5-(pyridin-3-yl)-3-(trifluoromethyl)-1H-pyrazol-1-yl)-2-azaspiro [3.3] heptane (Int-5)

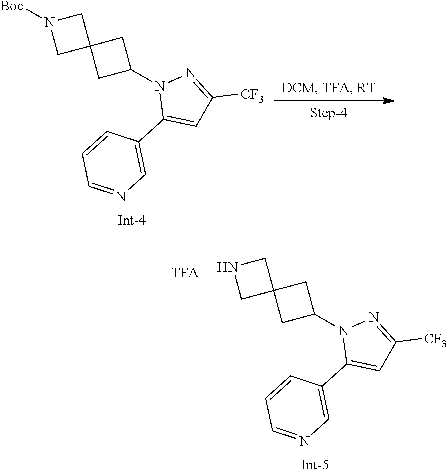 [SMILES: CC(C)(C)OC(=O)N1CC2(CC(n3nc(C(F)(F)F)cc3-c3cccnc3)C2)C1 ; FC(F)(F)c1cc(-c2cccnc2)n(C2CC3(CNC3)C2)n1]

To a well stirred reaction mixture of int-4 tert-butyl 6-(5-(pyridin-3-yl)-3-(trifluoromethyl)-1H-pyrazol-1-yl)-2-azaspiro[3.3]heptane-2-carboxylate (1.0 g, 2.43 mmol, 1.0 eq) in dichloromethane (5 mL) was added trifluoro acetic acid (2.0 mL) dropwise at 0° C. After completion of reaction as monitored by TLC, the reaction mixture was concentrated to get 6-(5-(pyridin-3-yl)-3-(trifluoromethyl)-1H-pyrazol-1-yl)-2-azaspiro [3.3]heptane (Int-5) (0.710 g, 94.06%). LCMS: m/z 308.83 [M]⁺.

### Step-4A: Synthesis of 6-(3-(pyridin-3-yl)-5-(trifluoromethyl)-1H-pyrazol-1-yl)-2-azaspiro [3.3] heptane (Int-5A)

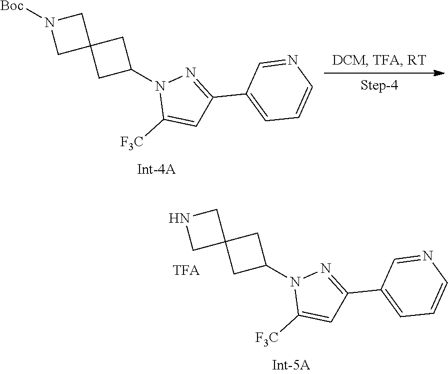 [SMILES: CC(C)(C)OC(=O)N1CC2(CC(n3nc(-c4cccnc4)cc3C(F)(F)F)C2)C1 ; FC(F)(F)c1cc(-c2cccnc2)nn1C1CC2(CNC2)C1]

To a well stirred reaction mixture of int-4A tert-butyl 6-(3-(pyridin-3-yl)-5-(trifluoromethyl)-1H-pyrazol-1-yl)-2-azaspiro[3.3]heptane-2-carboxylate (0.65 g, 1.58 mmol, 1.0 eq.) in dichloromethane (5 mL) was added Trifluoro acetic acid (1.2 mL) dropwise at 0° C. After completion of reaction as monitored by TLC, the reaction mixture was concentrated to get 6-(3-(pyridin-3-yl)-5-(trifluoromethyl)-1H-pyrazol-1-yl)-2-azaspiro [3.3]heptane (int-5A) (0.420 g, 85.60%). LCMS: m/z 309.26 [M+1]⁺.

### Step-5: Synthesis of (2-fluoro-5-hydroxyphenyl)(6-(5-(pyridin-3-yl)-3-(trifluoromethyl)-1H-pyrazol-1-yl)-2-azaspiro[3.3]heptan-2-yl)methanone (Compound 3)

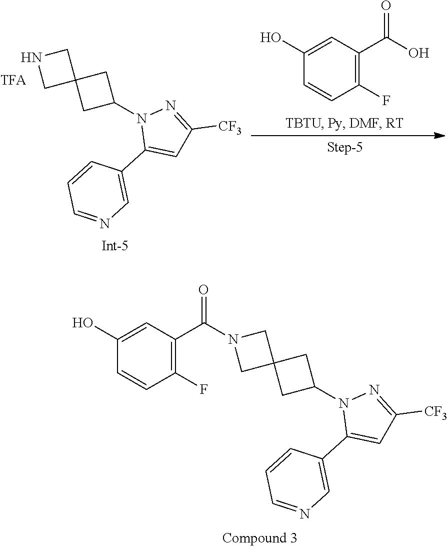 [SMILES: FC(F)(F)c1cc(-c2cccnc2)n(C2CC3(CNC3)C2)n1 ; O=C(O)c1cc(O)ccc1F ; O=C(c1cc(O)ccc1F)N1CC2(CC(n3nc(C(F)(F)F)cc3-c3cccnc3)C2)C1]

To a well stirred reaction mixture of int-5 6-(5-(pyridin-3-yl)-3-(trifluoromethyl)-1H-pyrazol-1-yl)-2-azaspiro[3.3]heptane (0.71 g, 2.29 mmol, 1.2 eq.), Pyridine (0.66 g, 9.42 mmol, 5.0 eq.), int-D (0.29 g, 1.90 mmol, 1.0 eq.) in N,N-dimethyl formamide (5 mL) was added TBTU (0.93 g, 2.86 mmd, 1.5 eq) at 0° C. The reaction mass was stirred at room temperature for 3-4 hr. After completion of reaction as monitored by TLC, the reaction mixture was poured in water (100 mL) and extracted with ethyl acetate (50 mL*3). The organic layer was washed with saturated solution of sodium carbonate (50 mL) and brine (100 mL), dried over sodium sulphate and concentrated under reduce pressure to get residue. The residue was purified by combiflash using 40-50% ethyl acetate in n-hexane as eluent. The crude was submitted to prep HPLC for purification. (Compound 3) (0.086 g, 2.66%). LCMS: m/z 447.24 [M+1]⁺. ¹H NMR (400 MHz, DMSO-d₆) δ 9.70 (s, 1H), 9.11 (d, J=16.9 Hz, 1H), 8.59 (d, J=3.8 Hz, OH), 8.26 (dd, J=22.5, 7.4 Hz, 1H), 7.59 (s, 1H), 7.54-7.46 (m, 1H), 7.10 (d, J=8.7 Hz, 1H), 6.88 (s, 1H), 6.82 (s, 1H), 4.99-4.90 (m, 1H), 4.16 (dd, J=19.4, 8.7 Hz, 4H), 2.88-2.78 (m, 4H).

### Step-5A: Synthesis of (2-fluoro-5-hydroxyphenyl)(6-(3-(pyridin-3-yl)-5-(trifluoromethyl)-1H-pyrazol-1-yl)-2-azaspiro[3.3]heptan-2-yl)methanone (Compound 4)

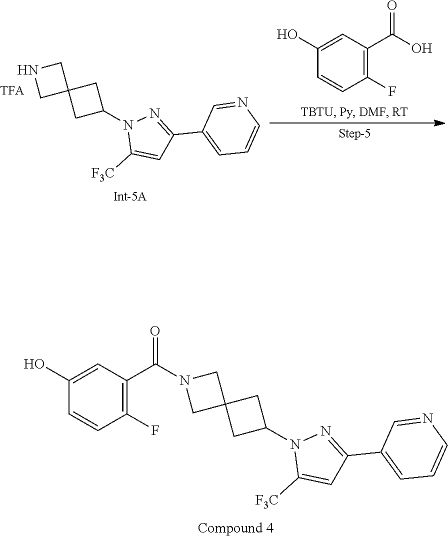 [SMILES: FC(F)(F)c1cc(-c2cccnc2)nn1C1CC2(CNC2)C1 ; O=C(O)c1cc(O)ccc1F ; O=C(c1cc(O)ccc1F)N1CC2(CC(n3nc(-c4cccnc4)cc3C(F)(F)F)C2)C1]

To a well stirred reaction mixture of 6-(3-(pyridin-3-yl)-5-(trifluoromethyl)-1H-pyrazol-1-yl)-2-azaspiro[3.3]heptane (0.42 g, 1.56 mmol, 1.2 eq.), Pyridine (0.45 g, 6.50 mmol, 5.0 eq.), int-D (0.255 g, 1.30 mmol, 1.0 eq.) in N,N-dimethyl formamide (5 mL) was added TBTU (0.63 g, 1.95 mmol, 1.5 eq.) at 0° C. The reaction mass was stirred at room temperature for 3-4 hr. After completion of reaction as monitored by TLC, the reaction mixture was poured in water (100 mL) and extracted with ethyl acetate (50 mL*3). The organic layer was washed with saturated solution of sodium carbonate (50 mL) and brine (100 mL), dried over sodium sulphate and concentrated under reduce pressure to get residue. The residue was purified by combiflash using 80% ethyl acetate in hexane as eluent. The crude was submitted to prep HPLC for purification. Compound 4 (0.092 g, 4.81%). LCMS: m/z 447.24 [M+1]⁻. ¹H NMR (400 MHz, DMSO-d6) δ 8.72 (t, J=9.2 Hz, 2H), 8.00-7.89 (m, 1H), 7.65-7.53 (m, 1H), 7.13-7.02 (m, 2H), 6.87 (s, 2H), 4.89 (dt, J=22.3, 7.8 Hz, 1H), 4.08 (t, J=10.9 Hz, 4H), 2.74 (dt, J=17.9, 9.9 Hz, 4H).

### Example 9: Synthesis of (2-fluoro-5-hydroxyphenyl)(6-(5-(pyridin-2-yl)-3-(trifluoromethyl)-1H-pyrazol-1-yl)-2-azaspiro[3.3]heptan-2-yl)methanone [Compound 5] and (2-fluoro-5-hydroxyphenyl)(6-(3-(pyridin-2-yl)-5-(trifluoromethyl)-1H-pyrazol-1-yl)-2-azaspiro[3.3]heptan-2-yl)methanone [Compound 6]

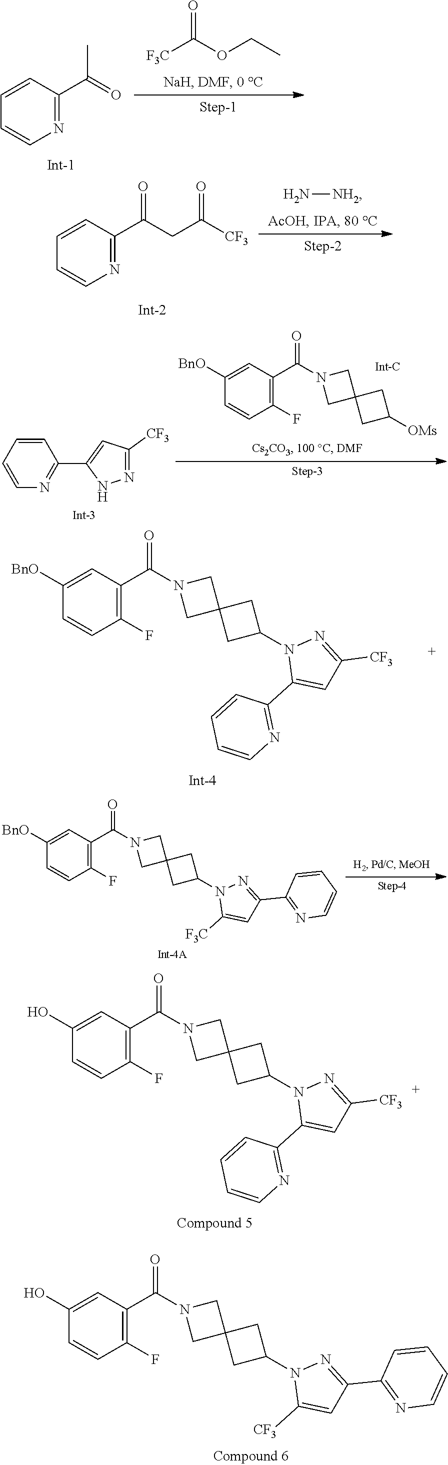

### Step-1: Synthesis of 4,4,4-trifluoro-1-(pyridin-2-yl)butane-1,3-dione (Int-2)

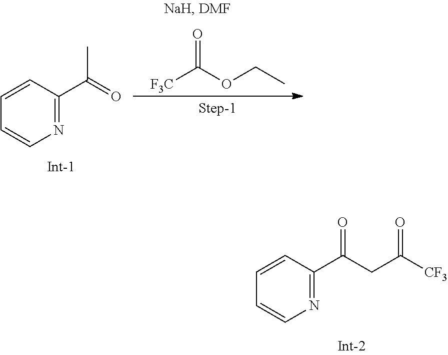

In a dry flask, a mixture of 1-(pyridin-2-yl)ethan-1-one (5.00 g, 41.2745 mmol, 1.0 eq.) in dry Tetrahydrofuran (50 mL) was stirred at 0° C. Sodium hydride (2.47 g, 61.91 mmol, 1.5 eq.) was added portion wise into the well stirred reaction mixture. Reaction mixture was stirred for 10 min then, ethyl 2,2,2-trifluoroacetate (8.79 g, 61.91 mmol, 1.5 eq.) was dropwise added into reaction mixture at 0° C. Reaction mass was stirred at room temperature for 4 to 5 hr. After completion of reaction, the reaction mixture was poured into water (20 mL), and the solution was extracted with ethyl acetate (3-20 mL). The combined organic layers were washed with brine (20 mL), dried over sodium sulfate, filtered and concentrated under reduced pressure. The residue was purified by flash column chromatography on silica gel with n-hexane/ethyl acetate to get pure product 4,4,4-trifluoro-1-(pyridin-2-yl)butane-1,3-dione (Int-2). Yield 8.80 g (98.21%). LCMS: m/z 218.2 [M+1]⁺.

### Step-2: Synthesis of 2-(3-(trifluoromethyl)-1H-pyrazol-5-yl) pyridine (Int-3)

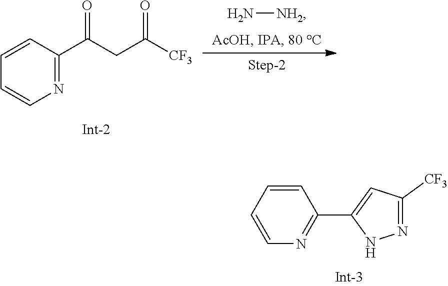

To a solution of 4,4,4-trifluoro-1-(pyridin-2-yl)butane-1,3-dione (8.800 g, 40.52 mmol, 1.0 eq.) in ethanol (80 mL) was added hydrazine hydrate (3.039 g, 60.7873 mmol, 1.5 eq.). The mixture was stirred at room temperature for 1 hr and then, at 80° C.; for 15 hr. After removal of the solvent, the residue was purified by chromatography using 60% to 100% ethyl acetate in n-hexane as eluent to give the title compound 2-(3-(trifluoromethyl)-1H-pyrazol-5-yl) pyridine (Int-3). Yield 7.00 g (81.03%). LCMS: m/z 214.06 [M+1]⁺.

### Step-3. Synthesis of 5-(benzyloxy)-2-fluorophenyl)(6-(5-(pyridin-2-yl)-3-(trifluoromethyl)-1H-pyrazol-1-yl)-2-azaspiro[3.3]heptan-2-yl)methanone (Int-4) and 5-(benzyloxy)-2-fluorophenyl)(6-(3-(pyridin-2-yl)-5-(trifluoromethyl)-1H-pyrazol-1-yl)-2-azaspiro[3.3]heptan-2-yl)methanone (Int-4A)

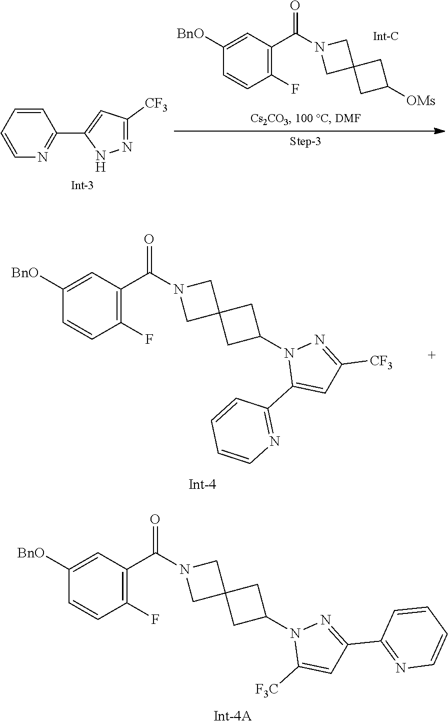 [SMILES: CS(=O)(=O)OC1CC2(C1)CN(C(=O)c1cc(OCc3ccccc3)ccc1F)C2 ; FC(F)(F)c1cc(-c2ccccn2)[nH]n1 ; O=C(c1cc(OCc2ccccc2)ccc1F)N1CC2(CC(n3nc(C(F)(F)F)cc3-c3ccccn3)C2)C1 ; O=C(c1cc(OCc2ccccc2)ccc1F)N1CC2(CC(n3nc(-c4ccccn4)cc3C(F)(F)F)C2)C1]

To a solution of 2-(3-(trifluoromethyl)-1H-pyrazol-5-yl)pyridine (Int-3) (0.30 g, 1.40 mmol, 1.0 eq.) in N,N-dimethyl formamide (6.0 mL), 2-(5-(benzyloxy)-2-fluorobenzoyl)-2-azaspiro[3.3]heptan-6-yl methane sulfonate (Int-C) (0.590 g, 1.40 mmol, 1.0 eq.) and cesium carbonate (Cs₂CO₃) (0.688 g, 2.11 mmol, 1.5 eq.) were added, and the reaction mixture was heated at 100° C. for 16 h. After cooling to room temperature, the reaction mixture was poured into water (60 mL), and the solution was extracted with ethyl acetate (3×50 mL). The combined organic layers were washed with brine (50 ml.), dried over sodium sulfate, filtered and concentrated under reduced pressure. The residue was purified by flash column chromatography on silica gel with n-hexane/ethyl acetate to get two region isomers of (5-(benzyloxy)-2-fluorophenyl)(6-(5-(pyridin-2-yl)-3-(trifluoromethyl)-1H-pyrazol-1-yl)-2-azaspiro[3.3]heptan-2-yl)methanone (Int-4) and (5-(benzyloxy)-2-fluorophenyl)(6-(3-(pyridin-2-yl)-5-(trifluoromethyl)-1H-pyrazol-1-yl)-2-azaspiro[3.3]heptan-2-yl)methanone (Int-4A) Yield: 0.700 g (92.71%). LCMS: 537.26 m/z [M+1]⁺.

### Step-4. Synthesis of (2-fluoro-5-hydroxyphenyl)(6-(5-(pyridin-2-yl)-3-(trifluoromethyl)-1H-pyrazol-1-yl)-2-azaspiro[3.3]heptan-2-yl)methanone (Compound 5) and (2-fluoro-5-hydroxyphenyl)(6-(3-(pyridin-2-yl)-5-(trifluoromethyl)-1H-pyrazol-1-yl)-2-azaspiro[3.3]heptan-2-yl)methanone (Compound 6)

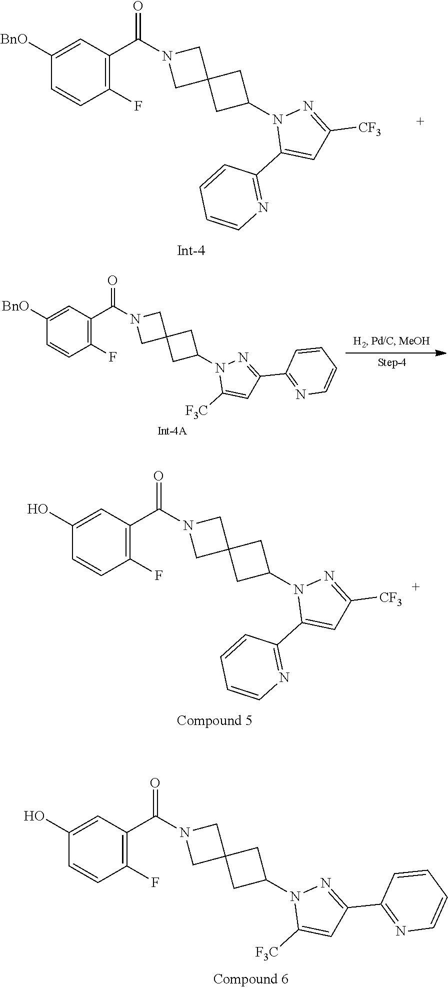 [SMILES: O=C(c1cc(OCc2ccccc2)ccc1F)N1CC2(CC(n3nc(C(F)(F)F)cc3-c3ccccn3)C2)C1 ; O=C(c1cc(OCc2ccccc2)ccc1F)N1CC2(CC(n3nc(-c4ccccn4)cc3C(F)(F)F)C2)C1 ; O=C(c1cc(O)ccc1F)N1CC2(CC(n3nc(C(F)(F)F)cc3-c3ccccn3)C2)C1 ; O=C(c1cc(O)ccc1F)N1CC2(CC(n3nc(-c4ccccn4)cc3C(F)(F)F)C2)C1]

To a solution of (5-(benzyloxy)-2-fluorophenyl)(6-(5-(pyridin-2-yl)-3-(trifluoromethyl)-1H-pyrazol-1-yl)-2-azaspiro[3.3]heptan-2-yl)methanone (Int-4) and (5-(benzyloxy)-2-fluorophenyl)(6-(3-(pyridin-2-yl)-5-(trifluoromethyl)-1H-pyrazol-1-yl)-2-azaspiro[3.3]heptan-2-yl)methanone (Int-4A) (0.700 g, 1.30 mmol, 1.0 eq.) in methanol (10 mL), 10% Pd/C with 50% moisture (0.034 g, 0.32 mmol, 0.25 eq.) was added, hydrogen gas was purged continuously and the reaction mixture was stirred at room temperature for 4 hr. After completion of reaction, reaction mixture was filtered through celite bed. Filtrate was distilled out to obtain crude product. Crude was purified with prep HPLC to obtain (2-fluoro-5-hydroxyphenyl)(6-(5-(pyridin-2-yl)-3-(trifluoromethyl)-1H-pyrazol-1-yl)-2-azaspiro[3.3]heptan-2-yl)methanone (Compound 5) and (2-fluoro-5-hydroxyphenyl)(6-(3-(pyridin-2-yl)-5-(trifluoromethyl)-1H-pyrazol-1-yl)-2-azaspiro[3.3]heptan-2-yl)methanone (Compound 6). [Yield: 0.570 g, 97.93%] which was submitted for prep-HPLC purification.

Fraction-1: (2-fluoro-5-hydroxyphenyl)(6-(3-(pyridin-2-yl)-5-(trifluoromethyl)-1H-pyrazol-1-yl)-2-azaspiro[3.3]heptan-2-yl)methanone (Compound 6). 0.176 g (Yield: 30.24%). LCMS: m/z 445.4 [M−H]⁻. ¹H NMR (400 MHz, DMSO-d) δ 9.70 (s, 1H), 8.68-8.60 (m, 1H), 8.06-7.93 (ddd, J=27.6, 13.4, 7.7 Hz, 2H), 7.46-7.36 (m, 2H), 7.12 (t, J=8.7 Hz, 1H), 6.92-6.80 (m, 2H), 4.96 (dt, J=16.1, 7.7 Hz, 1H), 4.16 (dd, J=22.0, 8.1 Hz, 4H), 2.90-2.83 (dd, J=23.5, 10.0 Hz, 4H).

Fraction-2: (2-fluoro-5-hydroxyphenyl)(6-(5-(pyridin-2-yl)-3-(trifluoromethyl)-1H-pyrazol-1-yl)-2-azaspiro[3.3]heptan-2-yl)methanone (Compound 5). 0.177 g (Yield: 30.41%). LCMS: 447.19 m/z [M+1]⁺. ¹H NMR (400 MHz, DMSO-d₆) δ 9.77 (s, 1H), 8.77 (s, 1H), 8.01-7.92 (m, 1H), 7.85 (d, J=4.9 Hz, 1H), 7.49 (dd, J=10.3, 5.6 Hz, 1H), 7.29 (s, 1H), 7.10 (t, J=9.0 Hz, 1H), 6.87 (s, 1H), 6.80 (s, 1H), 5.62 (dt, J=16.0, 7.8 Hz, 1H), 4.11 (dd, J=27.7, 9.1 Hz, 4H), 2.77-2.70 (m, 4H).

### Example 10: Synthesis of (2-cyclopropoxy-4-fluorophenyl) (6-((3-(2-fluorophenyl)-1H-pyrazol-5-yl)oxy)-2-azaspiro[3.3]heptan-2-yl) methanone [Compound 45], (2-cyclopropoxy-4-fluorophenyl) (6-((5-(2-fluorophenyl)-1-methyl-1H-pyrazol-3-yl)oxy)-2-azaspiro[3.3]heptan-2-yl) methanone [Compound 46] and (2-cyclopropoxy-4-fluorophenyl) (6-(3-(2-fluorophenyl)-1-methyl-1H-pyrazol-5-yl)oxy)-2-azaspiro[3.3]heptan-2-yl) methanone [Compound 47]

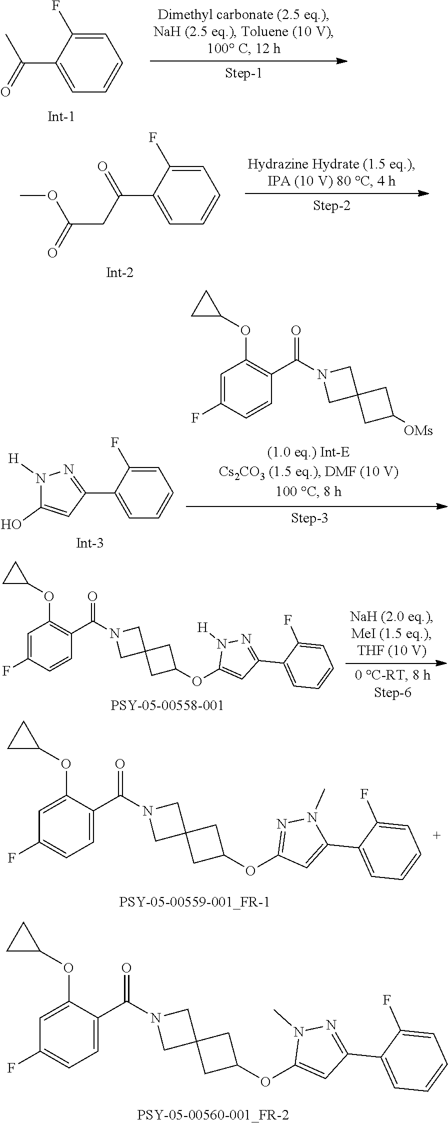

### Synthetic Scheme:

### Step-1: methyl 3-(2-fluorophenyl)-3-oxopropanoate (Int-2)

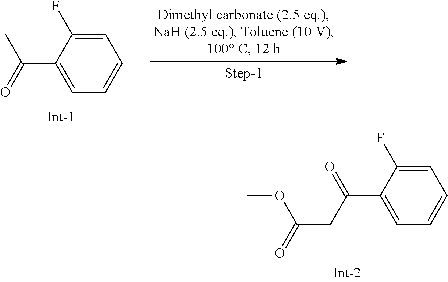

A solution of the 1-(2-fluorophenyl) ethan-1-one (Int-1) (5.0 g, 36.23 mmol, 1.0 eq.) in Toluene (50 mL, 10 V), were added Sodium hydride (60% suspension in mineral oil) (3.62 g, 90.57 mmol, 2.5 eq.) at 0° C. under N2 g) atmosphere, followed by addition of dimethyl Carbonate (8.15 g, 90.57 mmol, 2.5 eq.) and allowed to stir the reaction mass at room temperature for 12 h. After completion of reaction as monitored by TLC, reaction mixture was acidified with 10% dil. hydrochloric acid and extracted with ethyl acetate (3*40 mL). The organic layer was washed with brine, dried over sodium sulphate, concentrated and purified by flash chromatography using 3-4% ethyl acetate in n-hexane to get methyl 3-(2-fluorophenyl)-3-oxopropanoate (Int-2) (5.45 g, 76.75%).

### Step-2: 3-(2-fluorophenyl)-1H-pyrazol-5-ol (Int-3)

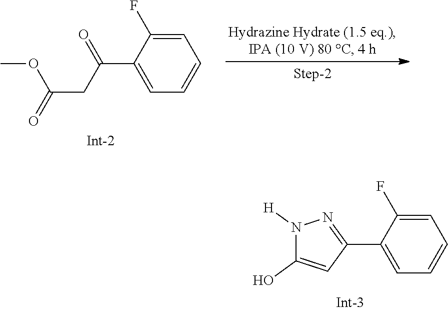

A mixture of the methyl 3-(2-fluorophenyl)-3-oxopropanoate (Int-2) (5.45 g, 27.80 mmol, 1.0 eq.) in Isopropyl alcohol (55 mL, 10 V) were added hydrazine hydrate (2.07 g, 41.70 mmol, 1.5 eq.). The resulting mixture was heated at 80° C. for 4 h. After completion of reaction as monitored by TLC, reaction mixture was concentrated wider vacuum and purified by flash chromatography using 0-25% ethyl acetate in n-hexane to get 3-(2-fluorophenyl)-1H-pyrazol-5-ol (Int-3) (4.3 g, 86.88%).

### Step-3: (2-cyclopropoxy-4-fluorophenyl) (6-((3-(2-fluorophenyl)-1H-pyrazol-5-yl) oxy)-2-azaspiro[3.3]heptan-2-yl)methanone (Compound 45)

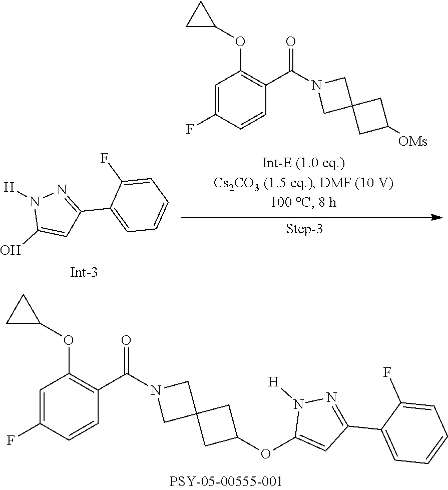 [SMILES: CS(=O)(=O)OC1CC2(C1)CN(C(=O)c1ccc(F)cc1OC1CC1)C2 ; Oc1cc(-c2ccccc2F)n[nH]1 ; O=C(c1ccc(F)cc1OC1CC1)N1CC2(CC(Oc3cc(-c4ccccc4F)n[nH]3)C2)C1]

A mixture of 3-(2-fluorophenyl)-1H-pyrazol-5-ol (Int-3) (4.3 g, 24.15 mmol, 1.0 eq.) in N,N-Dimethylformamide (43 mL, 10 V) were added Cesium carbonate (11.7 g, 36.23 mmol, 1.5 eq.) and Int-E (7.1 g, 19.32 mmol, 0.8 eq.) at room temperature. The resulting mixture was heated at 100° C. for 8 h. After completion of reaction as monitored by TLC, the reaction mixture was poured into water and extracted with ethyl acetate (3*40 mL). The organic layer was washed with brine (100 mL), dried over Sodium sulfate and concentrated under reduce pressure to obtain (2.2 g, crude). Crude (0.7 g) was purified by Prep-HPLC to get (2-cyclopropoxy-4-fluorophenyl) (6-((3-(2-fluorophenyl)-1H-pyrazol-5-yl) oxy)-2-azaspiro [3.3]heptan-2-yl)methanone (PSY-05-00555-001) (0.15 g, 13.07%).

### Step-4: (2-cyclopropoxy-4-fluorophenyl) (6-((5-(2-fluorophenyl)-1-methyl-1H-pyrazol-3-yl)oxy)-2-azaspiro[3.3]heptan-2-yl)methanone (Compound 46) and (2-cyclopropoxy-4-fluorophenyl(6-((3-(2-fluorophenyl)-1-methyl-1H-pyrazol-5-yl)oxy)-2-azaspiro[3.3]heptan-2-yl)methanone (Compound 47)

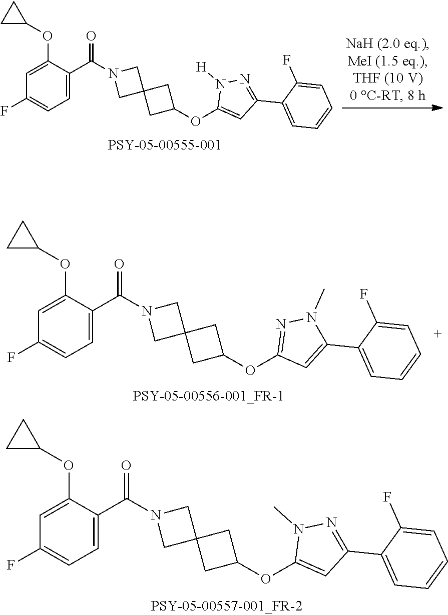 [SMILES: O=C(c1ccc(F)cc1OC1CC1)N1CC2(CC(Oc3cc(-c4ccccc4F)n[nH]3)C2)C1 ; Cn1nc(OC2CC3(C2)CN(C(=O)c2ccc(F)cc2OC2CC2)C3)cc1-c1ccccc1F ; Cn1nc(-c2ccccc2F)cc1OC1CC2(C1)CN(C(=O)c1ccc(F)cc1OC1CC1)C2]

To a solution of the (2-cyclopropoxy-4-fluorophenyl) (6-((3-(2-fluorophenyl)-1H-pyrazol-5-yl) oxy)-2-azaspiro[3.3]heptan-2-yl)methanone (PSY-05-00555-001) (1.0 g, 2.21 mmol, 1.0 eq.) in Tetrahydrofuran (10 mL, 10V), were added sodium hydride (60% suspension in mineral oil) (0.17 g, 4.43 mmol, 2.0 eq.) at 0° C. under N_{2(g)} atmosphere, followed by dropwise addition of methyl iodide (0.46 g, 6.65 mmol 1.5 eq.). Then reaction mass allowed to stir at room temperature for 8 h. After completion of reaction as monitored by TLC. The crude was diluted with water (100 mL) and extracted with ethyl acetate (50 mL*3). The organic layer was washed with brine (100 mL), dried over Sodium sulfate and concentrated under reduce pressure to obtain crude. Which was purified by prep-HPLC to obtained two fraction.

Fraction-1: (2-cyclopropoxy-4-fluorophenyl)(6-((5-(2-fluorophenyl)-1-methyl-1H-pyrazol-3-yl) oxy)-2-azaspiro[3.3]heptan-2-yl)methanone (Compound 46) (0.055 g, 5.33%).

Fraction-2: (2-cyclopropoxy-4-fluorophenyl)(6-((3-(2-fluorophenyl)-1-methyl-1H-pyrazol-5-yl)oxy)-2-azaspiro[3.3]heptan-2-yl)methanone (Compound 472) (0.040 g, 3.88%).

| PSY Compounds | |
|---|---|
| 45, 46 and 47 | MS/NMR Description |
| Step-1: Int-2 | methyl 3-(2-fluorophenyl)-3-oxopropanoate (Int-2) |
| | LCMS: m/z 197.08 [M + 1]⁺. |
| | NMR: ¹H NMR (400 MHz, DMSO-d₆) δ 7.88 (td, J = 7.8, 1.8 Hz, 1H), |
| | 7.71 (dddd, J = 9.2, 7.2, 5.2, 1.9 Hz, 1H), 7.42-7.31 (m, 2H), 4.10 (d, J = |
| | 3.1 Hz, 2H), 3.65 (s. 3H). |
| Step-2: Int-3 | 3-(2-fluorophenyl)-1H-pyrazol-5-ol (Int-3) |
| | LCMS: m/z 179.16 [M + 1]⁺. |
| Step-3: | (2-cyclopropoxy-4-fluorophenyl) (6-((3-(2-fluorophenyl)-1H- |
| (Compound 45). | pyrazol-5-yl) oxy)-2-azaspiro [3.3] heptan-2-yl)methanone |
| | (PSY-05-00555-001). |
| | LCMS: m/z 452.05 [M + 1]⁺. |
| | NMR: ¹H NMR (400 MHz, DMSO-d₆) δ 7.76 (s, 1H), 7.40 (s, 1H), |
| | 7.33 (d, J = 8.2 Hz, 3H), 7.22 (s, 1H), 6.85 (s, 1H), 6.03 (s, 1H), |
| | 4.78-4.67 (m, 1H), 4.04 (s, 1H), 3.96 (d, J = 12.6 Hz, 2H), 3.88 |
| | (s, 1H), 3.81 (s, 1H), 2.66 (d, J = 14.5 Hz, 2H), 2.31-2.21 (m, 2H), |
| | 0.86 (s, 2H), 0.71 (d, J = 12.2 Hz, 2H). |
| Step-4: | Fraction-1: (2-cyclopropoxy-4-fluorophenyl) (6-((5-(2- |
| (Compound 46) | fluorophenyl)-1-methyl-1H-pyrazol-3-yl) oxy)-2-azaspiro [3.3] |
| (Compound 47) | heptan-2-yl) methanone (PSY-05-00556-001) |
| | LCMS: m/z 466.40 [M + 1]⁺. |
| | NMR: ¹H NMR (400 MHz, DMSO-dₑ) δ 7.54 (d, J = 7.4 Hz, 1H), 7.46 |
| | (d. J = 7.3 Hz, 1H), 7.36 (dd, J = 19.2, 9.0 Hz, 3H), 7.21 (dd, J = 19.2, 9.0 |
| | Hz, 1H), 6.84 (m, 1H), 5.80 (s, 1H), 4.70 (dd, J = 15.3, 6.8 Hz, 1H), 4.03 |
| | (s, IH), 3.96 (s, 2H), 3.86 (s, 1H), 3.80 (s, 1H), 3.54 (s, 3H), 2.64 (d, J = |
| | 16.5 Hz, 2H), 2.24 (s, 2H), 0.85 (s, 2H), 0.71 (d, J = 11.4 Hz, 2H). |
| | Fraction-2: (2-cyclopropoxy-4-fluorophenyl)(6-((3-(2- |
| | fluorophenyl)-1-methyl-1H-pyrazol-5-yl)oxy)-2-azaspiro |
| | [3.3]heptan-2-yl)methanone (PSY-05-00557-001). |
| | LCMS: m/z 466.40 [M + 1]⁺. |
| | NMR: ¹H NMR (400 MHz, DMSO-d₆) δ 7.86 (t, J = 7.5 Hz, 1H), 7.33 (d, |
| | J = 7.7 Hz, 2H), 7.28-7.19 (m, 3H), 6.94 (dd, J = 14.8, 3.6 Hz, 1H), 5.91 |
| | (dd, J = 14.8, 3.6 Hz, 1H), 4.75-4.64 (m, 1H), 4.04 (s, 1H), 3.99 (s, 1H), |
| | 3.93 (s, 1H), 3.88 (s, 1H), 3.82 (s, 1H), 3.60 (d, J = 8.3 Hz, 3H), 2.80-2.64 |
| | (d, J = 16.5 Hz, 2H), 2.37-2.24 (m, 2H), 0.86 (t, J = 5.8 Hz, 2H), 0.71 (d, |
| | ,J = 12.2 Hz, 2H). |

### Example 11: Synthesis of (2-cyclopropoxy-4-fluorophenyl) (6-((3-(2-(trifluoromethyl)phenyl)-4,5-dihydro-1H-pyrazol-5-yl)oxy)-2-azaspiro[3.3]heptan-2-yl) methanone [Compound 48]; (2-cyclopropoxy-4-fluorophenyl) (6-((1-methyl-5-(2-(trifluoromethyl)phenyl)-1H-pyrazol-3-yloxy)-2-azaspiro[3.3]heptan-2-yl) methanone [Compound 49] and (2-cyclopropoxy-4-fluorophenyl) (6-((1-methyl-3-(2-(trifluoromethyl)phenyl)-1H-pyrazol-5-yl)oxy)-2-azaspiro[3.3]heptan-2-yl) methanone [Compound 50]

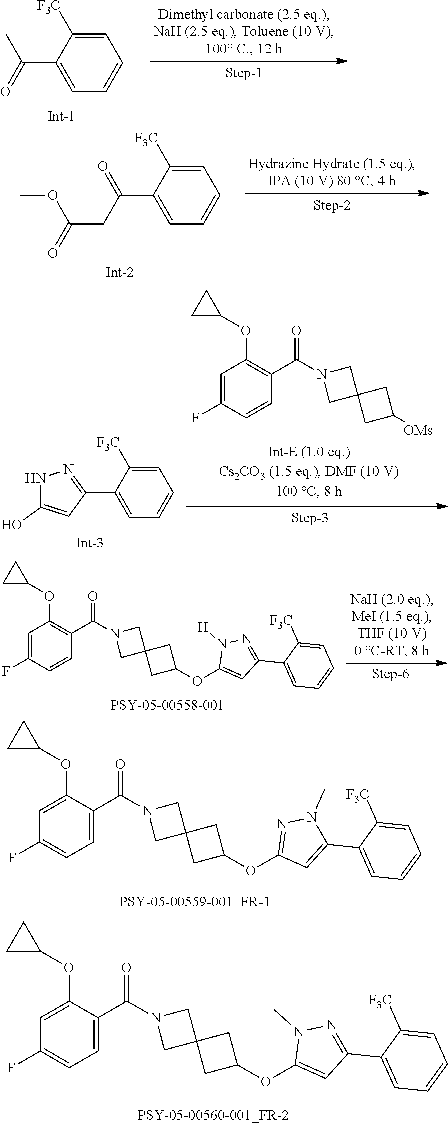

### Step-1 methyl 3-oxo-3-(2-(trifluoromethyl)phenyl)propanoate (Int-2)

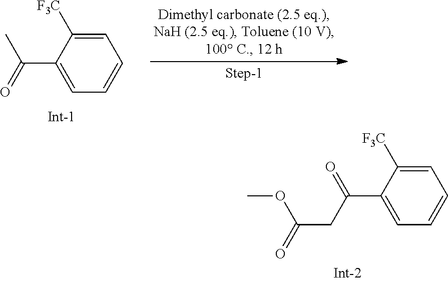 [SMILES: CC(=O)c1ccccc1C(F)(F)F ; COC(=O)CC(=O)c1ccccc1C(F)(F)F]

A mixture of the 1-(2-(trifluoromethyl)phenyl)ethan-1-one (Int-1) (5.0 g, 26.59 mmol, 1.0 eq.) in Toluene (50 mL, 10 V), were added Sodium hydride (60% suspension in mineral oil) (2.65 g, 66.48 mmol, 2.5 eq.) at 0° C. under N_{2(g)} atmosphere, followed by addition of dimethyl Carbonate (5.98 g, 66.48 mmol, 2.5 eq.). Then reaction mass allowed to stir at room temperature for 12 h. After completion of reaction as monitored by TLC, reaction mixture was acidified with 10% dil. hydrochloric acid and extracted with ethyl acetate (3*40 mL). The organic layer was washed with brine, dried over sodium sulphate, concentrated, and purified by flash chromatography using 3-4% ethyl acetate in n-hexane to get methyl 3-oxo-3-(2-(trifluoromethyl)phenyl)propanoate (Int-2) (5.45 g, 83.30%).

### Step-2: 3-(2-(trifluoromethyl)phenyl)-1H-pyrazol-5-ol (Int-3)

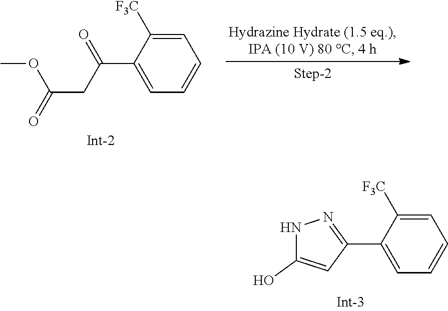 [SMILES: COC(=O)CC(=O)c1ccccc1C(F)(F)F ; Oc1cc(-c2ccccc2C(F)(F)F)n[nH]1]

A mixture of the methyl 3-oxo-3-(2-(trifluoromethyl)phenyl)propanoate (Int-2) (5.45 g, 22.15 mmol, 1.0 eq.) in Isopropyl alcohol (55 mL, 10 V) and hydrazine hydrate (1.66 g, 33.23 mmol, 1.5 eq.) was added to reaction mixture. The resulting mixture was heated at 80° C. for 4 h After completion of reaction as monitored by TLC, reaction mixture was concentrated under vacuum and purified by flash chromatography using 25-28% ethyl acetate in n-hexane to get 3-(2-(trifluoromethyl)phenyl)-1H-pyrazol-5-ol (Int-3) (4.3 g, 85.13%).

### Step-3: (2-cyclopropoxy-4-fluorophenyl)(6-((3-(2-(trifluoromethyl)phenyl)-4,5-dihydro-1H-pyrazol-5-yl)oxy)-2-azaspiro[3.3]heptan-2-yl)methanone (Compound 48)

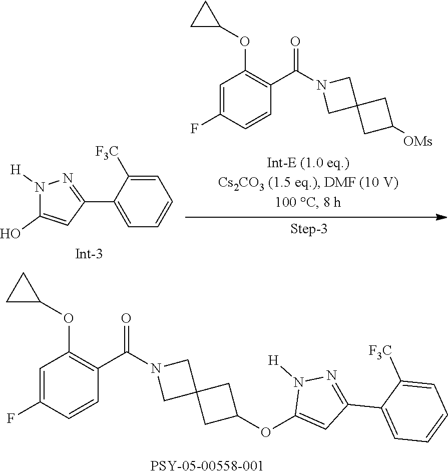

A mixture of 3-(2-(trifluoromethyl)phenyl)-1H-pyrazol-5-ol (Int-3) (4.3 g, 18.85 mmol, 1.0 eq.) in N,N-Dimethylformamide (45 mL, 10 V) were added Cesium carbonate (9.21 g, 28.28 mmol, 1.5 eq.) and (Int-E) (5.56 g, 15.08 mmol, 0.8 eq.) at room temperature. The resulting mixture was heated at 100° C. for 8 h. After completion of reaction as monitored by TLC, the reaction mixture was poured into water and extracted with ethyl acetate (3*40 mL). The organic layer was washed with brine (100 mL), dried over Sodium sulfate and concentrated under reduce pressure to obtain crude (2.2 g, crude). Crude (0.7 g) was purified by Prep-HPLC to get (2-cyclopropoxy-4-fluorophenyl)(6-((3-(2-(trifluoromethyl)phenyl)-4,5-dihydro-1H-pyrazol-5-yl)oxy)-2-azaspiro[3.3]heptan-2-yl)methanone (Compound 48) (0.11 g, 7.12%).

### Step-4: (2-cyclopropoxy-4-fluorophenyl)(6-((1-methyl-5-(2-(trifluoromethyl)phenyl)-1H-pyrazol-3-yl)oxy)-2-azaspiro[3.3]heptan-2-yl)methanone (Compound 49) and (2-cyclopropoxy-4-fluorophenyl)(6-((1-methyl-3-(2-(trifluoromethyl)phenyl)-1H-pyrazol-5-yl)oxy)-2-azaspiro[3.3]heptan-2-yl)methanone (Compound 50)

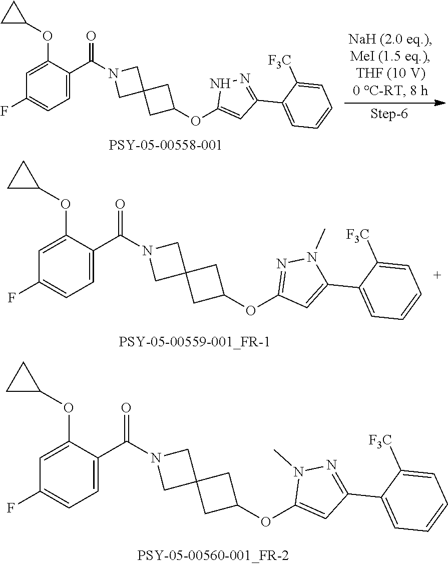 [SMILES: O=C(c1ccc(F)cc1OC1CC1)N1CC2(CC(Oc3cc(-c4ccccc4C(F)(F)F)n[nH]3)C2)C1 ; Cn1nc(OC2CC3(C2)CN(C(=O)c2ccc(F)cc2OC2CC2)C3)cc1-c1ccccc1C(F)(F)F ; Cn1nc(-c2ccccc2C(F)(F)F)cc1OC1CC2(C1)CN(C(=O)c1ccc(F)cc1OC1CC1)C2]

To a solution of the (2-cyclopropoxy-4-fluorophenyl)(6-((3-(2-(trifluoromethyl)phenyl)-4,5-dihydro-1H-pyrazol-5-yl)oxy)-2-azaspiro[3.3]heptan-2-yl)methanone (PSY-05-00558-001) (1.0 g, 1.99 mmol, 1.0 eq.) in Tetrahydrofuran (10 mL, 10V), were added sodium hydride (60% suspension in mineral oil) (0.15 g, 3.99 mmol, 2.0 eq.) at 0° C., followed by dropwise addition of methyl iodide (0.42 g, 2.99 mmol, 1.5 eq.). The reaction was stirred at room temperature for 8 h. After completion of reaction as monitored by TLC. The crude was diluted with water (100 mL) and extracted with ethyl acetate (50 mL*3). The organic layer was washed with brine (100 mL), dried over Sodium sulfate and concentrated under reduce pressure to obtain crude. Which was purified by prep-HPLC to obtained two fractions.

Fraction-1: (2-cyclopropoxy-4-fluorophenyl)(6-((1-methyl-5-(2-(trifluoromethyl)phenyl)-1H-pyrazol-3-yl)oxy)-2-azaspiro[3.3]heptan-2-yl)methanone (PSY-05-00559-001_FR-1) (0.06 g, 5.84%)

Fraction-2: (2-cyclopropoxy-4-fluorophenyl)(6-((1-methyl-3-(2-(trifluoromethyl) phenyl)-1H-pyrazol-5-yl)oxy)-2-azaspiro[3.3]heptan-2-yl)methanone (PSY-05-00560-001_FR-2) (0.05 g, 4.86%).

| PSY Compounds | |
|---|---|
| Compound 48 | |
| Compound 49 | |
| Compound 50 | MS/NMR Description |
| Step-1: Int-2 | Methyl 3-oxo-3-(2-(trifluoromethyl)phenyl) propanoate (Int-2). |
| | Mass: m/z 247.17 [M + 1]]⁺. |
| | NMR: ¹H NMR (400 MHz, Chloroform-d₁) δ 7.76 (d, J = 7.3 Hz, 1H), |
| | 7.70-7.50 (m, 3H), 3.94 (s, 2H), 3.76 (s, 3H). |
| Step-2: Int-3 | 3-(2-(trifluoromethyl)phenyl)-1H-pyrazol-5-ol (Int-3). |
| | LCMS: m/z 229.21 [M + 1]⁺. |
| | NMR: ¹H NMR (400 MHz, DMSO-d₆) δ 11.95 (s, 1H), 9.89 (s, 1H), 7.83 |
| | (d, J = 7.7 Hz, 1H), 7.73 (t, J = 7.4 Hz, 1H), 7.61 (d, J = 18.5 Hz, 2H), |
| | 5.64 (s, 1H). |
| Step-3: | (2-cyclopropoxy-4-fluorophenyl)(6-((3-(2-(trifluoromethyl) |
| (Compound 48) | phenyl)-4,5-dihydro-1H-pyrazol-5-yl)oxy)-2-azaspiro [3.3] |
| | heptan-2-yl)methanone (PSY-05-00558-001). |
| | LCMS: m/z 500.20 [M − 1]⁻. |
| | NMR: ¹H NMR (400 MHz, DMSO-d6) δ 12.24 (s, 1H), 7.86 (d, J = |
| | 7.5 Hz, 1H), 7.76 (s, 1H), 7.67-7.57 (d, J = 7.3 Hz, 2H), 7.27 (m, |
| | 1H), 7.21 (d, J = 11.3 Hz, 1H), 6.84 (m, 1H), 5.78 (s, 1H), 4.73- |
| | 4.67 (m, 1H), 4.03 (s, 1H), 3.95 (d, J = 11.0 Hz, 2H), 3.86 (s, 1H), |
| | 3.80 (s, 1H), 2.72 (d, J = 12.1 Hz, 2H), 2.31-2.17 (d, J = 12.1 Hz, |
| | 2H), 0.86 (s, 2H), 0.71 (d, J = 10.5 Hz, 2H). |
| Step-4: | Fraction-1: (2-cyclopropoxy-4-fluorophenyl)(6-((1-methyl-5-(2- |
| Compound 49 | (trifluoromethyl)phenyl)-1H-pyrazol-3-yl)oxy)-2- |
| Compound 50 | azaspiro[3.3]heptan-2-yl)methanone (PSY-05-00559-001) |
| | LCMS: m/z 516.21 [M + 1]⁺. |
| | NMR: ¹H NMR (400 MHz, DMSO-d6) δ 7.79 (d, J = 5.6 Hz, IH), 7.62 |
| | (td, J = 20.1, 17.4, 7.2 Hz, 3H), 7.34 (m, 1H), 7.21 (d, J = 10.9 Hz, 1H), |
| | 6.85 (d, J = 7.9 Hz, 1H), 5.75 (d, J = 14.9 Hz, 1H), 4.66 (dt, J = 14.5, 6.8 |
| | Hz, 1H), 4.01 (d, J = 14.9 Hz. 2H), 3.93 (s, 1H), 3.84 (d. J = 16.2 Hz, 2H), |
| | 3.59 (d, J = 8.3 Hz, 3H), 2.78-2.67 (m, 2H), 2.32-2.21 (m, 1H), 0.84 |
| | (s, 2H), 0.69 (s, 2H). |
| | Fraction-2: (2-cyclopropoxy-4-fluorophenyl)(6-((1-methyl-3-(2- |
| | (trifluoromethyl) phenyl)-1H-pyrazol-5-yl)oxy)-2-azaspiro[3.3] |
| | heptan-2-yl)methanone (PSY-05-00560-001). |
| | LCMS: m/z 516.18 [M + 1]⁺. |
| | NMR: ¹H NMR (400 MHz, DMSO-d6) δ 7.89 (d, J = 7.4 Hz, 1H), 7.75 |
| | (dd, J = 18.5, 7.3 Hz, 2H), 7.55 (d, J = 7.3 Hz, 1H), 7.32 (m, 1H), 7.20 (d, |
| | J = 10.9 Hz, 1H), 6.84 (s, 1H), 5.71 (s, 1H), 4.70 (dd, J = 15.7, 7.0 Hz, |
| | 1H), 4.02 (s, 1H), 3.96 (s, 2H), 3.86 (s, 1H), 3.79 (s, 1H), 3.54-3.33 (m, |
| | 3H), 2.69-2.59 (m, 2H), 2.31-2.25 (d, J = 10.3 Hz, 2H), 0.85 (s, 2H), |
| | 0.70 (d, J = 8.9 Hz, 2H). |

### Example 12: Synthesis of (2-fluoro-5-hydroxyphenyl) (6-(3-(2-(trifluoromethyl)phenyl) pyrrolidin-1-yl)-2-azaspiro[3.3]heptan-2-yl) methanone [Compound 51]; (S)-(2-fluoro-5-hydroxyphenyl) (6-(3-(2-(trifluoromethyl)phenyl) pyrrolidin-1-yl)-2-azaspiro[3.3]heptan-2-yl) methanone [Compound 52]; (R)-(2-fluoro-5-hydroxyphenyl) (6-(3-(2-(trifluoromethyl)phenyl) pyrrolidin-1-yl)-2-azaspiro[3.3]heptan-2-yl) methanone [Compound 53]

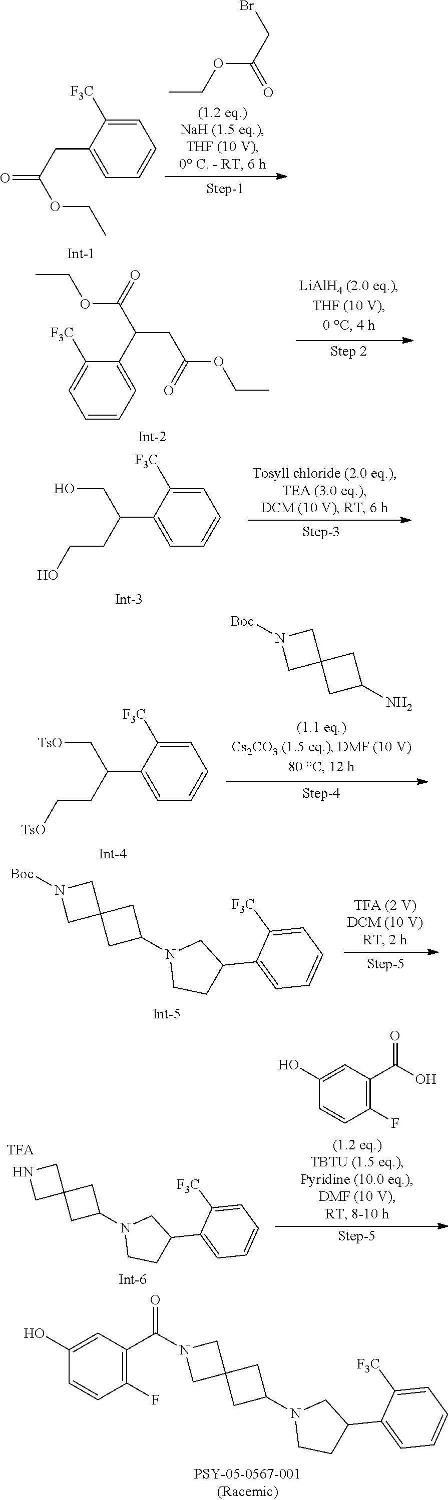

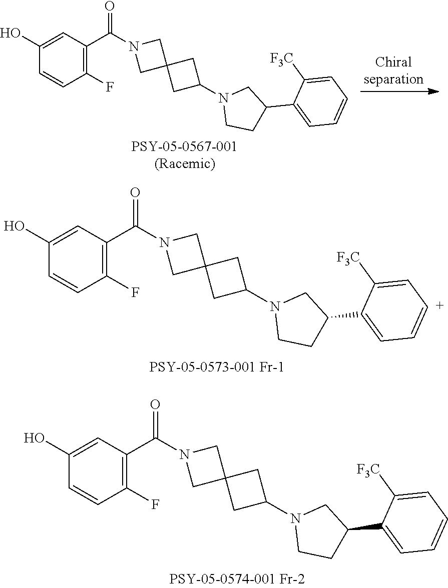 [SMILES: O=C(c1cc(O)ccc1F)N1CC2(CC(N3CCC(c4ccccc4C(F)(F)F)C3)C2)C1 ; O=C(c1cc(O)ccc1F)N1CC2(CC(N3CC[C@@H](C4=CC=[C+]C=C4C(F)(F)F)C3)C2)C1 ; O=C(c1cc(O)ccc1F)N1CC2(CC(N3CC[C@H](c4ccccc4C(F)(F)F)C3)C2)C1]

### Step-1: Diethyl 2-(2-(trifluoromethyl) phenyl) succinate (Int-2)

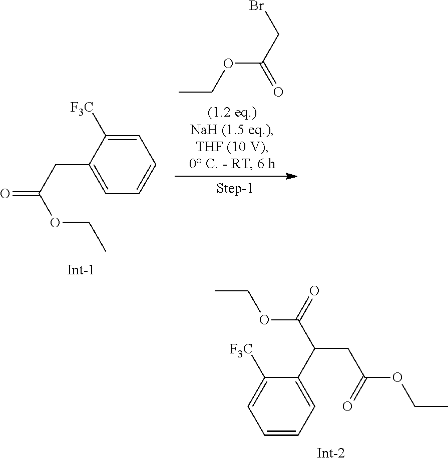

A solution of ethyl 2-(2-(trifluoromethyl) phenyl) acetate (Int-1) (10.0 g, 43.10 mmol, 1.0 eq.) in Tetrahydrofuran (100 mL, 10 V) was added Sodium Hydride (NaH 60% Suspension in mineral oil) (2.6 g, 64.65 mmol, 1.5 eq.) portion-wise at 0° C. and stirred for 10 mins. Ethyl 2-bromoacetate (8.6 g, 51.72 mmol, 1.2 eq.) was added drop-wise to the reaction mixture at 0° C. and allowed to stir at room temperature for 6 h. After completion of reaction as monitored by TLC, the reaction mixture was poured into water and acidified with 10% dil. hydrochloric acid and extracted by ethyl acetate (50 mL*3). The organic layer was washed with brine soln, dried over sodium sulphate and concentrated under vacuum to obtained crude product. Which was purified by flash chromatography using 3-4% ethyl acetate in n-hexane to get diethyl 2-(2-(trifluoromethyl) phenyl) succinate (Int-2) (11.5 g, 83.9%).

### Step-2: 2-(2-(trifluoromethyl) phenyl) butane-1,4-diol (Int-3)

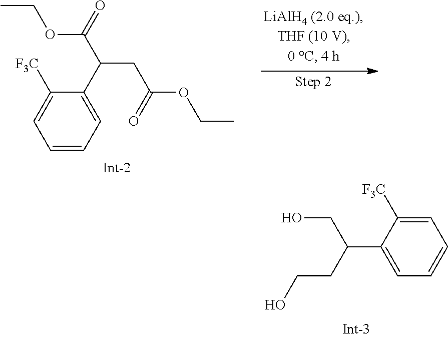 [SMILES: CCOC(=O)CC(C(=O)OCC)c1ccccc1C(F)(F)F ; OCCC(CO)c1ccccc1C(F)(F)F]

A solution of diethyl 2-(2-(trifluoromethyl) phenyl) succinate (Int-2) (11.5 g, 36.16 mmol, 1.0 eq.) in Tetrahydrofuran (115 mL, 10 V) was added lithium aluminum hydride 1M solution in THF (LiAlH₄) (72.32 mL, 72.32 mmol, 2.0 eq.) at 0° C. and allowed to stir for 4 h at 0° C. After completion of reaction as monitored by TLC, the reaction mixture was quenched in aqueous solution of NH₄Cl and extracted with ethyl acetate (50 mL*3). The organic layer was washed with brine solⁿ, dried over sodium sulphate and concentrated under vacuum to get crude product. Which was purified by flash chromatography using 50-60% ethyl acetate in n-hexane to get 2-(2-(trifluoromethyl) phenyl) butane-1,4-diol (Int-3) (6.9 g, 81.54%).

### Step-3: 2-(2-(trifluoromethyl) phenyl) butane-1,4-diyl bis(4-methylbenzenesulfonate) (Int-4)

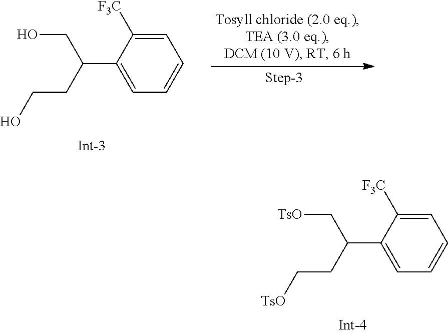 [SMILES: OCCC(CO)c1ccccc1C(F)(F)F ; Cc1ccc(S(=O)(=O)OCCC(COS(=O)(=O)c2ccc(C)cc2)c2ccccc2C(F)(F)F)cc1]

A solution of 2-(2-(trifluoromethyl) phenyl) butane-1,4-diol (1.0 g, 4.27 mmol, 1.0 eq.) in Dichloromethane (10 mL, 10 V) was added Triethyl amine (TEA) (1.3 g, 12.82 mmol, 3.0 eq.) at 0° C. Followed by portion wise addition of Tosyl Chloride (1.6 g, 8.54 mmol, 2.0 eq.) at 0° C. to reaction mixture and allowed to stir reaction at room temperature for 6 h. After completion of reaction as monitored by TLC, The reaction mixture was diluted with water and product was extracted by ethyl acetate (50 mL*3). The organic layer was washed with brine solⁿ, dried over sodium sulphate and concentrated under vacuum to get 2-(2-(trifluoromethyl) phenyl) butane-1,4-diyl bis(4-methylbenzenesulfonate) (Int-4) (1.2 g, 51.80%). Which was used in next step without further purification.

### Step-4: Tert-butyl 6-(3-(2-(trifluoromethyl) phenyl) pyrrolidin-1-yl)-2-azaspiro [3.3] heptane-2-carboxylate (Int-5)

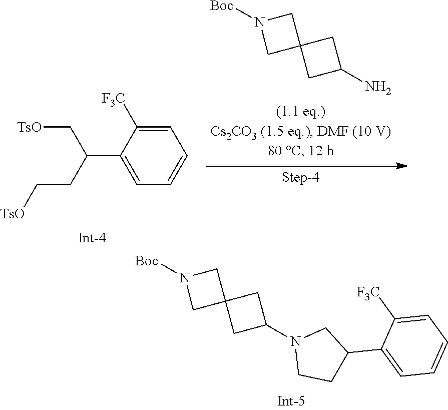 [SMILES: CC(C)(C)OC(=O)N1CC2(CC(N)C2)C1 ; Cc1ccc(S(=O)(=O)OCCC(COS(=O)(=O)c2ccc(C)cc2)c2ccccc2C(F)(F)F)cc1 ; CC(C)(C)OC(=O)N1CC2(CC(N3CCC(c4ccccc4C(F)(F)F)C3)C2)C1]

A solution of the 2-(2-(trifluoromethyl) phenyl) butane-1,4-diyl bis(4-methyl benzenesulfonate) (Int-4) (1.2 g, 2.21 mmol, 1.0 eq.) in N, N-Dimethylformamide (15 mL, 10 V), were added Cesium carbonate (1.08 g, 3.32 mmol, 1.5 eq.) and tert-butyl 6-amino-2-azaspiro [3.3]heptane-2-carboxylate (0.51 g, 2.43 mmol, 1.1 eq.). The reaction mixture was heated at 80° C. for 12 h. After completion of reaction as monitored by TLC, the reaction mixture was poured into water and extracted with ethyl acetate (50 mL*3). The organic layer was washed with brine solⁿ, dried over sodium sulphate and concentrated to get crude product. Which was purified by flash chromatography using 30-35% ethyl acetate in n-hexane to get tert-butyl 6-(3-(2-(trifluoromethyl) phenyl) pyrrolidin-1-yl)-2-azaspiro [3.3]heptane-2-carboxylate (Int-5) (0.61 g, 67.19%).

### Step-5: 6-(3-(2-(trifluoromethyl) phenyl) pyrrolidin-1-yl)-2-azaspiro [3.3] heptane (Int-6)

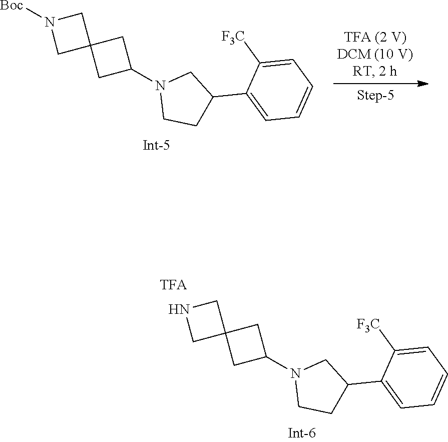 [SMILES: CC(C)(C)OC(=O)N1CC2(CC(N3CCC(c4ccccc4C(F)(F)F)C3)C2)C1 ; FC(F)(F)c1ccccc1C1CCN(C2CC3(CNC3)C2)C1]

To a solution of the tert-butyl 6-(3-(2-(trifluoromethyl) phenyl) pyrrolidin-1-yl)-2-azaspiro [3.3]heptane-2-carboxylate (Int-5) (0.61 g, 1.48 mmol, 1.0 eq.) in Dichloromethane (6 mL, 10 V), was added Trifluroacetic acid (1.2 mL, 2.0 V) drop-wise at room temperature and allowed to stir at room temperature for 2 h. After completion of reaction as monitored by TLC, the reaction mixture was concentrated under reduce pressure to obtain (Int-6) (0.4 g, 86.73%, as a TFA Salt). Which was used in next step without further purification.

### Step-6: (2-fluoro-5-hydroxyphenyl) (6-(3-(2-(trifluoromethyl) phenyl) pyrrolidin-1-yl)-2-azaspiro [3.3] heptan-2-yl) methanone (Compound 51)

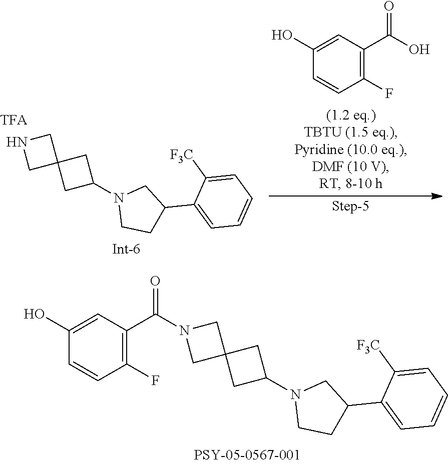 [SMILES: O=C(O)c1cc(O)ccc1F ; FC(F)(F)c1ccccc1C1CCN(C2CC3(CNC3)C2)C1 ; O=C(c1cc(O)ccc1F)N1CC2(CC(N3CCC(c4ccccc4C(F)(F)F)C3)C2)C1]

To a well stirred reaction mixture of 6-(3-(2-(trifluoromethyl) phenyl) pyrrolidin-1-yl)-2-azaspiro [3.3]heptane (Int-6)(0.600 g, 1.93 mmol, 1.0 eq.) in N,N-Dimethylformamide (6 mL, 10N), were added Pyridine (1.53 g, 19.35 mmol, 10.0 eq.), 2-fluoro-5-hydroxybenzoic acid (0.36 g, 2.32 mmol, 1.2 eq.) and followed by addition of 2-(1H-Benzotriazole-1-yl)-1,1,3,3-tetramethylaminium tetra fluoroborate (TBTU) (0.93 g, 2.90 mmol, 1.5 eq.) under N2 atmosphere at 0° C. The reaction mixture was stirred at room temperature for 8-10 h. After completion of reaction as monitored by TLC, the reaction mixture was poured into ice cold water and basified with saturated solution sodium bicarbonate. Product was extracted with ethyl acetate (3*50 mL). The organic layer was washed with brine, dried over sodium sulphate and concentrated under reduce pressure to get residue. The residue was submitted to Prep-HPLC purification (2-fluoro-5-hydroxyphenyl) (6-(3-(2-(trifluoromethyl) phenyl) pyrrolidin-1-yl)-2-azaspiro [3.3] heptan-2-yl) methanone (Compound 51) (0.11 g, 12.70%).

Compound 51 (racemic) was further purified by chiral-HPLC for enantiomer separation.

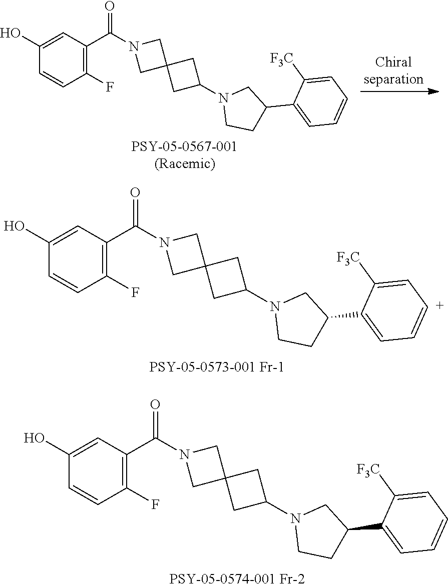 [SMILES: O=C(c1cc(O)ccc1F)N1CC2(CC(N3CCC(c4ccccc4C(F)(F)F)C3)C2)C1 ; O=C(c1cc(O)ccc1F)N1CC2(CC(N3CC[C@@H](C4=CC=[C+]C=C4C(F)(F)F)C3)C2)C1 ; O=C(c1cc(O)ccc1F)N1CC2(CC(N3CC[C@H](c4ccccc4C(F)(F)F)C3)C2)C1]

Fraction-1: Compound 52: (S)-(2-fluoro-5-hydroxyphenyl) (6-(3-(2-(trifluoromethyl) phenyl) pyrrolidin-1-yl)-2-azaspiro [3.3]heptan-2-yl) methanone (Compound 52) (0.026 g, 2.88%).

Fraction-2: Compound 53: (R)-(2-fluoro-5-hydroxyphenyl) (6-(3-(2-(trifluoromethyl) phenyl) pyrrolidin-1-yl)-2-azaspiro [3.3]heptan-2-yl) methanone (Compound 53) (0.025 g, 2.88%).

| PSY Compounds | |
|---|---|
| Compound 51 | |
| Compound 52 | |
| Compound 53 | MS/NMR Description |
| Step-1: Int-2 | Diethyl 2-(2-(trifluoromethyl) phenyl) succinate (Int-2). |
| | LCMS: m/z 304.27 [M + 1]⁺. |
| Step-2: Int-3 | 2-(2-(trifluoromethyl) phenyl) butane-1,4-diol (Int-3). |
| | ¹H NMR (400 MHz, DMSO-d₆) δ 7.65-7.58 (m, J = 7.6, 7.2, 8, 7.6 Hz, |
| | 3H), 7.40-7.37 (dd, J = 6.8, 6.8, 6 Hz, 1H), 4.80-4.78 (t, 1H), 4.39 (t, 1H), |
| | 3.53 (t, 2H), 3.22-3.13 (m, 2H), 2.45-2.28 (m, 1H), 1.94 (m, 1H), 1,66 |
| | (m, 1H). |
| Step-6: | (2-fluoro-5-hydroxyphenyl) (6-(3-(2-(trifluoromethyl) phenyl) |
| PSY-05-567-001 | pyrrolidin-1-yl)-2-azaspiro [3.3] heptan-2-yl) methanone |
| (Racemic) | (Racemic). |
| | LCMS: m/z 449.2 [M + 1]⁺. |
| | NMR: ¹H NMR (400 MHz, DMSO-d6) δ 7.76-7.59 (m, 3H), 7.48 (d, J = |
| | 5.7 Hz, 1H), 7.10 (td, J = 9.3, 3.5 Hz, 1H), 6.85 (d, J = 3.0 Hz, 1H), 6.76 |
| | (d, J = 3.0 Hz, 1H), 4.02-3.93 (dd, J = 28.6, 10.9 Hz, 4H), 3.54 (s, 1H), |
| | 2.855-2.76 (m, 2H), 2.67 (t, J = 8.6 Hz, 1H), 2.50 (t, J = 6.6 Hz, 2H), |
| | 2.45-2.41 (m, 3H), 2.24-2.13 (m, 2H), 1.74 (s, 1H). |
| Step-6: | (S)-(2-fluoro-5-hydroxyphenyl) (6-(3-(2-(trifluoromethyl) |
| PSY-05-00573-001 | phenyl) pyrrolidin-1-yl)-2-azaspiro [3.3] heptan-2-yl) |
| | methanone (PSY-05-00573-001) |
| | LCMS: m/z 449.1 [M + 1]⁺. |
| | NMR: ¹H NMR (400 MHz, DMSO-d6) δ 9.55 (s, 1H), 7.80-7.57 (m, |
| | 3H), 7.39 (d. J = 5.7 Hz, 1H), 7.08 (td, J = 9.3, 3.5 Hz, 1H), 6.87 (d, J = |
| | 3.0 Hz, 1H), 6.82 (d, J = 3.0 Hz, 1H), 4.05-3.98 (dd, J = 28.6, 10.9 Hz, |
| | 4H), 3.53 (s, 1H), 2.92-2.76 (m, 2H), 2.71 (t. J = 8.6 Hz, 1H), 2.54 (s, |
| | 2H), 2.35-2.23 (m, 3H), 2.14-2.05 (m, 2H), 1 74 (m, 1H). |
| Step-6: | (R)-(2-fluoro-5-hydroxyphenyl) (6-(3-(2-(trifluoromethyl) |
| PSY-05-00574-001 | phenyl) pyrrolidin-1-yl)-2-azaspiro [3.3] heptan-2-yl) |
| | methanone (PSY-05-00574-001). |
| | LCMS: m/z 449.1 [M + 1]⁺. |
| | ¹H NMR (400 MHz, DMSO-d6) δ 9.52 (s, 1H), 7.76 (d, J = 8.0 Hz, |
| | 1H), 7.74-7.59 (m, 2H), 7.39 (d, J = 5.9 Hz, 1H), 7.05 (m, 1H), |
| | 6.84 (s, 1H), 6.76 (s, 1H), 4.05-3.98 (dd, J = 28.7, 11.0 Hz, 4H), |
| | 3.55 (s, 1H), 2.90-2.78 (m, 2H), 2.77-2.69 (t, J = 8.5 Hz, 1H), |
| | 2.46 (s, 2H), 2.31-2.24 (m, 3H), 2.09 (d, J = 10.0 Hz, 2H), 1.71 (s, |
| | 1H). |

### Example 13: Synthesis of (2-cyclopropoxy-4-fluorophenyl) (6-(3-(2-(trifluoromethyl) phenyl) pyrrolidin-1-yl)-2-azaspiro [3.3]heptan-2-yl) methanone [Compound 54]; (S)-(2-cyclopropoxy-4-fluorophenyl) (6-(3-(2-(trifluoromethyl) phenyl) pyrrolidin-1-yl)-2-azaspiro [3.3]heptan-2-yl) methanone [Compound 55]; and (R)-(2-cyclopropoxy-4-fluorophenyl) (6-(3-(2-(trifluoromethyl) phenyl) pyrrolidin-1-yl)-2-azaspiro [3.3]heptan-2-yl) methanone [Compound 56]

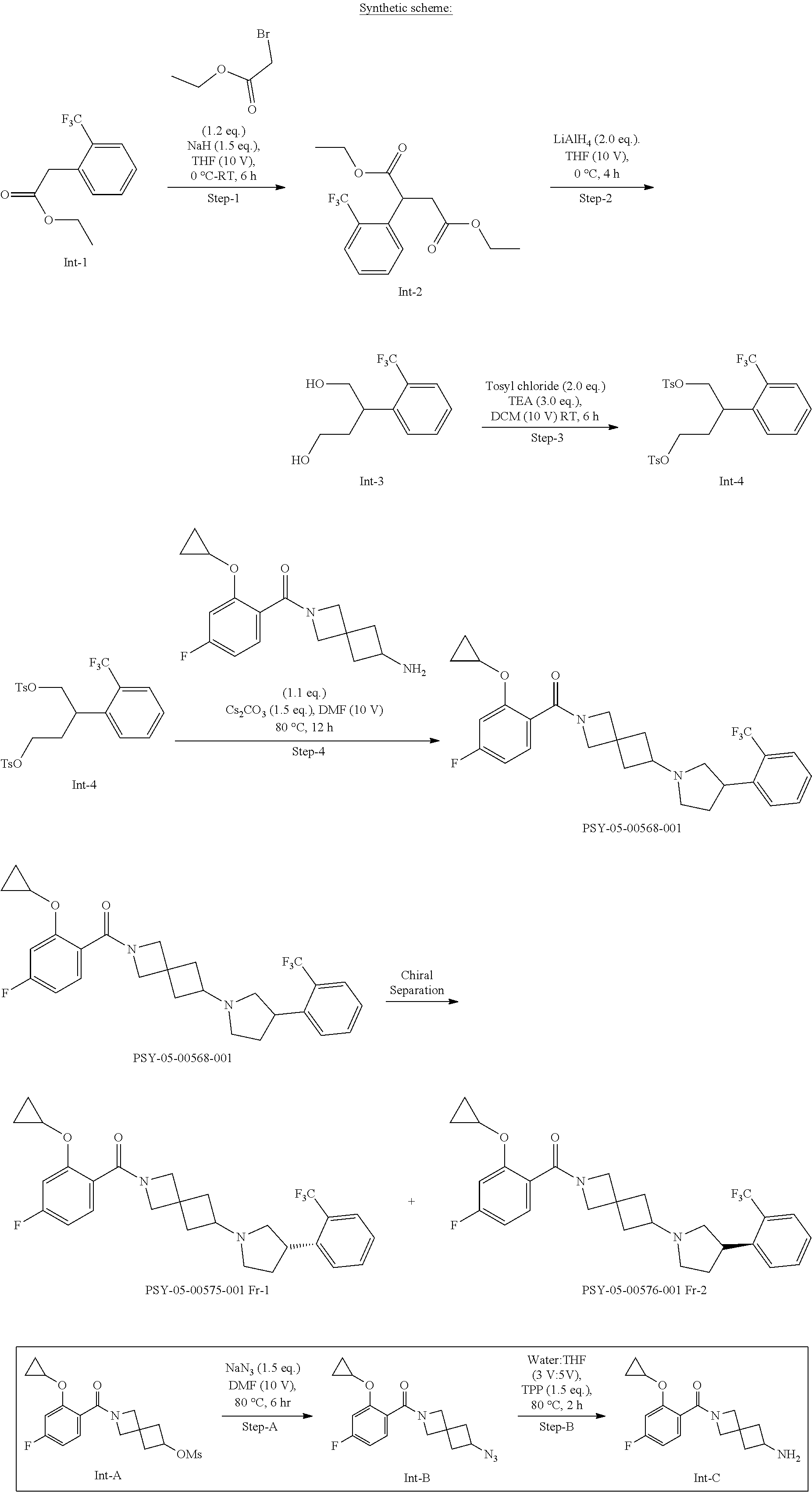

### Step-A: (6-azido-2-azaspiro [3.3] heptan-2-yl) (2-cyclopropoxy-4-fluorophenyl) methanone (Int-B)

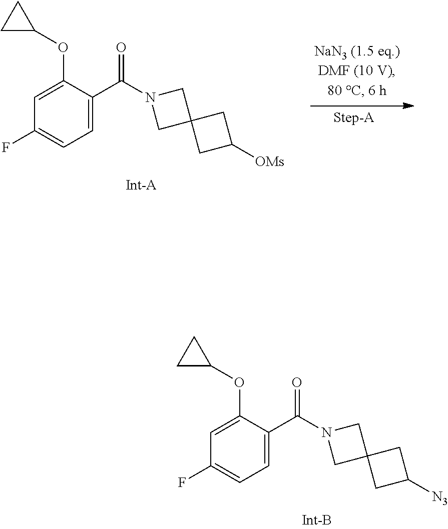 [SMILES: CS(=O)(=O)OC1CC2(C1)CN(C(=O)c1ccc(F)cc1OC1CC1)C2 ; [N-]=[N+]=NC1CC2(C1)CN(C(=O)c1ccc(F)cc1OC1CC1)C2]

A solution of the 2-(2-cyclopropoxy-4-fluorobenzoyl)-2-azaspiro [3.3]heptan-6-yl methane sulfonate (Int-1) (3.0 g, 8.13 mmol, 1.0 eq.) in N,N-Dimethylformamide (30 mL, 10 V), was added Sodium azide (0.8 g, 12.19 mmol, 1.5 eq.) portion-wise at room temperature. The reaction mixture was heated at 80° C. for 6 h. After completion of reaction as monitored by TLC, the reaction mixture was poured into water and product was extracted by ethyl acetate (50 mL*3). The organic layer was washed with brine solⁿ, dried over sodium sulphate and concentrated under reduce pressure to obtained crude product. Which was purified by flash chromatography using 30-40% ethyl acetate in n-hexane to get (6-azido-2-azaspiro [3.3]heptan-2-yl) (2-cyclopropoxy-4-fluorophenyl) methanone (Int-B) (1.4 g, 54.50%).

### Step-B: (6-amino-2-azaspiro [3.3]heptan-2-yl) (2-cyclopropoxy-4-fluorophenyl) methanone (Int-C)

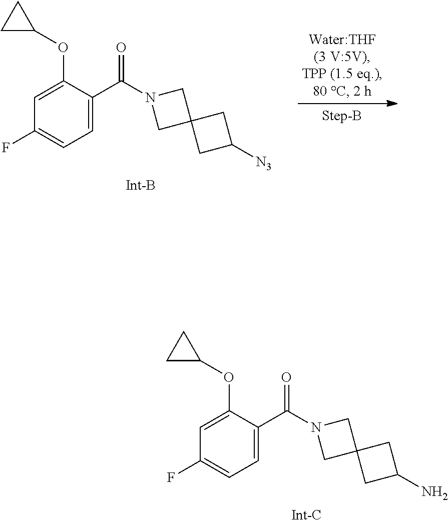 [SMILES: [N-]=[N+]=NC1CC2(C1)CN(C(=O)c1ccc(F)cc1OC1CC1)C2 ; NC1CC2(C1)CN(C(=O)c1ccc(F)cc1OC1CC1)C2]

A mixture of the (6-azido-2-azaspiro [3.3]heptan-2-yl) (2-cyclopropoxy-4-fluorophenyl) methanone (Int-1) (1.4 g, 4.42 mmol, 1.0 eq.) in Water: Tetrahydrofuran (4.2:7 mL, 3:5 V), was added Triphenylphosphine (1.7 g, 6.63 mmol, 1.5 eq.) portion-wise at room temperature. The reaction was heated at 80° C. for 2 h. After completion of reaction as monitored by TLC, The reaction mixture was poured into water and extracted by ethyl acetate (50 mL*3) The organic layer was washed with brine solⁿ, dried over sodium sulphate and concentrated under vacuum to obtained crude product. Which was purified by flash chromatography, the product was eluted in 3-4% Methanol in Dichloromethane to get (6-amino-2-azaspiro [3.3]heptan-2-yl) (2-cyclopropoxy-4-fluorophenyl) methanone (Int-C) (0.6 g, 46.69%).

### Step-1: diethyl 2-(2-(trifluoromethyl) phenyl) succinate (Int-2)

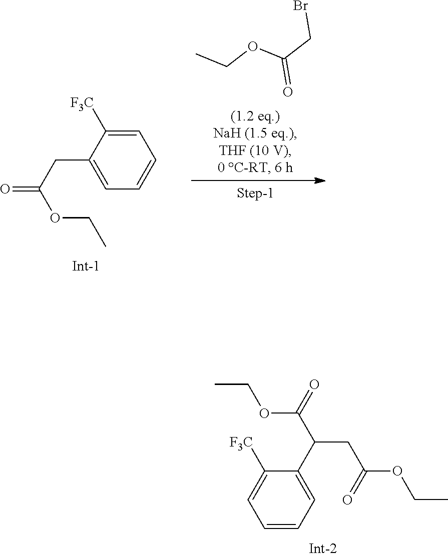

A solution of ethyl 2-(2-(trifluoromethyl) phenyl) acetate (Int-1) (10.0 g, 43.10 mmol, 1.0 eq.) in Tetrahydrofuran (100 mL, 10 V), was added Sodium Hydride (NaH 60% Suspension in mineral oil) (2.6 g, 64.65 mmol, 1.5 eq.) portion-wise at 0° C. Ethyl 2-bromoacetate (8.6 g, 51.72 mmol, 1.2 eq.) was added drop-wise to the reaction mixture at 0° C. Reaction was stirred at room temperature for 6 h. After completion of reaction as monitored by TLC. The reaction mixture was poured into water and acidified with 10% dil. hydrochloric acid and extracted by ethyl acetate (50 mL*3). The organic layer was washed with brine soln, dried over sodium sulphate and concentrated under vacuum to obtained crude product. Which was purified by flash chromatography using 3-4% ethyl acetate in n-hexane to get diethyl 2-(2-(trifluoromethyl) phenyl) succinate (Int-2) (11.5 g, 83.9%).

### Step-2: 2-(2-(trifluoromethyl) phenyl) butane-1,4-diol (Int-3)

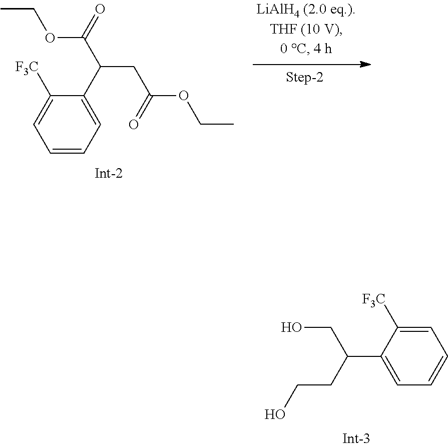 [SMILES: CCOC(=O)CC(C(=O)OCC)c1ccccc1C(F)(F)F ; OCCC(CO)c1ccccc1C(F)(F)F]

A solution of diethyl 2(2-(trifluoromethyl) phenyl) succinate (Int-2) (11.5 g, 36.15 mmol, 1.0 eq.) in Tetrahydrofuran (115 mL, 10 V), was added lithium aluminum hydride 1M solution in THF (LiAlH₄) (72.36 mL, 72.36 mmol, 2.0 eq.) at 0° C. and allowed to stir for 4 h at 0° C. After completion of reaction as monitored by TLC, the reaction mixture was quenched in aqueous solution of NH₄Cl and extracted with ethyl acetate (50 mL*3). The organic layer was washed with brine solⁿ, dried over sodium sulphate and concentrated under vacuum to obtained crude product. Which was purified by flash chromatography, product was eluted in 50-60% ethyl acetate in n-hexane to get 2-(2-(trifluoromethyl) phenyl) butane-1,4-diol (Int-3) (6.9 g, 81.54%).

### Step-3; 2-(2-(trifluoromethyl) phenyl) butane-1,4-diyl bis(4-methylbenzenesulfonate) (Int-4)

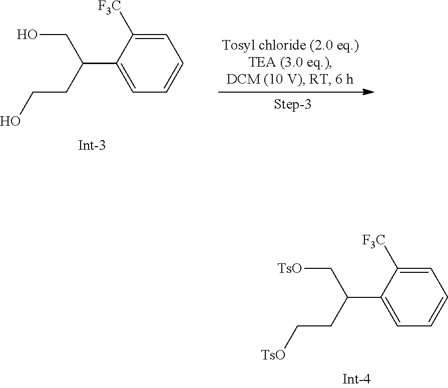 [SMILES: OCCC(CO)c1ccccc1C(F)(F)F ; Cc1ccc(S(=O)(=O)OCCC(COS(=O)(=O)c2ccc(C)cc2)c2ccccc2C(F)(F)F)cc1]

A solution of 2-(2-(trifluoromethyl) phenyl) butane-1,4-diol (1.0 g, 4.27 mmol, eq.) in Dichloromethane (10 mL, 10 V), was added Triethyl amine (TEA) (1.3 g, 12.82 mmol, 3.0 eq.). Followed by portion wise addition of Tosyl Chloride (1.6 g, 8.54 mmol, 2.0 eq.) at 0° C. Stir the reaction at room temperature for 6 h. After completion of reaction as monitored by TLC, The reaction mixture diluted with water and extracted by ethyl acetate (50 mL*3). The organic layer was washed with brine solⁿ, dried over sodium sulphate and concentrated under vacuum to get 2-(2-(trifluoromethyl) phenyl) butane-1,4-diyl bis(4-methylbenzenesulfonate) (Int-4) (1.2 g, 51. 80%). Which was used in next step without further purification.

### Step-4: (2-cyclopropoxy-4-fluorophenyl) (6-(3-(2-(trifluoromethyl) phenyl) pyrrolidin-1-yl)-2-azaspiro [3.3] heptan-2-yl) methanone (PSY-05-00568-001)

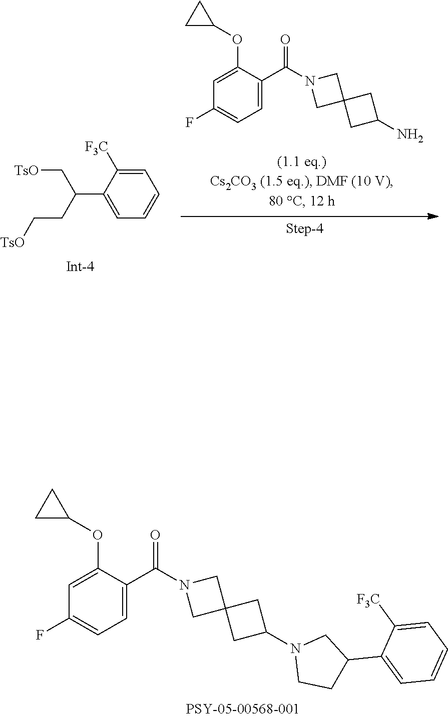 [SMILES: NC1CC2(C1)CN(C(=O)c1ccc(F)cc1OC1CC1)C2 ; Cc1ccc(S(=O)(=O)OCCC(COS(=O)(=O)c2ccc(C)cc2)c2ccccc2C(F)(F)F)cc1 ; O=C(c1ccc(F)cc1OC1CC1)N1CC2(CC(N3CCC(c4ccccc4C(F)(F)F)C3)C2)C1]

A solution of 2-(2-(trifluoromethyl) phenyl) butane-1,4-diyl bis(4-methylbenzenesulfonate) (Int-4) (1.0 g, 1.84 mmol, 1.0 eq.) in N,N-Dimethylformamide (10 mL, 10 V), were added Cesium carbonate (0.9 g, 2.76 mmol, 1.5 eq.), and (6-amino-2-azaspiro[3.3]heptan-2-yl)(2-cyclopropoxy-4-fluorophenyl)methanone (Int-C)(0.59 g, 2.02 mmol, 1.1 eq.) at room temperature. The reaction mixture was heated at 80° C. for 12 h After completion of reaction as monitored by TLC, The reaction mixture was poured into water and product was extracted with ethyl acetate (50 mL*3). The organic layer was washed with brine soln, dried over sodium sulphate and concentrated under reduce pressure to get residue. The residue was submitted to Prep-HPLC for purification (2-cyclopropoxy-4-fluorophenyl) (6-(3-(2-(trifluoromethyl) phenyl) pyrrolidin-1-yl)-2-azaspiro [3.3] heptan-2-yl) methanone (PSY-05-00568-001) (0.13 g, 24.02%.).

PSY-05-00569-001 was further purified by chiral-HPLC for enantiomer separation.

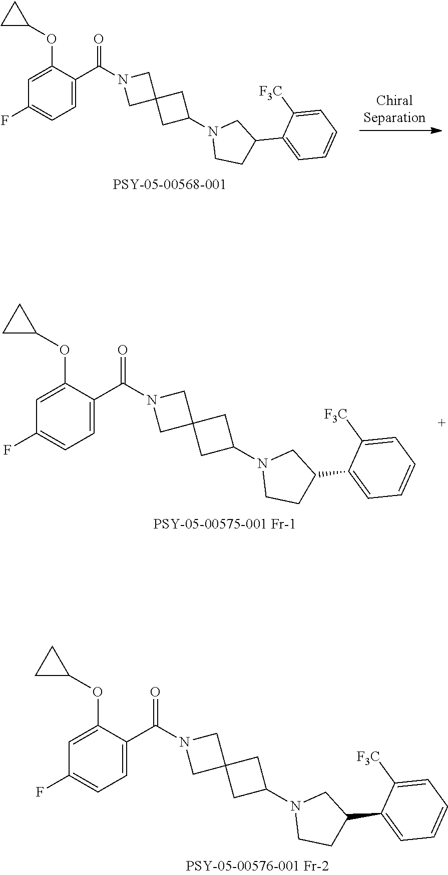

Fraction-1: (S)-(2-cyclopropoxy-4-fluorophenyl) (6-(3-(2-(trifluoromethyl) phenyl) pyrrolidin-1-yl)-2-azaspiro [3.3]heptan-2-yl) methanone (PSY-05-00575-071) (0.025 g, 4.62%).

Fraction-2: (R)-(2-cyclopropoxy-4-fluorophenyl) (63-(2 (2-(trifluoromethyl) phenyl) pyrrolidin-1-yl)-2-azaspiro [3.3]heptan-2-yl) methanone (PSY-05-00576-001) (0.022 g, 4.07%)

| | |
|---|---|
| Step-1: Int-2 | Diethyl 2-(2-(trifluoromethyl) phenyl) succinate (Int-2). |
| | LCMS: m/z 304.27 [M + 1]⁺. |
| Step-2: Int-3 | 2-(2-(trifluoromethyl) phenyl) butane-1,4-diol (Int-3). |
| | ¹H NMR (400 MHz, DMSO-d₆) δ 7.65-7.58 (m, J = 7.6, 7.2, 8, 7.6 Hz, |
| | 3H), 7.40-7.37 (dd, J = 6.8, 6.8, 6 Hz, 1H), 4.80-4.78 (t, 1H), 4.39 (t, 1H), |
| | 3.53 (t, 2H), 3.22-3.13 (m, 2H), 2.45-2.28 (m, 1H), 1.94 (m, 1H), 1.66 (m, |
| | 1H). |
| Step-6: | (2-cyclopropoxy-4-fluorophenyl) (6-(3-(2-(trifluoromethyl) phenyl) |
| PSY-05-00568-001 | pyrrolidin-1-yl)-2-azaspiro [3.3] heptan-2-yl) methanone (PSY-05- |
| (Racemic) | 00568-001). |
| | LCMS: m/z 489.3 [M + 1]⁺. |
| | NMR: ¹H NMR (400 MHz, DMSO-d₆) δ 7.76-7.60 (m, J = 8, 7.2, 7.2, 11.2, |
| | 6.8 Hz, 3H), 7.38 (dd, J =4 Hz, 1H), 7.32 (dd, J = 6.4, 6.4 Hz, 1H), 7.21 |
| | (dd, J = 8 Hz, 1H), 6.85-6.81 (dd, J = 8, 6.4 Hz, 1H), 3.97-3.90 (d, 3H), |
| | 3.81 (s, 1H), 3.73 (s, 1H), 3.65 (s, 1H), 2.86-2.75 (m, 3H), 2.66-2.45 (m, |
| | 2H), 2.25-2.20 (m, 3H), 2.13-2.01(m, 2H), 1.70 (s, 1H), 0.86 (d, 2H), 0.69 |
| | (d, 2H). |
| Step-6: | (S)-(2-cyclopropoxy-4-fluorophenyl) (6-(3-(2-(trifluoromethyl) phenyl) |
| PSY-05-00575-001 | pyrrolidin-1-yl)-2-azaspiro [3.3] heptan-2-yl) methanone (PSY-05- |
| | 00575-001) |
| | LCMS: m/z 489.14 [M + 1]⁺. |
| | NMR: ¹H NMR (400 MHz, DMSO-d₆) δ 7.78-7.68 (m, J = 8, 7.2, 7.2, 11.2, |
| | 6.8 Hz, 3H), 7.38-7.28 (dd, J = 4 Hz, 2H), 7.22 (dd, J = 6.4, 6.4 Hz, 1H), |
| | 6.84 (dd, J = 8 Hz, 1H), 3.98 (d, 3H), 3.83 (s, 1H), 3.75 (s, 1H), 3.64 (s, |
| | 1H), 2.85-2.77 (m, 3H), 2.69 (s, 2H), 2.45 (s, 3H), 2.27 (d, 2H), 1.72 (s, |
| | 1H), 0.86 (s, 2H), 0.69 (s, 2H). |
| Step-6: | (R)-(2-cyclopropoxy-4-fluorophenyl) (6-(3-(2-(trifluoromethyl) phenyl) |
| PSY-05-00576-001 | pyrrolidin-1-yl)-2-azaspiro [3.3] heptan-2-yl) methanone (PSY-05- |
| | 00576-001) |
| | LCMS: m/z 489.23 [M + 1]⁺. |
| | NMR: ¹H NMR (400 MHz, DMSO-d₆) δ 7.78-7.68 (m, J = 8, 7.2, 7.2, 11.2, |
| | 6.8 Hz, 3H), 7.38 (dd, J = 4 Hz, 1H), 7.32 (dd, J = 6.4, 6.4 Hz, 1H), 7.22 |
| | (dd, J = 6.4, 6.4 Hz, 1H), 6.84 (dd, J = 8 Hz, 1H), 4.05-3.90 (d, 3H), 3.85 |
| | (s, 1H), 3.76 (s, 1H), 3.64 (s, 1H), 2.82 (s, 1H), 2.75-2.67 (m, 4H), 2.35 |
| | (s, 3H), 2.27-2.07 (d, J = 6 Hz, 2H), 1.78 (s, 1H), 0.84 (s, 2H), 0.68 (s, |
| | 2H). |

### Example A: Measuring MAGL Inhibition Potency (IC₅₀)

The potency of certain compounds for inhibiting MAGL were obtained using the following assays.

The monoacylglycerol lipase inhibitor screening assay kit from Cayman Chemical was used to measure the MAGL potency for the compounds in Tables A-1, A-2, A-3.

Cayman's Monoacylglycerol Lipase Inhibitor Screening Assay provides a method for screening human MAGL inhibitors. MAGL hydrolyzes 4-nitrophenylacetate resulting in a yellow product, 4-nitrophenol, with an absorbance of 405-412 nm.

MAGL Inhibition was measured by the following assay. Monoacylglycerol Lipase (MAGL) inhibition was measured using recombinant MAGL enzyme (aa 2-303 RBC, internal preparation) and the substrate 4-Nitrophenyl acetate (4NPA) (Sigma-Aldrich, N8130). Hydrolysis of the substrate in the presence of the enzyme was measured by absorbance at 405 nm. 10 μL of assay buffer (10 mM Tris pH 7.5, 1 mM EDTA, 0.9% DMSO) was added to a black 384-well non-binding plate with clear bottom (Greiner, 781906) for each reaction. Compounds were dispensed using an acoustic liquid handler (Echo, Beckman) at 45 nL (0.1% DMSO). Test compounds and control for MAGL inhibitor JZL-184 (Caymen Chemical, 13158) were tested in 10-concentration IC50 mode with 3-fold serial dilution at a starting concentration of 10 PM. DMSO control wells were included for reference. A 10.8 nM (1.8×) MAGL mix in assay buffer was prepared, with 25 μL added to each reaction well, for a final assay concentration of 15 nM. No enzyme wells received 25 μL of buffer. Plate was incubated at room temperature for 30 minutes. 35 mM solution of 4NPA in methanol was prepared daily A 4.5×4NPA substrate solution was prepared in assay buffer and 10 μL was added to each reaction well, for a final assay concentration of 0.25 mM. Plate was spun for 1 minute at 1000 rpm before measuring absorbance using a CLARIOstar plate reader (BMG Labtech). A kinetic reading at 405 nm was done every minute for 30 minutes. Data was analyzed using the linear slope of the reaction progress curve and the average of the no-enzyme wells (background) was subtracted from the data. The background-subtracted slope data was converted to % activity using the average of wells with enzyme and DMSO vehicle. IC50s were calculated using GraphPad software (Sigmoidal dose response, variable slope equation).

Tables A-1, A-2 and A-3 provide exemplary compounds of Formula (I) and their potency for MAGL inhibition measured using the Potency Assay of Example A above, with the following modifications described in Table C below. Table 8 provides potency measurements of MAGL inhibition measured using the Potency Assay of Example A above, with the following modifications described in Table C.

TABLE C
Methods Used to Measure MAGL Inhibition with 4NPA Substrate

| Method | | |
|---|---|---|
| No. | Name | Modification to Assay of Example A |
| 1 | MAGL (6 nM) Reaction | Reaction Biology's intemal MAGL enzyme prep was used. |
| | Biology Enzyme | Concentration of MAGL enzyme in the assay was 6 nM. |
| 2 | MAGL (10 nM) Cayman | Cayman Chemical's MAGL enzyme prep was used. |
| | Chemical Enzyme | Concentration of MAGL enzyme in the assay was 10 nM. |
| 3 | MAGL (10 nM) Reaction | Reaction Biology's intemal MAGL enzyme prep was used. |
| | Biology Enzyme | Concentration of MAGL enzyme in the assay was 10 nM. |
| 4 | MAGL (15 nM) Cayman | Cayman Chemical's MAGL enzyme prep was used. |
| | Chemical Enzyme | Concentration of MAGL enzyme in the assay was 15 nM. |
| 5 | MAGL (25 nM) Cayman | Cayman Chemical's MAGL enzyme prep was used. |
| | Chemical Enzyme | Concentration of MAGL enzyme in the assay was 25 nM. |
| 6 | MAGL IC50 Enzyme | Reaction Biology's internal MAGL enzyme prep was used. |
| | Conc 6, Reaction Biology | Concentration of MAGL enzyme in the assay was 6 nM. |
| 7 | MAGL IC50 Enzyme | Reaction Biology's intemal MAGL enzyme prep was used. |
| | Conc 10, Reaction | Concentration of MAGL enzyme in the assay was 10 nM. |
| | Biology | |
| 8 | MAGL IC50 Enzyme | Cayman Chemical's MAGL enzyme prep was used. |
| | Conc 10, Cayman | Concentration of MAGL enzyme in the assay was 10 nM. |
| | Chemical | |
| 9 | MAGL IC50 Enzyme | Cayman Chemical's MAGL enzyme prep was used. |
| | Conc 15, Cayman | Concentration of MAGL enzyme in the assay was 15 nM. |
| | Chemical | |
| 10 | MAGL IC50 Enzyme | Cayman Chemical's MAGL enzyme prep was used. |
| | Conc 25, Cayman | Concentration of MAGL enzyme in the assay was 25 nM. |
| | Chemical | |
| 11 | MAGL IC50 Enzyme | Reaction Biology's intemal MAGL enzyme prep was used. |
| | Conc 20, Reaction | Concentration of MAGL enzyme in the assay was 25 nM. |
| | Biology | |
| 12 | MAGL IC50 Enzyme | Reaction Biology's intemal MAGL enzyme prep was used. |
| | Conc 15, Reaction | Concentration of MAGL enzyme in the assay was 15 nM. |
| | Biology | |
| 13 | MAGL IC50 Enzyme | Reaction Biology's intemal MAGL enzyme prep was used. |
| | Conc 12. Reaction | Concentration of MAGL enzyme in the assay was 12 nM. |
| | Biology | |
| 14 | MAGL IC50 Enzyme | Reaction Biology's internal MAGL enzyme prep was used. |
| | Conc “0”, Source “0” | Concentration of MAGL enzyme in the assay was 6 nM. |

Results of potency measurements in Tables A-1, A-2 and A-3 are expressed as the following ranges: “A” refers to an IC₅₀ measurement of <50 nM, “B” refers to an IC₅₀ measurement of between 50 nM and 150 nM, “C” refers to an IC₅₀, measurement of greater than 150 nM and less than 500 nM, “D” refers to an IC₅₀ measurement of 500 nM to 1 micromolar, and “E” refers to an IC₅₀ measurement of greater than 1 micromolar up to 5.1 micromolar. The number after each letter for potency measurement value indicates the Method from Table C that was used to obtain that measurement value (e.g. “A(1)” indicates an IC₅₀ measurement of <50 nM obtained from the Method 1 of Example A in Table C).

TABLE A-1

| | | | MAGL |
|---|---|---|---|
| | | | Potency |
| | Structure | | (Detection |
| Compound | (IUPAC name) | LCMS/NMR | Method) |
| 33 | 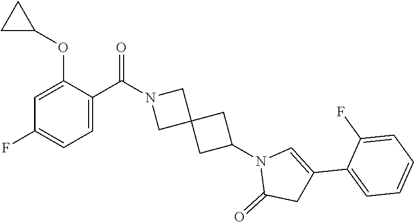 [SMILES: O=C(c1ccc(F)cc1OC1CC1)N1CC2(CC(N3C=C(c4ccccc4F)CC3=O)C2)C1] | LCMS: m/z 451.1 [M + 1]⁺ NMR: ¹H NMR (400 MHz, DMSO-d₆) δ 7.74 (m, 1H), 7.61 (m, 1H), 7.41-7.32 (m, 3H), 7.21 (d, J = 11.1 Hz, 1H), 6.85 (s, 1H), 6.47 (s, 1H), 4.59 (s, 1H), 4.57- 4.44 (m, 2H), 4.08 (s, 1H), 3.94 (d, J = 18.7 Hz, 3H), 3.79 (s, 1H), 0.86 (s, 2H), 0.71 (d, J = 10.0 Hz, 2H). | E (12) |
| | 1-{2-(2-cyclopropoxy-4- | | |
| | fluorobenzoyl)-2-aza-6- | | |
| | spiro[3.3]heptyl}-3-(o- | | |
| | fluorophenyl)-2-pyrrolin-5-one | | |
| 42 | 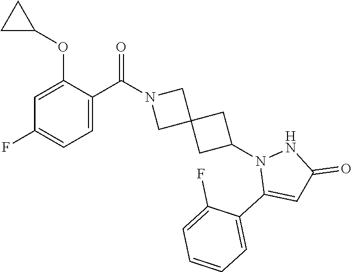 [SMILES: O=C(c1ccc(F)cc1OC1CC1)N1CC2(CC(n3[nH]c(=O)cc3-c3ccccc3F)C2)C1] | LCMS: m/z 452.14 [M + 1]⁺ ¹H NMR (400 MHz, DMSO-d6) δ 8.06 (t, J = 6.0 Hz, 1H), 7.68- 7.51 (m, 3H), 7.47-7.33 (m, 2H), 7.23 (dd, J = 11.3, 2.2 Hz, 1H), 6.86 (td, J = 8.4, 2.3 Hz, 1H), 5.36 (s, 1H), 4.73 (t, J = 4.8 Hz, 1H), 3.95 (d. J = 11.3 Hz, 3H), 3.37 (s, 2H), 2.41 (s, 2H), 1.72 (dd, J = 7.4, 2.3 Hz, 2H), 0.84-0.76 (m, 2H), 0.64 (s, 2H). | |
| | 1-{2-(2-cyclopropoxy-4- | | |
| | fluorobenzoyl)-2-aza-6- | | |
| | spiro[3.3]heptyl}-5-(o- | | |
| | fluorophenyl)-1,2-dihydro-3H- | | |
| | pyrazol-3-one | | |
| 43 | 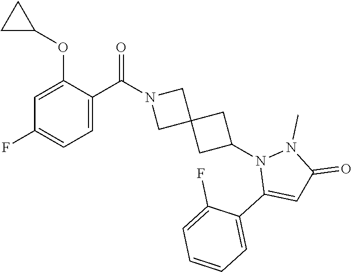 [SMILES: Cn1c(=O)cc(-c2ccccc2F)n1C1CC2(C1)CN(C(=O)c1ccc(F)cc1OC1CC1)C2] | LCMS: m/z 466.39 [M + 1]⁺ ¹H NMR (400 MHz, DMSO-d₆) δ 7.69-7.53 (m, 2H), 7.45-7.32 (m, 2H), 7.21-7.12 (m, 2H), 6.89 (dt, J = 16.9, 8.2 Hz, 1H), 5.39 (s, 1H), 4.76 (t, J = 4.6 Hz, 1H), 4.00-3.94 (m, 3H), 3.90-3.84 (m, 1H), 3.21 (s, 1H), 2.69 (s, 3H), 2.33 (m, 2H), 1.78 (s, 1H), 1.70 (m, 1H), 0.83- 0.77 (m, 2H), 0.50 (s, 2H). | |
| | 1-{2-(2-cyclopropoxy-4- | | |
| | fluorobenzoyl)-2-aza-6- | | |
| | spiro[3.3]heptyl}-5-(o- | | |
| | fluorophenyl)-2-methyl-1,2- | | |
| | dihydro-3H-pyrazol-3-one | | |
| 44 | 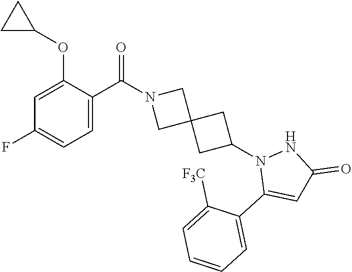 [SMILES: O=C(c1ccc(F)cc1OC1CC1)N1CC2(CC(n3[nH]c(=O)cc3-c3ccccc3C(F)(F)F)C2)C1] | LCMS: m/z 502.40 [M + 1]⁺ ¹H NMR (400 MHz, DMSO-d₆) δ 8.04-7.90 (m, 2H), 7.84 (t, J = 7.4 Hz, 1H), 7.81-7.68 (m, 2H), 7.58- 7.49 (m, 1H), 7.23 (dd, J = 11.3, 2.3 Hz, 1H), 6.85 (td, J = 8.4, 2.3 Hz, 1H), 5.21 (s, 1H), 4.71 (t, J = 4.8 Hz, 1H), 3.97-3.90 (m, 1H), 3.72 (s, 2H), 3.35 (s, 2H), 2.46-2.39 (m, 2H), 1.69 (s, 2H), 0.80 (d, J = 7.2 Hz, 2H), 0.63 (s, 2H). | |
| | 1-{2-(2-cyclopropoxy-4- | | |
| | fluorobenzoyl)-2-aza-6- | | |
| | spiro[3.3]heptyl}-5-[o- | | |
| | (trifluoromethyl)phenyl]-1,2- | | |
| | dihydro-3H-pyrazol-3-one | | |

TABLE A-2
Exemplary Compounds.

| | | | MAGL |
|---|---|---|---|
| | | | Potency |
| | Structure | | (Detection |
| Compound | (IUPAC name) | LCMS/NMR | Method) |
| 9 | 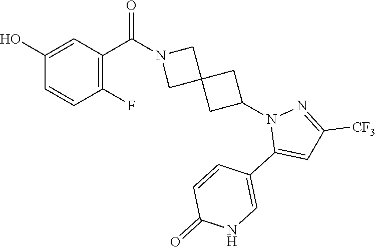 [SMILES: O=C(c1cc(O)ccc1F)N1CC2(CC(n3nc(C(F)(F)F)cc3-c3ccc(=O)[nH]c3)C2)C1] | LCMS: m/z 463.5 [M + H]⁺. ¹H NMR (400 MHz, DMSO- d₆) δ 11.87 (s, 1H), 9.66 (s, 1H), 7.93 (dd, J = 24.2, 10.6 Hz, 2H), 7.30 (s, 1H), 7.09 (t, J = 9.2 Hz, 1H), 6.89-6.76 (m, 2H), 6.42 (dd, J = 19.7, 9.8 Hz, 1H), 4.89-4.79 (m, 1H), 4.11 (dd, J = 23.3, 8.8 Hz, 4H), 2.81-2.71 (m, 4H). | C (12) |
| | 5-(1-{2-(2-fluoro-5- | | |
| | hydroxybenzoyl)-2-aza-6- | | |
| | spiro[3.3]heptyl}-3- | | |
| | (trifluoromethyl)-5-pyrazolyl)- | | |
| | 2(1H)-pyridinone | | |
| 10 | 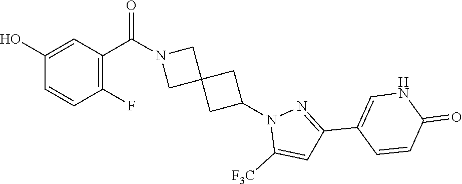 [SMILES: O=C(c1cc(O)ccc1F)N1CC2(CC(n3nc(-c4ccc(=O)[nH]c4)cc3C(F)(F)F)C2)C1] | LCMS: m/z 463.5 [M + H]⁺. ¹H NMR (400 MHz, DMSO- d₆) δ 10.52-9.60 (s, 2H), 7.61-7.44 (m, 2H), 7.09 (t, J = 9.3 Hz, 1H), 6.90-6.76 (m, 3H), 6.45 (dd, J = 15.1, 9.5 Hz, 1H), 4.84 (dt, J = 22.7, 8.0 Hz, 1H), 4.08 (dd, J = 20.4, 9.7 Hz, 4H), 2.69 (p, J = 10.8 Hz, 4H). | A (12) |
| | 5-(1-{2-(2-fluoro-5- | | |
| | hydroxybenzoyl)-2-aza-6- | | |
| | spiro[3.3]heptyl}-5- | | |
| | (trifluoromethyl)-3-pyrazolyl)- | | |
| | 2(1H)-pyridinone | | |
| 11 | 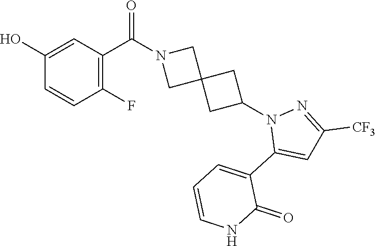 [SMILES: O=C(c1cc(O)ccc1F)N1CC2(CC(n3nc(C(F)(F)F)cc3-c3ccc[nH]c3=O)C2)C1] | LCMS: m/z 463.41 [M + H]⁺ ¹H NMR (400 MHz, DMSO- d₆) δ 11.92 (s, 1H), 9.64 (s, 1H), 8.20-8.11 (d, J = 6.9 Hz, 1H), 7.48 (d, J = 13.2 Hz, 2H), 7.09 (t, J = 9.4 Hz, 1H), 6.80 (s, 2H), 6.40 (m, 1H), 4.94-4.85 (m, 1H), 4.13 (dd, J = 23.9, 7.7 Hz, 4H), 2.84- 2.74 (m, 4H). | C (12) |
| | 3-(1-{2-(2-fluoro-5- | | |
| | hydroxybenzoyl)-2-aza-6- | | |
| | spiro[3.3]heptyl}-3- | | |
| | (trifluoromethyl)-5-pyrazolyl)- | | |
| | 2(1H)-pyridinone | | |
| 12 | 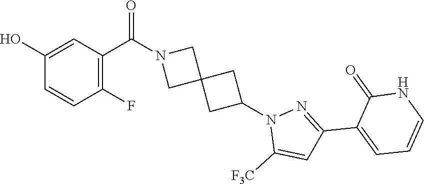 [SMILES: O=C(c1cc(O)ccc1F)N1CC2(CC(n3nc(-c4ccc[nH]c4=O)cc3C(F)(F)F)C2)C1] | LCMS: m/z 463.30 [M + H]⁺. ¹H NMR (400 MHz, DMSO- d₆) δ 11.83 (s, 1H), 9.65 (s, 1H), 7.59 (dt, J = 12.6, 6.4 Hz, 2H), 7.09 (t, J = 8.1 Hz, 1H), 6.79 (d, J = 2.4 Hz, 1H), 6.74 (s, 1H), 6.71 (s, 1H), 6.34 (dt, J = 13.3, 6.6 Hz, 1H), 4.75-4.61 (m, 1H), 4.06 (t, J = 9.9 Hz, 4H), 2.67 (s, 4H). | A (12) |
| | 3-(1-(2-(2-fluoro-5- | | |
| | hydroxybenzoyl)-2-aza-6- | | |
| | spiro[3.3]heptyl}-5- | | |
| | (trifluoromethyl)-3-pyrazolyl)- | | |
| | 2(1H)-pyridinone | | |
| 29 | 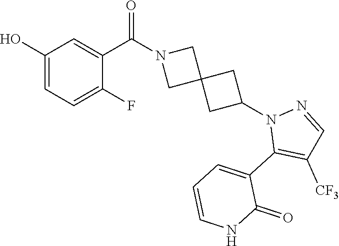 [SMILES: O=C(c1cc(O)ccc1F)N1CC2(CC(n3ncc(C(F)(F)F)c3-c3ccc[nH]c3=O)C2)C1] | LCMS: m/z 463.1 [M + 1]⁺ ¹H NMR (400 MHz, DMSO- d₆) δ 11.77 (s, 1H), 9.68 (s, 1H), 8.45 (d, J = 7.9 Hz, 1H), 7.44 (d, J = 6.7 Hz, 2H), 7.08 (d, J = 5.6 Hz, 1H), 6.71-6.91 (m, 2H), 6.22 (d, J = 6.9 Hz, 1H), 4.81 (dt, J = 15.9, 8.0 Hz, 1H), 4.14 (d, J = 10.8 Hz, 2H), 4.03 (d, J = 9.0 Hz, 2H), 2.76-2.65 (m, 4H). | E (12) |
| | 3-(1-{2-(2-fluoro-5- | | |
| | hydroxybenzoyl)-2-aza-6- | | |
| | spiro[3.3]heptyl}-4- | | |
| | (trifluoromethyl)-5-pyrazolyl)- | | |
| | 2(1H)-pyridinone | | |
| 30 | 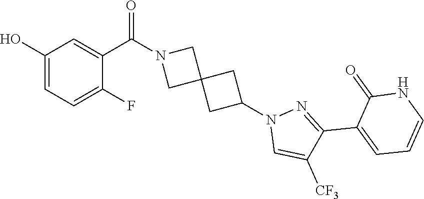 [SMILES: O=C(c1cc(O)ccc1F)N1CC2(CC(n3cc(C(F)(F)F)c(-c4ccc[nH]c4=O)n3)C2)C1] | LCMS: m/z 463.1 [M + 1]⁺ ¹H NMR (400 MHz, DMSO- d₆) δ 12.02 (s, 1H), 9.72 (s, 1H), 7.96 (d, J = 9.5 Hz, 1H), 7.60 (dd, J = 12.1, 5.5 Hz, 1H), 7.52 (d, J = 6.6 Hz, 1H), 7.06 (q, J = 9.2 Hz, 1H), 6.91-6.74 (m, 2H), 6.37- 6.28 (m, 1H), 4.54-4.46 (m, 1H), 4.04 (d, J = 9.3 Hz, 4H), 2.74-2.57 (m, 4H). | B (12) |
| | 3-(1-{2-(2-fluoro-5- | | |
| | hydroxybenzoyl)-2-aza-6- | | |
| | spiro[3.3]heptyl}-4- | | |
| | (trifluoromethyl)-3-pyrazolyl)- | | |
| | 2(1H)-pyridinone | | |
| 31 | 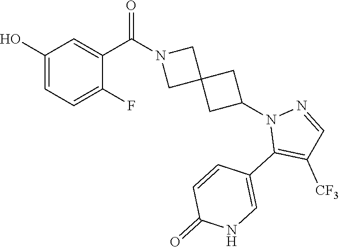 [SMILES: O=C(c1cc(O)ccc1F)N1CC2(CC(n3ncc(C(F)(F)F)c3-c3ccc(=O)[nH]c3)C2)C1] | LCMS: m/z 463.1 [M + 1]⁺ ¹H NMR (400 MHz, DMSO- d₆) δ 11.83 (s, 1H), 9.68 (s, 1H), 8.51 (d, J = 9.0 Hz, 1H), 7.73-7.62 (m, 1H), 7.53 (d, J = 11.1 Hz, 1H), 7.15-7.05 (m, 1H), 6.83 (dt, J = 24.4, 4.8 Hz, 2H), 6.45 (t, J = 10.2 Hz, 1H), 4.83 (dp, J = 23.9, 7.9 Hz, 1H), 4.14 (d, J = 11.1 Hz, 2H), 4.06 (d, J = 9.0 Hz, 2H), 2.73 (q, J = 12.1, 9.8 Hz, 4H). | E (12) |
| | 5-(1-{2-(2-fluoro-5- | | |
| | hydroxybenzoyl)-2-aza-6- | | |
| | spiro[3.3]heptyl}-4- | | |
| | (trifluoromethyl)-5-pyrazolyl)- | | |
| | 2(1H)-pyridinone | | |
| 32 | 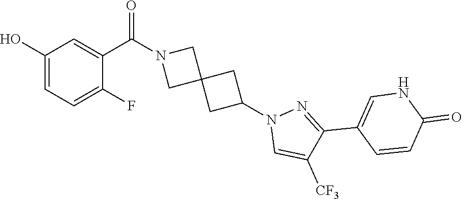 [SMILES: O=C(c1cc(O)ccc1F)N1CC2(CC(n3cc(C(F)(F)F)c(-c4ccc(=O)[nH]c4)n3)C2)C1] | LCMS: m/z 463.1 [M + 1]⁺ ¹H NMR (400 MHz, DMSO- d₆) δ 12.04 (s, 1H), 9.75 (s, 1H), 8.01 (d, J = 9.0 Hz, 1H), 7.53-7.50 (m, 1H), 7.53 (d, J = 11.1 Hz, 1H), 7.40 (d, J = 11.1 Hz, 1H), 7.09-7.00 (m, 1H), 6.83-6.70 (dt, J = 24.4, 4.8 Hz, 2H), 6.45 (t, J = 10.2 Hz, 1H), 4.83 (dp, J = 23.9, 7.9 Hz, 1H), 4.14-4.06 (m, 4H), 2.73 (q, J = 12.1, 9.8 Hz, 4H). | B (12) |
| | 5-(1-{2-(2-fluoro-5- | | |
| | hydroxybenzoyl)-2-aza-6- | | |
| | spiro[3.3]hepty]}-4- | | |
| | (trifluoromethyl)-3-pyrazolyl)- | | |
| | 2(1H)-pyridinone | | |
| 41 | 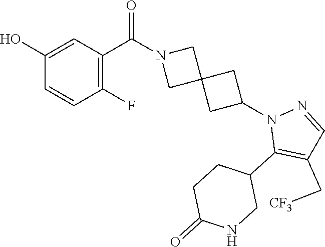 [SMILES: O=C1CCC(c2c(CC(F)(F)F)cnn2C2CC3(C2)CN(C(=O)c2cc(O)ccc2F)C3)CN1] | LCMS: m/z 467.35 [M + 1]⁺ ¹H NMR (400 MHz, DMSO-d₆) δ 9.71 (s, 1H), 8.40 (d, J = 6.7 Hz, 1H), 7.54 (d, J = 10.5 Hz, 1H), 7.09 (t, J = 8.1 Hz, 1H), 6.89-6.82 (m, 1H), 6.81-6.75 (m, 1H), 4.74 (dq, J = 24.6, 8.4 Hz, 1H), 4.15-3.99 (m, 4H), 3.34-3.21 (m, 2H), 3.10 (m, 1H), 2.74- 2.67 (dq, J = 35.6, 12.0 Hz, 4H), 2.28 (t, J = 11.5 Hz, 2H), 1.94 (d, J = 19.9 Hz, 2H). | C (12) |
| | 5-(1-{2-(2-fluoro-5-hydroxybenzoyl)- | | |
| | 2-aza-6-spiro[3.3]heptyl}-4- | | |
| | (trifluoromethyl)-5-pyrazolyl)-2- | | |
| | piperidinone | | |

TABLE A-3
Exemplary Compounds.

| | | | MAGL |
|---|---|---|---|
| | | | Potency |
| | Structure | | (Detection |
| Compound | (IUPAC name) | LCMS/NMR | Method) |
| 1 | 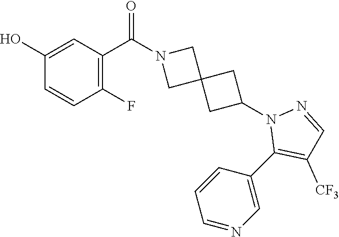 [SMILES: O=C(c1cc(O)ccc1F)N1CC2(CC(n3ncc(C(F)(F)F)c3-c3cccnc3)C2)C1] | LCMS: m/z 447.12 [M + 1]⁺. ¹H NMR (400 MHz, DMSO-d₆) δ 9.67 (d, J = 4.4 Hz, 1H), 8.82 (m, 1H), 8.67-8.58 (m, 2H), 8.01- 7.91 (m, 1H), 7.57- 7.47 (m, 1H), 7.09 (td, J = 9.2, 5.0 Hz, 1H), 6.89- 6.77 (m, 2H), 4.90 (dt, J = 23.6, 8.0 Hz, 1H), 4.15 (d, J = 11.5 Hz, 2H), 4.07 (d, J = 9.4 Hz, 2H), 2.76 (q, J = 10.9, 9.3 Hz, 4H). | C (12) |
| | (2-fluoro-5-hydroxyphenyl){6-[5-(3- | | |
| | pyridyl)-4-(trifluoromethyl)-1- | | |
| | pyrazolyl]-2-aza-2- | | |
| | spiro[3.3]heptyl}methanone | | |
| 2 | 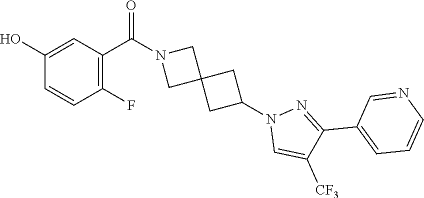 [SMILES: O=C(c1cc(O)ccc1F)N1CC2(CC(n3cc(C(F)(F)F)c(-c4cccnc4)n3)C2)C1] | LCMS: m/z 447.12 [M + 1]⁺. ¹H NMR (400 MHz, DMSO-d₆) δ 9.66 (s, 1H), 8.75 (dd, J = 11.5, 4.3 Hz, 1H), 8.58 (d, J = 9.5 Hz, 1H), 8.10 (d, J = 10.0 Hz, 1H), 7.86 (t, J = 8.9 Hz, 1H), 7.59 (dt, J = 13.0, 6.6 Hz, 1H), 7.06 (q, J = 9.5 Hz, 1H), 6.94-6.81 (m, 2H), 4.57 (dt, J = 16.4, 7.8 Hz, 1H), 4.01 (d, J = | A (12) |
| | (2-fluoro-5-hydroxyphenyl){6-[3-(3- | 8.8 Hz, 4H), 2.73 (dt, J = | |
| | pyridyl)-4-(trifluoromethyl)-1- | 19.6, 9.9 Hz, 2H), 2.67- | |
| | pyrazolyl]-2-aza-2- | 2.52 (m, 2H). | |
| | spiro[3.3]heptyl}methanone | | |
| 3 | 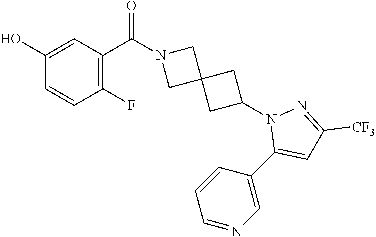 [SMILES: O=C(c1cc(O)ccc1F)N1CC2(CC(n3nc(C(F)(F)F)cc3-c3cccnc3)C2)C1] | LCMS: m/z 447.24 [M + 1]⁺. NMR: ¹H NMR (400 MHz, DMSO-d₆) δ 9.70 (s, 1H), 9.11 (d, J = 16.9 Hz, 1H), 8.59 (d, J = 3.8 Hz, 0H), 8.26 (dd, J = 22.5, 7.4 Hz, 1H), 7.59 (s, 1H), 7.54-7.46 (m, 1H), 7.10 (d, J = 8.7 Hz, 1H), 6.88 (s, 1H), 6.82 (s, 1H), 4.99-4.90 (m, 1H), 4.16 (dd, J = 19.4, 8.7 Hz, 4H), 2.88-2.78 (m, 4H). | B (12) |
| | (2-fluoro-5-hydroxyphenyl){6-[5-(3- | | |
| | pyridyl)-3-(trifluoromethyl)-1- | | |
| | pyrazolyl]-2-aza-2- | | |
| | spiro[3.3]heptyl}methanone | | |
| 4 | 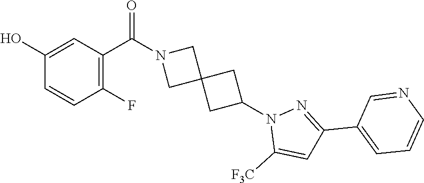 [SMILES: O=C(c1cc(O)ccc1F)N1CC2(CC(n3nc(-c4cccnc4)cc3C(F)(F)F)C2)C1] | LCMS: m/z 447.24 [M + 1]⁺. NMR: ¹H NMR (400 MHz, DMSO-d6) δ 8.72 (t, J = 9.2 Hz, 2H), 8.00-7.89 (m, 1H), 7.65-7.53 (m, 1H), 7.13-7.02 (m, 2H), 6.87 (s, 2H), 4.89 (dt, J = 22.3, 7.8 Hz, 1H), 4.08 (t. J = 10.9 Hz, 4H), 2.74 (dt, J = 17.9, 9.9 Hz, 4H). | A (12) |
| | (2-fluoro-5-hydroxyphenyl){6-[3-(3- | | |
| | pyridyl)-5-(trifluoromethyl)-1- | | |
| | pyrazolyl]-2-aza-2- | | |
| | spiro[3.3]heptyl}methanone | | |
| 5 | 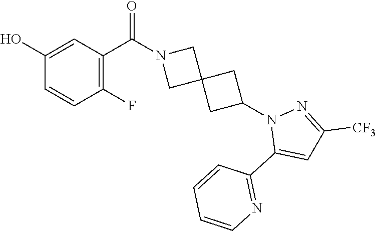 [SMILES: O=C(c1cc(O)ccc1F)N1CC2(CC(n3nc(C(F)(F)F)cc3-c3ccccn3)C2)C1] | LCMS: 447.19 m/z [M + 1]⁺. ¹H NMR (400 MHz, DMSO-d₆) δ 9.77 (s, 1H), 8.77 (s, 1H), 8.01-7.92 (m, 1H), 7.85 (d, J = 4.9 Hz, 1H), 7.49 (dd, J = 10.3, 5.6 Hz, 1H), 7.29 (s, 1H), 7.10 (t, J = 9.0 Hz, 1H), 6.87 (s, 1H), 6.80 (s, 1H), 5.62 (dt, J = 16.0, 7.8 Hz, 1H), 4.11 (dd, J = 27.7, 9.1 Hz, 4H), 2.77 2.70 (m, 4H). | A (12) |
| | (2-fluoro-5-hydroxyphenyl){6-[5-(2- | | |
| | pyridyl)-3-(trifluoromethyl)-1- | | |
| | pyrazolyl]-2-aza-2- | | |
| | spiro[3.3]heptyl}methanone | | |
| 6 | 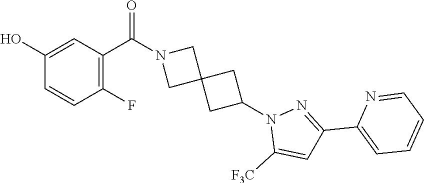 [SMILES: O=C(c1cc(O)ccc1F)N1CC2(CC(n3nc(-c4ccccn4)cc3C(F)(F)F)C2)C1] | LCMS: m/z 445.4 [M − H]⁻. ¹H NMR (400 MHz, DMSO-d₆) δ 9.70 (s, 1H), 8.68-8.60 (m, 1H), 8.06- 7.93 (ddd, J = 27.6, 13.4, 7.7 Hz, 2H), 7.46-7.36 (m, 2H), 7.12 (t, J = 8.7 Hz, 1H), 6.92-6.80 (m, 2H), 4.96 (dt, J = 16.1, 7.7 Hz, 1H), 4.16 (dd, J = 22.0, 8.1 Hz, 4H), 2.90- | C (12) |
| | (2-fluoro-5-hydroxyphenyl){6-[3-(2- | 2.83 (dd, J = 23.5, | |
| | pyridyl)-5-(trifluoromethyl)-1- | 10.0 Hz, 4H). | |
| | pyrazolyl]-2-aza-2- | | |
| | spiro[3.3]heptyl}methanone | | |
| 7 | 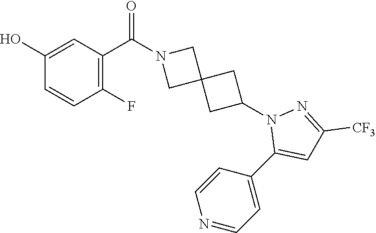 [SMILES: O=C(c1cc(O)ccc1F)N1CC2(CC(n3nc(C(F)(F)F)cc3-c3ccncc3)C2)C1] | LCMS: m/z 445.91 [M − 1]⁻. NMR: ¹H NMR (400 MHz, DMSO-d₆) δ 9.68 (s, 1H), 8.70-8.61 (m, 2H), 7.91- 7.81 (m, 2H), 7.66 (s, 1H), 7.11 (t, J = 9.2 Hz, 1H), 6.90-6.79 (m, 2H), 5.00- 4.91 (m, 1H), 4.15 (dd, J = 19.2, 6.3 Hz, 4H), 2.84 (d, J = 7.6 Hz, 4H). | C (12) |
| | (2-fluoro-5-hydroxyphenyl){6-[5-(4- | | |
| | pyridyl)-3-(trifluoromethyl)-1- | | |
| | pyrazolyl]-2-aza-2- | | |
| | spiro[3.3]heptyl}methanone | | |
| 8 | 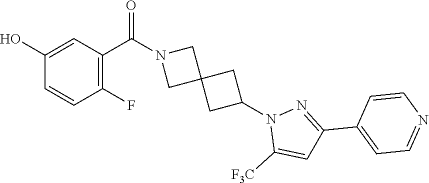 [SMILES: O=C(c1cc(O)ccc1F)N1CC2(CC(n3nc(-c4ccncc4)cc3C(F)(F)F)C2)C1] | LCMS: m/z 447.30 [M + 1]⁺. NMR: ¹H NMR (400 MHz, DMSO-d₆) δ 9.64 (s, 1H), 8.73 (d. J = 14.4 Hz, 2H), 7.52 (d, J = 11.5 Hz, 2H), 7.06 (d, J = 16.3 Hz, 2H), 6.84 (s, 1H), 6.77 (s, 1H), 4.96 (s, 1H), 4.06 (dd, J = 17.7, 9.9 Hz, 4H), 2.72 (s, 4H). | A (12) |
| | (2-fluoro-5-hydroxyphenyl){6-[3-(4- | | |
| | pyridyl)-5-(trifluoromethyl)-1- | | |
| | pyrazolyl]-2-aza-2- | | |
| | spiro[3.3]heptyl}methanone | | |
| 13 | 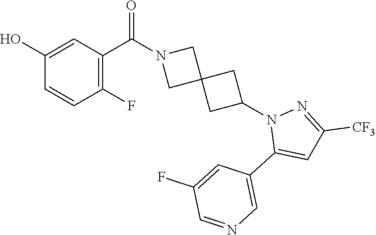 [SMILES: O=C(c1cc(O)ccc1F)N1CC2(CC(n3nc(C(F)(F)F)cc3-c3cncc(F)c3)C2)C1] | LCMS: m/z 465.30 [M + 1]⁺. NMR: ¹H NMR (400 MHz, DMSO-d6) δ 9.65 (d, J = 4.3 Hz, 1H), 8.99 (d, J = 19.3 Hz, 1H), 8.59 (d, J = 10.7 Hz, 1H), 8.16 (dd, J = 20.6, 9.2 Hz, 1H), 7.63 (s, 1H), 7.08 (d, J = 6.4 Hz, 1H), 6.80 (s, 1H), 6.75 (s, 1H), 4.93 (s, 1H), 4.13 (dd, J = 18.0, 8.9 Hz, 4H), 2.84-2.78 (s, 4H). | C (12) |
| | (2-fluoro-5-hydroxyphenyl){6-[5-(5- | | |
| | fluoro-3-pyridyl)-3- | | |
| | (trifluoromethyl)-1-pyrazoly]]-2-aza- | | |
| | 2-spiro[3.3]heptyl}methanone | | |
| 14 | 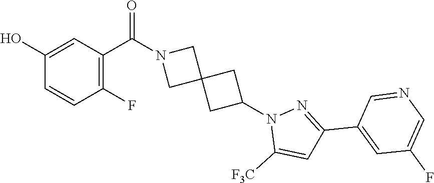 [SMILES: O=C(c1cc(O)ccc1F)N1CC2(CC(n3nc(-c4cncc(F)c4)cc3C(F)(F)F)C2)C1] | LCMS: m/z 465.40 [M + 1]⁺. NMR: ¹H NMR (400 MHz, DMSO-d6) δ 9.66 (s, 1H), 8.74 (dd, J = 11.4, 2.6 Hz, 1H), 8.57 (d, J = 10.9 Hz, 1H), 8.00- 7.89 (m, 1H), 7.07-7.05 (dd, J = 18.4, 2.6 Hz, 1H), 6.87-6.73 (m, 2H), 4.93 (dq, J = 25.0, 7.8 Hz, 1H), 4.05 (dd, J = | B (12) |
| | (2-fluoro-5-hydroxyphenyl){6-[3-(5- | 16.7, 9.5 Hz, 4H), 2.70 | |
| | fluoro-3-pyridyl)-5- | (h, J = 12.1 Hz, 4H). | |
| | (trifluoromethyl)-1-pyrazolyl]-2-aza- | | |
| | 2-spiro[3.3]heptyl}methanone | | |
| 15 | 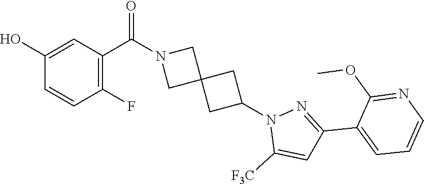 [SMILES: COc1ncccc1-c1cc(C(F)(F)F)n(C2CC3(C2)CN(C(=O)c2cc(O)ccc2F)C3)n1] | LCMS: m/z 477.40 [M + H]⁺ ¹H NMR (400 MHz, DMSO-d₆) 9.66 (d, J = 2.2 Hz, 1H), 8.36 (m, 1H), 7.77 (t, J = 7.2 Hz, 1H), 7.22-7.12 (m, 1H), 7.09 (dt, J = 17.0, 7.9 Hz, 1H), 6.88-6.75 (m, 3H), 4.57 (dt, J = 15.5, 7.8 Hz, 1H), 4.06 (d, J = 7.3 Hz, 4H), 3.90 (d, J = 13.9 Hz, 3H), | A (12) |
| | (2-fluoro-5-hydroxyphenyl){6-[3-(2- | 2.71-2.54 (m, 4H). | |
| | methoxy-3-pyridyl)-5- | | |
| | (trifluoromethyl)-1-pyrazolyl]-2-aza- | | |
| | 2-spiro[3.3]heptyl}methanone | | |
| 16 | 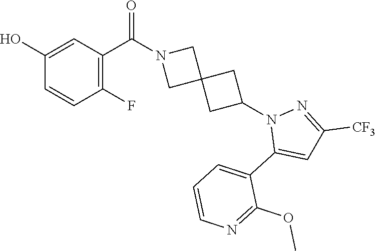 [SMILES: COc1ncccc1-c1cc(C(F)(F)F)nn1C1CC2(C1)CN(C(=O)c1cc(O)ccc1F)C2] | LCMS: m/z 477.10 [M + H]⁺ ¹H NMR (400 MHz, DMSO-d₆) δ 9.66 (d, J = 2.2 Hz, 1H), 8.34 (dd, J = 11.5, 4.2 Hz, 1H), 8.21 (m, 1H), 7.22-7.12 (m, 1H), 7.09-7.20 (dt, J = 17.0, 7.9 Hz, 2H), 6.88- 6.75 (m, 2H), 4.97 (dt, J = 15.5, 7.8 Hz, 1H), 4.06- 4.16 (d, J = 7.3 Hz, 4H), 3.90 (d, J = 13.9 Hz, 3H), 2.74 (d, J = 12.2 Hz, 4H). | B (12) |
| | (2-fluoro-5-hydroxyphenyl){6-[5-(2- | | |
| | methoxy-3-pyridyl)-3- | | |
| | (trifluoromethyl)-1-pyrazoly]]-2-aza- | | |
| | 2-spiro[3.3]heptyl}methanone | | |
| 17 | 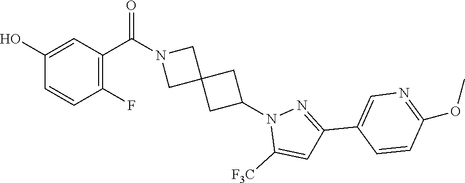 [SMILES: COc1ccc(-c2cc(C(F)(F)F)n(C3CC4(C3)CN(C(=O)c3cc(O)ccc3F)C4)n2)cn1] | LCMS: m/z 477.41 [M + H]⁺. ¹H NMR (400 MHz, DMSO-d₆) δ 9.73 (s, 1H), 8.27 (d, J = 10.8 Hz, 1H), 7.86 (t, J = 10.4 Hz 1H), 7.09-7.04 (m, 1H), 7.00 (s, 1H), 6.91 (s, 1H), 6.84 (t, J = 4.4 Hz, 1H), 6.80 (s, 1H), 4.79-4.71 (m, 1H), 4.47- 4.38 (m, 4H), 3.29 (s, 3H), 2.32 (d, J = 17.6 Hz, 4H). | A (12) |
| | (2-fluoro-5-hydroxyphenyl){6-[3-(6- | | |
| | methoxy-3-pyridyl)-5- | | |
| | (trifluoromethyl)-1-pyrazolyl]-2-aza- | | |
| | 2-spiro[3.3]heptyl}methanone | | |
| 18 | 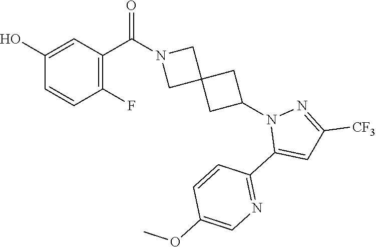 [SMILES: COc1ccc(-c2cc(C(F)(F)F)nn2C2CC3(C2)CN(C(=O)c2cc(O)ccc2F)C3)nc1] | LCMS: m/z 477.41 [M + H]⁺. ¹H NMR (400 MHz, DMSO-d₆) δ 9.70 (s, 1H), 8.69 (d, J = 16.6 Hz, 1H), 8.18-8.13 (dd, J = 7.2 Hz, 1H), 7.45 (s, 1H), 7.11- 7.09 (t, J = 8.8 Hz, 1H), 6.92 (dd, J = 17.9, 8.9 Hz, 1H), 6.81 (s, 1H), 6.80 (s, 1H), 4.91 (dt, J = 16.1, 7.7 Hz, 1H), 4.15 (dd, J = 19.7, 10.2 Hz, 4H), 3.91 (d, J = 7.0 Hz, 3H), 2.86- 2.77 (m, 4H). | C (12) |
| | (2-fluoro-5-hydroxyphenyl){6-[5-(6- | | |
| | methoxy-3-pyridyl)-3- | | |
| | (trifluoromethyl)-1-pyrazoly]]-2-aza- | | |
| | 2-spiro[3.3]heptyl}methanone | | |
| 19 | 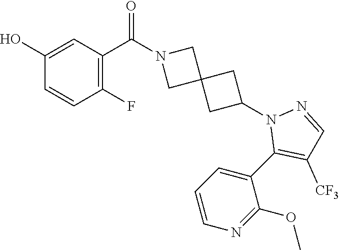 [SMILES: COc1ncccc1-c1c(C(F)(F)F)cnn1C1CC2(C1)CN(C(=O)c1cc(O)ccc1F)C2] | LCMS: m/z 477.4 [M + 1]⁺. ¹H NMR (400 MHz, DMSO-d₆) δ 9.67 (s, 1H), 8.53 (d, J = 9.8 Hz, 1H), 8.25 (d, J = 3.3 Hz, 1H), 7.70 (dd, J = 12.5, 7.3 Hz, 1H), 7.07 (p, J = 8.1, 7.3 Hz, 2H), 6.85-6.71 (m, 2H), 4.85 (dt, J = 23.9, 7.8 Hz, 1H), 4.14 (d, J = 9.8 Hz, 2H), 4.03 (d, J = 7.9 Hz, 2H), 3.81 (d, J = 7.9 Hz, 3H), 2.78-2.67 (m, 4H). | A (12) |
| | (2-fluoro-5-hydroxyphenyl){6-[5-(2- | | |
| | methoxy-3-pyridyl)-4- | | |
| | (trifluoromethyl)-1-pyrazolyl]-2-aza- | | |
| | 2-spiro[3.3]heptyl}methanone | | |
| 20 | 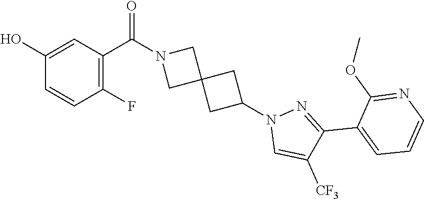 [SMILES: COc1ncccc1-c1nn(C2CC3(C2)CN(C(=O)c2cc(O)ccc2F)C3)cc1C(F)(F)F] | LCMS: m/z 477.5 [M + 1]⁺. ¹H NMR (400 MHz, DMSO-d₆) δ 9.66 (d, J = 5.8 Hz, 1H), 8.41-8.32 (m, 1H), 8.04 (d, J = 9.5 Hz, 1H), 7.72 (t, J = 6.7 Hz, 1H), 7.21-7.00 (m. 2H), 6.83-6.69 (d, J = 4.3 Hz, 2H), 4.44-4.35 (m, 1H), 4.06-3.97 (m, 4H), 3.85 (d, J = 9.9 Hz, 3H), 2.79-2.68 (dd, J = 23.6, 11.8 Hz, 4H). | C (12) |
| | (2-fluoro-5-hydroxyphenyl){6-[3-(2- | | |
| | methoxy-3-pyridyl)-4- | | |
| | (trifluoromethyl)-1-pyrazolyl]-2-aza- | | |
| | 2-spiro[3.3]hepty]}methanone | | |
| 21 | 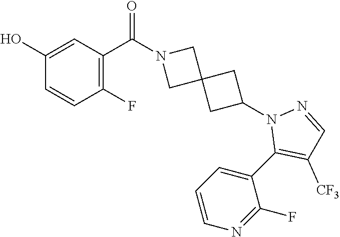 [SMILES: O=C(c1cc(O)ccc1F)N1CC2(CC(n3ncc(C(F)(F)F)c3-c3cccnc3F)C2)C1] | LCMS: m/z 465.0 [M + 1]⁺ ¹H NMR (400 MHz, DMSO-d₆) δ 9.73 (s, 1H), 8.68 (d, J = 9.6 Hz, 1H), 8.37 (s, 1H), 8.08 (q, J = 9.7, 8.7 Hz, 1H), 7.51 (d, J = 6.9 Hz, 1H), 7.16- 7.04 (m, 1H), 6.90-6.78 (m, 2H), 4.92 (dt, J = 24.0, 8.0 Hz, 1H), 4.16 (d, J = 10.9 Hz, 2H), 4.06 (d, J = 9.0 Hz, 2H), 2.78 (q, J = 14.5, 12.0 Hz, 4H). | C (12) |
| | (2-fluoro-5-hydroxyphenyl){6-[5-(2- | | |
| | fluoro-3-pyridyl)-4- | | |
| | (trifluoromethyl)-1-pyrazoly]]-2-aza- | | |
| | 2-spiro[3.3]heptyl}methanone | | |
| 22 | 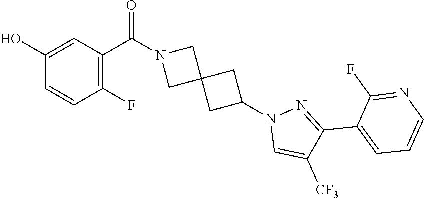 [SMILES: O=C(c1cc(O)ccc1F)N1CC2(CC(n3cc(C(F)(F)F)c(-c4cccnc4F)n3)C2)C1] | LCMS: m/z 465.4 [M + 1]⁺ ¹H NMR (400 MHz, DMSO-d₆) δ 9.98 (s, 1H), 8.51 (d, J = 12.2 Hz, 1H), 8.14 (dd, J = 20.2, 9.1 Hz, 2H), 7.64-7.56 (m, 1H), 7.07 (q, J = 9.8 Hz, 1H), 6.84-6.78 (s, 2H), 4.59 (dt, J = 16.2, 7.5 Hz, 1H), 4.05 (s, 4H), 2.80-2.62 (dd, J = 26.1, 10.0 Hz, 1H), 2.60-2.51 (dd, J = 26.1, 10.0 Hz, 2H). | A (12) |
| | (2-fluoro-5-hydroxyphenyl){6-[3-(2- | | |
| | fluoro-3-pyridyl)-4- | | |
| | (trifluoromethyl)-1-pyrazolyl]-2-aza- | | |
| | 2-spiro[3.3]heptyl}methanone | | |
| 27 | 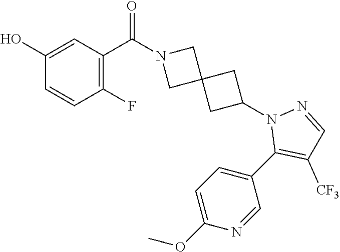 [SMILES: COc1ccc(-c2c(C(F)(F)F)cnn2C2CC3(C2)CN(C(=O)c2cc(O)ccc2F)C3)cn1] | LCMS: m/z 477.4 [M + 1]⁺ ¹H NMR (400 MHz, DMSO-d₆) δ 9.67 (s, 1H), 8.57 (d, J = 9.9 Hz, 1H), 8.36 (d, J = 10.8 Hz, 1H), 7.93-7.83 (m, 1H), 7.09 (td, J = 9.4, 3.9 Hz, 1H), 6.94 (t, J = 8.8 Hz, 1H), 6.89-6.77 (m, 2H), 4.87 (dt, J = 22.9, 7.9 Hz, 1H), 4.14 (d, J = 11.9 Hz, 2H), 4.06 (d, J = 10.3 Hz, 2H), 3.89 (d, J = 4.3 Hz, 3H), 2.79-2.69 (m, 4H). | B (12) |
| | (2-fluoro-5-hydroxyphenyl){6-[5-(6- | | |
| | methoxy-3-pyridyl)-4- | | |
| | (trifluoromethyl)-1-pyrazoly]]-2-aza- | | |
| | 2-spiro[3.3]heptyl}methanone | | |
| 28 | 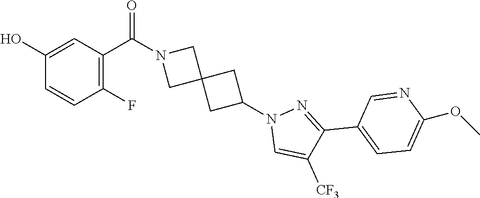 [SMILES: COc1ccc(-c2nn(C3CC4(C3)CN(C(=O)c3cc(O)ccc3F)C4)cc2C(F)(F)F)cn1] | LCMS: m/z 477.4 [M + 1]⁺ ¹H NMR (400 MHz, DMSO-d₆) δ 9.66 (s, 1H), 8.20 (d, J = 10.2 Hz, 1H), 8.07 (d, J = 9.8 Hz, 1H), 7.80-7.68 (m, 1H), 7.14- 7.04 (m, 1H), 7.01 (dd, J = 13.9, 8.7 Hz, 1H), 6.89-6.78 (d, J = 4.5 Hz, 2H), 4.68-4.54 (m, 1H), 4.04 (d, J = 7.3 Hz, 4H), 3.95 (d, J = 10.1 Hz, 3H), 2.82-2.67 (m, 2H), 2.69- | A (12) |
| | (2-fluoro-5-hydroxyphenyl){6-[3-(6- | 2.54 (m, 2H). | |
| | methoxy-3-pyridyl)-4- | | |
| | (trifluoromethyl)-1-pyrazolyl]-2-aza- | | |
| | 2-spiro[3.3]heptyl}methanone | | |

In addition, the method of Example A was used to measure MAGL Inhibition potency of >10,000 mM for each of the comparator compounds in the table below.

TABLE C
Comparator Compounds.

| Comparator | Structure | Potency |
|---|---|---|
| C1 | 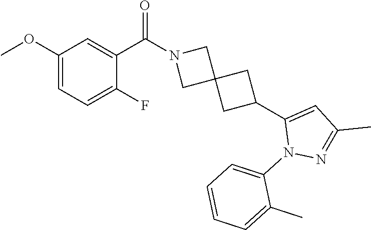 | >10,000 nM (2) |
| C2 | 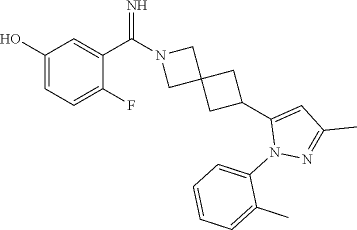 [SMILES: Cc1cc(C2CC3(C2)CN(C(=N)c2cc(O)ccc2F)C3)n(-c2ccccc2C)n1] | >10,000 nM (2) |
| C3 | 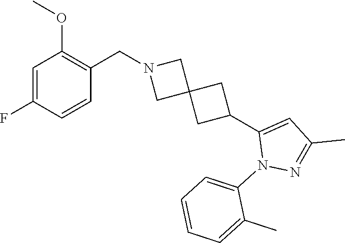 [SMILES: COc1cc(F)ccc1CN1CC2(CC(c3cc(C)nn3-c3ccccc3C)C2)C1] | >10,000 nM (2) |
| C4, C5 | 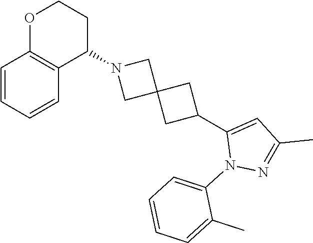 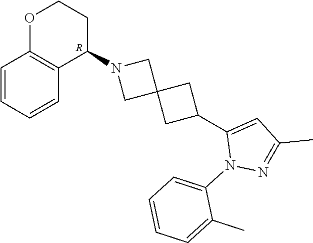 | >10,000 nM (2) |
| C6, C7 | 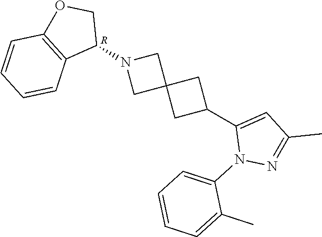 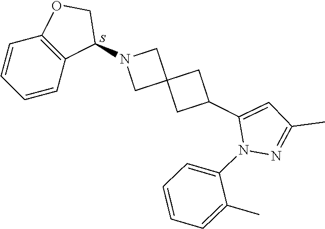 | >10,000 nM (2) |
| C8 | 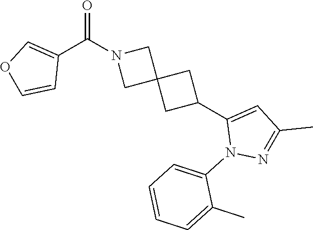 [SMILES: Cc1cc(C2CC3(C2)CN(C(=O)c2ccoc2)C3)n(-c2ccccc2C)n1] | >10,000 nM (2) |
| C9 | 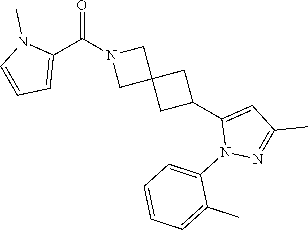 [SMILES: Cc1cc(C2CC3(C2)CN(C(=O)c2cccn2C)C3)n(-c2ccccc2C)n1] | >10,000 nM (2) |
| C10 | 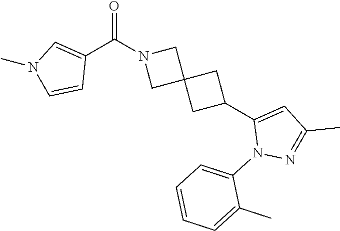 | >10,000 nM (2) |
| C11 | 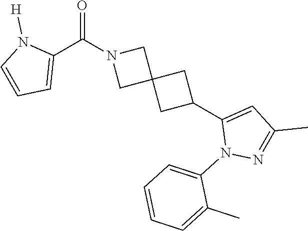 [SMILES: Cc1cc(C2CC3(C2)CN(C(=O)c2ccc[nH]2)C3)n(-c2ccccc2C)n1] | >10,000 nM (2) |
| C12 | 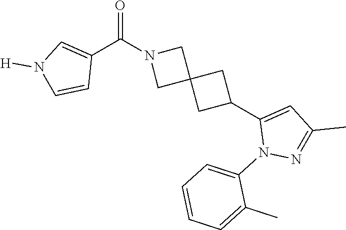 [SMILES: Cc1cc(C2CC3(C2)CN(C(=O)c2cc[nH]c2)C3)n(-c2ccccc2C)n1] | >10,000 nM (2) |
| C13 | 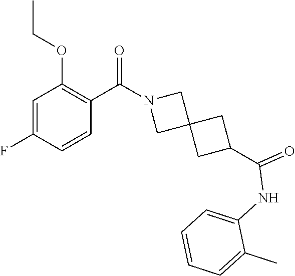 [SMILES: CCOc1cc(F)ccc1C(=O)N1CC2(CC(C(=O)Nc3ccccc3C)C2)C1] | >10,000 nM (2) |
| C14 | 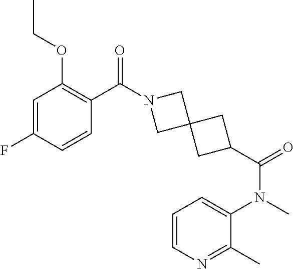 [SMILES: CCOc1cc(F)ccc1C(=O)N1CC2(CC(C(=O)N(C)c3cccnc3C)C2)C1] | >10,000 nM (4) |
| C15 | 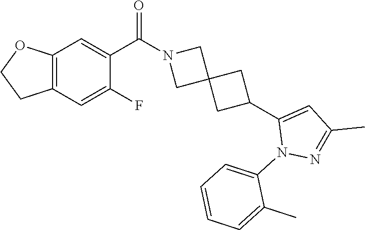 [SMILES: Cc1cc(C2CC3(C2)CN(C(=O)c2cc4c(cc2F)CCO4)C3)n(-c2ccccc2C)n1] | >10,000 nM (14) |
| C16 | 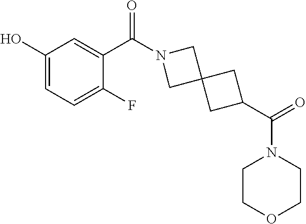 [SMILES: O=C(c1cc(O)ccc1F)N1CC2(CC(C(=O)N3CCOCC3)C2)C1] | >10,000 nM (14) |
| C17 | 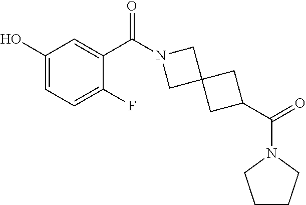 [SMILES: O=C(c1cc(O)ccc1F)N1CC2(CC(C(=O)N3CCCC3)C2)C1] | >10,000 nM (14) |
| C18 | 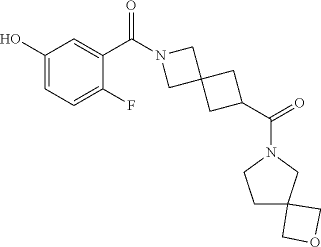 [SMILES: O=C(c1cc(O)ccc1F)N1CC2(CC(C(=O)N3CCC4(COC4)C3)C2)C1] | >10,000 nM (14) |
| C19 | 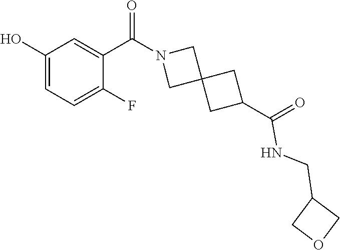 [SMILES: O=C(NCC1COC1)C1CC2(C1)CN(C(=O)c1cc(O)ccc1F)C2] | >10,000 nM (14) |
| C20 | 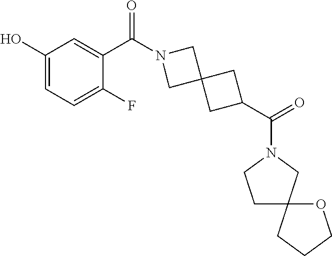 [SMILES: O=C(c1cc(O)ccc1F)N1CC2(CC(C(=O)N3CCC4(CCCO4)C3)C2)C1] | >10,000 nM (14) |
| C21 | 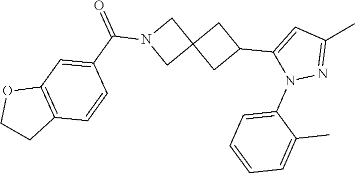 [SMILES: Cc1cc(C2CC3(C2)CN(C(=O)c2ccc4c(c2)OCC4)C3)n(-c2ccccc2C)n1] | >10,000 nM (14) |
| C22 | 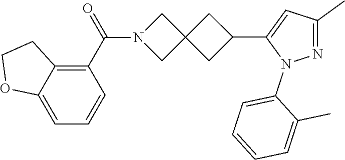 [SMILES: Cc1cc(C2CC3(C2)CN(C(=O)c2cccc4c2CCO4)C3)n(-c2ccccc2C)n1] | >10,000 nM (14) |
| C23 | 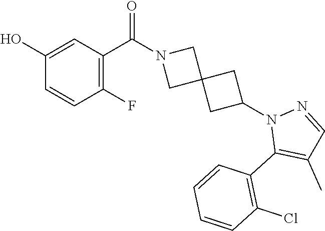 [SMILES: Cc1cnn(C2CC3(C2)CN(C(=O)c2cc(O)ccc2F)C3)c1-c1ccccc1Cl] | >10,000 nM (6) |
| C24 | 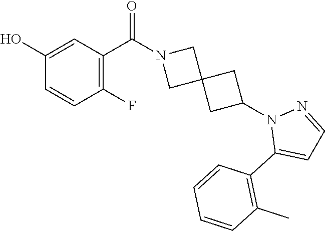 [SMILES: Cc1ccccc1-c1ccnn1C1CC2(C1)CN(C(=O)c1cc(O)ccc1F)C2] | >10,000 nM (6) |
| C25 | 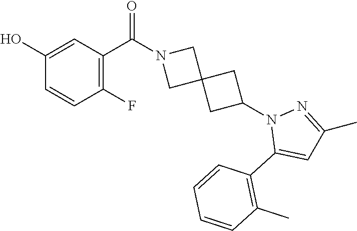 [SMILES: Cc1cc(-c2ccccc2C)n(C2CC3(C2)CN(C(=O)c2cc(O)ccc2F)C3)n1] | >10,000 nM (6) |
| C26 | 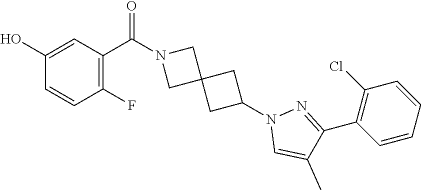 [SMILES: Cc1cn(C2CC3(C2)CN(C(=O)c2cc(O)ccc2F)C3)nc1-c1ccccc1Cl] | >10,000 nM (6) |
| C27 | 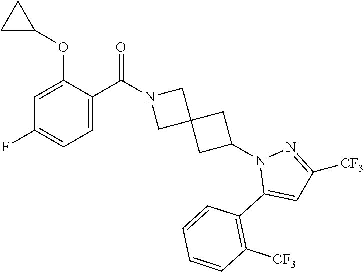 [SMILES: O=C(c1ccc(F)cc1OC1CC1)N1CC2(CC(n3nc(C(F)(F)F)cc3-c3ccccc3C(F)(F)F)C2)C1] | >10,000 nM (6) |
| C28 | 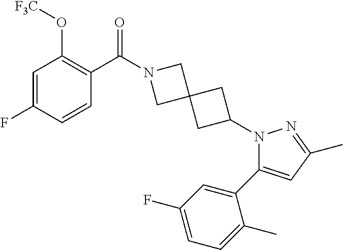 [SMILES: Cc1cc(-c2cc(F)ccc2C)n(C2CC3(C2)CN(C(=O)c2ccc(F)cc2OC(F)(F)F)C3)n1] | >10,000 nM (11) |
| C29 | 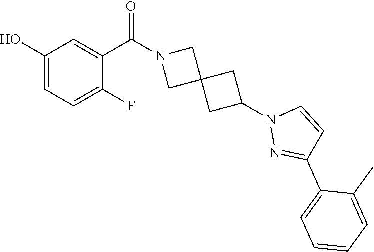 [SMILES: Cc1ccccc1-c1ccn(C2CC3(C2)CN(C(=O)c2cc(O)ccc2F)C3)n1] | >10,000 nM (6) |
| C30 | 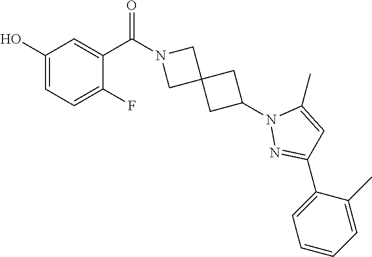 [SMILES: Cc1ccccc1-c1cc(C)n(C2CC3(C2)CN(C(=O)c2cc(O)ccc2F)C3)n1] | >10,000 nM (6) |
| C31 | 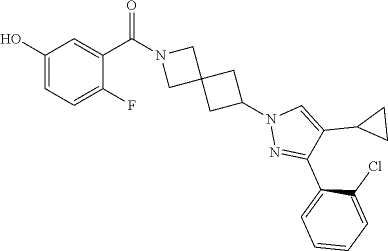 | >10,000 nM (6) |
| C32 | 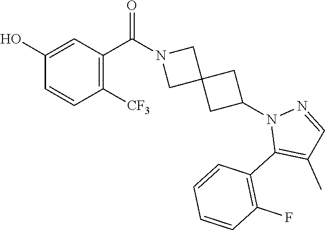 [SMILES: Cc1cnn(C2CC3(C2)CN(C(=O)c2cc(O)ccc2C(F)(F)F)C3)c1-c1ccccc1F] | >10,000 nM (12) |
| C33 | 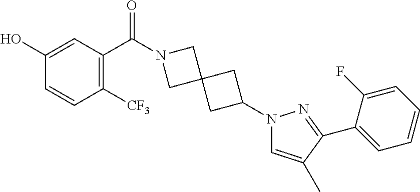 [SMILES: Cc1cn(C2CC3(C2)CN(C(=O)c2cc(O)ccc2C(F)(F)F)C3)nc1-c1ccccc1F] | >10,000 nM (12) |
| C34 | 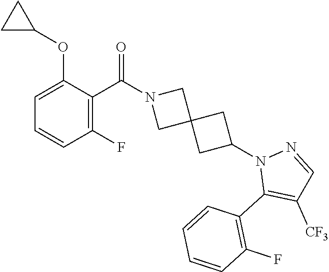 [SMILES: O=C(c1c(F)cccc1OC1CC1)N1CC2(CC(n3ncc(C(F)(F)F)c3-c3ccccc3F)C2)C1] | >10,000 nM (12) |

### Example B: Measuring FAAH Inhibition Potency (IC₅₀)

Comparative compound potency at FAAH can be obtained with the following assay. A “Selective MAGL Inhibitor Compound” refers to a compound that selectively inhibits MAGL with an IC₅₀ that is at least 10× the IC₅₀ for its inhibition of fatty acid amide hydrolase (FAAH), and that has an IC₅₀ of 100 nM or less for MAGL inhibition (according to the MAGL Potency assay of Example A).

MAGL inhibitor compounds were also counter-screened for FAAH inhibition potency using the following assay. Assessment of FAAH inhibition was performed using Fatty Acid Amide Hydrolase Inhibitor Screening Assay Kit (Cayman Item No. 10005196) following manufacture's instruction with some modifications. The kit utilizes human recombinant FAAH and the fluorescent substrate, AMC Arachidonoyl amide (AAMCA). 5 μL of assay buffer (125 mM Tris, pH 9.0, 1 mM EDTA, i.e. ethylenediaminetetraacetic acid) was added to a 384-well black plate (Corning, 3573) Test compounds and control inhibitor JZL-195 (Cayman Chemical, 13668) were tested in 10-concentration IC50 mode with 3-fold serial dilution at a starting concentration of 100 μM and 10 μM, respectively. 300 nL or 30 nL of test compounds were delivered into a 384-well black plate (Corning, 3573) using a Labcyte Echo, followed by addition of 15 μL of FAAH enzyme (Cayman, 700302) in assay buffer. After a 5-minute pre-incubation at room temperature, 10 μL of AAMCA was added in assay buffer to start the reaction. Final concentration of FAAH enzyme is not specified and AAMCA substrate was used at the 20 uM. After these dilutions, the final concentration of the test compounds ranged from 100 μM to 5.08 nM or 10 μM down to 0.508 nM. The reaction was allowed to progress for 60 minutes, while the plate was read on an Envision plate reader at an Ex/Em of 350/460 nm with readings every minute. The data was analyzed in Microsoft Excel, using the slope between 30 and 59 minutes. The average of the no-enzyme wells (background) was subtracted from the data. The background-subtracted slope data was converted to % activity using the average of wells with enzyme and DMSO vehicle. IC₅₀ values were calculated using GraphPad software (Sigmoidal dose response, variable slope equation).

Compounds listed in Table D were tested in the FAAH Counter-screen of Example B.

TABLE D
FAAH Counter Screen

| Method | Compound | Structure | FAAH IC₅₀ (nM) |
|---|---|---|---|
| A | Comparator | 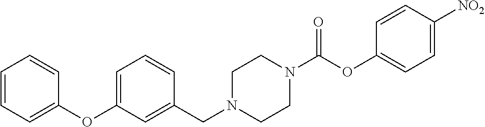 [SMILES: O=C(Oc1ccc([N+](=O)[O-])cc1)N1CCN(Cc2cccc(Oc3ccccc3)c2)CC1] | 459 |
| A | Comparator | 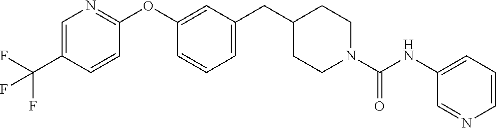 [SMILES: O=C(Nc1cccnc1)N1CCC(Cc2cccc(Oc3ccc(C(F)(F)F)cn3)c2)CC1] | 14 |
| B | 5 | 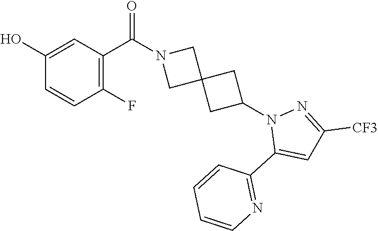 [SMILES: O=C(c1cc(O)ccc1F)N1CC2(CC(n3nc(C(F)(F)F)cc3-c3ccccn3)C2)C1] | >10,000 |
| B | 10 | 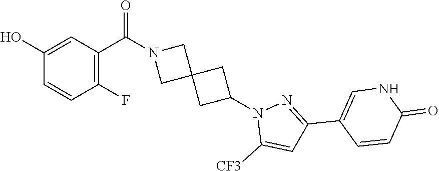 [SMILES: O=C(c1cc(O)ccc1F)N1CC2(CC(n3nc(-c4ccc(=O)[nH]c4)cc3C(F)(F)F)C2)C1] | >10,000 |
| B | 12 | 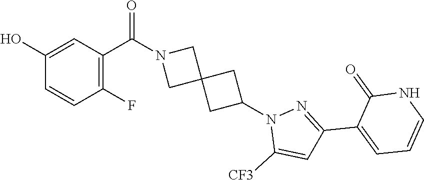 [SMILES: O=C(c1cc(O)ccc1F)N1CC2(CC(n3nc(-c4ccc[nH]c4=O)cc3C(F)(F)F)C2)C1] | >10,000 |
| B | 15 | 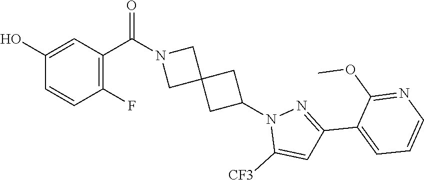 [SMILES: COc1ncccc1-c1cc(C(F)(F)F)n(C2CC3(C2)CN(C(=O)c2cc(O)ccc2F)C3)n1] | >10,000 |
| B | 17 | 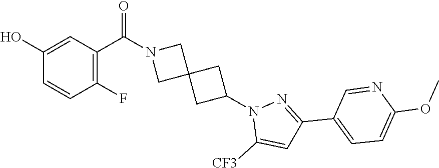 [SMILES: COc1ccc(-c2cc(C(F)(F)F)n(C3CC4(C3)CN(C(=O)c3cc(O)ccc3F)C4)n2)cn1] | >10,000 |

### Example C: Measuring Reversible MAGL Inhibition (IC₅₀)

The reversible mechanism of MAGL inhibition of a test compound of Formula (I) can be determined. Flag-tagged MAGL enzyme will be immobilized on anti-Flag beads Immobilized enzyme will be incubated+/−inhibitor at a dose that produces complete inhibition. Colorimetric substrate (4-NPA) will be added and the reaction monitored on a plate reader for 30 minutes to verify complete inhibition. Immobilized enzyme will then be washed thoroughly to remove the inhibitor, and fresh substrate will be added. The reaction will be monitored for an additional 30 minutes; returning enzymatic activity will indicate reversibility of inhibition.

To confirm the hypothesized reversible mechanism of inhibition, the effects of dilution and preincubation on the MAGL inhibitory activity of a compound can be evaluated using methods disclosed in J. Med. Chem. 2019, 62, 1932-1958, 1942. In the presence of an irreversible mechanism of inhibition, the potency should not decrease after dilution, whereas for a reversible inhibition, the potency level should be strongly reduced after dilution. Therefore, the inhibition produced by incubation with a 4000 nM concentration of a test compound can be measured after a 40× dilution and compared to the potency observed by a 4000 and a 100 nM of the test compound. A reversible mechanism of inhibition can be identified when the inhibition produced by 100 nM of the test compound is similar to that obtained after a 40× dilution and was considerably lower than that produced by the same compound at a concentration of 4000 nM.

As a second assay to identify or confirm a reversible MAGL inhibitory activity of a test compound, the inhibition activity of a test compound can be measured at different preincubation times with MAGL. The test compound can be preincubated with the enzyme for 0, 30, and 60 min before adding the substrate to start the enzymatic reaction. An irreversible inhibition should produce a higher potency after longer incubation times, whereas a reversible inhibitor should produce a constant inhibition potency over all the different incubation times.

### Determining MAGL Reversible Inhibition:

MAGL enzyme was incubated for 30 minutes in the presence of 40× the IC50 concentration of inhibitor. Enzyme+inhibitor mix was then diluted 40-fold so that the final concentration of the inhibitor equaled the IC50 concentration. Substrate was added and the reaction was monitored for 30 minutes. For a reversible inhibitor, percent inhibition after dilution to the IC50 concentration should be 50+15%.

The MAGL reversible inhibition assay of Example C, paragraph [0001], was performed to test reversibility of inhibition by compounds depicted in Table E Column A shows the degree to which MAGL enzymatic activity returned following washout of test compounds (pre-washout compound concentration: 1 μM), reflecting reversibility of inhibition, as compared to the complete lack of return of MAGL enzymatic activity after washout of the irreversible inhibitor comparator compound JZL-184 (pre-washout compound concentration: 1 μM). Column B shows % MAGL activity returning after washout. % Activity returning after washout represents the amount of MAGL enzymatic activity, relative to control (no inhibitor before washout) reaction amounts, that occurred immediately following a 30 minute washout of the test compound. For some test compounds, % activity returning after washout may be less than 100% if complete compound unbinding takes longer than 30 minutes.

TABLE E
Reversible MAGL inhibition

| Compound | Structure | A | B |
|---|---|---|---|
| JZL-184 | 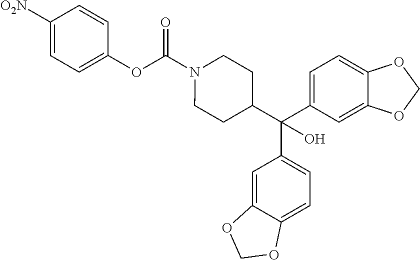 [SMILES: O=C(Oc1ccc([N+](=O)[O-])cc1)N1CCC(C(O)(c2ccc3c(c2)OCO3)c2ccc3c(c2)OCO3)CC1] | | <1% |
| 5 | 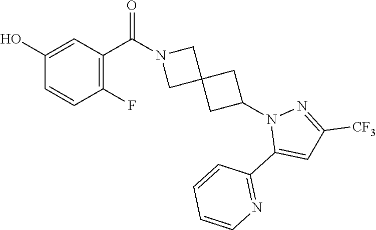 [SMILES: O=C(c1cc(O)ccc1F)N1CC2(CC(n3nc(C(F)(F)F)cc3-c3ccccn3)C2)C1] | 123 | >50% |
| 10 | 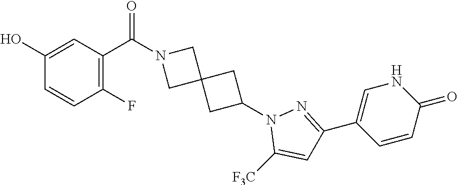 [SMILES: O=C(c1cc(O)ccc1F)N1CC2(CC(n3nc(-c4ccc(=O)[nH]c4)cc3C(F)(F)F)C2)C1] | 122 | >50% |
| 12 | 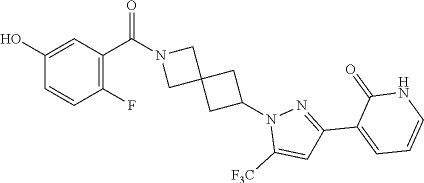 [SMILES: O=C(c1cc(O)ccc1F)N1CC2(CC(n3nc(-c4ccc[nH]c4=O)cc3C(F)(F)F)C2)C1] | 135 | >50% |
| 15 | 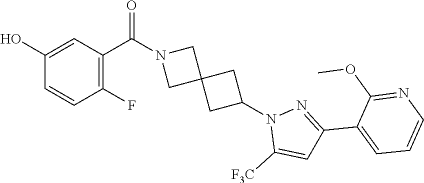 [SMILES: COc1ncccc1-c1cc(C(F)(F)F)n(C2CC3(C2)CN(C(=O)c2cc(O)ccc2F)C3)n1] | 99 | >50% |
| 17 | 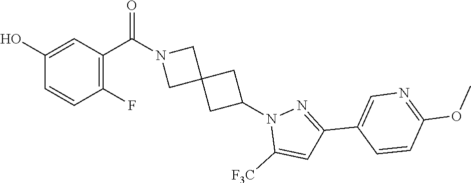 [SMILES: COc1ccc(-c2cc(C(F)(F)F)n(C3CC4(C3)CN(C(=O)c3cc(O)ccc3F)C4)n2)cn1] | 89 | >50% |

It should be understood that this disclosure is not limited to the particular methodology, protocols, and reagents, etc., provided herein and as such may vary. The terminology used herein is for the purpose of describing particular embodiments only, and is not intended to limit the scope of the present disclosure, which is defined solely by the claims.

Further, although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow. Further, to the extent not already indicated, it will be understood by those of ordinary skill in the art that any one of the various embodiments herein described and illustrated can be further modified to incorporate features shown in any of the other embodiments disclosed herein.

1. A compound of Formula (I) or a pharmaceutically acceptable salt thereof, 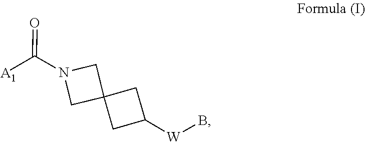
wherein:
A₁ is 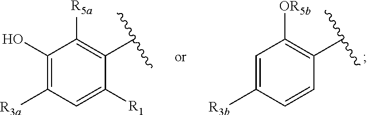
R₁ is halogen, hydrogen, or cyano;
R₃ₐ is hydrogen, halogen, or optionally substituted C₁₋₄ alkyl;
R₅ₐ is hydrogen, halogen, or optionally substituted C₁₋₄ alkyl;
R_{3b} is halogen;
R_{5b} is optionally substituted C₃₋₆ cycloalkyl, optionally substituted C₁₋₄ alkyl, or optionally substituted C₁₋₄ haloalkyl;
W is a pyrazolone, pyrrole or diazole, each independently optionally substituted with halogen, optionally substituted C₁₋₄ alkyl, optionally substituted C₃₋₆ cycloalkyl, optionally substituted C₁₋₄ alkoxy, or optionally substituted C₁₋₄ haloalkyl; and
B is an optionally substituted pyridone, or a 6-member heteroaryl comprising at least one nitrogen atom and optionally substituted with one or more halogen, optionally substituted C₁₋₄ alkyl, optionally substituted C₁₋₄ haloalkyl, or optionally substituted C₁₋₄ alkoxy.

2. The compound of claim 1, wherein
R₁ is F;
R₃ₐ is hydrogen;
R_{3b} is F;
R₅ₐ is hydrogen; and
R_{5b} is cyclopropyl.

3. The compound of claim 1, wherein W is selected from the group consisting of: 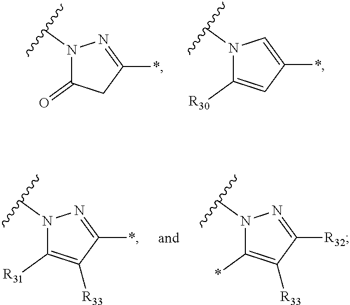
* is the bond to B;
B is an optionally substituted pyridone, or a pyridine optionally substituted with halogen, C₁₋₄ alkoxy, C₁₋₄ alkyl, or C₁₋₄ haloalkyl;
R₃₀ is hydrogen, halogen, C₁₋₄ alkyl, C₁₋₄ alkoxy, C₁₋₄ haloalkyl, or C₃₋₆ cycloalkyl; and
R₃₁, R₃₂ and R₃₃ are each independently hydrogen, halogen, C₁₋₄ alkyl, C₁₋₄ alkoxy, C₁₋₄ haloalkyl, or C₃₋₆ cycloalkyl, provided that at least one of R₃₁ and R₃₃ is not hydrogen, and provided that at least one of R₃₂ and R₃₃ is not hydrogen.

4. The compound of claim 3, wherein R₃₀, R₃₁, R₃₂ and R₃₃ are each independently hydrogen, F, methyl, methoxy, methyl substituted with one or more F, or cyclopropyl, provided that at least one of R₃₁ and R₃₃ is not hydrogen, and provided that at least one of R₃₂ and R₃₃ is not hydrogen.

5. (canceled)

6. (canceled)

7. The compound of claim 1, wherein B is 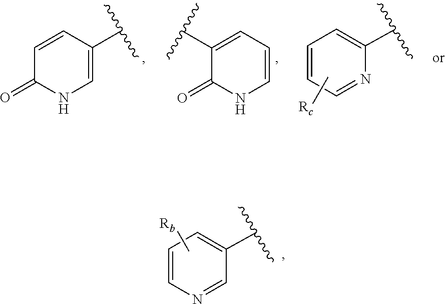
wherein
R_{b} and R_{c} are each independently hydrogen, fluoro, methoxy, or CF₃.

8. The compound of claim 7, wherein B is 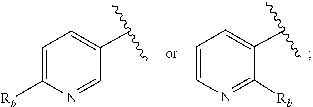
and R_{b} is hydrogen, fluoro or methoxy.

9. (canceled)

10. The compound of claim 7, wherein A₁ is

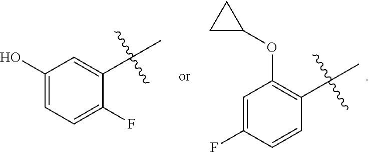 [SMILES: *c1cc(O)ccc1F ; *c1ccc(F)cc1OC1CC1]

11. The compound of claim 10, wherein W is selected from the group consisting of: 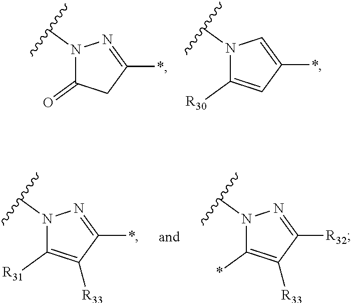
is the bond to B;
R₃₀ is hydrogen, halogen, C₁₋₄ alkyl, C₁₋₄ alkoxy, C₁₋₄ haloalkyl, or C₃₋₆ cycloalkyl; and
R₃₁, R₃₂ and R₃₃ are each independently hydrogen, halogen, C₁₋₄ alkyl, C₁₋₄ alkoxy, C₁₋₄ haloalkyl, or C₃₋₆ cycloalkyl, provided that at least one of R₃₁ and R₃₃ is not hydrogen, and provided that at least one of R₃₂ and R₃₃ is not hydrogen.

12. The compound of claim 11, wherein B is 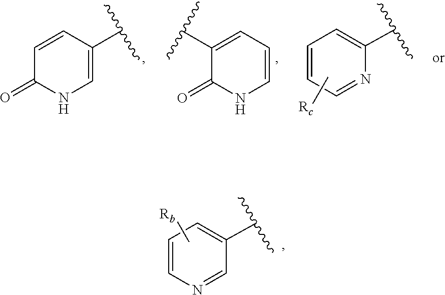
wherein
R_{b} and R_{c} are each independently hydrogen, fluoro, methoxy, or CF₃.

13. The compound of claim 12, wherein
R₃₀ is CF₃ or hydrogen when W is 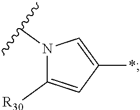
R₃₁ and R₃₃ is CF₃ and one of R₃₁ and R₃₃ is hydrogen when W is 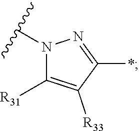
and
R₃₂ and R₃₃ is CF₃ and one of R₃₂ and R₃₃ is hydrogen when W is

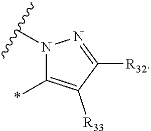

14. The compound of claim 13, wherein
B is 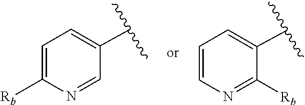
wherein R_{b} is hydrogen, fluoro or methoxy; or
B is

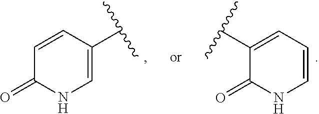 [SMILES: *c1ccc(=O)[nH]c1 ; *c1ccc[nH]c1=O]

15. The compound of claim 4, wherein A₁ is

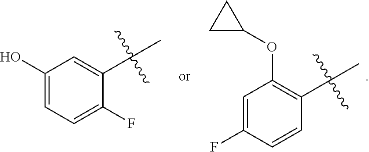 [SMILES: *c1cc(O)ccc1F ; *c1ccc(F)cc1OC1CC1]

16. (canceled)

17. The compound of claim 15, wherein W is

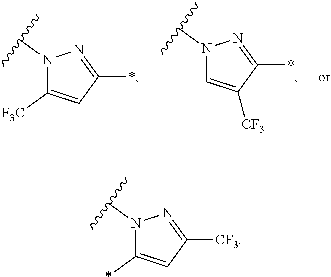

18. The compound of claim 17, wherein B is pyridine optionally substituted with F or methoxy.

19. The compound of claim 18, wherein B is

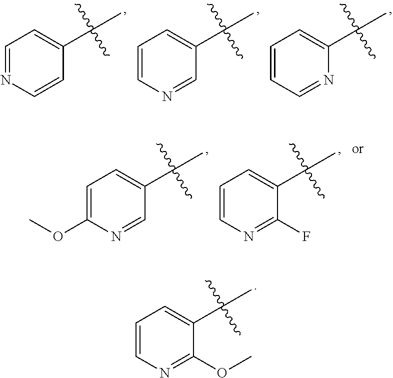

20. The compound of claim 19, wherein B is an unsubstituted 2-pyridone.

21. The compound of claim 20, wherein B is

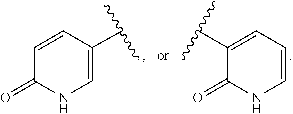 [SMILES: *c1ccc(=O)[nH]c1 ; *c1ccc[nH]c1=O]

22. The compound of claim 1, wherein the compound is selected from the group consisting of: 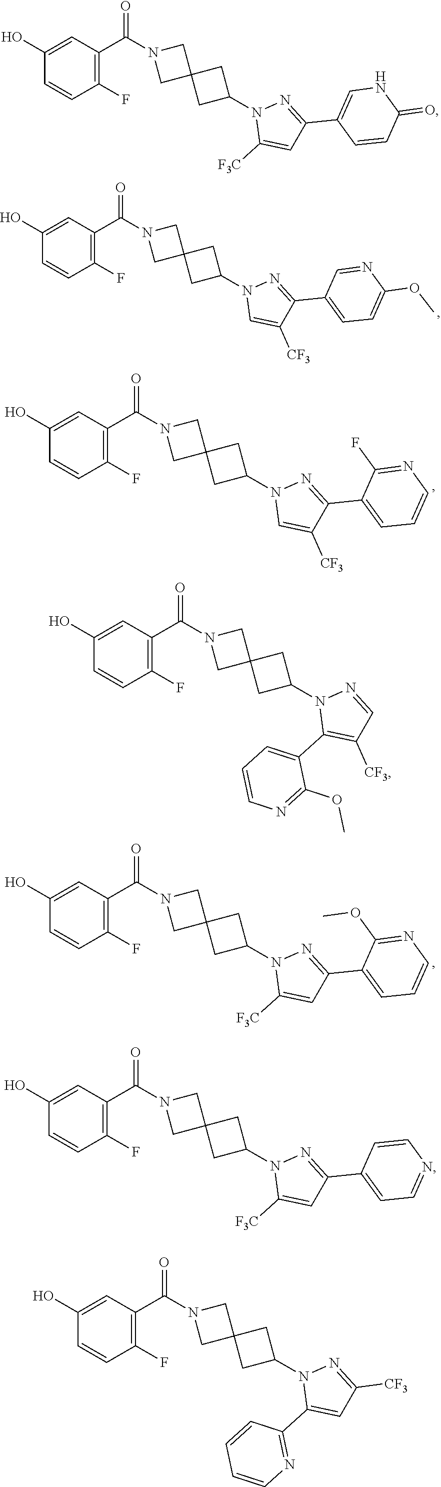 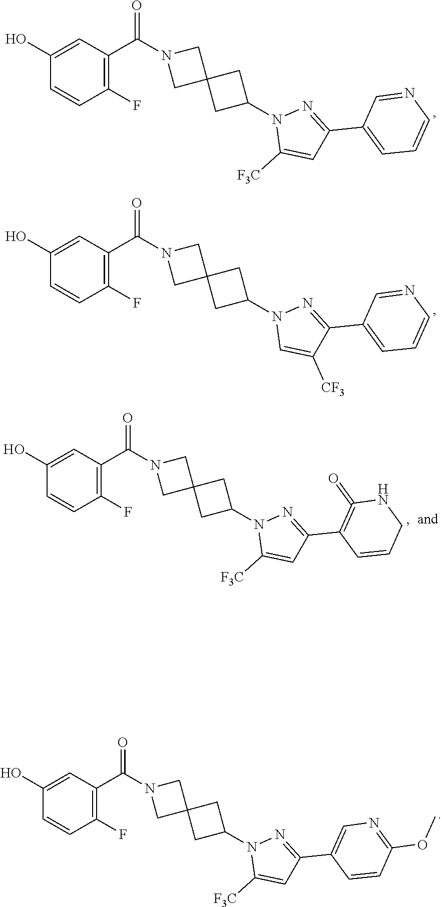
or a pharmaceutically acceptable salt thereof.

23.-32. (canceled)

33. A pharmaceutical composition comprising a compound of claim 1, or a pharmaceutically acceptable salt thereof as the active pharmaceutical ingredient (API).

34. A method of inhibiting monoacylglycerol lipase (MAGL) in a subject comprising the step of administering to a subject in need thereof a therapeutically effective amount of a compound of claim 1.

35. (canceled)
